(19) United States
(12) Patent Application Publication
(10) Pub. No.: US 2003/0236287 A1
(43) Pub. Date: Dec. 25, 2003

(54) Positive allosteric modulators of the nicotinic acetylcholine receptor

(76) Inventors: David W. Piotrowski, Portage, MI (US); Bruce N. Rogers, Portage, MI (US); William W. McWhorter JR., Parchment, MI (US); Daniel Patrick Walker, Kalamazoo, MI (US); Jeffrey W. Corbett, Portage, MI (US); Vincent E. Groppi JR., Kalamazoo, MI (US); Daniel G. Rudmann, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/423,062

(22) Filed: Apr. 25, 2003

Related U.S. Application Data
(60) Provisional application No. 60/377,364, filed on May 3, 2002.
(60) Provisional application No. 60/441,750.
(60) Provisional application No. 60/456,941, filed on Mar. 24, 2003.

(51) Int. Cl. A61K 31/433; A61K 31/42; A61K 31/426; A61K 31/17; C07C 335/18; C07C 275/32; C07D 285/12

(52) U.S. Cl. 514/362; 514/363; 514/370; 514/378; 514/585; 514/596; 548/138; 548/190; 548/245; 564/26; 564/47

(57) ABSTRACT

The invention provides compounds of Formula I:

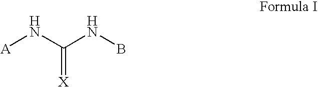

These compounds may be in the form of pharmaceutical salts or compositions, may be in pure enantiomeric form or racemic mixtures, and are useful in pharmaceuticals used to treat diseases or conditions in which α7 nAChR is known to be involved.

### CROSS-REFERENCE TO RELATED APPLICATIONS

This application claims the benefit of U.S. provisional application Serial No. 60/377364 filed on 3 May 2002, under 35 USC 119(e)(i), and U.S. provisional application Serial No. 60/456,941 filed on 24 Mar., 2003, under 35 USC 119(e)(i), both of which are incorporated herein by reference in their entirety.

### FIELD OF INVENTION

This invention relates to the use of certain urea and thiourea compounds as positive allosteric modulators of nicotinic acetylcholine receptors. It also relates to novel urea and thiourea compounds and to pharmaceutical compositions containing them.

### BACKGROUND OF THE INVENTION

Nicotinic acetylcholine receptors (nAChRs) play a large role in central nervous system (CNS) activity and in different tissue throughout the body. They are known to be involved in functions, including, but not limited to, cognition, learning, mood, emotion, and neuroprotection. There are several types of nicotinic acetylcholine receptors, and each one appears to have a different role. Some nicotinic receptors regulate CNS function, including, but not limited to, attention, learning and memory; some regulate pain, inflammation, cancer, and diabetes by controlling tumor necrosis factor alpha (TNF-α); and some regulate vascular angiogenesis; for example, the binding of nicotine to the alpha-7 nAChR stimulates DNA synthesis and proliferation of vascular endothelial cells in vitro (Villablanca, A. C., 1998, J. Appl. Physiol., 84(6):2089-2098) and induces angiogenesis in vivo (Heeschen C., et al. 2002, J. Clin. Invest., 110:527-535; Heeschen, C., et al. 2001, Nature Medicine, 7(7): 833-839). Nicotine affects all such receptors, and has a variety of activities. Unfortunately, not all of the activities are desirable. In fact, undesirable properties of nicotine include its addictive nature and the low ratio between efficacy and safety. The compounds of the present invention activate the α7 nAChR by acting as positive allosteric modulators (PAMs) of this ion channel. These molecules activate the α7 nAChR to enhance the activity of agonists at this receptor, including, but not limited to, acetylcholine (ACh) that is the endogenous neurotransmitter that activates this receptor.

Cell surface receptors are, in general, excellent and validated drug targets. nAChRs comprise a large family of ligand-gated ion channels that control neuronal activity and brain function. These receptors have a pentameric structure. In mammals, this gene family is composed of nine alpha and four beta subunits that co-assemble to form multiple subtypes of receptors that have a distinctive pharmacology. Acetylcholine is the endogenous regulator of all of the subtypes, while nicotine non-selectively activates all nAChRs.

The α7 nAChR is one receptor system that has proved to be a difficult target for testing. Native α7 nAChR is not routinely able to be stably expressed in most mammalian cell lines (Cooper and Millar, J. Neurochem., 1997, 68(5):2140-51). Another feature that makes functional assays of α7 nAChR challenging is that the receptor is rapidly (100 milliseconds) inactivated. This rapid inactivation greatly limits the functional assays that can be used to measure channel activity.

Both agonist and positive allosteric modulator activity of the α7 nAChR are assayed using a cell-based, calcium flux assay on FLIPR. SHEP-1 cells expressing a novel, mutated form of the α7 nAChR that permitted stable cell surface expression are used for these assays. The details of the mutated form of the α7 nAChR are described in WO 00/73431. See, e.g, U.S. Pat. Nos. 6,479,510 and 6,492,385 regarding α7 nAChR agonists.

U.S. Pat. No. 6,410,586 discloses modulators of protein tyrosine phosphatases.

U.S. Pat. No. 6,358,945 discloses compounds useful as anti-inflammatory agents.

U.S. Pat. No. 6,262,113 discloses IL-8 receptor antagonists.

U.S. Pat. No. 5,814,646 discloses inhibitors of amyloid beta protein production.

U.S. Pat. No. 5,185,358 discloses 3-heteroatom containing urea and thiourea ACAT inhibitors.

U.S. Pat. No. 5,162,360 discloses 2-heteroatom containing urea and thiourea ACAT inhibitors.

U.S. Pat. No. 5,059,614 discloses novel isoxazole and isoxazoline compounds with anticonvulsant activity, processes for their preparation, and therapeutic compositions containing them.

U.S. Pat. No. 4,062,861 discloses 3-isoxazolylurea derivatives.

U.S. Pat. No. 3,990,879 discloses discloses a method of controlling aquatic weeds. Example 13 in U.S. Pat. No. 3,990,879 is p-methoxyphenyl)-3-[5-trifluormethyl)-1,3,4-thiadiazol-2-yl]urea. Example 23 herein is N-(4-methoxyphenyl)-N′-[5-(trifluoromethyl)-1,3,4-thiadiazol-2-yl]urea.

WO 02/14311 discloses discloses urea compounds and methods of use.

WO 02/00651 discloses Factor XA inhibitors.

WO 01/68568 discloses IL-8 receptor antagonists.

WO 01/68605 discloses polycyclic aryl and heteroaryl substituted benzenes useful for selective inhibition of the coagulation cascade.

WO 01/53274 discloses amide compounds for inhibiting protein kinases.

WO 01/43697 discloses analogs of galanthamine and lycoramine as modulators of nicotinic receptors.

WO 01/32620 discloses positive modulators of nicotinic receptor agonists.

WO 01/32619 discloses positive modulators of nicotinic receptor agonists.

WO 00/35455 discloses heteroaryl-aryl ureas as GF-1 receptor antagonists.

WO 00/26203 discloses 2-ureido-thiazole derivatives, process for their preparation, and their use as antitumor agents, Pharmacia and Upjohn S.P.A. is assignee. Example 29 in WO 00/26203 is N-(5-isopropyl-1,3-thiazol-2-yl)-N′-(4-hydroxy-phenyl)-urea.

WO 99/56745 discloses pharmaceutical compositions comprising a positive modulator of a nicotinic receptor agonist, said positive modulator having the capacity to increase the efficacy of the said nicotinic receptor agonist.

WO 99/32106 discloses inhibition of RAF kinase using substituted heterocyclic ureas.

WO 99/28309 discloses 1,3,4-thiadiazoles derivatives as KYN-OH inhibitors.

WO 94/14801 discloses heterocyclic urea derivatives as 5HT_{2C} and 5HT_{2B} antagonists.

WO 93/18028 discloses indole derivatives as 5HT_{1C} antagonists.

Eur. J. Med. Chem., 22 (1987) 467-471 discloses search for structural parameters influencing the anthelmintic activity of thiadiazolyl urea derivatives. Example 1(e) is N-(4-butoxyphenyl)-N′-[5-(trifluoromethyl)-1,3,4-thiadiazol-2-yl]urea, which is Example 24 herein.

A positive allosteric modulator of α7 nAChR will effectively activate the endogenous α7 nAChR if there is sufficient agonist in the brain and elsewhere within the body to at least partially stimulate this receptor. Therefore, a positive allosteric modulator of α7 nAChR can be used alone to treat, or used alone to prepare a medicament to treat, CNS diseases or conditions as discussed herein. In certain diseases, however, it is possible that the full therapeutic efficacy of a positive allosteric modulator of α7 nAChR will be limited by suboptimal levels of agonist which in turn leads to a suboptimal activation of the endogenous α7 nAChR in the presence of a positive allosteric modulator. In such cases, the positive allosteric modulator of α7 nAChR is administered in combination with another agent that affects the level of agonist in one or more medicaments to treat the diseases or conditions discussed herein.

The activation of the α7 nAChR is also useful to treat, or used to prepare a medicament used to treat, diseases or conditions where a mammal receives symptomatic relief from the decrease of levels of TNF-α. The compounds of the present invention are useful to treat, or are used to prepare a medicament to treat, diseases or conditions where a mammal receives symptomatic relief from the stimulation of vascular angiogenesis.

### SUMMARY OF THE INVENTION

The present invention discloses compounds of the Formula I:

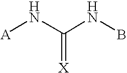

wherein X is O or S;

A is

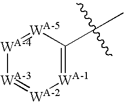

wherein each W^{A-1}, W^{A-2}, W^{A-3}, W^{A-4}, and W^{A-5} are independently N or CR_{A}, provided that no more than four of W^{A-1}, W^{A-2}, W^{A-3}, W^{A-4}, or W^{A-5} are simultaneously N;

Each R_{A} is independently H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₈, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂, provided that at least one R_{A} is other than H;

or when two R_{A} are on adjacent carbon atoms, the two R_{A} may combine to form a 5-8-membered ring fused to the 6-membered ring, wherein the 5-8-membered ring is saturated or unsaturated having up to two heteroatoms selected from —O—, —S—, —N(R_{A-2})—, or —N═ and further having substitution where valency allows on the 5-8-membered ring with up to 2 substitutents independently selected from R_{A-1};

Each R_{A-1} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;

R_{A-2} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

B is a five or six-membered aromatic ring having up to 4 heteroatoms selected from —O—, —N(R_{B-3})—, ═N—, or —S—, wherein B is

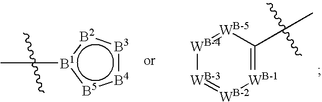

wherein B¹ is N, or C;

B², B³, B⁴, and B⁵ are independently N, O, S, C, provided that when valency allows, the N can have a third bond to R_{B-3}, and further provided that when valency allows, the C can have a fourth bond to R_{B-1};

Each R_{B-1} is independently H, halogen, alkyl, haloalkyl, substituted alkyl, cycloalkyl, halocycloalkyl, substituted cycloalkyl, alkenyl, haloalkenyl, substituted alkenyl, alkynyl, haloalkynyl, substituted alkynyl, heterocycloalkyl, haloheterocycloalkyl, substituted heterocycloalkyl, aryl, —CN, —N₃, —NO₂, —COR₁₀, —CO₂R₇, —CON(R₉)₂, —C(R₁₀)═NOR₇, —SCN, —OR₇, —N(R₉)₂, —SR₈, —SOR₈, —SO₂R₈, —SN(R₉)₂, —SON(R₉)₂, —SO₂N(R₉)₂;

when two R_{B-1} are on adjacent carbon atoms, the two R_{B-1} may combine to form a 5-7-membered ring fused to the 5 or 6 membered ring giving a fused-bicyclic-ring system; wherein the 5-7-membered ring is saturated or unsaturated having up to two heteroatoms selected from —O—, —S—, —N(R_{B-3})—, or —N═ and further having substitution where valency allows on the 5-7-membered ring with up to 2 substitutents independently selected from R_{B-2};

Each R_{B-2} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;

R_{B-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each W^{B-1}, W^{B-2}, W^{B-3}, W^{B-4}, and W^{B-5} are independently N or CR_{B-1}, provided that no more than 4 of W^{B-1}, W^{B-2}, W^{B-3}, W^{B-4}, or W^{B-5} are simultaneously N;

R₇ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₈ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each R₉ is independently H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₁₀ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

or pharmaceutical composition, pharmaceutically acceptable salt, racemic mixture, or pure enantiomer thereof.

Embodiments of the invention may include one or more or combination of the following.

The compounds of Formula I are used to treat, or are used to make a medicament to treat, a mammal where the mammal receives symptomatic relief from activation of an alpha 7 nAChR; these diseases or conditions, include, but are not limited to, any one or more or combination of the following: cognitive and attention deficit symptoms of Alzheimer's, neurodegeneration associated with diseases such as Alzheimer's disease, pre-senile dementia (mild cognitive impairment), senile dementia, schizophrenia or psychosis and cognitive deficits associated therewith, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, or symptoms associated with pain. The compounds of Formula I are also useful to treat or useful to prepare a medicament to treat diseases or conditions where a mammal would receive symptomatic relief from the administration of a compound of Formula I to decrease levels of TNF-α; these diseases or conditions, including, but are not limited to, any one or more or combination of the following: inflammation; pain; cancer; or diabetes. Types of inflammation and/or pain that are to be treated include, but are not limited to, any one or more of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, and HIV-3; cytomegalovirus (CMV); influenza; adenovirus; a herpes virus (including HSV-1, HSV-2); or herpes zoster. Types of cancer that are to be treated include, but are not limited to, any one or more of the following: multiple myeloma; acute and chronic myelogenous leukemia; or cancer-associated cachexia. The compounds of the present invention can be used to treat, or be used to prepare a medicament to treat, the TNF-α aspects associated with pancreatic beta cell destruction; or type I and type II diabetes. The compounds of the present invention are also useful to treat, or to prepare a medicament to be used to treat, diseases or conditions where a mammal would receive symptomatic relief from the increase in vascular angiogenesis; these disease include, but are not limited to, any one or more of the following: wound healing (healing burns, and wounds in general including from surgery), bone fracture healing, ischemic heart disease, and stable angina pectoris.

In another aspect, the invention includes treating, or making medicament(s) to treat, a mammal suffering from schizophrenia or psychosis and cognitive deficits associated with them by administering compounds of Formula I in conjunction with antipsychotic drugs (also called anti-psychotic agents), and also with an agonist of the alpha 7 nAChR, especially when levels of an endogenous agonist are suboptimal. There can be one or more than one medicament. One medicament (pharmaceutical composition) can comprise the compound of formula I, an antipsychotic agent, and/or an alpha 7 nAChR agonist, or there can be a separate medicament for each separately or any combination, e.g., one medicament could have the compound of Formula I and an alpha 7 nAChR agonist and the other medicament could have the antispsychotic agent.

The compounds of the present invention can also be administered in combination with other agents, e.g., the compound of Formula I and the other agent(s) are “co-administered” when treating diseases or conditions discussed herein. For treating the diseases or conditions discussed herein, medicament(s) and pharmaceutical compositions can be prepared comprising a compound of formula I. The same medicament (pharmaceutical composition) or separate medicament(s) (pharmaceutical composition(s)), can be used comprising the other agent(s). For example, but not limitation, co-administration can be used to administer the compounds of the present invention and an alpha 7 nAChR agonist. The compounds of the present invention and an alpha 7 nAChR agonist can also be co-administered with the other agents dicussed herein.

Another aspect of the present invention includes for example, but not limitation, co-administration can be used when treating symptoms associated with infection, inflammation, cancer, or diabetes. The same medicament or separate medicament(s), can be used comprising a compound of Formula I and any one of the following: an antibacterial and antiviral agent for treating infection; an anticancer agent and/or antiemetic agent for treating cancer; or at least one agent to treat diabetes for treating diabetes. For example, the compound of Formula I can be co-administered with an antibacterial or antiviral agent, as one medicament or as two separate medicament, to treat an infection, for example, but not limiting, rhinitis. The compound of Formula I can also be co-administered with an anticancer agent and/or antiemetic agent when the disease or condition being treated is cancer, so there could be one medicament or separate medicaments for each agent. And, the compound of Formula I can be co-administered with agents to treat diabetes in one medicament or as separate medicaments.

In a combination therapy, the compounds of Formula I and the other agent(s) can be co-administered simultaneously or at separate intervals. When co-administered simultaneously, the compounds of Formula I and the other agent(s) can be incorporated into a single pharmaceutical composition, e.g., a pharmaceutical combination therapy composition. Alternatively, more than one, e.g., two, separate compositions, i.e., one containing a compound of Formula I and the other containing, for example, the psychostimulant, can be administered.

A pharmaceutical combination therapy composition can also be used to treat ADHD, using, for example, but not for limitation, psychostimulants and/or monoamine reuptake inhibitors. This composition can also include an alpha 7 nAChR agonist. While psychostimulants and monoamine reuptake inhibitors control the activity level, and attention, they are not effective in treating the co-morbid or concomitant deficit in cognition that is associated with ADHD. The combination therapy will be more effective at treating this disease because the ability of the mammal to regulate an α7 nAChR agonist will treat the underlying cognitive dysfunction in the disorder and the other two classes of drugs will treat the behavioral problems associated with ADHD. Psychostimulants used for these compositions include, but are not limited to: methylphenidate (Ritalin) administered at about 0.01 to about 0.85 mg/kg/day; dextroamphetamine (Dexedrine) administered at about 0.07 to about 0.85 mg/kg/day; amphetamine (Adderall) administered at about 0.05 to about 0.6 mg/kg/day; and pemoline (Cylert) administered at about 0.1 to about 1.6 mg/kg/day. Monoamine Reuptake inhibitors for these compositions include, but are not limited to: desipramine (Norpramin) administered at about 0.5 to about 5.0 mg/kg/day; nortriptyline administered at about 0.1 to about 3.0 mg/kg/day; atomoxetine (Strattera) administered at about 0.1 to about 3.0 mg/kg/day; reboxetine administered at about 0.03 to about 3.0 mg/kg/day; fluoxetine (Prozac) administered at about 0.2 to about 20 mg/kg/day; tomoxetine administered at about at about 0.1 to about 1.1 mg/kg/day; bupropion (Welibutrin) administered at about at about 1.0 to about 1.1 mg/kg/day; or modaphonil (Provigil) administered at about at about 1.0 to about 5.7 mg/kg/day. The medicament(s) used to treat ADHD can comprise any combination or single item of the following: a compound of formula I, a psychostimulant, a monoamine reuptake inhibitor and/or an alpha 7 nAChR agonist, or separate medicament(s) can be prepared comprising a any combination of them.

There are also three forms of combination therapies to enhance the activity of a positive allosteric modulator in the presence of an agonist of the α7 nAChR. The first combination therapy is to use a positive allosteric modulator of the α7 nAChR with drugs such as Aricept and Reminyl that inhibit the activity of acetylcholinesterase. Acetylcholinesterase is the enzyme that is primarily responsible for degrading ACh. Drugs such as Aricept and Reminyl which are used to treat Alzheimer's disease, increase ACh levels. The increase in ACh levels leads to an increase in the activity of α7 nAChR and other nicotinic and muscarinic receptors. Thus treating with both acetylcholinesterase inhibitors and a positive allosteric modulator of α7 nAChR will selectively enhance the activity of the α7 nAChR which could provide significant therapeutic benefit for the patient.

The second combination therapy is to use a positive allosteric modulator of the α7 nAChR with a drug that directly activates the α7 nAChR. Drugs that act as receptor agonists and directly activate the α7 nAChR have therapeutic potential but they also carry the liability that prolonged exposure may lead to a loss of efficacy. Using a direct acting agonist of the α7 nAChR in combination with a positive allosteric modulator of the α7 nAChR make both classes of drugs more effective.

The third combination therapy is to use a positive allosteric modulator of α7 nAChR in combination with nutritional supplements including phosphotidylserine, phosphotidylycholine, or choline that act by increasing levels of ACh in the brain. As previously mentioned, an increase in ACh leads to an increase in the activity of α7 nAChR and other nicotinic and muscarinic receptors. Thus, treating with cholinergic nutritional supplements and a positive allosteric modulator of α7 nAChR will selectively enhance the activity of the α7 nAChR to provide significant therapeutic benefit for the patient.

A pharmaceutical combination therapy composition can include therapeutically effective amounts of the compounds of Formula I, and a therapeutically effective amount of the other drug(s)/agent(s). These compositions may be formulated with common excipients, diluents or carriers, and compressed into tablets, or formulated elixirs or solutions for convenient oral administration or administered by intramuscular intravenous routes. The compounds can be administered rectally, topically, orally, or sublingually.

In a combination therapy, the compounds of Formula I and the other drug(s) can be administered simultaneously or at separate intervals. When administered simultaneously the compounds of Formula I and the other drug(s) can be incorporated into a single pharmaceutical composition, e.g., a pharmaceutical combination therapy composition. Alternatively, two or more separate compositions, i.e., one containing compounds of Formula I and the other containing the other drug(s), can be administered simultaneously.

When separately administered, therapeutically effective amounts of compositions containing compounds of Formula I and the other drug(s) are administered on a different schedule. One may be administered before the other as long as the time between the two administrations falls within a therapeutically effective interval. A therapeutically effective interval is a period of time beginning when one of either (a) the compounds of Formula I, or (b) the other drug(s) is administered to a human and ending at the limit of the beneficial effect in the treatment of the disease or condition using the combination of (a) and (b). The methods of administration of the compounds of Formula I and the other drug(s) may vary. Thus, either agent or both agents may be administered rectally, topically, orally, sublingually, or parenterally.

The amount of therapeutically effective compound of Formula I that is administered and the dosage regimen for treating a disease or condition with the compounds and/or compositions of this invention depends on a variety of factors, including the age, weight, sex and medical condition of the subject, the severity of the disease, the route and frequency of administration, and the particular compound(s) employed, and thus may vary widely. The compositions contain well know carriers and excipients in addition to a therapeutically effective amount of compounds of Formula I. The pharmaceutical compositions may contain the compound of Formula I in the range of about 0.001 to 100 mg/kg/day for an adult, preferably in the range of about 0.01 to about 50 mg/kg/day for an adult. A total daily dose of about 1 to 1000 mg of a compound of Formula I may be appropriate for an adult. The daily dose can be administered in one to four doses per day. These compositions may be formulated with common excipients, diluents or carriers, and compressed into tablets, or formulated elixirs or solutions for convenient oral administration or administered by intramuscular intravenous routes. The compounds of Formula I can be administered rectally, topically, orally, sublingually, or parenterally and maybe formulated as sustained relief dosage forms and the like.

The combined administration of the compounds of Formula I and the other agent(s) is expected to require less of the generally-prescribed dose for either agent when used alone and or is expected to result in less frequent administration of either or both agents. The skilled clinician may in fact learn that behavioral problems are secondary to the cognitive problems and can be treated with lower dosages of the other agent(s). Determining such dosages and routes of administration should be a routine determination by one skilled in the art of treating patients with the diseases or conditions discussed herein.

Another group of compounds of Formula I includes compounds where X is O or S. Another group of compounds of Formula I includes compounds where A and B have any definition discussed herein.

Another group of compounds of Formula I includes compounds where each R_{A} includes any one of the following: H, halogen, methyl, ethyl, i-propyl, n-propyl, haloalkyl, —OH, —NO₂, —CN, —C(alkyl)═N(O-alkyl), —O-alkyl, —O-(substituted alkyl), —O-alkenyl, —O-(heterocycloalkyl), —O-(substituted heterocycloalkyl), —S-alkyl, —SO-alkyl, —SO₂-alkyl, C(═O)-(lower alkyl), —O-haloalkyl, —SO-haloalkyl, —SO-haloalkyl, —SO₂-haloalkyl, cycloalkyl, or heterocycloalkyl. Another group of compounds of Formula I includes compounds where each R_{B-1} independently includes any one of the following: H, Cl, Br, CN, methyl, ethyl, i-propyl, n-propyl, cyclopropyl, haloalkyl, —CF₃, —CF₂CF₃, —OMe, —OCF₃, —OEt, —SOMe, —SO₂Me, or —SO₂CF₃.

Another group of compounds of Formula I includes compounds where each R_{A} is independently any one or more of the following: H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₈, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂, provided that at least one R_{A} is other than H, wherein R₇, R₈, each R₉, and R₁₀ have any definition discussed herein.

Another group of compounds of Formula I includes compounds where two R_{A} are on adjacent carbon atoms combine to form a fused-bicyclic-ring system giving a 6-membered ring from the phenyl fused to a 5-8-membered saturated or unsaturated ring system having up to two heteroatoms selected from —O—, —S—, —N(R_{A-N})—, or —N═ and further having substitution where valency allows on the 5-8-membered ring with 1-2 substitutents independently selected from R_{A-1}, which can have any definition as discussed herein.

Another group of compounds of Formula I includes compounds where each R_{B-1} is independently any one or more of the following: H, halogen, alkyl excluding t-butyl, haloalkyl, substituted alkyl, cycloalkyl, halocycloalkyl, substituted cycloalkyl, alkenyl, haloalkenyl, substituted alkenyl, alkynyl, haloalkynyl, substituted alkynyl, heterocycloalkyl, haloheterocycloalkyl, substituted heterocycloalkyl, aryl, —CN, —N₃, —NO₂, —COR₁₀, —CO₂R₇, —CON(R₉)₂, —C(R₁₀)═NOR₇, —SCN, —OR₇, —N(R₉)₂, —SOR₈, —SO₂R₈, —SN(R₉)₂, —SON(R₉)₂, —SO₂N(R₉)₂. Another group of compound Formula I includes compounds where two R_{B-1} are on adjacent carbon atoms may combine to form a fused-bicyclic-ring system having B fused to a 5-7-membered saturated or unsaturated ring system having up to two heteroatoms selected from —O—, —S—, —N(R_{B-N})—, or —N═ and further having substitution where valency allows on the 5-7-membered ring with 1-2 substitutents independently selected from R_{B-2}, which can have any definition discussed herein. Another group of compounds of Formula I includes compounds where each R_{B-3} is independently any one or more of the following: H, alkyl, haloalkyl, —OH, —O-alkyl, or —O-haloalkyl.

Another group of compounds of Formula I includes compounds where each R₃ is independently any one or more or combination of the following: H, alkyl, haloalkyl, alkenyl, haloalkenyl, alkynyl, haloalkynyl, cycloalkyl, halocycloalkyl, heterocycloalkyl, haloheterocycloalkyl, or phenyl optionally substituted with 0-3 halogens and 0-1 substituent selected from alkyl, —CF₃, —CN, —NH₂, —NO₂, and —OH.

Another group of compounds of Formula I includes compounds where R₄ is any one or more of the following: H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, or aryl.

Another group of compounds of Formula I includes compounds where R₅ is any one or more of the following: alkyl, alkenyl, alkynyl, cycloalkyl, heterocycloalkyl, haloalkyl, haloalkenyl, haloalkynyl, halocycloalkyl, haloheterocycloalkyl, —CN, —NO₂, —OR₃, —SR₃, —N(R₃)₂, —C(O)R₃, —C(O)N(R₃)₂, —NR₃C(O)R₃, —S(O)₂N(R₃)₂, —NR₃S(O)₂R₃, alkyl substituted with 1-4 substituent(s) independently selected from F, Cl, Br, I, or R₆, cycloalkyl substituted with 1-4 substituent(s) independently selected from F, Cl, Br, I, or R₆, or heterocycloalkyl substituted with 1-4 substituent(s) independently selected from F, Cl, Br, I, or R₆.

Another group of compounds of Formula I includes compounds where R₆ is any one or more of the following: —CF₃, —CN, —NO₂, —OR₃, —SR₃, —N(R₃)₂, —C(O)R₃, —C(O)N(R₃)₂, —NR₃C(O)R₃, —S(O)₂N(R₃)₂, or —NR₃S(O)₂R₃.

One of ordinary skill in the art will recognize that where alkyl, substituted alkyl, haloalkyl, alkenyl, substituted alkenyl, haloalkenyl are allowed, lower alkyl, lower substituted alkyl, lower haloalkyl, lower alkenyl, lower substituted alkenyl, and lower haloalkenyl, respectively, are also allowed. Therefore, alkyl would include lower alkyl, which would include, but not be limited to, methyl and ethyl. And, —O-alkyl would inlcude —O-lower alkyl, including, but not limited to, —O-methyl or —O-ethyl, and —O-haloalkyl would allow —O-lower haloalkyl, including, but not limited to, —O-trifluoromethyl, —O-1,1,1-trifluoroeth-2-yl, and —O-pentafluoroethyl.

Another group of compounds of Formula I includes compounds where each R₇, R₈, R₉, or R₁₀ is independently any one or more of the following: H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl.

Another group of compounds of Formula I includes compounds where A is phenyl or pyridinyl, wherein W^{A-2} is CH or N. Another group of compounds of Formula I includes compounds where W^{A-1} is C—R_{A}, where R_{A} is any one or more of the following: H, lower alkyl, O-lower alkyl, S-lower alkyl, S(O)-lower alkyl, NO₂, C(O)-lower alkyl, and C(═N—O-(lower alkyl))-(lower alkyl). Another group of compounds of Formula I includes compounds where W^{A-3} is C—R_{A}, where R_{A} is any one or more of the following: H, O-lower alkyl, O-sec-butyl, —O-(heterocycloalkyl), —O-(substituted heterocycloalkyl), and ethoxy substituted on C-2 with any one of the following: OH, OMe, OEt, SMe, SEt, S(O)Me, S(O)₂Me, NH-(lower alkyl), N-(lower alkyl)₂, NHC(O)-lower alkyl, NHS(O)₂-(lower alkyl)₂, morpholinyl, thiomorpholinyl, 1,1-dioxidothiomorpholinyl, piperazinyl, pyrrolidinyl, 1H-pyrazolyl, and piperidinyl. Another group of compounds of Formula I includes compounds where W^{A-4} is C—R_{A}, where R_{A} is any one or more of the following: H, lower alkyl, O-lower alkyl, or halogen.

Another group of compounds of Formula I includes compounds where W^{A-1} is C—R_{A}, where R_{A} is any one or more of the following: H, Me, OMe, OEt, O-isopropyl, O-n-propyl, SMe, SOMe, SEt, S(O)Et, NO₂, C(O)Me, and C(═N—OMe)CH₃. Another group of compounds of Formula I includes compounds where W^{A-3} is C—R_{A}, where R_{A} is any one or more of the following: H, O-lower alkyl, O-sec-butyl, —O-(heterocycloalkyl), —O-(substituted heterocycloalkyl), and ethoxy substituted on C-2 with OH, OMe, OEt, SMe, SEt, S(O)Me, S(O)₂Me, NHMe, N(Me)₂, NHEt, N(Et)₂, N(Me)(Et), NHC(O)Me, NHS(O)₂(Me), morpholin-4-yl, thiomorpholin-4-yl, 1,1-dioxidothiomorpholin-4-yl, piperazin-1-yl, pyrrolidin-1-yl, 1H-pyrazol-1-yl, and piperidin-1-yl. Another group of compounds of Formula I includes compounds where W^{A-4} is C—R_{A}, where R_{A} is any one or more of the following: H, Me, OMe, F, Cl, and Br. Lower alkyl can include, but is not limited to, any one or more of the following: methyl, ethyl, n-propyl, and i-propyl. Heterocycloalkyl can include, but is not limited to, any one or more of the following: O-tetrahydrofuranyl, O-oxetanyl, O-1,1-dioxidothietanyl, and O-azetidinyl with the bond between O and the heterocycloalkyl being at any atom where valency allows.

Another group of compounds of Formula I includes compounds where B is isoxazol-3-yl optionally substituted at C-5 with any one or more of the following: lower alkyl, lower haloalkyl, lower cycloalkyl, halogen, and CN. Another group of compounds of Formula I includes compounds where B is isoxazol-3-yl is optionally substituted at C-5 with any one or more of the following: CH₃, CF₃, CH₂F, CHF₂, CH₂OCH₃, CH₂CH₃, CF₂CF₃, cyclopropyl, Cl, Br, or CN. Another group of compounds of Formula I includes compounds where B is isoxazol-5-yl substituted at C-3 with any one or more of the following: lower alkyl, lower haloalkyl, CN, and halogen. Another group of compounds of Formula I includes compounds where B is isoxazol-5-yl substituted at C-3 with any one or more of the following: CH₃, CF₃, CH₂F, CHF₂, CF₂CF₃, CN, Cl, and Br. Another group of compounds of Formula I includes compounds where B is 1,3,4-thiadiazol-2-yl substituted at C-5 with any one or more of the following: lower alkyl, lower haloalkyl, CN, and halogen. Another group of compounds of Formula I includes compounds where B is 1,3,4-thiadiazol-2-yl substituted at C-5 with any one or more of the following: CH₃, CF₃, CH₂F, CHF₂, CF₂CF₃, CN, Cl, and Br.

Another group of compounds of Formula I includes compounds where B is any one of the following: isothiazol-3-yl and 1,3-thiazol-2-yl, either of which is optionally substituted at C-5, 1,3-thiazol-5-yl optionally substituted at C-2 and also pyridin-3-yl optionally substituted at C-6, where the optional substitutent is any one or more of the following: methyl, trifluoromethyl, chloro, bromo, and cyano. Another group of compounds of Formula I includes compounds where W^{A-1} is C—R_{A}, where R_{A} is any one or more of the following: H, methyl, OMe, OEt, SMe, nitro, or C(O)Me. Another group of compounds of Formula I includes compounds where W^{A-2} is N or CH. Another group of compounds of Formula I includes compounds where W^{A-3} is C—R_{A}, where R_{A} is any one or more of the following: H, methoxy, ethoxy, O-allyl, and 2-methoxyethoxy. Another group of compounds of Formula I includes compounds where W^{A-4} is C—R_{A}, where R_{A} is any one or more of the following: H, methyl, methoxy, fluoro, chloro, and bromo.

Another group of compounds of Formula I includes compounds where B is any one of the following: thiazolyl, 3-trifluorophenyl, and 3-phenyl-1,2,4-thiadiazolyl where the phenyl off of the thiadiazolyl is optionally substituted with up to 3 substituents being lower alkyl, lower haloalkyl, O-(lower alkyl) and halogen. Another group of compounds of Formula I includes compounds where B is thiazol-2-yl substituted at C-4 with any one of the following: methyl, trifluoromethyl, ethyl, and pentafluoroeth-1-yl.

Another group of compounds of Formula I includes compounds where A includes, but is not limited to, compounds wherein up to four of W^{A-1}, W^{A-2}, W^{A-3}, W^{A-4}, and W^{A-5} can be N to include the following moieties:

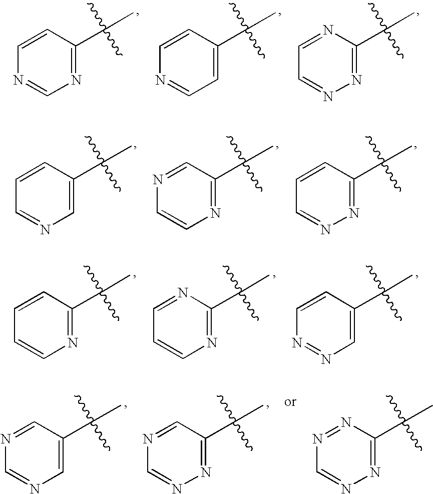

optionally substituted as valency allows and as R_{A} is defined herein.

Another group of compounds of Formula I includes all compounds except compounds wherein when W^{A-1}, W-^{A-2}, W^{A-3}, W^{A-4}, and W^{A-5} are all CR_{A-1}, and the R_{A}'s of W^{A-3} and W^{A-4} form a 5-membered ring to make an indol-5-yl moiety and wherein the R_{A} of W^{A-5} is H or alkyl and wherein B is a mono-cyclic 5-membered ring.

Another group of compounds of Formula I includes compounds where B includes, but is not limited to, compounds wherein W^{B-1}, W^{B-2}, W^{B-3}, W^{B-4}, and W^{B-5} can be N or CR_{B-1} to include the following moieties:

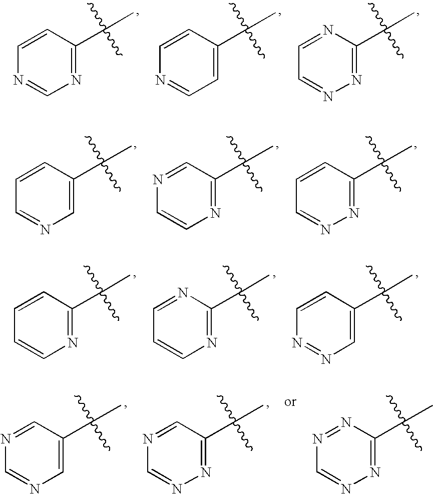

optionally substituted as valency and the definition of Formula I allow and with any definition of R_{B-1} as discussed herein.

Another group of compounds of Formula I includes compounds wherein B includes, but is not limited to, the following moieties that one of ordinary skill in the art can recognize as fitting within the scope of the structures drawn for B:

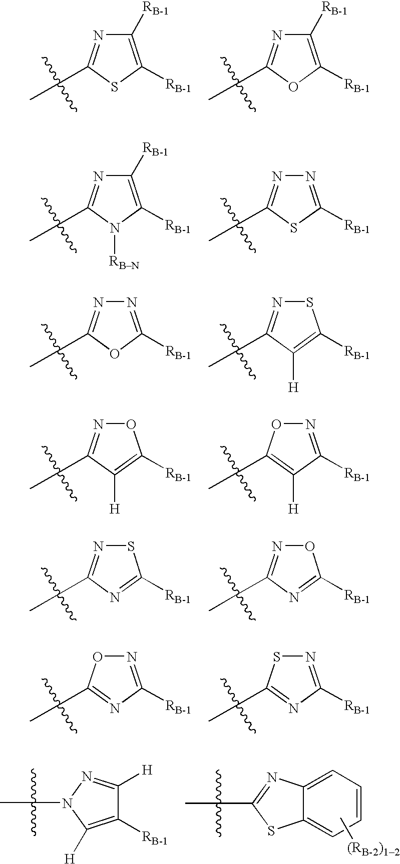

where each R_{B-1}, and R_{B-2} have any definition discussed herein and can occur at any carbon where valency allows, and where R_{B-N} has any definition discussed herein and can occur at any nitrogen where valency allows.

Another group of compounds of Formula I includes compounds wherein B is thiadiazolyl, and when A is phenyl, at least one R_{A} is selected from other than H, methyl, isopropyl, —NO₂, —CF₃, methoxy, —OH, —CN, or halogen.

Another group of compounds of Formula I includes compounds wherein B is benzimidazolyl and benzthiazolyl, provided that A is not a phenyl moiety optionally substituted with 1-3 substituents selected from halogen.

Another group of compounds for Formula I includes compounds wherein B is isoxazol-3-yl optionally substituted at the four position with trifluoromethyl, O—C₁₋₄alkyl, or alkyl substituted with hydroxy, provided that A is not phenyl substituted in each ortho position with alkyl, trifluoromethyl or halo.

Another group of compounds of Formula I includes any one or more or combination of the following:

N-(4-methoxy-2-methylphenyl)-N′-[3-(trifluoromethyl)phenyl]urea;

N-(5-chloro-2,4-dimethoxyphenyl)-N′-isoxazol-3-ylurea;

N-(5-chloro-2,4-dimethoxyphenyl)-N′-(5-methyl-1,3,4-thiadiazol-2-yl)urea;

N-(5-chloro-2,4-dimethoxyphenyl)-N′-(5-methylisoxazol-3-yl)urea;

N-(5-chloro-2,4-dimethoxyphenyl)-N′-(5-ethyl-1,3,4-thiadiazol-2-yl)urea;

N-(5-chloro-2,4-dimethoxyphenyl)-N′-[5-(trifluoromethyl)-1,3,4-thiadiazol-2-yl]urea;

N-(5-chloro-2,4-dimethoxyphenyl)-N′-(5-cyclopropyl-1,3,4-thiadiazol-2-yl)urea;

N-(5-chloro-2,4-dimethoxyphenyl)-N′-[5-(difluoromethyl)-1,3,4-thiadiazol-2-yl]urea;

N-(5-chloro-2,4-dimethoxyphenyl)-N′-(5-methoxy-1,3,4-thiadiazol-2-yl)urea;

N-(5-bromo-1,3,4-thiadiazol-2-yl)-N′-(5-chloro-2,4-dimethoxyphenyl)urea;

N-(5-chloro-1,3,4-thiadiazol-2-yl)-N′-(5-chloro-2,4-dimethoxyphenyl)urea;

N-(5-chloro-2,4-dimethoxyphenyl)-N′-[5-(2-phenylethyl)-1,3,4-thiadiazol-2-yl]urea;

N-(5-chloro-2,4-dimethoxyphenyl)-N′-(5-ethoxy-1,3,4-thiadiazol-2-yl)urea;

N-(5-chloro-2,4-dimethoxyphenyl)-N′-(1,3,4-thiadiazol-2-yl)urea;

N-(4-ethoxyphenyl)-N′-(5-methylisoxazol-3-yl)urea;

N-(2,4-dimethoxyphenyl)-N′-(5-methylisoxazol-3-yl)urea;

N-(2,4-dimethoxyphenyl)-N′-[5-(trifluoromethyl)-1,3,4-thiadiazol-2-yl]urea;

N-(5-chloro-2-methoxyphenyl)-N′-(5-methylisoxazol-3-yl)urea;

N-(5-chloro-2-methoxyphenyl)-N′-(5-methyl-1,3,4-thiadiazol-2-yl)urea;

N-(4-isopropoxyphenyl)-N′-[5-(trifluoromethyl)-1,3,4-thiadiazol-2-yl]urea;

N-(2-ethoxyphenyl)-N′-[5-(trifluoromethyl)-1,3,4-thiadiazol-2-yl]urea;

N-(4-ethoxyphenyl)-N′-[5-(trifluoromethyl)-1,3,4-thiadiazol-2-yl]urea;

N-(4-methoxyphenyl)-N′-[5-(trifluoromethyl)-1,3,4-thiadiazol-2-yl]urea;

N-(4-butoxyphenyl)-N′-[5-(trifluoromethyl)-1,3,4-thiadiazol-2-yl]urea;

N-(2,3-dihydro-1,4-benzodioxin-6-yl)-N′-[5-(trifluoromethyl)-1,3,4-thiadiazol-2-yl]urea;

N-(2,3-dihydro-1-benzofuran-5-yl)-N′-[5-(trifluoromethyl)-1,3,4-thiadiazol-2-yl]urea;

N-(4-ethyl-1,3-thiazol-2-yl)-N′-(4-methoxy-2-methylphenyl)urea;

N-(5-chloro-2,4-dimethoxyphenyl)-N′-(1H-imidazol-2-yl)urea;

N-(5-bromo-1,3-thiazol-2-yl)-N′-(5-chloro-2,4-dimethoxyphenyl)urea;

N-(5-chloro-2,4-dimethoxyphenyl)-N′-(5-methyl-1,3-thiazol-2-yl)urea;

N-(5-chloro-2,4-dimethoxyphenyl)-N′-(3-methylisoxazol-5-yl)urea;

N-(5-chloro-2,4-dimethoxyphenyl)-N′-(6-cyanopyridin-3-yl)urea;

N-(5-chloro-2,4-dimethoxyphenyl)-N′-(5-chloro-1,3-thiazol-2-yl)urea;

N-(5-chloro-1,3-thiazol-2-yl)-N′-(2,4-dimethoxy-5-methylphenyl)urea;

N-(5-bromo-2,4-dimethoxyphenyl)-N′-(5-chloro-1,3-thiazol-2-yl)urea;

N-(5-bromo-1,3-thiazol-2-yl)-N′-(5-fluoro-2,4-dimethoxyphenyl)urea;

N-(5-chloro-1,3-thiazol-2-yl)-N′-(5-fluoro-2,4-dimethoxyphenyl)urea;

N-(3-chloro-4-fluorophenyl)-N′-(5-methylisoxazol-3-yl)urea;

N-(3-chloro-4-fluorophenyl)-N′-[5-(trifluoromethyl)-1,3,4-thiadiazol-2-yl]urea;

N-(2-ethoxyphenyl)-N′-(5-methylisoxazol-3-yl)urea;

N-(2-methoxyphenyl)-N′-[5-(trifluoromethyl)-1,3,4-thiadiazol-2-yl]urea;

N-(2-fluoro-4-methoxyphenyl)-N′-[5-(trifluoromethyl)-1,3,4-thiadiazol-2-yl]urea;

N-(5-chloro-2,4-dimethoxyphenyl)-N′-(5-mercapto-1,3,4-thiadiazol-2-yl)urea;

N-(4,5-dimethoxy-2-methylphenyl)-N′-[5-(trifluoromethyl)-1,3,4-thiadiazol-2-yl]urea;

N-(4-hydroxy-2-nitrophenyl)-N′-[5-(trifluoromethyl)-1,3,4-thiadiazol-2-yl]urea;

N-(2,6-dimethoxypyridin-3-yl)-N′-(5-methyl-1,3-thiazol-2-yl)urea;

N-(4-ethoxy-2-nitrophenyl)-N′-(5-methylisoxazol-3-yl)urea;

N-(4-methoxy-2-methylphenyl)-N′-[2-(trifluoromethyl) pyridin-4-yl]urea;

N-(5-fluoro-2,4-dimethoxyphenyl)-N′-(5-methylisoxazol-3-yl)urea;

N-(5-fluoro-2,4-dimethoxyphenyl)-N′-(5-methyl-1,3-thiazol-2-yl)urea;

N-(5-fluoro-2,4-dimethoxyphenyl)-N′-(5-methyl-1,3,4-thiadiazol-2-yl)urea;

N-(5-fluoro-2,4-dimethoxyphenyl)-N′-[5-(trifluoromethyl)-1,3,4-thiadiazol-2-yl]urea;

N-(5-bromo-2,4-dimethoxyphenyl)-N′-(5-methylisoxazol-3-yl)urea;

N-(5-bromo-2,4-dimethoxyphenyl)-N′-(5-methyl-1,3-thiazol-2-yl)urea;

N-(5-bromo-2,4-dimethoxyphenyl)-N′-(5-methyl-1,3,4-thiadiazol-2-yl)urea;

N-(5-bromo-2,4-dimethoxyphenyl)-N′-[5-(trifluoromethyl)-1,3,4-thiadiazol-2-yl]urea;

N-(3,5-difluoro-2,4-dimethoxyphenyl)-N′-(5-methylisoxazol-3-yl)urea;

N-(3,5-difluoro-2,4-dimethoxyphenyl)-N′-[5-(trifluoromethyl)-1,3,4-thiadiazol-2-yl]urea;

N-(3,5-difluoro-2,4-dimethoxyphenyl)-N′-(5-methyl-1,3-thiazol-2-yl)urea;

N-(3,5-difluoro-2,4-dimethoxyphenyl)-N′-(5-methyl-1,3,4-thiadiazol-2-yl)urea;

N-[2,4-dimethoxy-5-(trifluoromethyl)phenyl]-N′-[5-(trifluoromethyl)-1,3,4-thiadiazol-2-yl]urea;

N-[2,4-dimethoxy-5-(trifluoromethyl)phenyl]-N′-(5-methylisoxazol-3-yl)urea;

N-[2,4-dimethoxy-5-(trifluoromethyl)phenyl]-N′-(5-methyl-1,3-thiazol-2-yl)urea;

N-[2,4-dimethoxy-5-(trifluoromethyl)phenyl]-N′-(5-methyl-1,3,4-thiadiazol-2-yl)urea;

N-(5-chloro-2,4-diethoxyphenyl)-N′-(5-methylisoxazol-3-yl)urea;

N-(5-chloro-2,4-diethoxyphenyl)-N′-(5-methyl-1,3-thiazol-2-yl)urea;

N-(5-chloro-2,4-diethoxyphenyl)-N′-(5-methyl-1,3,4-thiadiazol-2-yl)urea;

N-(5-chloro-2,4-diethoxyphenyl)-N′-[5-(trifluoromethyl)-1,3,4-thiadiazol-2-yl]urea;

N-(5-chloro-2,4-dipropoxyphenyl)-N′-[5-(trifluoromethyl)-1,3,4-thiadiazol-2-yl]urea;

N-(5-chloro-2,4-dipropoxyphenyl)-N′-(5-methylisoxazol-3-yl)urea;

N-(5-chloro-2,4-diisopropoxyphenyl)-N′-(5-methyl-1,3,4-thiadiazol-2-yl)urea;

N-(5-chloro-2,4-diisoprooxyphenyl)-N′-[5-(trifluoromethyl)-1,3,4-thiadiazol-2-yl]urea;

N-(5-chloro-2,4-diisoprooxyphenyl)-N′-(5-methylisoxazol-3-yl)urea;

N-(2-chloro-4-methoxy-5-methylphenyl)-N′-(5-methylisoxazol-3-yl)urea;

N-(2-chloro-4-methoxy-5-methylphenyl)-N′-(5-methyl-1,3-thiazol-2-yl)urea;

N-(2-chloro-4-methoxy-5-methylphenyl)-N′-(5-methyl-1,3,4-thiadiazol-2-yl)urea;

N-(2-chloro-4-methoxy-5-methylphenyl)-N′-[5-(trifluoromethyl)-1,3,4-thiadiazol-2-yl]urea;

N-(2,4-dimethoxy-5-methylphenyl)-N′-(5-methylisoxazol-3-yl)urea;

N-(2,4-dimethoxy-5-methylphenyl)-N′-(5-methyl-1,3-thiazol-2-yl)urea;

N-(2,4-dimethoxy-5-methylphenyl)-N′-(5-methyl-1,3,4-thiadiazol-2-yl)urea;

N-(2,4-dimethoxy-5-methylphenyl)-N′-[5-(trifluoromethyl)-1,3,4-thiadiazol-2-yl]urea;

N-(4-ethoxy-2-methoxy-5-methylphenyl)-N′-[5-(trifluoromethyl)-1,3,4-thiadiazol-2-yl]urea;

N-(5-acetyl-2,4-dimethoxyphenyl)-N′-[5-(trifluoromethyl)-1,3,4-thiadiazol-2-yl]urea;

N-(5-acetyl-2,4-dimethoxyphenyl)-N′-(5-methyl-1,3,4-thiadiazol-2-yl)urea;

N-(5-acetyl-2,4-dimethoxyphenyl)-N′-(5-methylisoxazol-3-yl)urea;

N-(2,4-dimethoxy-5-nitrophenyl)-N′-[5-(trifluoromethyl)-1,3,4-thiadiazol-2-yl]urea;

N-(2,4-dimethoxy-5-nitrophenyl)-N′-(5-methyl-1,3-thiazol-2-yl)urea;

N-(2,4-dimethoxy-5-nitrophenyl)-N′-(5-methylisoxazol-3-yl)urea;

N-[2-methoxy-5-methyl-4-(2,2,2-trifluoroethoxy)phenyl]-N′-[5-(trifluoromethyl)-1,3,4-thiadiazol-2-yl]urea;

N-(5-chloro-2,4-dimethoxyphenyl)-N′-(2-methyl-1,3-thiazol-5-yl)urea;

N-(2-methoxy-4-nitrophenyl)-N′-[5-(trifluoromethyl)-1,3,4-thiadiazol-2-yl]urea;

N-[5-(trifluoromethyl)-1,3,4-thiadiazol-2-yl]-N′-(2,4,5-trimethoxyphenyl)urea;

N-[4-methoxy-2-(methylthio)phenyl]-N′-[5-(trifluoromethyl)-1,3,4-thiadiazol-2-yl]urea;

N-(4-{[(1R)-1-methylpropy]oxy}phenyl)-N′-[5-(trifluoromethyl)-1,3,4-thiadiazol-2-yl]urea;

N-[4-(allyloxy)phenyl]-N′-[5-(trifluoromethyl)-1,3,4-thiadiazol-2-yl]urea;

N-(4-propoxyphenyl)-N′-[5-(trifluoromethyl)-1,3,4-thiadiazol-2-yl]urea;

N-(2-ethoxypyridin-3-yl)-N′-[5-(trifluoromethyl)-1,3,4-thiadiazol-2-yl]urea;

N-(4-methoxy-2-methylphenyl)-N′-[4-(trifluoromethyl)-1,3-thiazol-2-yl]urea;

N-(4-methoxy-2-methylphenyl)-N′-(3-phenyl-1,2,4-thiadiazol-5-yl)urea;

N-(5-ethyl-4-phenyl-1,3-thiazol-2-yl)-N′-(4-methoxy-2-methylphenyl)urea;

N-(4-hydroxy-2-methylphenyl)-N′-[3-(trifluoromethyl)phenyl]urea;

N-(4-hydroxyphenyl)-N′-[3-(trifluoromethyl)phenyl]urea;

N-[2-methyl-4-(methylthio)phenyl]-N′-[3-(trifluoromethyl)phenyl]urea;

N-(2-ethyl-4-hydroxyphenyl)-N′-[3-(trifluoromethyl)phenyl]urea;

N-(4-amino-2-methylphenyl)-N′-[3-(trifluoromethyl)phenyl]urea;

N-(4-methoxyphenyl)-N′-[3-(trifluoromethyl)phenyl]urea;

N-(5-hydroxy-2-methylphenyl)-N′-[3-(trifluoromethyl)phenyl]urea;

N-(5-chloro-2,4-dimethoxyphenyl)-N′-[4-(trifluoromethyl)-1H-pyrazol-1-yl]urea;

N-(4-bromo-1H-pyrazol-1-yl)-N′-(5-chloro-2,4-dimethoxyphenyl)urea;

N-(4-methoxy-2-methylphenyl)-N′-[3-(trifluoromethyl)phenyl]thiourea;

N-(4-hydroxy-2-methylphenyl)-N′-[3-(trifluoromethyl)phenyl]thiourea;

N-(2,4-dimethoxy-5-methylphenyl)-N′-[3-(trifluoromethyl)isoxazol-5-yl]urea;

N-(4-ethoxyphenyl)-N′-[3-(trifluoromethyl)isoxazol-5-yl]urea;

N-(2-ethoxyphenyl)-N′-[3-(trifluoromethyl)isoxazol-5-yl]urea;

N-(2,4-dimethoxyphenyl)-N′-[3-(trifluoromethyl)isoxazol-5-yl]urea;

N-(2,6-dimethoxypyridin-3-yl)-N′-(3-methylisoxazol-5-yl)urea;

N-(5-fluoro-2,4-dimethoxyphenyl)-N′-(3-methyl isoxazol-5-yl)urea;

N-(2,4-dimethoxyphenyl)-N′-(3-methylisoxazol-5-yl)urea;

N-(5-chloro-2,4-dimethoxyphenyl)-N′-[3-(trifluoromethyl)isoxazol-5-yl]thiourea;

N-(5-chloro-2,4-diethoxyphenyl)-N′-[3-(trifluoromethyl)isoxazol-5-yl]urea;

N-(4-methoxy-2-nitrophenyl)-N′-[3-(trifluoromethyl)isoxazol-5-yl]urea;

N-(4-methoxy-2-methylphenyl)-N′-[4-(trifluoromethyl)-1,3-thiazol-2-yl]thiourea;

N-(5-chloro-2,4-dimethoxyphenyl)-N′-[5-(trifluoromethyl)-1,3,4-thiadiazol-2-yl]thiourea;

N-(5-fluoro-2,4-dimethoxyphenyl)-N′-[5-(trifluoromethyl)-1,3,4-thiadiazol-2-yl]thiourea;

N-[2-methoxy-4-(2-methoxyethoxy)phenyl]-N′-[5-(trifluoromethyl)-1,3,4-thiadiazol-2-yl]urea;

N-(4-ethoxy-2-nitrophenyl)-N′-[3-(trifluoromethyl)isoxazol-5-yl]thiourea;

N-(4-hydroxy-2-methylphenyl)-N′-[4-(trifluoromethyl)-1,3-thiazol-2-yl]thiourea;

N-(3-chloro-4-methoxyphenyl)-N′-[5-(trifluoromethyl)-1,3,4-thiadiazol-2-yl]urea;

N-(5-chloro-2-methoxyphenyl)-N′-[5-(trifluoromethyl)-1,3,4-thiadiazol-2-yl]urea;

N-(4-methoxy-2-nitrophenyl)-N′-[5-(trifluoromethyl)-1,3,4-thiadiazol-2-yl]urea;

N-(4-ethoxy-2-nitrophenyl)-N′-[5-(trifluoromethyl)-1,3,4-thiadiazol-2-yl]urea;

N-(4-methoxy-2-methylphenyl)-N′-[5-(trifluoromethyl)-1,3,4-thiadiazol-2-yl]urea;

N-(2,6-dimethoxypyridin-3-yl)-N′-[5-(trifluoromethyl)-1,3,4-thiadiazol-2-yl]urea;

N-(4-hydroxyphenyl)-N′-[5-(trifluoromethyl)-1,3,4-thiadiazol-2-yl]urea;

N-(5-ethoxypyridin-2-yl)-N′-[5-(trifluoromethyl)-1,3,4-thiadiazol-2-yl]urea;

N-(4-ethoxy-2-morpholin-4-ylphenyl)-N′-[5-(trifluoromethyl)-1,3,4-thiadiazol-2-yl]urea;

tert-butyl 4-{5-ethoxy-2-[({[5-(trifluoromethyl)-1,3,4-thiadiazol-2-yl]amino}carbonyl) amino]phenyl}piperazine-1-carboxylate;

N-(2-chloro-6-methoxy-pyridin-3-yl)-N′-[5-(trifluoromethyl)-1,3,4-thiadiazol-2-yl]urea;

N-[6-methoxy-2-(methylthio)pyridin-3-yl]-N′-[5-(trifluoromethyl)-1,3,4-thiadiazol-2-yl]urea;

N-[6-methoxy-2-(methylsulfonyl)pyridin-3-yl]-N′-[5-(trifluoromethyl)-1,3,4-thiadiazol-2-yl]urea;

N-[2-methoxy-4-(2-methoxyethoxy)phenyl]-N′-(5-methylisoxazol-3-yl)urea;

N-(4-hydroxy-2-methylphenyl)-N′-[4-(trifluoromethyl)-1,3-thiazol-2-yl]urea;

N-(4-hydroxyphenyl)-N′-[4-(trifluoromethyl)-1,3-thiazol-2-yl]urea;

N-(5-hydroxy-2-methylphenyl)-N′-[4-(trifluoromethyl)-1,3-thiazol-2-yl]urea;

N-(3-hydroxy-2-methylphenyl)-N′-[4-(trifluoromethyl)-1,3-thiazol-2-yl]urea;

N-(6-cyanopyridin-3-yl)-N′-(5-fluoro-2,4-dimethoxyphenyl)urea;

N-(4-hydroxy-2-methylphenyl)-N′-[3-(trifluoromethoxy)phenyl]urea;

N-(4-hydroxy-2-methylphenyl)-N′-[4-(pentafluoroethyl)-1,3-thiazol-2-yl]urea;

N-(5-bromo-2,4-dimethoxyphenyl)-N′-(6-cyanopyridin-3-yl)urea;

N-(5-chloro-2,4-dimethoxyphenyl)-N′-[3-(trifluoromethyl)isoxazol-5-yl]urea;

N-(5-fluoro-2,4-dimethoxyphenyl)-N′-[3-(trifluoromethyl)isoxazol-5-yl]urea;

N-(2,6-dimethoxypyridin-3-yl)-N′-[3-(trifluoromethyl)isoxazol-5-yl]urea;

N-(4-ethoxy-2-nitrophenyl)-N′-[3-(trifluoromethyl)isoxazol-5-yl]urea;

N-(5-chloro-2,4-dimethoxyphenyl)-N′-(5-chloroisoxazol-3-yl)urea;

N-(5-chloro-4-methoxy-2-nitrophenyl)-N′-(5-methylisoxazol-3-yl )urea;

N-(5-fluoro-4-methoxy-2-nitrophenyl)-N′-(5-methylisoxazol-3-yl)urea;

N-[5-chloro-4-methoxy-2-(methylthio)phenyl]-N′-(5-methylisoxazol-3-yl)urea;

N-(5-chloro-2,4-dimethoxyphenyl)-N′-[5-(fluoromethyl)isoxazol-3-yl]urea;

N-(2,6-dimethoxypyridin-3-yl)-N′-[5-(fluoromethyl)isoxazol-3-yl]urea;

N-(5-fluoro-2,4-dimethoxyphenyl)-N′-[5-(fluoromethyl)isoxazol-3-yl]urea;

N-(5-bromo-2,4-dimethoxyphenyl)-N′-[5-(fluoromethyl)isoxazol-3-yl]urea;

N-(4-ethoxy-2-nitrophenyl)-N′-[5-(fluoromethyl)isoxazol-3-yl]urea;

N-(2,4-dimethoxy-5-methylphenyl)-N′-[5-(fluoromethyl)isoxazol-3-yl]urea;

N-(5-bromo-2,4-dimethoxyphenyl)-N′-[5-(hydroxymethyl)isoxazol-3-yl]urea;

N-(5-fluoro-2,4-dimethoxyphenyl)-N′-(5-isopropylisoxazol-3-yl)urea;

N-(5-chloro-2,4-dimethoxyphenyl)-N′-(5-isopropylisoxazol-3-yl)urea;

N-(5-bromo-2,4-dimethoxyphenyl)-N′-(5-isopropylisoxazol-3-yl)urea;

N-(2,6-dimethoxypyridin-3-yl)-N′-(5-isopropylisoxazol-3-yl)urea;

N-(2,4-dimethoxy-5-methylphenyl)-N′-(5-isopropylisoxazol-3-yl)urea;

N-(5-fluoro-2,4-dimethoxyphenyl)-N′-[5-(methoxymethyl)isoxazol-3-yl]urea;

N-(5-chloro-2,4-dimethoxyphenyl)-N′-[5-(methoxymethyl)isoxazol-3-yl]urea;

N-(5-bromo-2,4-dimethoxyphenyl)-N′-[5-(methoxymethyl)isoxazol-3-yl]urea;

N-(4-ethoxy-2-nitrophenyl)-N′-[5-(methoxymethyl)isoxazol-3-yl]urea;

N-(5-chloro-2,4-dimethoxyphenyl)-N′-(5-cyclopropylisoxazol-3-yl)urea;

N-(5-cyclopropylisoxazol-3-yl)-N′-(5-fluoro-2,4-dimethoxyphenyl)urea;

N-(5-bromo-2,4-dimethoxyphenyl)-N′-(5-cyclopropylisoxazol-3-yl)urea;

N-(5-cyclopropylisoxazol-3-yl)-N′-(2,4-dimethoxy-5-methylphenyl)urea;

N-(5-cyclopropylisoxazol-3-yl)-N′-(4-ethoxy-2-nitrophenyl)urea;

N-(5-cyclopropylisoxazol-3-yl)-N′-(2,6-dimethoxypyridin-3-yl)urea;

N-(5-chloro-2,4-dimethoxyphenyl)-N′-(5-ethylisoxazol-3-yl)urea;

N-(5-ethylisoxazol-3-yl)-N′-(5-fluoro-2,4-dimethoxyphenyl)urea;

N-(5-bromo-2,4-dimethoxyphenyl)-N′-(5-ethylisoxazol-3-yl)urea;

N-(2,4-dimethoxy-5-methylphenyl)-N′-(5-ethylisoxazol-3-yl)urea;

N-(2,6-dimethoxypyridin-3-yl)-N′-(5-ethylisoxazol-3-yl)urea;

N-(4-ethoxy-2-nitrophenyl)-N′-(5-ethylisoxazol-3-yl)urea;

N-(5-amino-2,4-dimethoxyphenyl)-N′-[5-(trifluoromethyl)-1,3,4-thiadiazol-2-yl]urea;

N-(5-azido-2,4-dimethoxyphenyl)-N′-[5-(trifluoromethyl)-1,3,4-thiadiazol-2-yl]urea;

N-(5-iodo-2,4-dimethoxyphenyl)-N′-[5-(trifluoromethyl)-1,3,4-thiadiazol-2-yl]urea;

N-(4-amino-2-methoxyphenyl)-N′-[5-(trifluoromethyl)-1,3,4-thiadiazol-2-yl]urea;

N-(4-methoxy-2-methylphenyl)-N′-[4-(pentafluoroethyl)-1,3-thiazol-2-yl]urea;

N-(5-chloro-2,4-dimethoxyphenyl)-N′-[5-(trifluoromethyl)isoxazol-3-yl]urea;

N-(4-hydroxyphenyl)-N′-(5-isopropyl-1,3-thiazol-2-yl)urea;

N-(4-ethoxyphenyl)-N′-(5-methyl-1,3-thiazol-2-yl)urea;

N-[4-(4-chlorophenyl)-1,3-thiazol-2-yl]-N′-(4-iodophenyl)urea;

N-(2,6-dimethylphenyl)-N′-(6-fluoro-1,3-benzothiazol-2-yl)urea;

N-(4-chloro-1,3-benzothiazol-2-yl)-N′-(2-methoxyphenyl)urea;

N-(5-bromo-1,3-thiazol-2-yl)-N′-(2-ethoxyphenyl)urea;

N-(5-bromo-1,3-thiazol-2-yl)-N′-(2,4-dimethoxyphenyl)urea;

N-(5-chloro-1,3-thiazol-2-yl)-N′-(4-methoxy-2-methylphenyl)urea;

N-(5-bromo-1,3-thiazol-2-yl)-N′-(4-methoxy-2-methylphenyl)urea;

N-(5-chloro-2,4-dimethoxyphenyl)-N′-(1,3-thiazol-2-yl)urea;

N-(4-methoxy-2-methylphenyl)-N′-(4-methyl-1,3-thiazol-2-yl)urea;

N-(4-chloro-1,3-benzothiazol-2-yl)-N′-(4-methoxy-2-methylphenyl)urea;

ethyl 2-({[(4-methoxy-2-methylphenyl)amino]carbonyl}amino)-4-phenyl-1,3-thiazole-5-carboxylate;

ethyl 2-[({[4-(butoxycarbonyl)phenyl]amino}carbonyl)amino]-4-phenyl-1,3-thiazole-5-carboxylate;

N-(5-bromo-1,3-thiazol-2-yl)-N′-(4-ethoxyphenyl)urea;

N-[2-methoxy-4-(tetrahydrofuran-3-yloxy)phenyl]-N′-[3-(trifluoromethyl)isoxazol-5-yl]urea;

N-[2-methoxy-4-(oxetan-3-yloxy)phenyl]-N′-[5-(trifluoromethyl)-1,3,4-thiadiazol-2-yl]urea;

N-[2-methoxy-4-(oxetan-3-yloxy)phenyl]-N′-[3-(trifluoromethyl)isoxazol-5-yl]urea;

N-[2-methoxy-4-(tetrahydrofuran-3-yloxy)phenyl]-N′-[5-(trifluoromethyl)-1,3,4-thiadiazol-2-yl]urea;

N-[2-methoxy-4-(tetrahydrofuran-3-yloxy)phenyl]-N′-[3-(trifluoromethyl)isoxazol-5-yl]urea; or a pharmaceutically acceptable salt thereof.

The present invention includes, but is not limited to, the examples provided herein, the compounds identified in the tables provided herein and compounds named herein as the free base or a pharmaceutically acceptable salt there.

The present invention also includes isotopically labeled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine iodine, and chlorine, such as ²H, ³H, ¹³C, ¹¹C, ¹⁴C ¹⁵N ¹⁸O, ¹⁷O, ³¹P, ³²P, ³⁵S, ¹⁸F, ¹²³I, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances.

Isotopically labeled compounds of Formula I can generally be prepared by carrying out the synthetic procedures described herein by substituting an isotopically labeled reagent for a non-isotopically labeled reagent. Isotopically labeled reagents are described, for example, by Langstrom in Acta Chem. Scand. S37: 147 (1990). Introducing ¹¹C-labeled agonists of nAChR has been described in Dolle, Frederic, et al, J. Labelled Cps Radiopharm., 2001; 44: 785-795. For a general discussion of nuclear imaging, see, “Nuclear Imaging in Drug Discovery, Development, and Approval, H. D. Burns, et al. (Eds).

The present invention also includes compounds for use in photoaffinity labeling experiments. One technique for the biochemical characterization of receptors is photoaffinity labeling using a photolabile molecule, or probe, which binds with high affinity to a receptor and can be irreversibly incorporated into the receptor under the influence of ultraviolet light. In order to have an effective and useful photoaffinity probe, several requirements must be met. First, the probe must have good biological activity at the same target protein relative to the parent compounds of interest. Second, it must have a reactive group which can covalently bond to the target site upon photoactivation. For example, the azido group is chemically inert until photoactivated by UV light. Upon photolysis it generates a highly reactive nitrene which inserts into either the peptide backbone or the amino acid side chains of the protein to which it is bound. This insertion forms a covalent linkage between the photoprobe and the protein allowing it to be permanently tagged for identification.

Further aspects and embodiments of the invention may become apparent to those skilled in the art from a review of the following detailed description, taken in conjunction with the examples and the appended claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION OF INVENTION

Surprisingly, we have found that compounds of Formula I:

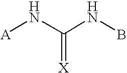

wherein X is O or S;

A is

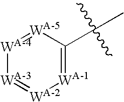

wherein each W^{A-1}, W^{A-2}, W^{A-3}, W^{A-4}, and W^{A-5} are independently N or CR_{A}, provided that no more than four of W^{A-1}, W^{A-2}, W^{A-3}, W^{A-4}, or W^{A-5} are simultaneously N;

Each R_{A} is independently H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₈, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂provides that at least one R_{A} is other than H;

or when two R_{A} are on adjacent carbon atoms, the two R_{A} may combine to form a 5-8-membered ring fused to the 6-membered ring, wherein the 5-8-membered ring is saturated or unsaturated having up to two heteroatoms selected from —O—, —S—, —N(R_{A-2})—, or —N═ and further having substitution where valency allows on the 5-8-membered ring with up to 2 substitutents independently selected from R_{A-1};

Each R_{A-1} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;

R_{A-2} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

B is a five or six-membered aromatic ring having up to 4 heteroatoms selected from —O—, —N(R_{B-3})—, ═N—, or —S—, wherein B is

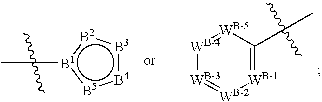

wherein B¹ is N, or C;

B², B³, B⁴, and B⁵ are independently N, O, S, C, provided that when valency allows, the N can have a third bond to R_{B-3}, and further provided that when valency allows, the C can have a fourth bond to R_{B-1};

Each R_{B-1} is independently H, halogen, alkyl, haloalkyl, substituted alkyl, cycloalkyl, halocycloalkyl, substituted cycloalkyl, alkenyl, haloalkenyl, substituted alkenyl, alkynyl, haloalkynyl, substituted alkynyl, heterocycloalkyl, haloheterocycloalkyl, substituted heterocycloalkyl, aryl, —CN, —N₃, —NO₂, —COR₁₀, —CO₂R₇, —CON(R₉)₂, —C(R₁₀)═NOR₇, —SCN, —OR₇, —N(R₉)₂, —SR₈, —SOR₈, —SO₂R₈, —SN(R₉)₂, —SON(R₉)₂, —SO₂N(R₉)₂;

when two R_{B-1} are on adjacent carbon atoms, the two R_{B-1} may combine to form a 5-7-membered ring fused to the 5 or 6 membered ring giving a fused-bicyclic-ring system; wherein the 5-7-membered ring is saturated or unsaturated having up to two heteroatoms selected from —O—, —S—, —N(R_{B-3})—, or —N═ and further having substitution where valency allows on the 5-7-membered ring with up to 2 substitutents independently selected from R_{B-2};

Each R_{B-2} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;

R_{B-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each W^{B-1}, W^{B-2}, W^{B-3}, W^{B-4}, and W^{B-5} are independently N or CR_{B-1}, provided that no more than 4 of W^{B-1}, W^{B-2}, W^{B-3}, W^{B-4}, or W^{B-5} are simultaneously N;

Halogen (used interchangeably with “halo”) is F, Br, Cl, or I;

Alkyl is both straight- and branched-chain moieties having from 1-6 carbon atoms, provided that when alkyl is a substituent off of B, then alkyl does not include t-butyl;

Lower alkyl is both straight- and branched-chain moieties having from 1-4 carbon atoms, provided that when lower alkyl is a substituent off of B, then lower alkyl does not include t-butyl;

Haloalkyl is an alkyl moiety having from 1-6 carbon atoms and having 1 to (2n+1) substituent(s) independently selected from F, Cl, Br, or I, where n is the maximum number of carbon atoms in the moiety;

Lower haloalkyl is lower alkyl having 1 to (2n+l) substituent(s) independently selected from F, Cl, Br, or I, where n is the maximum number of carbon atoms in the moiety;

Substituted alkyl is an alkyl moiety from 1-6 carbon atoms and having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃(O)₂R₃, phenyl, or substituted phenyl;

Lower substituted alkyl is lower alkyl having 0-3 substituents independently selected from F, Cl, Br, or I, and further having I substituent selected from —CN, —NO₂, —OR₃ —SR₃, —N(R₃)₂, —C(O)R₃ —C(O)N(R₃)₂, —NR₃(O)R₃ —S(O)₂N(R₃)₂, —NR₃(O)₂R₃, phenyl, or substituted phenyl;

Alkenyl is straight- and branched-chain moieties having from 2-6 carbon atoms and having at least one carbon-carbon double bond;

Lower alkenyl is straight- and branched-chain moieties having from 2-4 carbon atoms and having at least one carbon-carbon double bond;

Haloalkenyl is an alkenyl moiety having from 2-6 carbon atoms and having 1 to (2n-1) substituent(s) independently selected from F, Cl, Br, or I where n is the maximum number of carbon atoms in the moiety;

Lower haloalkenyl is lower alkenyl having 1 to (2n-1) substituent(s) independently selected from F, Cl, Br, or I where n is the maximum number of carbon atoms in the moiety;

Substituted alkenyl is an unsaturated alkenyl moiety having from 2-6 carbon atoms and having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substitute phenyl;

Lower substituted alkenyl is lower alkenyl having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Alkynyl is straight- and branched-chained moieties having from 2-6 carbon atoms and having at least one carbon-carbon triple bond;

Haloalkynyl is an alkynyl moiety having from 2-6 carbon atoms and having 1 to (2n-3) substituent(s) independently selected from F, Cl, Br, or I where n is the maximum number of carbon atoms in the moiety;

Substituted alkynyl is an unsaturated alkynyl moiety having from 2-6 carbon atoms and having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substitute phenyl;

Cycloalkyl is a cyclic alkyl moiety having from 3-6 carbon atoms;

Lower cycloalkyl is a cyclic alkyl moiety having from 3-4 carbon atoms;

Halocycloalkyl is a cyclic moiety having from 3-6 carbon atoms and having 1-4 substituents independently selected from F, Cl, Br, or I;

Substituted cycloalkyl is a cycloalkyl moiety from 3-6 carbon atoms and having 0-3 substituents independently selected from F, Cl, Br, or I and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Heterocycloalkyl is a cyclic moiety having 4-7 atoms with 1-2 atoms within the ring being —S—, —N(R₄)—, or —O—;

Haloheterocycloalkyl is a cyclic moiety having from 4-7 atoms with 1-2 atoms within the ring being —S—, —N(R4)—, or —O—, and having 1-4 substituents independently selected from F, Br, Cl, or I;

Substituted heterocycloalkyl is a cyclic moiety having from 4-7 atoms with 1-2 atoms within the ring being —S—, —N(R₄)—, or —O— and having 0-3 substituents independently selected from F, Br, Cl, or I, further having up to 2 oxo (═O) on separate carbon atoms with sufficient valency, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Aryl is phenyl, substituted phenyl, naphthyl, or substituted naphthyl;

Substituted phenyl is a phenyl either having 1-4 substituents independently selected from F, Cl, Br, or I, or having I substituent selected from R₅ and 0-3 substituents independently selected from F, Cl, Br, or I;

Substituted naphthyl is a naphthalene moiety either having 1-4 substituents independently selected from F, Cl, Br, or I, or having I substituent selected from R₅ and 0-3 substituents independently selected from F, Cl, Br, or I, where the substitution can be independently on either only one ring or both rings of said naphthalene moiety;

Each R₃ is independently H, alkyl, haloalkyl, alkenyl, haloalkenyl, alkynyl, haloalkynyl, cycloalkyl, halocycloalkyl, heterocycloalkyl, haloheterocycloalkyl, or phenyl optionally substituted with 0-3 halogens and 0-1 substituent selected from alkyl, —CF₃, —CN, —NH₂, —NO₂, and —OH;

R₄ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, or aryl;

R₅ is alkyl, alkenyl, alkynyl, cycloalkyl, heterocycloalkyl, haloalkyl, haloalkenyl, haloalkynyl, halocycloalkyl, haloheterocycloalkyl, —CN, —NO₂, —OR₃, —SR₃, —N(R₃)₂, —C(O)R₃, —C(O)N(R₃)₂, —NR₃C(O)R₃, —S(O)₂N(R₃)₂, —NR₃S(O)₂R₃aryl substituted with 1-4 substituent(s) independently selected from F, Cl, Br, I, or R₆, cycloalkyl substituted with 1-4 substituent(s) independently selected from F, Cl, Br, I, or R₆, or heterocycloalkyl substituted with 1-4 substituent(s) independently selected from F, Cl, Br, I, or R₆;

R₆ is —CF₃, —CN, —NO₂, —OR₃, —SR₃, —N(R₃)₂, —C(O)R₃, —C(O)N(R₃)₂, —NR₃C(O)R₃, —S(O)₂N(R₃)₂, or —NR₃S(O)₂R₃;

R₇ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₈ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each R₉is independently H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₁₀ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

or pharmaceutical composition, pharmaceutically acceptable salt, racemic mixture, or pure enantiomer thereof useful to treat any one of or combination of cognitive and attention deficit symptoms of Alzheimer's, neurodegeneration associated with diseases such as Alzheimer's disease, pre-senile dementia (mild cognitive impairment), senile dementia, schizophrenia or psychosis including the cognitive deficitis associated therewith, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, symptoms associated with pain; pain and inflammation (rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, and HIV-3; cytomegalovirus (CMV); influenza; adenovirus; a herpes virus (including HSV-1, HSV-2); or herpes zoster); cancer (multiple myeloma; acute and chronic myelogenous leukemia; or cancer-associated cachexia); diabetes (pancreatic beta cell destruction; or type I and type II diabetes); wound healing (healing burns, and wounds in general including from surgery); bone fracture healing; ischemic heart disease, or stable angina pectoris.

In another aspect, the invention includes a combination therapy for treating a mammal or preparing a medicament to treat a mammal as discussed herein. The compounds of Formula I and the other drug(s)/agent(s) can be administered simultaneously or at separate intervals. When administered simultaneously the compounds of Formula I and the other drug(s)/agent(s) can be incorporated into a single pharmaceutical composition. Alternatively, separate compositions, i.e., one containing compounds of Formula I and one or more containing the other drug(s), can be administered during a therapeutic interval.

A positive allosteric modulator of α7 nAChR will effectively activate the endogenous α7 nAChR if there is sufficient agonist in the brain to at least partially stimulate this receptor. Therefore, a positive allosteric modulator of α7 nAChR can be administered alone to treat the disease or conditions discussed herein. In certain diseases, however, it is possible that the full therapeutic efficacy of a positive allosteric modulator of α7 nAChR will be limited by suboptimal levels of agonist which in turn leads to a suboptimal activation of the endogenous α7 nAChR in the presence of a positive allosteric modulator. In such cases, the positive allosteric modulator of α7 nAChR is administered in combination with another agent that affects the level of agonist.

The present invention includes the intermediates, the processes to make them and the compounds of the present invention and salts thereof, pharmaceutical compositions containing the active compounds of the present invention, and methods to treat the identified diseases.

The compounds of Formula I exist in tautomeric or enantiomeric forms, all of which are included within the scope of the invention. The various optical isomers may be isolated by separation of a racemic mixture of the compounds using conventional techniques, e.g., fractional crystallization, or chiral HPLC. Alternatively, the individual enantiomers may be made by reaction of the appropriate optically active starting materials under reaction conditions which will not cause racemization.

Abbreviations which are well known to one of ordinary skill in the art may be used (e.g., “Ph” for phenyl, “Me” for methyl, “Et” for ethyl, “h” or “hr” or “hrs” for hour or hours, “min” for minute or minutes, and “rt” for room temperature).

All temperatures are in degrees Centigrade.

Room temperature is within the range of 15-25 degrees Celsius.

Pre-senile dementia is also known as mild cognitive impairment.

ACh refers to acetylcholine.

AChR refers to acetylcholine receptor.

nAChR refers to nicotinic acetylcholine receptor.

mAChR refers to muscarinic acetylcholine receptor.

PAM refers to positive allosteric modulator.

5HT₃R refers to the serotonin-type 3 receptor.

α-btx refers to α-bungarotoxin.

FLIPR refers to a device marketed by Molecular Devices, Inc. designed to precisely measure cellular fluorescence in a high throughput whole-cell assay. (Schroeder et. al., J. Biomolecular Screening, 1(2), p 75-80, 1996).

MLA refers to methyllycaconitine.

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to N,N-dimethylformamide.

EtOAc refers to ethyl acetate.

TMS refers to tetramethylsilane.

TEA refers to triethylamine.

DIEA refers to diisopropylethylamine.

NaHMDS refers to sodium bis(trimethylsilyl)amide.

KHMDS refers to potassium bis(trimethylsilyl)amide.

DMAP refers to 4-(dimethylamino)pyridine.

PTFE-lined cap is a cap made from polytetrafluoroethylene material.

Ether refers to diethyl ether.

50% saturated 1:1 NaCl/NaHCO₃ means a solution made by making a solution of 1:1 saturated NaCl/NaHCO₃ and adding an equal volume of water.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms.

Mammal denotes human and other mammals.

Brine refers to an aqueous saturated sodium chloride solution.

Equ means molar equivalents.

IR refers to infrared spectroscopy.

Lv refers to leaving groups within a molecule, including Cl, OH, or mixed anhydride.

Parr refers to the name of the company who sells the jars used for conducting reactions under pressure.

PSI means pound per square inch.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. [M+H]⁺ refers to an ion composed of the parent plus a proton. [M−H]⁻ refers to an ion composed of the parent minus a proton. [M+Na]⁺ refers to an ion composed of the parent plus a sodium ion. [M+K]⁺ refers to an ion composed of the parent plus a potassium ion. EI refers to electron impact. ESI refers to electrospray ionization. CI refers to chemical ionization. FAB refers to fast atom bombardment.

Non-inclusive examples of heterocycloalkyl include, but are not limited to, oxetano, tetrahydrofurano, tetrahydropyrano, pyrrolidino, piperidino, piperazino, morpholino, thiomorpholino, pyrazolo, 1,1-dioxidothietano, 1,1-dioxidothio-morpholino, azetidino, azetidinono, oxindolo, dihydroimidazolo, and pyrrolidinono.

The compounds of the present invention are useful in treating, or preparing medicaments to treat, diseases or disorders as described herein in mammals. Typically, the mammal is a human being, but the compounds of the present invention can be used to treat, or to prepare medicaments to treat, other mammals, such as food animals (e.g., cows, pigs, sheep, goats, deer, poultry, etc.), companion animals (e.g., dogs, cats, horses, birds, and fish), or other mammals. The compounds may be administered in their native form, or with a pharmaceutically acceptable excipient. The compounds may also be administered as a pharmaceutically acceptable salt.

Compounds of the present invention may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N, N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydroiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

By the term “effective amount” of a compound as provided herein is meant a nontoxic but sufficient amount of the compound(s) to provide the desired effect. As pointed out below, the exact amount required will vary from subject to subject, depending on the species, age, and general condition of the subject, the severity of the disease that is being treated, the particular compound(s) used, the mode of administration, and the like. Thus, it is not possible to specify an exact “effective amount.” However, an appropriate effective amount may be determined by one of ordinary skill in the art using only routine experimentation.

The amount of therapeutically effective compound(s) that is administered and the dosage regimen for treating a disease condition with the compounds and/or compositions of this invention depends on a variety of factors, including the age, weight, sex and medical condition of the subject, the severity of the disease, the route and frequency of administration, and the particular compound(s) employed, and thus may vary widely. The compositions contain well know carriers and excipients in addition to a therapeutically effective amount of compounds of Formula I. The pharmaceutical compositions may contain active ingredient in the range of about 0.001 to 100 mg/kg/day for an adult, preferably in the range of about 0.01 to about 50 mg/kg/day for an adult. A total daily dose of about 1 to 1000 mg of active ingredient may be appropriate for an adult. The daily dose can be administered in one to four doses per day.

In addition to the compound(s) of Formula I, the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, EtOH, corn oil, cottonseed oil, peanut oil, sesame oil, benzyl alcohol, sodium chloride, and/or various buffers. Other adjuvants and modes of administration are well and widely known in the pharmaceutical art.

Compounds of the present invention can enhance the efficacy of agonists at nicotinic receptors, and, are, therefore, referred to as “positive allosteric modulators.” Cholinergic receptors normally bind the endogenous neurotransmitter ACh. AChRs in the mammalian central nervous system can be divided into mAChR and nAChR subtypes based on the agonist activities of muscarine and nicotine, respectively. The nAChRs are ligand-gated ion channels containing five subunits. Members of the nAChR gene family have been divided into two groups based on their sequences: α and β. Three of the α subunits (α7, α8, and α9) form functional receptors when expressed alone and presumably form homooligomeric receptors.

α7 nAChR is a ligand-gated Ca⁺⁺ channel formed by a homopentamer of α7 subunits. Previous studies have established that in the central nervous system α-btx binds selectively to this homopetameric, α7 nAChR subtype, and that α7 nAChR has a high affinity binding site for both α-btx and MLA. α7 nAChR is expressed at high levels in the hippocampus, ventral tegmental area and ascending cholinergic projections from nucleus basilis to thalamocortical areas. α7 nAChR agonists increase neurotransmitter release, and increase cognition, arousal, attention, learning and memory.

The serotonin type 3 receptor (5HT₃R) is a member of a superfamily of ligand-gated ion channels, which includes the muscle and neuronal nAChR, the glycine receptor, and the γ-aminobutyric acid type A receptor. Like the other members of this receptor superfamily, the 5HT₃R exhibits a sequence homology with α7 nAChR but functionally the two ligand-gated ion channels are very different. For example, α7 nAChR is rapidly desensitized, is highly permeable to calcium and is activated by acetylcholine and nicotine. 5HT₃R is desensitized slowly, is relatively impermeable to calcium and is activated by serotonin. The pharmacology of the α7 nAChR and 5HT₃R channels is very different. For example, Ondansetron, a highly selective 5HT₃R antagonist, has little activity at the α7 nAChR. The converse is also true. For example, GTS-21, a highly selective α7 nAChR agonist, has little activity at the 5HT₃R.

An allosteric transition state model of the nAChR involves at least a resting state (closed), an activated state (open), and a “desensitized” closed channel state (Changeux, J. and Edelstein, S. J., Curr. Opin. Neurobiolo. 2001 11(3): 369-77; Itier, V. and Bertrand, D., FEBS Lett 2001, 504(3): 118-25). Different nAChR ligands can, therefore, differentially stabilize the conformational state to which they preferentially bind. For example, the agonists ACh and (−)-nicotine drive the nAChR to a desensitized state.

Data from human and animal pharmacological studies establish that nicotinic cholinergic neuronal pathways control many important aspects of cognitive function including attention, learning and memory (Levin, E. D., Psychopharmacology, 108:417-31, 1992; Levin, E. D. and Simon B. B., Psychopharmacology, 138:217-30, 1998). For example, it is well known that nicotine increases cognition and attention in humans. ABT-418, a compound that activates α4β2 and α7 nAChR, improves cognition and attention in clinical trials of Alzheimer's disease and attention-deficit disorders (Potter, A. et. al., Psychopharnacology (Berl)., 142(4):334-42, March 1999; Wilens, T. E. et. al., Am. J. Psychiatry, 156(12):1931-7, December 1999). It is also clear that nicotine and selective but weak α7 nAChR agonists increase cognition and attention in rodents and non-human primates.

However, treatment with nicotinic receptor agonists which act at the same site as ACh is problematic because ACh not only activates, but also blocks receptor activity through processes which include desensitization and uncompetitive blockade (open-channel block). Forman & Miller (1988) Biophysical J. 54(1):149-158. Furthermore, prolonged activation may up regulate receptor expression and induce a long-lasting inactivation (Olale, F., et al., J. Pharmacol. Exp. Ther. 1997, 283(2):675-83; Kuryatov, A. et al., Eur. J. Pharmacol. 2000, 393(1-3):11-21; Kawai, H. and Berg, D. K., J. Neurochem. 2001, 78(6):1367-78; Buisson, B. and Bertrand, D., J. Neurosci. 2001, 21(6):1819-29). Therefore, agonists of nAChRs can be expected to reduce activity as well as enhance it. At nicotinic receptors in general, and, of particular note, at the α7-nicotinic receptor, desensitization limits the duration that the channel remains in the active state during agonist application.

The present invention provides a means to increase α7 nAChR function in the brain and other organs, tissues and cells of the body by making these receptors more sensitive to activation by an agonist, including, but not limited to, ACh which is the endogenous agonist. Galantamine, an alkaloid originally obtained from bulbs of snowdrops, is a weak cholinesterase inhibitor and is reported to be a positive allosteric modulator of some nicotinic receptors (Santos, M. D., et al, Mol. Pharmacol. 2002, 61(5):1222-1234). The advantage of this invention is that a drug that works as a PAM of the α7 nAChR will provide long-lasting therapeutic value and will have a minimal risk of loss of therapeutic efficacy because of receptor desensitization. A PAM will also be a relatively safe drug because it acts to amplify the actions of an endogenous neurotransmitter.

Schizophrenia is a complex multifactorial illness caused by genetic and non-genetic risk factors that produce a constellation of positive and negative symptoms. The positive symptoms include delusions and hallucinations and the negative symptoms include deficits in affect, attention, cognition and information processing. No single biological element has emerged as a dominant pathogenic factor in this disease. Indeed, it is likely that schizophrenia is a syndrome that is produced by the combination of many low penetrance risk factors. Pharmacological studies established that dopamine receptor antagonists are efficacious in treating the overt psychotic features (positive symptoms) of schizophrenia such as hallucinations and delusions. Clozapine, an “atypical” antipsychotic drug, is novel because it is effective in treating both the positive and some of the negative symptoms of this disease. Clozapine's utility as a drug is greatly limited because continued use leads to an increased risk of agranulocytosis and seizure. No other antipsychotic drug is effective in treating the negative symptoms of schizophrenia. This is significant because the restoration of cognitive functioning is the best predictor of a successful clinical and functional outcome of schizophrenic patients (Green, M. F., Am J Psychiatry, 153:321-30, 1996). By extension, it is clear that better drugs are needed to treat the cognitive disorders of schizophrenia in order to restore a better state of mental health to patients with this disorder.

One aspect of the cognitive deficit of schizophrenia can be measured by using the auditory event-related potential (P50) test of sensory gating. In this test, electroencepholographic (EEG) recordings of neuronal activity of the hippocampus are used to measure the subject's response to a series of auditory “clicks” (Adler, L. E. et. al., Biol. Psychiatry, 46:8-18, 1999). Normal individuals respond to the first click with greater degree than to the second click. In general, schizophrenics and schizotypal patients respond to both clicks nearly the same (Cullum, C. M. et. al., Schizophr. Res., 10:131-41, 1993). These data reflect a schizophrenic's inability to “filter” or ignore unimportant information. The sensory gating deficit appears to be one of the key pathological features of this disease (Cadenhead, K. S. et. al., Am. J. Psychiatry, 157:55-9, 2000). Multiple studies show that nicotine normalizes the sensory deficit of schizophrenia (Adler, L. E. et. al., Am. J. Psychiatry, 150:1856-61, 1993). Pharmacological studies indicate that nicotine's effect on sensory gating is via the α7 nAChR (Adler, L. E. et. al., Schizophr. Bull., 24:189-202, 1998). Indeed, the biochemical data indicate that schizophrenics have 50% fewer of α7 nAChR receptors in the hippocampus, thus giving a rationale to partial loss of α7 nAChR functionality (Freedman, R. et. al., Biol. Psychiatry, 38:22-33, 1995). Interestingly, genetic data indicate that a polymorphism in the promoter region of the α7 nAChR gene is strongly associated with the sensory gating deficit in schizophrenia (Freedman, R. et. al., Proc. Nat'l Acad. Sci. USA, 94(2):587-92, 1997; Myles-Worsley, M. et. al., Am. J. Med. Genet, 88(5):544-50, 1999). To date, no mutation in the coding region of the α7 nAChR has been identified. Thus, schizophrenics express the same α7 nAChR as non-schizophrenics.

Selective α7 nAChR agonists may be found using a functional assay on FLIPR (see WO 00/73431 A2). FLIPR is designed to read the fluorescent signal from each well of a 96 or 384 well plate as fast as twice a second for up to 30 minutes. This assay may be used to accurately measure the functional pharmacology of α7 nAChR and 5HT₃R. To conduct such an assay, one uses cell lines that expressed functional forms of the α7 nAChR using the α7/5-HT₃ channel as the drug target and cell lines that expressed functional 5HT₃R. In both cases, the ligand-gated ion channel was expressed in SH-EP1 cells. Both ion channels can produce robust signal in the FLIPR assay.

A positive allosteric modulator of α7 nAChR will effectively activate the endogenous α7 nAChR if there is sufficient agonist in the brain to at least partially stimulate this receptor. Therefore, a positive allosteric modulator of α7 nAChR can be administered alone to treat the disease or conditions discussed herein.

In certain diseases, however, it is possible that the full therapeutic efficacy of a positive allosteric modulator of α7 nAChR will be limited by suboptimal levels of agonist which in turn leads to a suboptimal activation of the endogenous α7 nAChR in the presence of a positive allosteric modulator. For example but not limitation, it is well established that in Alzheimer's disease, there is a loss of ACh from the brains of the patients with this disease and this loss is correlated with disease progression. In this case, the primary role of combination therapy is to treat patients with therapeutic agents that directly activate the endogenous of α7 nAChR in combination with a positive allosteric modulator of α7 nAChR to achieve maximal efficacy. Thus, in Alzheimer's disease, it is likely that the full therapeutic efficacy of a positive allosteric modulator of α7 nAChR could be enhanced if combination therapy is used. This combination therapy applies to other diseases or conditions discussed herein where there is a loss of ACh. One of ordinary skill in the art would recognize for which disease or conditions this combination therapy would be useful.

The compounds of the present invention are α7 nAChR PAMs and may be used to treat a wide variety of diseases. For example, they may be used in treating schizophrenia, or psychosis.

Schizophrenia is a disease having multiple aspects. Currently available drugs are generally aimed at controlling the positive aspects of schizophrenia, such as delusions. One drug, Clozapine, is aimed at a broader spectrum of symptoms associated with schizophrenia. This drug has many side effects and is thus not suitable for many patients. Thus, there is a need for a drug to treat the cognitive and attention deficits associated with schizophrenia. Similarly, there is a need for a drug to treat the cognitive and attention deficits associated with schizoaffective disorders, or similar symptoms found in the relatives of schizophrenic patients.

Psychosis is a mental disorder characterized by gross impairment in the patient's perception of reality. The patient may suffer from delusions, and hallucinations, and may be incoherent in speech. His behavior may be agitated and is often incomprehensible to those around him. In the past, the term psychosis has been applied to many conditions that do not meet the stricter definition given above. For example, mood disorders were named as psychoses.

There are a variety of antipsychotic drugs. The conventional antipsychotic drugs include Chlorpromazine, Fluphenazine, Haloperidol, Loxapine, Mesoridazine, Molindone, Perphenazine, Pimozide, Thioridazine, Thiothixene, and Trifluoperazine. These drugs all have an affinity for the dopamine 2 receptor.

These conventional antipsychotic drugs have several side effects, including sedation, weight gain, tremors, elevated prolactin levels, akathisia (motor restlessness), dystonia and muscle stiffness. These drugs may also cause tardive dyskinesia. Unfortunately, only about 70% of patients with schizophrenia respond to conventional antipsychotic drugs. For these patients, atypical antipsychotic drugs are available.

Atypical antipsychotic drugs generally are able to alleviate positive symptoms of psychosis while also improving negative symptoms of the psychosis to a greater degree than conventional antipsychotics. These drugs may improve neurocognitive deficits. Extrapyramidal (motor) side effects are not as likely to occur with the atypical antipsychotic drugs, and thus, these atypical antipsychotic drugs have a lower risk of producing tardive dyskinesia. Finally these atypical antipsychotic drugs cause little or no elevation of prolactin. Unfortunately, these drugs are not free of side effects. Although these drugs each produce different side effects, as a group the side effects include: agranulocytosis; increased risk of seizures, weight gain, somnolence, dizziness, tachycardia, decreased ejaculatory volume, and mild prolongation of QTc interval.

In a combination therapy to treat multiple symptoms of diseases such as schizophrenia, the compounds of Formula I and the anti-psychotic drugs can be administered simultaneously or at separate intervals. When administered simultaneously the compounds of Formula I and the anti-psychotic drugs can be incorporated into a single pharmaceutical composition, e.g., a pharmaceutical combination therapy composition. Alternatively, two separate compositions, i.e., one containing compounds of Formula I and the other containing anti-psychotic drugs, can be administered simultaneously. Examples of anti-psychotic drugs, in addition to those listed above, include, but are not limited to, Thorazine, Mellaril, Trilafon, Navane, Stelazine, Permitil, Prolixin, Risperdal, Zyprexa, Seroquel, ZELDOX, Acetophenazine, Carphenazine, Chlorprothixene, Droperidol, Loxapine, Mesoridazine, Molindone, Ondansetron, Pimozide, Prochlorperazine, and Promazine.

A pharmaceutical combination therapy composition can include therapeutically effective amounts of the compounds of Formula I, noted above, and a therapeutically effective amount of anti-psychotic drugs. These compositions may be formulated with common excipients, diluents or carriers, and compressed into tablets, or formulated elixirs or solutions for convenient oral administration or administered by intramuscular intravenous routes. The compounds can be administered rectally, topically, orally, sublingually, or parenterally and maybe formulated as sustained relief dosage forms and the like.

When separately administered, therapeutically effective amounts of compositions containing compounds of Formula I and anti-psychotic drugs are administered on a different schedule. One may be administered before the other as long as the time between the two administrations falls within a therapeutically effective interval. A therapeutically effective interval is a period of time beginning when one of either (a) the compounds of Formula I, or (b) the anti-psychotic drugs is administered to a human and ending at the limit of the beneficial effect in the treatment of schizophrenia or psychosis of the combination of (a) and (b). The methods of administration of the compounds of Formula I and the anti-psychotic drugs may vary. Thus, either agent or both agents may be administered rectally, topically, orally, sublingually, or parenterally.

As discussed, the compounds of the present invention are α7 nAChR PAMs. Therefore, as another aspect of the present invention, the compounds of the present invention may be used to treat a variety of diseases including cognitive and attention deficit symptoms of Alzheimer's, neurodegeneration associated with diseases such as Alzheimer's disease, pre-senile dementia (also known as mild cognitive impairment), and senile dementia.

Alzheimer's disease has many aspects, including cognitive and attention deficits. Currently, these deficits are treated with cholinesterase inhibitors. These inhibitors slow the break down of acetylcholine, and thereby provide a general nonspecific increase in the activity of the cholinergic nervous system. Since the drugs are nonspecific, they have a wide variety of side effects. Thus, there is a need for a drug that stimulates a portion of the cholinergic pathways and thereby provides improvement in the cognitive and attention deficits associated with Alzheimer's disease without the side effects created by nonspecific stimulation of the cholinergic pathways.

Neurodegeneration is a common problem associated with diseases such as Alzheimer's disease. While the current drugs treat some of the symptoms of this disease, they do not control the underlying pathology of the disease. Accordingly, it would be desirable to provide a drug that can slow the progress of Alzheimer's disease.

Pre-senile dementia (mild cognitive impairment) concerns memory impairment rather than attention deficit problems and otherwise unimpaired cognitive functioning. Mild cognitive impairment is distinguished from senile dementia in that mild cognitive impairment involves a more persistent and troublesome problem of memory loss for the age of the patient. There currently is no medication specifically identified for treatment of mild cognitive impairment, due somewhat to the newness of identifying the disease. Therefore, there is a need for a drug to treat the memory problems associated with mild cognitive impairment.

Senile dementia is not a single disease state. However, the conditions classified under this name frequently include cognitive and attention deficits. Generally, these deficits are not treated. Accordingly, there is a need for a drug that provides improvement in the cognitive and attention deficits associated with senile dementia.

As discussed, the compounds of the present invention are α7 nAChR PAMs. Therefore, other diseases to be treated with compounds of the present invention include treating the cognitive and attention deficits as well as the neurodegeneration associated with attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, or symptoms associated with pain.

Attention deficit disorder is generally treated with methylphenidate, an amphetamine-like molecule that has some potential for abuse. Accordingly, it would be desirable to provide a drug that treats attention deficit disorder while having fewer side effects than the currently used drug.

Attention deficit hyperactivity disorder, otherwise known as ADHD, is a neurobehavioral disorder affecting 3-5% of all American children. ADHD concerns cognitive alone or both cognitive and behavioral actions by interfering with a person's ability to stay on a task and to exercise age-appropriate inhibition. Several types of ADHD exist: a predominantly inattentive subtype, a predominantly hyperactive-impulsive subtype, and a combined subtype. Treatment may include medications such as methylphenidate, dextroamphetamine, or pemoline, which act to decrease impulsivity and hyperactivity and to increase attention. No “cure” for ADHD currently exists. Children with the disorder seldom outgrow it; therefore, there is a need for appropriate medicaments.

The compounds of the present invention can also be combined with a psychostimulant or a monoamine reuptake inhibitor and optionally combined with an alpha7 nAChR agonist, especially when endogenous agonist is suboptimal.

By combination is meant the administration of the two agents within a month or two or less of each other, preferably within a week and more preferably at about the same time or within a day or two or less of each other.

In a combination therapy to treat ADHD, the compounds of Formula I and the psychostimulant or inhibitor can be administered simultaneously or at separate intervals. When administered simultaneously the compounds of Formula I and the psychostimulants or monoamine reuptake inhibitors can be incorporated into a single pharmaceutical composition, e.g., a pharmaceutical combination therapy composition. Alternatively, two separate compositions, i.e., one containing compounds of Formula I and the other containing the psychostimulants or monoamine reuptake inhibitors.

A pharmaceutical combination therapy composition can include therapeutically effective amounts of the compounds of Formula I, noted herein, and a therapeutically effective amount of the psychostimulants or monoamine reuptake inhibitors. While psychostimulants and monoamine reuptake inhibitors control the activity level, and attention, they are not effective in treating the co-morbid or concomitant deficit in cognitive that is associated with ADHD. The combination therapy will be more effective at treating this disease because a PAM and optionally an α7 nAChR agonist will treat the underlying cognitive dysfunction in the disorder and the other two classes of drugs will treat the behavioral problems associated with ADHD. The combined administration of the compounds of Formula I and optionally an agonist and the psychostimulant or monoamine reuptake inhibitor is expected to require less of the generally-prescribed dose for either agent when used alone and or is expected to result in less frequent administration of either or both agents. The skilled clinician may in fact learn that behavioral problems are secondary to the cognitive problems and can be treated with lower dosages of the inhibitors. Determining such dosages should be a routine determination by one skilled in the art of treating patients with ADHD.

Mood and affective disorders fall within a large group of diseases, including monopolar depression and bi-polar mood disorder. These diseases are treated with three major classes of compounds. The first group is the heterocyclic antidepressant (HCA's). This group includes the well-known tricyclic antidepressants. The second group of compounds used to treat mood disorders is the monoamine oxidase inhibitors (MAOI's) that are used in particular types of diseases. The third drug is lithium. Common side effects from HCA's are sedation and weight gain. In elderly patients with organic brain disease, the side effects of HCA's can also include seizures and behavioral symptoms. The main side effects from using MAOI's occur from dietary and drug interactions. Benign side effects from the use of lithium include, but are not limited to, weight gain, nausea, diarrhea, polyuria, polydipsia, and tremor. Toxic side effects from lithium can include persistent headache, mental confusion, and may reach seizures and cardiac arrhythmias. Therefore, agents with less side effects or interactions with food or other medications would be useful.

Depression is a mood disorder of varying lengths of normally several months to more than two years and of varying degrees of feelings involving sadness, despair, and discouragement. The heterocyclic antidepressants (HCA's) are currently the largest class of antidepressants, but monoamine oxidase inhibitors (MAOI's) are used in particular types of depression. Common side effects from HCA's are sedation and weight gain. In elderly patients with organic brain disease, the side effects from HCA's can also include seizures and behavioral symptoms. The main side effects from using MAOI's occur from dietary and drug interactions. Therefore, agents with fewer side effects would be useful.

Borderline personality disorder, although not as well known as bipolar disorder, is more common. People having borderline personality disorder suffer from a disorder of emotion regulation. Pharmaceutical agents are used to treat specific symptoms, such as depression or thinking distortions.

Acquired immune deficiency syndrome (AIDS) results from an infection with the human immunodeficiency virus (HIV). This virus attacks selected cells and impairs the proper function of the immune, nervous, and other systems. HIV infection can cause other problems such as, but not limited to, difficulties in thinking, otherwise known as AIDS dementia complex. Therefore, there is a need to drugs to relieve the confusion and mental decline of persons with AIDS.

Amyotrophic lateral sclerosis, also known as Lou Gehrig's disease, belongs to a class of disorders known as motor neuron diseases wherein specific nerve cells in the brain and spinal cord gradually degenerate to negatively affect the control of voluntary movement. Currently, there is no cure for amyotrophic lateral sclerosis although patients may receive treatment from some of their symptoms and although Riluzole has been shown to prolong the survival of patients. Therefore, there is a need for a pharmaceutical agent to treat this disease.

Traumatic brain injury occurs when the brain is damaged from a sudden physical assault on the head. Symptoms of the traumatic brain injury include confusion and other cognitive problems. Therefore, there is a need to address the symptoms of confusion and other cognitive problems.

Brain tumors are abnormal growths of tissue found inside of the skull. Symptoms of brain tumors include behavioral and cognitive problems. Surgery, radiation, and chemotherapy are used to treat the tumor, but other agents are necessary to address associated symptoms. Therefore, there is a need to address the symptoms of behavioral and cognitive problems.

Persons with Down's syndrome have in all or at least some of their cells an extra, critical portion of the number 21 chromosome. Adults who have Down's syndrome are known to be at risk for Alzheimer-type dementia. Currently, there is no proven treatment for Down's syndrome. Therefore, there is a need to address the dementia associated with Down's syndrome.

Genetically programmed degeneration of neurons in certain areas of the brain cause Huntington's disease. Early symptoms of Huntington's disease include mood swings, or trouble learning new things or remembering a fact. Most drugs used to treat the symptoms of Huntington's disease have side effects such as fatigue, restlessness, or hyperexcitability. Currently, there is no treatment to stop or reverse the progression of Huntington's disease. Therefore, there is a need of a pharmaceutical agent to address the symptoms with fewer side effects.

General anxiety disorder (GAD) occurs when a person worries about things such as family, health, or work when there is no reason to worry and is unable not to worry. About 3 to 4% of the U.S. population has GAD during the course of a year. GAD most often strikes people in childhood or adolescence, but can begin in adulthood, too. It affects women more often than men. Currently, treatment involves cognitive-behavioral therapy, relaxation techniques, and biofeedback to control muscle tension and medications such as benzodiazepines, imipramine, and buspirone. These drugs are effective but all have side-effect liabilities. Therefore, there is a need of a pharmaceutical agent to address the symptoms with fewer side effects.

Dementia with Lewy Bodies is a neurodegenerative disorder involving abnormal structures known as Lewy bodies found in certain areas of the brain. Symptoms of dementia with Lewy bodies include, but are not limited to, fluctuating cognitive impairment with episodic delirium. Currently, treatment concerns addressing the parkinsonian and psychiatric symptoms. However, medicine to control tremors or loss of muscle movement may actually accentuate the underlying disease of dementia with Lewy bodies. Therefore, there is a need of a pharmaceutical agent to treat dementia with Lewy bodies.

Age-related macular degeneration (AMD) is a common eye disease of the macula which is a tiny area in the retina that helps produce sharp, central vision required for “straight ahead” activities that include reading and driving. Persons with AMD lose their clear, central vision. AMD takes two forms: wet and dry. In dry AMD, there is a slow breakdown of light-sensing cells in the macula. There currently is no cure for dry AMD. In wet AMD, new, fragile blood vessels growing beneath the macula as dry AMD worsens and these vessels often leak blood and fluid to cause rapid damage to the macula quickly leading to the loss of central vision. Laser surgery can treat some cases of wet AMD. Therefore, there is a need of a pharmaceutical agent to address AMD.

Parkinson's disease is a neurological disorder characterized by tremor, hypokinesia, and muscular rigidity. Currently, there is no treatment to stop the progression of the disease. Therefore, there is a need of a pharmaceutical agent to address Parkinson's.

Tardive dyskinesia is associated with the use of conventional antipsychotic drugs. This disease is characterized by involuntary movements most often manifested by puckering of the lips and tongue and/or writhing of the arms or legs. The incidence of tardive dyskinesia is about 5% per year of drug exposure among patients taking conventional antipsychotic drugs. In about 2% of persons with the disease, tardive dyskinesia is severely disfiguring. Currently, there is no generalized treatment for tardive dyskinesia. Furthermore, the removal of the effect-causing drugs is not always an option due to underlying problems. Therefore, there is a need for a pharmaceutical agent to address the symptoms of tardive dyskinesia.

Pick's disease results from a slowly progressive deterioration of social skills and changes in personality with the resulting symptoms being impairment of intellect, memory, and language. Common symptoms include memory loss, lack of spontaneity, difficulty in thinking or concentrating, and speech disturbances. Currently, there is no specific treatment or cure for Pick's disease but some symptoms can be treated with cholinergic and serotonin-boosting antidepressants. In addition, antipsychotic medications may alleviate symptoms in FTD patients who are experiencing delusions or hallucinations. Therefore, there is a need for a pharmaceutical agent to treat the progressive deterioration of social skills and changes in personality and to address the symptoms with fewer side effects.

Post-traumatic stress disorder (PTSD) is a form of anxiety triggered by memories of a traumatic event that directly affected the patient or that the patient may have witnessed. The disorder commonly affects survivors of traumatic events including sexual assault, physical assault, war, torture, natural disasters, an automobile accident, an airplane crash, a hostage situation, or a death camp. The affliction also can affect rescue workers at an airplane crash or a mass shooting, someone who witnessed a tragic accident or someone who has unexpectedly lost a loved one. Treatment for PTSD includes cognitive-behavioral therapy, group psychotherapy, and medications such as Clonazepam, Lorazepam and selective serotonin-reuptake inhibitors such as Fluoxetine, Sertraline, Paroxetine, Citalopram and Fluvoxamine. These medications help control anxiety as well as depression. Various forms of exposure therapy (such as systemic desensitization and imaginal flooding) have all been used with PTSD patients. Exposure treatment for PTSD involves repeated reliving of the trauma, under controlled conditions, with the aim of facilitating the processing of the trauma. Therefore, there is a need for better pharmaceutical agents to treat Post traumatic stress disorder.

Dysregulation of food intake associated with eating disease, including bulemia nervosa and anorexia nervosa, involve neurophysiological pathways. Anorexia nervosa is hard to treat due to patients not entering or remaining in after entering programs. Currently, there is no effective treatment for persons suffering from severe anorexia nervosa. Cognitive behavioral therapy has helped patients suffering from bulemia nervosa; however, the response rate is only about 50% and current treatment does not adequately address emotional regulation. Therefore, there is a need for pharmaceutical agents to address neurophysiological problems underlying diseases of dysregulation of food intake.

Cigarette smoking has been recognized as a major public health problem for a long time. However, in spite of the public awareness of health hazard, the smoking habit remains extraordinarily persistent and difficult to break. There are many treatment methods available, and yet people continue to smoke. Administration of nicotine transdermally, or in a chewing gum base is common treatments. However, nicotine has a large number of actions in the body, and thus can have many side effects. It is clear that there is both a need and a demand of long standing for a convenient and relatively easy method for aiding smokers in reducing or eliminating cigarette consumption. A drug that could selectively stimulate only certain of the nicotinic receptors would be useful in smoke cessation programs.

Smoke cessation programs may involve oral dosing of the drug of choice. The drug may be in the form of tablets. However, it is preferred to administer the daily dose over the waking hours, by administration of a series of incremental doses during the day. The preferred method of such administration is a slowly dissolving lozenge, troche, or chewing gum, in which the drug is dispersed. Another drug in treating nicotine addiction is Zyban. This is not a nicotine replacement, as are the gum and patch. Rather, this works on other areas of the brain, and its effectiveness is to help control nicotine craving or thoughts about cigarette use in people trying to quit. Zyban is not very effective and effective drugs are needed to assist smokers in their desire to stop smoking. These drugs may be administered transdermally through the use of skin patches. In certain cases, the drugs may be administered by subcutaneous injection, especially if sustained release formulations are used.

Drug use and dependence is a complex phenomenon, which cannot be encapsulated within a single definition. Different drugs have different effects, and therefore different types of dependence. Drug dependence has two basic causes, that is, tolerance and physical dependence. Tolerance exists when the user must take progressively larger doses to produce the effect originally achieved with smaller doses. Physical dependence exists when the user has developed a state of physiologic adaptation to a drug, and there is a withdrawal (abstinence) syndrome when the drug is no longer taken. A withdrawal syndrome can occur either when the drug is discontinued or when an antagonist displaces the drug from its binding site on cell receptors, thereby counteracting its effect. Drug dependence does not always require physical dependence.

In addition drug dependence often involves psychological dependence, that is, a feeling of pleasure or satisfaction when taking the drug. These feelings lead the user to repeat the drug experience or to avoid the displeasure of being deprived of the drug. Drugs that produce strong physical dependence, such as nicotine, heroin and alcohol are often abused, and the pattern of dependence is difficult to break. Drugs that produce dependence act on the CNS and generally reduce anxiety and tension; produce elation, euphoria, or other pleasurable mood changes; provide the user feelings of increased mental and physical ability; or alter sensory perception in some pleasurable manner. Among the drugs that are commonly abused are ethyl alcohol, opioids, anxiolytics, hypnotics, cannabis (marijuana), cocaine, amphetamines, and hallucinogens. The current treatment for drug-addicted people often involves a combination of behavioral therapies and medications. Medications, such as methadone or LAAM (levo-alpha-acetyl-methadol), are effective in suppressing the withdrawal symptoms and drug craving associated with narcotic addiction, thus reducing illicit drug use and improving the chances of the individual remaining in treatment. The primary medically assisted withdrawal method for narcotic addiction is to switch the patient to a comparable drug that produces milder withdrawal symptoms, and then gradually taper off the substitute medication. The medication used most often is methadone, taken orally once a day. Patients are started on the lowest dose that prevents the more severe signs of withdrawal and then the dose is gradually reduced. Substitutes can be used also for withdrawal from sedatives. Patients can be switched to long-acting sedatives, such as diazepam or phenobarbital, which are then gradually reduced.

Gilles de la Tourette's Syndrome is an inherited neurological disorder. The disorder is characterized by uncontrollable vocal sounds called tics and involuntary movements. The symptoms generally manifest in an individual before the person is 18 years of age. The movement disorder may begin with simple tics that progress to multiple complex tics, including respiratory and vocal ones. Vocal tics may begin as grunting or barking noises and evolve into compulsive utterances. Coprolalia (involuntary scatologic utterances) occurs in 50% of patients. Severe tics and coprolalia may be physically and socially disabling. Tics tend to be more complex than myoclonus, but less flowing than choreic movements, from which they must be differentiated. The patient may voluntarily suppress them for seconds or minutes.

Currently simple tics are often treated with benzodiazepines. For simple and complex tics, Clonidine may be used. Long-term use of Clonidine does not cause tardive dyskinesia; its limiting adverse effect is hypotension. In more severe cases, antipsychotics, such as Haloperidol may be required, but side effects of dysphoria, parkinsonism, akathisia, and tardive dyskinesia may limit use of such antipsychotics. There is a need for safe and effective methods for treating this syndrome.

Glaucoma is within a group of diseases occurs from an increase in intraocular pressure causing pathological changes in the optical disk and negatively affects the field of vision. Medicaments to treat glaucoma either decrease the amount of fluid entering the eye or increase drainage of fluids from the eye in order to decrease intraocular pressure. However, current drugs have drawbacks such as not working over time or causing side effects so the eye-care professional has to either prescribe other drugs or modify the prescription of the drug being used. There is a need for safe and effective methods for treating problems manifesting into glaucoma.

Ischemic periods in glaucoma cause release of excitotoxic amino acids and stimulate inducible form of nitric oxide synthase (iNOS) leading to neurodegeneration. A PAM stimulates an agonist to affect the release of inhibitory amino acids such as GABA which will dampen hyperexcitablity. PAMs are also directly neuroprotective on neuronal cell bodies. Thus, PAMs have the potential to be neuroprotective in glaucoma.

Persons afflicted with pain often have what is referred to as the “terrible triad” of suffering from the pain, resulting in sleeplessness and sadness, all of which are hard on the afflicted individual and that individual's family. Pain can manifest itself in various forms, including, but not limited to, headaches of all severity, back pain, neurogenic, and pain from other ailments such as arthritis and cancer from its existence or from therapy to irradicate it. Pain can be either chronic (persistent pain for months or years) or acute (short-lived, immediate pain to inform the person of possible injury and need of treatment). Persons suffering from pain respond differently to individual therapies with varying degrees of success. There is a need for safe and effective methods for treating pain.

TNF-α is a pro-inflammatory cytokine secreted by a variety of cells, including monocytes and macrophages, in response to many inflammatory stimuli (e.g., lipopolysaccharide—LPS) or external cellular stresses (e.g., osmotic shock and peroxide). Elevated levels of TNF-α over basal levels have been implicated in mediating or exacerbating a number of diseases or conditions involving inflammation, pain, cancer, and diabetes. TNF-α is upstream in the cytokine cascade of inflammation. By decreasing levels of TNF-α, not only are levels of TNF-α minimized, but also elevated levels of other inflammatory and proinflammatory cytokines, such as IL-1, IL-6, and IL-8. TNF-α plays a role in head trauma, stroke, and ischemia. Shohami et al., J. Cereb. Blood Flow Metab., 14, 615 (1994). TNF-α promotes the infiltration of other cytokines (IL-1beta, IL-6) and also chemokines, which promote neutrophil infiltration into the infarct area. TNF-α plays a role in promoting certain viral life cycles and disease states associated with them; for instance, TNF-α secreted by monocytes induced elevated levels of HIV expression in a chronically infected T cell clone. Clouse et al., J. Immunol., 142, 431 (1989); Lahdevirte et al., Am. J. Med. 85, 289 (1988). TNF-α is associated with the HIV mediated states of cachexia due to cancer and muscle degradation.

TNF-α plays a role in pancreatic beta cell destruction and diabetes. Yoon J W, and Jun HS, Diabetologia, 44(3), 271-285 (2001). Pancreatic beta cells produce insulin which helps mediate blood-glucose homeostasis. Deterioration of pancreatic beta cells often accompanies type I diabetes. Pancreatic beta cell functional abnormalities may occur in patients with type II diabetes. Type II diabetes is characterized by a functional resistance to insulin. Further, type II diabetes is also often accompanied by elevated levels of plasma glucagon and increased rates of hepatic glucose production.

In rheumatoid arthritis, TNF-α induces synoviocytes and chondrocytes to produce collagenase and neutral proteases, which lead to tissue destruction within the arthritic joints. In a model of arthritis (collagen-induced arthritis (CIA) in rats and mice), intra-articular administration of TNF-α either prior to or after the induction of CIA led to an accelerated onset of arthritis and a more severe course of the disease. Brahn et al., Lymphokine Cytokine Res., 11, 253 (1992); and Cooper, Clin. Exp. Immunol., 898, 244 (1992). By reducing TNF-α levels, the resulting levels of synoviocytes and chondrocytes are also reduced to prevent or minimize the effects of rheumatoid arthritis.

The compounds of the present invention are useful to treat, or used to prepare a medicament used to treat, diseases or conditions where a mammal receives symptomatic relief from the decrease of levels of TNF-α; these diseases or conditions include, but are not limited to, any one or more or combination of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, or HIV-3; CMV; influenza, adenovirus, a herpes virus (including HSV-1, HSV-2); herpes zoster; multiple myeloma; acute and chronic myelogenous leukemia; cancer-associated cachexia; pancreatic beta cell destruction; type I or type II diabetes.

Some nicotinic receptors regulate vascular angiogenesis; for example, the binding of nicotine to the alpha-7 nAChR stimulates DNA synthesis and proliferation of vascular endothelial cells. Villablanca, supra. The compounds of the present invention are also useful to treat, or are used to prepare a medicament to treat, diseases or conditions where a mammal receives symptomatic relief from the stimulation of vascular angiogenesis; these diseases or conditions include, but not limited to, any one or more of the following: wound healing (healing burns, and wounds in general including from surgery), bone fracture healing, ischemic heart disease, and stable angina pectoris.

Compounds of Formula I can be prepared as shown in Scheme 1. The syntheses shown in the following schemes use intermediates where W^{A-1}, W^{A-2}, W^{A-3}, W^{A-4}, and W^{A-5} for the final compounds would be CR_{A}. One of ordinary skill in the art could make the corresponding compounds where up to four of W^{A-1}, W^{A-2}, W^{A-3}, W^{A-4} and W^{A-5} are N making non-critical changes to the methods discussed. The intermediates leading to the B moiety of Formula I can also be prepared by one of ordinary skill in the art with the methods discussed herein. The following discussion is not intended to limit the scope of the invention but is for exemplification only. Methods to synthesize ureas and thioureas of Formula I are well known to one skilled in the art. For example, aryl isocyanates or aryl isothiocyanates (II) or heteroaryl isocyanates or heteroaryl isothiocyanates (III) can be reacted with aminoheterocycles or anilines to provide the desired urea or thiourea using procedures described in J. Med. Chem. 1996, 39, 304; J. Med. Chem. 1999, 39, 4382; Pharmazie 1999, 54, 19; J. Chem. Soc. 1963, 40, 369; J. Chem. Soc. Perkin Trans. I 1977, 1616; and Synth. Commun. 2001, 31, 781. Alternatively, compounds of formula IV or V can be reacted with an aminoheterocycle or an aniline to provide the desired urea or thiourea using procedures described in J. Med. Chem. 1999, 39, 304; J. Med. Chem. (1995) 38, 855.

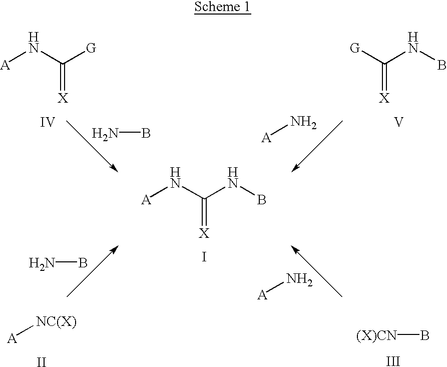

where G is 4-nitro-phenoxy, phenoxy, or imidazol-1-yl.

Compounds of Formula III can be prepared as shown in Scheme 2. Methods to synthesize isocyanates or isothiocyanates of Formula III are well known to one skilled in the art. For example, an aminoheterocycle can be reacted with excess phosgene (or phosgene equivalent) or thiophosgene in refluxing ethyl acetate to provide the heterocyclic isocyanate as described in U.S. Pat. No. 3,759,940. Alternatively, heterocyclic isocyanates III can be prepared from the corresponding carboxylic acid or acid derivative by treatment with an azide source such as sodium azide or diphenylphosphoryl azide (DPPA) followed by a Curtius-type rearrangement using procedures described in J. Org. Chem. 1985, 50, 5723; J. Org. Chem. 1997, 62, 3013. Compounds of Formula V can be synthesized using procedures well known to one skilled in the art (see DE 1816696; and Greene, T. W. and Wuts, P. G. M. “Protective Groups in Organic Synthesis”, 3rd Edition, p. 549, New York:Wiley, (1999)). The requisite aminoheterocycles or heterocyclic carboxylic acids can be obtained from commercial sources or can be synthesized by known procedures.

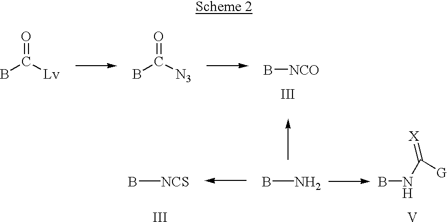

where G is as defined for Scheme 1 and Lv is OH, Cl, or —NH—NH₂.

It will be apparent to those skilled in the art that the aryl isocyanates or aryl isothiocyanates II can be obtained commercially or can be synthesized by known procedures. Compounds of Formula II can be prepared in a manner exactly analogous to the procedures used for the preparation of compounds of Formula III. The requisite substituted anilines can be purchased from commercial sources or prepared using procedures outlined in J. Org. Chem. 1997, 62, 6471. Alternatively, aryl isocyanates II can be prepared from the corresponding carboxylic acid or acid derivative by treatment with an azide source such as sodium azide or diphenylphosphoryl azide (DPPA) followed by a Curtius-type rearrangement using procedures described in Synth. Commun. 1993, 23, 335; or Heterocycles 1993, 36, 1305. Aryl isothiocyanates II can be prepared according to procedures in J. Org. Chem. 2000, 65, 6237. Compounds of Formula IV can be prepared in a manner exactly analogous to the procedures used for the preparation of compounds of Formula V. Scheme 3 depicts these transformations.

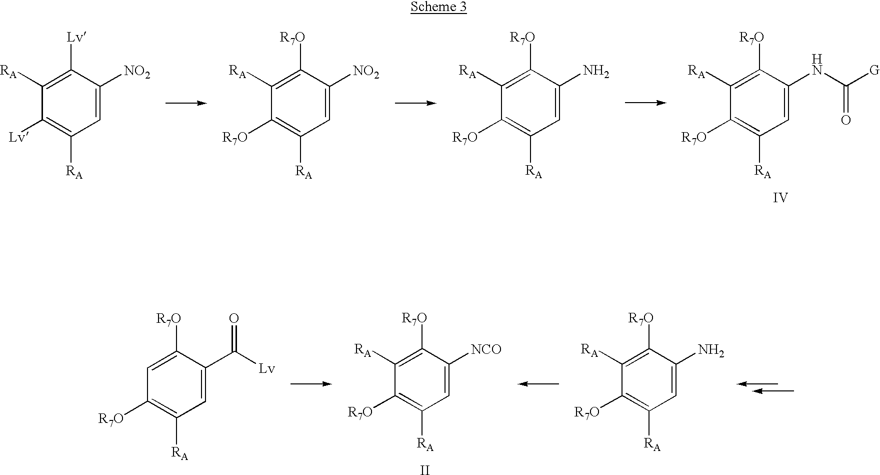

where G and Lv are as previously defined, and Lv′ is F, Cl, Br, SO₂Me.

The following examples are provided as examples and are not intended to limit the scope of this invention to only those provided examples and named compounds.

### METHOD A

### Example 1

N-(4-methoxy-2-methylphenyl)-N′-[3-(trifluoromethyl)phenyl]urea.

To a solution of the 4-methoxy-2-methylaniline (0.100 g, 0.73 mmol) in THF (5.0 ml) are added 3-(trifluoromethyl)phenylisocyanate (0.136 g, 0.73 mmol) and DMAP (0.0005 g, 0.04 mmol). The reaction mixture is stirred at 50° C. for 2 hr. The solution is concentrated under vacuum and the residue is crystallized from CH₃CN to give an off white solid 0.09 g (38%). HRMS (ESI) calcd for C₁₆H₁₅F₃N₂O₂+H 325.1164, found 325.1174.

The following compounds are made according to Method A, making non-critical variations. Examples 2-47 are made from an aminoheterocycle and an aryl isocyanate. For other examples, more details are provided for preparing the intermediates. If such details are not provided, the starting materials are readily available or can be made by one of ordinary skill in organic chemistry without undue experimentation.

### Example 2

N-(5-chloro-2,4-dimethoxyphenyl)-N′-isoxazol-3-ylurea. Yield 74%. HRMS (FAB) calculated for C₁₂H₁₂ClN₃O₄+H 298.0594, found 298.0595.

### Example 3

N-(5-chloro-2,4-dimethoxyphenyl)-N′-(5-methyl-1,3,4-thiadiazol-2-yl)urea. Yield 24%. MS (ESI) for C₁₂H₁₃ClN₄O₃S (M−H)⁻ m/z 327.

### Example 4

N-(5-chloro-2,4-dimethoxyphenyl)-N′-(5-methylisoxazol-3-yl)urea. Yield 75%. HRMS (FAB) calculated for C₁₃H₁₄ClN₃O₄+H 312.0751, found 312.0751.

### Example 5

N-(5-chloro-2,4-dimethoxyphenyl)-N′-(5-ethyl-1,3,4-thiadiazol-2-yl)urea. Yield 91%. MS (ESI) for C₁₃H₁₅ClN₄O₃S (M−H)⁻ m/z 341.

### Example 6

N-(5-chloro-2,4-dimethoxyphenyl)-N′-[5-(trifluoromethyl)-1,3,4-thiadiazol-2-yl]urea. Yield 72%. MS (ESI) for C₁₂H₁₀ClF₃N₄O₃S (M−H)⁻ m/z 381.

### Example 7

N-(5-chloro-2,4-dimethoxyphenyl)-N′-(5-cyclopropyl-1,3,4-thiadiazol-2-yl)urea. Yield 100%. HRMS (FAB) calculated for C₁₄H₁₅ClN₄O₃S+H 355.0631, found 355.0630.

### Example 8

N-(5-chloro-2,4-dimethoxyphenyl)-N′-[5-(difluoromethyl)-1,3,4-thiadiazol-2-yl]urea. Yield 83%. HRMS (FAB) calculated for C₁₂H₁₁ClF₂N₄O₃S+H 365.0287, found 365.0299.

### Example 9

N-(5-chloro-2,4-dimethoxyphenyl)-N′-(5-methoxy-1,3,4-thiadiazol-2-yl)urea. Yield 78%. HRMS (FAB) calculated for C₁₂H₁₃ClN₄O₄S+H 345.0424, found 345.0444.

### Example 10

N-(5-bromo-1,3,4-thiadiazol-2-yl)-N′-(5-chloro-2,4-dimethoxyphenyl)urea. Yield 77%. HRMS (FAB) calculated for C₁₁H₁₀BrClN₄O₃S+H 392.9424, found 392.9422.

### Example 11

N-(5-chloro-1,3,4-thiadiazol-2-yl)-N′-(5-chloro-2,4-dimethoxyphenyl)urea. Yield 67%. HRMS (FAB) calculated for C₁₁H₁₀Cl₂N₄O₃S+H 348.9929, found 348.9927.

### Example 12

N-(5-chloro-2,4-dimethoxyphenyl)-N′-[5-(2-phenylethyl)-1,3,4-thiadiazol-2-yl]urea. Yield 47%. HRMS (FAB) calculated for C₁₉H₁₉ClN₄O₃S+H 419.0945, found 419.0949.

### Example 13

N-(5-chloro-2,4-dimethoxyphenyl)-N′-(5-ethoxy-1,3,4-thiadiazol-2-yl)urea. Yield 91%. HRMS (FAB) calculated for C₁₃H₁₅ClN₄O₄S+H 359.0580, found 359.0590.

### Example 14

N-(5-chloro-2,4-dimethoxyphenyl)-N′-(1,3,4-thiadiazol-2-yl)urea. Yield 91%. HRMS (FAB) calculated for C₁₁H₁₁ClN₄O₃S+H 315.0319, found 315.0315.

### Example 15

N-(4-ethoxyphenyl)-N′-(5-methylisoxazol-3-yl)urea. Yield 41%. HRMS (FAB) calculated for C₁₃H₁₅N₃O₃+H 262.1191, found 262.1195.

### Example 16

N-(2,4-dimethoxyphenyl)-N′-(5-methylisoxazol-3-yl)urea. Yield 17%. HRMS (FAB) calculated for C₁₃H₁₅N₃O₄+H 278.1140, found 278.1152.

### Example 17

N-(2,4-dimethoxyphenyl)-N′-[5-(trifluoromethyl)-1,3,4-thiadiazol-2-yl]urea. Yield 43%. HRMS (FAB) calculated for C₁₂H₁₁F₃N₄O₃S+H 349.0582, found 349.0581.

### Example 18

N-(5-chloro-2-methoxyphenyl)-N′-(5-methylisoxazol-3-yl)urea. Yield 34%. HRMS (FAB) calculated for C₁₂H₁₂ClN₃O₃+H 282.0645, found 282.0638.

### Example 19

N-(5-chloro-2-methoxyphenyl)-N′-(5-methyl-1,3,4-thiadiazol-2-yl)urea. Yield 97%. HRMS (FAB) calculated for C₁₁H₁₁ClN₄O₂S+H 299.0369, found 299.0373.

### Example 20

N-(4-isopropoxyphenyl)-N′-[5-(trifluoromethyl)-1,3,4-thiadiazol-2-yl]urea. Yield 38%. HRMS (FAB) calculated for C₁₃H₁₃F₃N₄O₂S+H 347.0789, found 347.0786.

### Example 21

N-(2-ethoxyphenyl)-N′-[5-(trifluoromethyl)-1,3,4-thiadiazol-2-yl]urea. Yield 15%. HRMS (FAB) calculated for C₁₂H₁₁F₃N₄O₂S+H 332.0555, found 332.0547.

### Example 22

N-(4-ethoxyphenyl)-N′-[5-(trifluoromethyl)-1,3,4-thiadiazol-2-yl]urea. Yield 71%. HRMS (FAB) calculated for C₁₂H₁₁F₃N₄O₂S+H 333.0633, found 333.0637.

### Example 23

N-(4-methoxyphenyl)-N′-[5-(trifluoromethyl)-1,3,4-thiadiazol-2-yl]urea. Yield 36%. HRMS (FAB) calculated for C₁₁H₉F₃N₄O₂S+H 319.0476, found 319.0477.

### Example 24

N-(4-butoxyphenyl)-N′-[5-(trifluoromethyl)-1,3,4-thiadiazol-2-yl]urea. Yield 44%. HRMS (FAB) calculated for C₁₄H₁₅F₃N₄O₂S+360.0868, found 360.0855.

### Example 25

N-(2,3-dihydro-1,4-benzodioxin-6-yl)-N′-[5-(trifluoromethyl)-1,3,4-thiadiazol-2-yl]urea. Yield 70%. HRMS (FAB) calculated for C₁₂H₉F₃N₄O₃S+H 347.0425, found 347.0426.

### Example 26

N-(2,3-dihydro-1-benzofuran-5-yl)-N′-[5-(trifluoromethyl)-1,3,4-thiadiazol-2-yl]urea. Yield 13%. MS (ESI) for C₁₂H₉F₃N₄O₂S (M+H)⁺ m/z 331.2.

### Example 27

N-(4-ethyl-1,3-thiazol-2-yl)-N′-(4-methoxy-2-methylphenyl)urea.

4-Ethyl-1,3-thiazol-2-amine hydrobromide hydrate (0.1255 g, 0.553 mmol, prepared from thiourea and 1-bromo-2-butanone as described in Biotechnology and Bioengineering (Combinatorial Chemistry), 2000, 71(1), 9.) is partitioned between EtOAc and 1N NaOH. The layers are separated and the organic layer is dried (MgSO₄), filtered and concentrated to yield crude 4-ethyl-1,3-thiazol-2-amine (0.0706 g). Example 27 is obtained according Method A, making non-critical changes. Yield 70%. MS (ESI+) for C₁₄H₁₇N₃O₂S m/z 291.9 (M+H)⁺.

### Example 28

N-(5-chloro-2,4-dimethoxyphenyl)-N′-(1H-imidazol-2-yl)urea. MS (ESI) for C₁₂H₁₃ClN₄O₃ m/z 297.

### Example 29

N-(5-bromo-1,3-thiazol-2-yl)-N′-(5-chloro-2,4-dimethoxyphenyl)urea. Yield 5%. HRMS (ESI) calcd for C₁₂H₁₁BrClN₃O₃S+H 391.9472 found 391.9487.

### Example 30

N-(5-chloro-2,4-dimethoxyphenyl)-N′-(5-methyl-1,3-thiazol-2-yl)urea. Yield 90%. HRMS (ESI) calcd for C₁₃H₁₄ClN₃O₃S+H 328.0522, found 328.0532.

### Example 31

N-(5-chloro-2,4-dimethoxyphenyl)-N′-(3-methylisoxazol-5-yl)urea. Yield 14%. HRMS (ESI) calcd for C₁₃H₁₄ClN₃O₄+H 312.0751, found 312.0746.

### Example 32

N-(5-chloro-2,4-dimethoxyphenyl)-N′-(6-cyanopyridin-3-yl)urea. Yield 30%. HRMS (ESI) calcd for C₁₅H₁₃ClN₄O₃+H 333.0754, found 333.0752.

### Example 33

N-(5-chloro-2,4-dimethoxyphenyl)-N′-(5-chloro-1,3-thiazol-2-yl)urea. Yield 60%. HRMS calcd for C₁₂H₁₁Cl₂N₃O₃S+H 347.9976 found 347.9984.

### Example 34

N-(5-chloro-1,3-thiazol-2-yl)-N′-(2,4-dimethoxy-5-methylphenyl)urea. Yield 5%. HRMS calcd for C₁₃H₁₄ClN₃O₃S+H 328.0522 found 328.0518.

### Example 35

N-(5-bromo-2,4-dimethoxyphenyl)-N′-(5-chloro-1,3-thiazol-2-yl)urea. Yield 18%. HRMS calcd for C₁₂H₁₁BrClN₃O₃S+H 391.9472 found 391.9490.

### Example 36

N-(5-bromo-1,3-thiazol-2-yl)-N′-(5-fluoro-2,4-dimethoxyphenyl)urea. Yield 8%. HRMS calcd for C₁₂H₁₁BrFN₃O₃S+H 375.9767 found 375.9771.

### Example 37

N-(5-chloro-1,3-thiazol-2-yl)-N′-(5-fluoro-2,4-dimethoxyphenyl)urea. Yield 13%. HRMS calcd for C₁₂H₁₁ClFN₃O₃S+H 332.0272 found 332.0284.

### Example 38

N-(3-chloro-4-fluorophenyl)-N′-(5-methylisoxazol-3-yl)urea. Yield 66%. HRMS calcd for C₁₁H₉ClFN₃O₂+H 270.0445 found 270.0441.

### Example 39

N-(3-chloro-4-fluorophenyl)-N′-[5-(trifluoromethyl)-1,3,4-thiadiazol-2-yl]urea. Yield 61%. HRMS calcd for C₁₀H₅ClF₄N₄OS+H 340.9887 found 340.9896.

### Example 40

N-(2-ethoxyphenyl)-N′-(5-methylisoxazol-3-yl)urea. Yield 20%. HRMS calcd for C₁₃H₁₅N₃O₃+H 262.1191 found 262.1182.

### Example 41

N-(2-methoxyphenyl)-N′-[5-(trifluoromethyl)-1,3,4-thiadiazol-2-yl]urea. Yield 51%. HRMS calcd for C₁₁H₉F₃N₄O₂S+H 319.0476 found 319.0459.

### Example 42

N-(2-fluoro-4-methoxyphenyl)-N′-[5-(trifluoromethyl)-1,3,4-thiadiazol-2-yl]urea. Yield 34%. HRMS calcd for C₁₁H₈F₄N₄O₂S+H 337.0382 found 337.0374.

### Example 43

N-(5-chloro-2,4-dimethoxyphenyl)-N′-(5-mercapto-1,3,4-thiadiazol-2-yl)urea. Yield 23%. HRMS calcd for C₁₁H₁₁ClN₄O₃S₂+H 347.0039 found 347.0045.

### Example 44

N-(4,5-dimethoxy-2-methylphenyl)-N′-[5-(trifluoromethyl)-1,3,4-thiadiazol-2-yl]urea. Yield 35%. HRMS calcd for C₁₃H₁₃F₃N₄O₃S+H 363.0739 found 363.0739.

### Example 45

N-(4-hydroxy-2-nitrophenyl)-N′-[5-(trifluoromethyl)-1,3,4-thiadiazol-2-yl]urea. Yield 70%. HRMS calcd for C₁₀H₆F₃N₅O₄S+H 350.0171 found 350.0162.

### Example 46

N-(2,6-dimethoxypyridin-3-yl)-N′-(5-methyl-1,3-thiazol-2-yl)urea. Yield 55%. HRMS calcd for C₁₂H₁₄N₄O₃S+H 295.0861 found 295.0865.

### Example 47

N-(4-ethoxy-2-nitrophenyl)-N′-(5-methylisoxazol-3-yl)urea. Yield 78%. HRMS calcd for C₁₃H₁₄N₄O₅+H 323.0814 found 323.0806.

### Example 48

N-(4-methoxy-2-methylphenyl)-N′-[2-(trifluoromethyl) pyridin-4-yl]urea.

N-(4-methoxy-2-methylphenyl)-N′-[2-(trifluoromethyl)pyridin-4-yl]urea (from 1-isocyanato-4-methoxy-2-methylbenzene and 2-(trifluoromethyl)pyridin-4-amine, (see J. Med. Chem. 1993, 733-46) is prepared by following Method A, making non-critical modifications. The resulting residue is purified by silica gel chromatography (50% EtOAc/n-heptane) followed by trituration with EtOAc to afford a white solid 0.036 g (9%). MS (EI) m/z (rel intensity) 325 (M+,76), 163 (99), 162 (51), 148 (43), 137 (48), 122 (95), 120 (28), 93 (57), 66 (33), 65 (26).

### Example 50

N-(5-fluoro-2,4-dimethoxyphenyl)-N′-(5-methylisoxazol-3-yl)urea.

Sodium methoxide, prepared from sodium (1.43 g, 62.12 mmol) and MeOH, is added dropwise to solution of 1,2,4-trifluoro-5-nitrobenzene (5.0 g, 28.24 mmol) dissolved in 80 mL MeOH cooled to 0° C. The mixture is warmed to RT, heated under reflux for 2 h, then cooled. After standing at RT for 60 h, 1M citric acid is added and the solvent is removed in vacuo. The residue is taken up in ether, washed successively with 1M citric acid and brine, dried (MgSO₄), filtered and concentrated to provide 0.55 g of 1-fluoro-2,4-dimethoxy-5-nitrobenzene as a solid. The aqueous layer is further extracted with EtOAc. The organic layers are dried (MgSO₄), filtered and concentrated to provide 4.16 g of additional solid; overall yield 4.71 g (83% yield). MS for C₈H₈FNO₄ (ESI) (M+H)⁺ m/z 202.

1-Fluoro-2,4-dimethoxy-5-nitrobenzene (4.55 g, 22.62 mmol) and a catalytic amount of 10% Pd/C (200 mg) are mixed in EtOH/EtOAc and shaken on a Parr hydrogenator apparatus in the presence of 40 psi H₂. After 45 min, the mixture is filtered through a pad of Celite. The solvent is removed in vacuo to afford 3.84 g (100% yield) of 5-fluoro-2,4-dimethoxyaniline as a white solid. MS for C₈H₁₀FNO₂ (ESI) (M+H)⁺ m/z 172.

5-Fluoro-2,4-dimethoxyaniline (1.5 g, 8.76 mmol), dissolved in 60 mL EtOAc, is added dropwise over 1 h to excess phosgene (24.7 mL, 20% solution in toluene) dissolved in 25 mL EtOAc. The solution is heated under reflux for 30 min, cooled and concentrated in vacuo to provide 1.74 g (100% yield) of 1-fluoro-5-isocyanato-2,4-dimethoxybenzene as a solid. MS for C₉H₈FNO₃ (ESI) (M+H)⁺ m/z 198. Example 50 is obtained from 1-fluoro-5-isocyanato-2,4-dimethoxybenzene and 3-amino-5-methylisoxazole according to Method A, making non-critical variations. Yield 68%. HRMS (FAB) calculated for C₁₃H₁₄FN₃O₄+H 296.1046, found 296.1039.

The following compounds are made from 1-fluoro-5-isocyanato-2,4-methoxybenzene according to Method A, making non-critical variations.

### Example 51

N-(5-fluoro-2,4-dimethoxyphenyl)-N′-(5-methyl-1,3-thiazol-2-yl)urea. Yield 94%. HRMS (FAB) calculated for C₁₃H₁₄FN₃O₃S+H 312.0818, found 312.0827.

### Example 52

N-(5-fluoro-2,4-dimethoxyphenyl)-N′-(5-methyl-1,3,4-thiadiazol-2-yl)urea. Yield 87%. HRMS (FAB) calculated for C₁₂H₁₃FN₄O₃S+H 313.0771, found 313.0782.

### Example 53

N-(5-fluoro-2,4-dimethoxyphenyl)-N′-[5-(trifluoromethyl)-1,3,4-thiadiazol-2-yl]urea. Yield 76%. HRMS (FAB) calculated for C₁₂H₁₀F₄N₄O₃S+H 366.0410, found 366.0400.

### Example 75

N-(5-bromo-2,4-dimethoxyphenyl)-N′-(5-methylisoxazol-3-yl)urea.

Bromine (10.6 g, 66.3 mmol) dissolved in 40 mL CHCl₃ is added dropwise to a mixture of K₂CO₃ (20.8 g, 150.5 mmol) and 2,4-dimethoxyaniline (10.0 g, 65.3 mmol) dissolved in 60 mL CHC₃. After stirring the mixture for 4 h, water is added. The layers are separated and the organic layer is washed with water, dried (MgSO₄), filtered and concentrated. The residue is purified by column chromatography (Biotage Flash 40M column, 20% EtOAc/hexanes) and recrystallized from hexanes to provide 3.41 g (23% yield) of 5-bromo-2,4-dimethoxyaniline. MS for C₈H₁₀BrNO₂ (ESI) (M+H)⁺ m/z 232 and 234.

An EtOAc solution of 5-bromo-2,4-dimethoxyaniline (0.822 g, 3.54 mmol) is added to excess phosgene (5.0 mL, 20% solution in toluene). The solution is heated under reflux for 30 min, cooled and concentrated in vacuo. The residue is recrystallized from heptane to provide 0.81 g (89% yield) of 1-bromo-5-isocyanato-2,4-dimethoxybenzene.

Example 75 is obtained from 1-bromo-5-isocyanato-2,4-dimethoxybenzene according to Method A, making non-critical variations. Yield 40%. HRMS (FAB) calculated for C₁₃H₁₄BrN₃O₄+H 356.0246, found 356.0256.

The following compounds are made from 1-bromo-5-isocyanato-2,4-dimethoxybenzene according to Method A, making non-critical variations.

### Example 76

N-(5-bromo-2,4-dimethoxyphenyl)-N′-(5-methyl-1,3-thiazol-2-yl)urea. Yield 71%. HRMS (FAB) calculated for C₁₃H₁₄BrN₃O₃S+H 372.0018, found 371.0011.

### Example 77

N-(5-bromo-2,4-dimethoxyphenyl)-N′-(5-methyl-1,3,4-thiadiazol-2-yl)urea. Yield 60%. HRMS (FAB) calculated for C₁₂H₁₃BrN₄O₃S+H 372.9970, found 372.9984.

### Example 78

N-(5-bromo-2,4-dimethoxyphenyl)-N′-[5-(trifluoromethyl)-1,3,4-thiadiazol-2-yl]urea. Yield 61%. HRMS (FAB) calculated for C₁₂H₁₀BrF₃N₄O₃S+H 426.9688, found 426.9695.

### Example 90

N-(3,5-difluoro-2,4-dimethoxyphenyl)-N′-(5-methylisoxazol-3-yl)urea.

Sodium methoxide, prepared from sodium (1.29 g, 56.39 mmol) and 25 mL MeOH, is added dropwise to solution of 1,2,3,4-tetrafluoro-5-nitrobenzene (5.0 g, 25.63 mmol) dissolved in 5 mL MeOH cooled to 0° C. The mixture is warmed to RT and stirred overnight. The mixture is heated at 80° C. for 4 h, then cooled to RT. A 1M solution of citric acid (20 mL) is added and the solvent is removed in vacuo. The residue is diluted in EtOAc, washed with 1M citric acid. The aqueous layer is further extracted with EtOAc. The combined organic layers are washed with brine, dried (MgSO₄), filtered and concentrated to provide 5.60 g (100% yield) of 1,3-difluoro-2,4-dimethoxy-5-nitrobenzene a light yellow oil. MS for C₈H₇F₂NO₄ (ESI) (M+H)⁺ m/z 220.

1,3-Difluoro-2,4-dimethoxy-5-nitrobenzene (4.96 g, 22.63 mmol) and a catalytic amount of 10% Pd/C (200 mg) are mixed in EtOH/EtOAc and shaken on a Parr hydrogenator apparatus in the presence of 40 psi H₂. After 2 h, the mixture is filtered through a pad of Celite. The solvent is removed in vacuo to afford 4.32 g (100% yield) of 3,5-difluoro-2,4-dimethoxyaniline as a brown oil. MS for C₈H₉F₂NO₂ (ESI) (M+H)⁺ m/z 190.

3,5-Difluoro-2,4-dimethoxyaniline (2.17 g, 11.58 mmol), dissolved in 50 mL EtOAc, is added dropwise over 1 h to excess phosgene (42.9 mL, 20% solution in toluene) dissolved in 25 mL EtOAc. The solution is heated under reflux for 30 min, cooled and concentrated in vacuo to provide 2.40 g (96% yield) of 1,3-difluoro-5-isocyanato-2,4-dimethoxybenzene as a brown oil. MS for C₉H₇F₂NO₃ (ESI) (M+H)⁺ m/z 216. Example 90 is obtained from 1,3-difluoro-5-isocyanato-2,4-dimethoxybenzene and 3-amino-5-methylisoxazole according to Method A, making non-critical changes. Yield 54%. HRMS (FAB) calculated for C₁₃H₁₃F₂N₃O₄+H 314.0952, found 314.0949.

The following compounds are made from 1,3-difluoro-5-isocyanato-2,4-dimethoxybenzene according to Method A, making non-critical variations.

### Example 91

N-(3,5-difluoro-2,4-dimethoxyphenyl)-N′-[5-(trifluoromethyl)-1,3,4-thiadiazol-2-yl]urea. Yield 47%. HRMS (FAB) calculated for C₁₂H₉F₅N₄O₃S+H 385.0394, found 385.0390.

### Example 92

N-(3,5-difluoro-2,4-dimethoxyphenyl)-N′-(5-methyl-1,3-thiazol-2-yl)urea. Yield 75%. HRMS (FAB) calculated for C₁₃H₁₃F₂N₃O₃S+H 330.0724, found 330.0731.

### Example 93

N-(3,5-difluoro-2,4-dimethoxyphenyl)-N′-(5-methyl-1,3,4-thiadiazol-2-yl)urea. Yield 67%. HRMS (FAB) calculated for C₁₂H₁₂F₂N₄O₃S+H 331.0676, found 331.0690.

### Example 100

N-[2,4-dimethoxy-5-(trifluoromethyl)phenyl]-N′-[5-(trifluoromethyl)-1,3,4-thiadiazol-2-yl]urea.

Sodium methoxide, prepared from sodium (0.92 g, 40.13 mmol) and 50 mL MeOH, is added dropwise to solution of 1,5-dichloro-2-nitro-4-(trifluoromethyl)benzene (5.0 g, 19.23 mmol) dissolved in 25 mL MeOH cooled to 0° C. The mixture is warmed to RT and stirred overnight. The mixture is heated under reflux for 8 h, then cooled to RT. An additional amount of sodium methoxide (19.23 mmol) is added and the reaction is heated under reflux for 4 h. The reaction is cooled, quenched with 1M citric and concentrated in vacuo. The residue is diluted in EtOAc and washed with 1M citric acid. The aqueous layer is extracted with EtOAc. The combined organic layers are washed with brine, dried (MgSO₄), filtered and concentrated to provide 4.69 g (97% yield) of 1,5-dimethoxy-2-nitro-4-(trifluoromethyl)benzene. MS for C₉H₈F₃NO₄ (ESI) (M+H)⁺ m/z 252.

1,5-Dimethoxy-2-nitro-4-(trifluoromethyl)benzene (4.24 g, 16.88 mmol) and a catalytic amount of 10% Pd/C (200 mg) are mixed in EtOH/EtOAc and shaken on a Parr hydrogenator apparatus in the presence of H₂ (40 psi). After 1 h, the mixture is filtered through a pad of Celite. The solvent is removed in vacuo to afford 3.47 g (93% yield) of 2,4-dimethoxy-5-(trifluoromethyl)aniline as a light brown solid. MS for C₉H₁₀F₃NO₂ (ESI) (M+H)⁺ m/z 222.

2,4-Dimethoxy-5-(trifluoromethyl)aniline (1.80 g, 8.14 mmol), dissolved in 50 mL EtOAc, is added dropwise over 1 h to excess phosgene (34.4 mL, 20% solution in toluene) dissolved in 30 mL EtOAc. The solution is heated under reflux for 30 min, cooled and concentrated in vacuo to provide 1.70 g (85% yield) of 1-isocyanato-2,4-dimethoxy-5-(trifluoromethyl)benzene. MS for C₁₀H₈F₃NO₃ (ESI) (M+H)⁺ m/z 248. Example 100 is obtained from 1-isocyanato-2,4-dimethoxy-5-(trifluoromethyl)benzene and 5-(trifluoromethyl)-1,3,4-thiadiazol-2-amine according to Method A, making non-critical changes. Yield 49%. HRMS (FAB) calculated for C₁₃H₁₀F₆N₄O₃S+H 417.0456, found 417.0461.

The following compounds are made from 1-isocyanato-2,4-dimethoxy-5-(trifluoromethyl)benzene according to Method A, making non-critical variations.

### Example 101

N-[2,4-dimethoxy-5-(trifluoromethyl)phenyl]-N′-(5-methylisoxazol-3-yl)urea. Yield 69%. HRMS (FAB) calculated for C₁₄H₁₄F₃N₃O₄+H 346.1014, found 346.1024.

### Example 102

N-[2,4-dimethoxy-5-(trifluoromethyl)phenyl]-N′-(5-methyl-1,3-thiazol-2-yl)urea. Yield 79%. HRMS (FAB) calculated for C₁₄H₁₄F₃N₃O₃S+H 362.0786, found 362.0783.

### Example 103

N-[2,4-dimethoxy-5-(trifluoromethyl)phenyl]-N′-(5-methyl-1,3,4-thiadiazol-2-yl)urea. Yield 79%. HRMS (FAB) calculated for C₁₃H₁₃F₃N₄O₃S+H 363.0739, found 363.0738.

### Example 110

N-(5-chloro-2,4-diethoxyphenyl)-N′-(5-methylisoxazol-3-yl)urea.

Sodium ethoxide, prepared from sodium (1.1 2 g, mmol) and EtOH, is added dropwise to solution of 1,2,4-trichloro-5-nitrobenzene (5.0 g, 22.08 mmol) dissolved EtOH cooled to 0° C. The mixture is warmed to RT, heated under reflux for 2 h, then cooled. After standing at RT for 60 h, 1M citric acid is added and the solvent is removed in vacuo. The residue is diluted in CHCl₃ and washed successively with 1M citric acid and brine. The organic layer is dried (MgSO₄), filtered and concentrated to provide 5.53 g (>100% yield) of 1-chloro-2,4-diethoxy-5-nitrobenzene sufficiently pure for further use. MS for C₁₀H₁₂ClNO₄ (ESI) (M+H)⁺ m/z 246.

1-Chloro-2,4-diethoxy-5-nitrobenzene (g, 16.16 mmol) and a catalytic amount of 10% Pd/C (200 mg) are mixed in EtOH/EtOAc and shaken on a Parr hydrogenator apparatus in the presence of 40 psi H₂. After 90 min, the mixture is filtered through a pad of Celite and the solvent is removed in vacuo. The residue is purified by chromatography (Biotage 40S, 10% EtOAc/hexanes) followed by recrystallization from EtOAc/hexanes to afford 1.01 g (29% yield) of 5-chloro-2,4-diethoxyaniline. MS for C₁₀H₁₄ClNO₂ (ESI) (M+H)⁺ m/z 216 and 218.

5-Chloro-2,4-diethoxyaniline (0.895 g, 4.15 mmol), dissolved in 5 mL EtOAc, is added dropwise over 1 h to excess phosgene (17.2 mL, 20% solution in toluene) dissolved in 25 mL EtOAc. The solution is heated under reflux for 30 min, cooled and concentrated in vacuo to provide 1.17 g (100% yield) of 1-chloro-5-isocyanato-2,4-diethoxybenzene. MS for C₁₁H₁₂ClNO₃ (ESI) (M+H)⁺ m/z 242. Example 110 is obtained from 1-chloro-5-isocyanato-2,4-diethoxybenzene and 3-amino-5-methylisoxazole according to Method A, making non-critical changes. Yield 66%. HRMS (FAB) calculated for C₁₅H₁₈ClN₃O₄+H 340.1064, found 340.1061.

The following compounds are made starting from corresponding aminoheterocycle and the appropriate alcohol according to the procedure of Example 110, making non-critical variations.

### Example 111

N-(5-chloro-2,4-diethoxyphenyl)-N′-(5-methyl-1,3-thiazol-2-yl)urea. Yield 60%. HRMS (FAB) calculated for C₁₅H₁₈ClN₃O₃S+H 356.0836, found 356.0832.

### Example 112

N-(5-chloro-2,4-diethoxyphenyl)-N′-(5-methyl-1,3,4-thiadiazol-2-yl)urea. Yield 85%. HRMS (FAB) calculated for C₁₄H₁₇ClN₄O₃S+H 357.0788, found 357.0775.

### Example 113

N-(5-chloro-2,4-diethoxyphenyl)-N′-[5-(trifluoromethyl)-1,3,4-thiadiazol-2-yl]urea. Yield 60%. C₁₄H₁₄ClF₃N₄O₃S+H 410.0427, found 410.0444,

### Example 114

N-(5-chloro-2,4-dipropoxyphenyl)-N′-[5-(trifluoromethyl)-1,3,4-thiadiazol-2-yl]urea. Yield 25%. HRMS (FAB) calculated for C₁₆H₁₈ClF₃N₄O₃S+H 439.0818, found 439.0812.

### Example 115

N-(5-chloro-2,4-dipropoxyphenyl)-N′-(5-methylisoxazol-3-yl)urea. Yield 31%. HRMS (FAB) calculated for C₁₇H₂₂ClN₃O₄+H 368.1377, found 368.1374.

### Example 116

N-(5-chloro-2,4-diisopropoxyphenyl)-N′-(5-methyl-1,3,4-thiadiazol-2-yl)urea. Yield 18%. HRMS (FAB) calculated for C₁₆H₂₁ClN₄O₃S+H 385.1101, found 385.1094.

### Example 117

N-(5-chloro-2,4-diisoprooxyphenyl)-N′-[5-(trifluoromethyl)-1,3,4-thiadiazol-2-yl]urea. Yield 12%. HRMS (FAB) calculated for C₁₆H₁₈ClF₃N₄O₃S+H 439.0818, found 439.0805.

### Example 118

N-(5-chloro-2,4-diisoprooxyphenyl)-N′-(5-methylisoxazol-3-yl)urea. Yield 39%. HRMS (FAB) calculated for C₁₇H₂₂ClN₃O₄+H 368.1377, found 368.1361.

### Example 120

N-(2-chloro-4-methoxy-5-methylphenyl)-N′-(5-methylisoxazol-3-yl)urea.

Sodium methoxide, prepared from sodium (1.33 g, mmol) and MeOH, is added dropwise to solution of 1-fluoro-5-chloro-2-methyl-4-nitrobenzene (5.0 g, 26.37 mmol) dissolved MeOH cooled to 0° C. The mixture is warmed to RT, heated under reflux for 1.5 h. The reaction mixture is cooled, quenched with IM citric acid and concentrated in vacuo. The residue is diluted in EtOAc and 1M citric acid. The aqueous layer is extracted with EtOAc. The combined organic layers are washed with brine, dried (MgSO₄), filtered and concentrated to provide 5.32 g (100% yield) of 1-chloro-5-methoxy-3-methyl-2-nitrobenzene. ¹H NMR (CDCl₃, 400 MHz) δ 7.85, 6.89, 3.92, 2.23.

1-Chloro-5-methoxy-3-methyl-2-nitrobenzene (2.28 g, 11.31 mmol) and sodium dithionite (6.10 g, 35.06 mmol) are mixed in 5 mL THF and 12 mL water and heated under reflux. After 16 h, additional THF is added along with 5 mL EtOH. After 6 h of heating, the reaction mixture is cooled and the solvent is removed in vacuo. The residue is redissolved in aqueous EtOH, treated with sodium dithionite (6. 10 g, 35.06 mmol) and heated for 60 h. The reaction mixture is cooled. The solid is filtered, washed with water and dried (vacuum oven) to afford 0.683 g (35% yield) of 2-chloro-4-methoxy-5-methylaniline. MS for C₈H₁₀ClNO (ESI) (M+H)⁺ m/z 172.

2-Chloro-4-methoxy-5-methylaniline (0.58 g, 3.38 mmol), dissolved in 15 mL EtOAc, is added dropwise over 1 h to excess phosgene (13.4 mL, 20% solution in toluene) dissolved in 25 mL EtOAc. The solution is heated under reflux for 30 min, cooled and concentrated in vacuo to provide 0.655 g (98% yield) of 2-chloro-1-isocyanato-4-methoxy-5-methylbenzene. ¹H NMR (CDCl₃, 400 MHz) δ 6.88, 6.82, 3.80, 2.14. Example 120 is obtained from 2-chloro-1-isocyanato-4-methoxy-5-methylbenzene and 3-amino-5-methylisoxazole according to Method A, making non-critical changes. Yield 62%. HRMS (FAB) calculated for C₁₃H₁₄ClN₃O₃+H 296.0802, found 296.0813.

The following compounds are made from 2-chloro-1-isocyanato-4-methoxy-5-methylbenzene according to Method A, making non-critical variations:

### Example 121

N-(2-chloro-4-methoxy-5-methylphenyl)-N′-(5-methyl-1,3-thiazol-2-yl)urea. Yield 74%. HRMS (FAB) calculated for C₁₃H₁₄ClN₃O₂S+H 312.0573, found 312.0579.

### Example 122

N-(2-chloro-4-methoxy-5-methylphenyl)-N′-(5-methyl-1,3,4-thiadiazol-2-yl)urea. Yield 75%. HRMS (FAB) calculated for C₁₂H₁₃ClN₄O₂S+H 313.0526, found 313.0528.

### Example 123

N-(2-chloro-4-methoxy-5-methylphenyl)-N′-[5-(trifluoromethyl)-1,3,4-thiadiazol-2-yl]urea. Yield 58%. HRMS (FAB) calculated for C₁₂H₁₀ClF₃N₄O₂S+H 367.0243, found 367.0253.

### Example 125

N-(2,4-dimethoxy-5-methylphenyl)-N′-(5-methylisoxazol -3-yl)urea.

Sodium methoxide, prepared from sodium (0.32 g, mmol) and MeOH, is added dropwise to a solution of 1-chloro-5-methoxy-3-methyl-2-nitrobenzene (2.77 g, 13.74 mmol) dissolved MeOH cooled to 0° C. The mixture is warmed to RT and then heated under reflux until reaction is complete (as judged by TLC). The reaction mixture is cooled, quenched with 1M citric acid and concentrated in vacuo. The residue is diluted in EtOAc and 1M citric acid. The aqueous layer is extracted with EtOAc. The combined organic layers are washed with brine, dried (MgSO₄), filtered and concentrated to provide 2.94 g (>100% yield) of 1,5-dimethoxy-2-methyl-4-nitrobenzene sufficiently pure for further use. ¹H NMR (CDCl₃, 400 MHz) δ 7.85, 6.45, 3.98, 3.93, 2.16.

1,5-Dimethoxy-2-methyl-4-nitrobenzene (2.74 g, mmol) and a catalytic amount of 10% Pd/C (150 mg) are mixed in EtOH/EtOAc and shaken on a Parr hydrogenator apparatus in the presence of 40 psi H₂. After 60 min, the mixture is filtered through a pad of Celite. The solvent is removed in vacuo to afford 2.35 g (100% yield) of 2,4-dimethoxy-5-methylaniline. MS for C₉H₁₃NO₂ (ESI) (M+H)⁺ m/z 168.

2,4-Dimethoxy-5-methylaniline (1.03 g, 6.14 mmol), dissolved in 125 mL EtOAc, is added dropwise over 1 h to excess phosgene (27.7 mL, 20% solution in toluene) dissolved in EtOAc. The solution is heated under reflux for 30 min, cooled and concentrated in vacuo to provide 1.21 g (100% yield) of 1-isocyanato-2,4-dimethoxy-5-methylbenzene. MS for C₁₀H₁₁NO₃ (ESI) (M+H)⁺ m/z 194. Example 125 is obtained from 1-isocyanato-2,4-dimethoxy-5-methylbenzene and 3-amino-5-methylisoxazole according to Method A, making non-critical changes. Yield 39%. HRMS (FAB) calculated for C₁₄H₁₇N₃O₄+H 292.1297, found 292.1288.

The following compounds are made from 1-isocyanato-2,4-dimethoxy-5-methylbenzene according to Method A, making non-critical variations.

### Example 126

N-(2,4-dimethoxy-5-methylphenyl)-N′-(5-methyl-1,3-thiazol-2-yl)urea. Yield 59%. HRMS (FAB) calculated for C₁₄H₁₇N₃O₃S+H 308.1069, found 308.1082.

### Example 127

N-(2,4-dimethoxy-5-methylphenyl)-N′-(5-methyl-1,3,4-thiadiazol-2-yl)urea. Yield 72%. HRMS (FAB) calculated for C₁₃H₁₆N₄O₃S+H 309.1021, found 309.1032.

### Example 128

N-(2,4-dimethoxy-5-methylphenyl)-N′-[5-(trifluoromethyl)-1,3,4-thiadiazol-2-yl]urea. Yield 47%. HRMS (FAB) calculated for C₁₃H₁₃F₃N₄O₃S+H 363.0739, found 363.0740.

### Example 129

N-(4-ethoxy-2-methoxy-5-methylphenyl)-N′-[5-(trifluoromethyl)-1,3,4-thiadiazol-2-yl]urea.

Sodium ethoxide, prepared from sodium (0.12 g, 5.26 mmol) and EtOH, is added dropwise to solution of 1-fluoro-5-chloro-2-methyl-4-nitrobenzene (1.0 g, 5.26 mmol) dissolved in EtOH and cooled to 0° C. The mixture is warmed to RT, heated under reflux for 24 h. The reaction mixture is cooled, quenched with 1M citric acid and concentrated in vacuo. The residue is diluted in CHCl₃ and 1M citric acid. The aqueous layer is extracted with CHCl₃. The combined organic layers are washed with brine, dried (MgSO₄), filtered and concentrated to provide 1.16 g (100% yield) of 1-chloro-5-ethoxy-3-methyl-2-nitrobenzene. MS for C₉H₁₀ClNO₃ (ESI) (M+H)^{+m/z} 216.

Sodium methoxide, prepared from sodium (0.200 g, 8.6 mmol) and MeOH, is added dropwise to a solution of 1-chloro-5-ethoxy-3-methyl-2-nitrobenzene (0.93 g, 4.3 mmol) dissolved MeOH cooled to 0° C. The mixture is warmed to RT, heated under reflux until reaction is complete (as judged by TLC). The reaction mixture is cooled, quenched with 1M citric acid and concentrated in vacuo. The residue is diluted in EtOAc and lM citric acid. The aqueous layer is extracted with EtOAc. The combined organic layers are dried (MgSO₄), filtered and concentrated. The residue is purified by chromatography (Biotage 40S, 15% EtOAc/hexanes) to provide 0.31 g (32% yield) of 1-ethoxy-5-methoxy-2-methyl-4-nitrobenzene as a white solid. MS for C₁₀H₁₃NO₄ (ESI) (M+H)⁺ m/z 212.

1-Ethoxy-5-methoxy-2-methyl-4-nitrobenzene (0.31 g, 1.4 mmol) and a catalytic amount of 10% Pd/C (150 mg) are mixed in EtOH/EtOAc and shaken on a Parr hydrogenator apparatus in the presence of 40 psi H₂. After 24 h, the mixture is filtered through a pad of Celite. The solvent is removed in vacuo to afford 0.29 g (>100% yield) of 4-ethoxy-2-methoxy-5-methylaniline sufficiently pure for further use. ¹H NMR (CDCl₃, 400 MHz) δ 6.54, 6.44, 3.98-3.93, 3.82, 2.12, 1.40-1.36.

4-Ethoxy-2-methoxy-5-methylaniline (0.26 g, 1.4 mmol), dissolved in EtOAc, is added dropwise to excess phosgene (6.5 mL, 20% solution in toluene) dissolved in EtOAc. The solution is heated under reflux for 1 h, cooled and concentrated in vacuo to provide 0.28 g (94% yield) of 1-ethoxy-4-isocyanato-5-methoxy-2-methylbenzene. ¹H NMR (CDCl₃, 400 MHz) δ 6.76, 6.41, 4.04-3.99, 3.89, 2.10, 1.44-1.41. Example 129 is obtained from 1-ethoxy-4-isocyanato-5-methoxy-2-methylbenzene and 5-(trifluoromethyl)-1,3,4-thiadiazol-2-amine according to Method A, making non-critical changes. Yield 27%. HRMS (FAB) calculated for C₁₄H₁₅F₃N₄O₃S+H 377.0895, found 377.0878.

### Example 130

N-(5-acetyl-2,4-dimethoxyphenyl)-N′-[5-(trifluoromethyl)-1,3,4-thiadiazol-2-yl]urea.

Cupric nitrate (3.35 g, 13.87 mmol) is added portion-wise to a solution of 2,4-dimethoxyacetophenone in 80 mL acetic anhydride. After 3 h, the reaction is cooled to 0° C. and carefully treated with a 2:1 mixture of NH₄OH/NH₄Cl. The green-blue solution is extracted with EtOAc. A precipitate forms on standing. The solids are filtered and air-dried to provide 1.94 g (62% yield) of 1-(2,4-dimethoxy-5-nitrophenyl)ethanone. MS for C₁₀H₁₁NO₅ (ESI) (M+H)⁺ m/z 226.

1-(2,4-Dimethoxy-5-nitrophenyl)ethanone (1.87 g, 8.30 mmol) and a catalytic amount of Pt-black (200 mg) are mixed in EtOH/THF and shaken on a Parr hydrogenator apparatus in the presence of 40 psi H₂. After 24 h, the mixture is filtered through a pad of Celite. The solvent is removed in vacuo to afford 1.62 g (100% yield) of 1-(5-amino-2,4-dimethoxyphenyl)ethanone. MS for C₁₀H₁₃NO₃ (ESI) (M+H)⁺ m/z 196.

1-(5-Amino-2,4-dimethoxyphenyl)ethanone (1.52 g, mmol), dissolved in 75 mL EtOAc, is added dropwise over 1 h to excess phosgene (32.5 mL, 20% solution in toluene) dissolved in 35 mL EtOAc. The solution is heated under reflux for 30 min, cooled and concentrated in vacuo to provide 1.68 g (98% yield) of 1-isocyanato-2,4-dimethoxyacetophenone. MS for C₁₁H₁₁NO₄ (ESI) (M+H)⁺ m/z 222. Example 130 is obtained from 1-isocyanato-2,4-dimethoxyacetophenone and 5-(trifluoromethyl)-1,3,4-thiadiazol-2-amine according to Method A, making non-critical changes. Yield 60%. HRMS (FAB) calculated for C₁₄H₁₃F₃N₄O₄S+H 391.0688, found 391.0686.

The following compounds are made from 1-isocyanato-2,4-dimethoxyacetophenone according to Method A, making non-critical variations.

### Example 131

N-(5-acetyl-2,4-dimethoxyphenyl)-N′-(5-methyl-1,3,4-thiadiazol-2-yl)urea. Yield 74%. HRMS (FAB) calculated for C₁₄H₁₆N₄O₄S+H 337.0970, found 337.0966.

### Example 132

N-(5-acetyl-2,4-dimethoxyphenyl)-N′-(5-methylisoxazol-3-yl)urea. Yield 13%. HRMS (FAB) calculated for C₁₅H₁₇N₃O₅+H 320.1246, found 320.1243.

### Example 135

N-(2,4-dimethoxy-5-nitrophenyl)-N′-[5-(trifluoromethyl)-1,3,4-thiadiazol-2-yl]urea.

1,5-Difluoro-2,4-dinitrobenzene (10.1 g, 49 mmol) is added portion-wise to a solution sodium methoxide, prepared from sodium (2.6 g, 113 mmol) and 100 mL MeOH. After 18 h, the reaction is treated with water and CHCl₃. The pH is adjusted to pH 4 with conc. HCl. The mixture is extracted with CHCl₃, dried (MgSO4), filtered and concentrated to provide 9.9 g (88% yield) of 1,5-dimethoxy-2,4-dinitrobenzene as a light yellow solid. MS (ESI) for C₈H₈N₂O₆ (M−H) m/z 228.

A solution of sodium polysulfide, prepared from sodium sulfide nonahydrate (13.6 g, 56.6 mmol) and sulfur (3.4 g, 106.1 mmol) in 60 mL hot water is added dropwise to a suspension of 1,5-dimethoxy-2,4-dinitrobenzene (9.9 g, 43.4 mmol) in hot water. The yellow suspension turns dark orange upon heating under reflux. After 3 h, the water is removed. The residue is taken up in hot EtOAc and filtered. The solids are washed with hot EtOAc. The combined washes are concentrated and recrystallized from EtOH to provide 8.1 g of a dark solid that is an 8:1 mixture of 2,4-dimethoxy-5-nitroaniline and 1,5-dimethoxy-2,4-dinitrobenzene by ¹H NMR. ¹H NMR (DMSO, 400 MHz) δ 7.28, 6.75, 4.87, 3.93, 3.87. The mixture of compounds is sufficiently pure for further use.

A solution of the mixture of 2,4-dimethoxy-5-nitroaniline and 1,5-dimethoxy-2,4-dinitrobenzene (1.0 g, 5.05 mmol) in 135 mL EtOAc is added to excess phosgene (10 mL, 20% solution in toluene) in 30 mL EtOAc. After complete addition, the solution is heated under reflux for 30 min, cooled and concentrated in vacuo. The residue is dissolved in EtOAc and the solvent is removed (twice). Heptane is added and the solvent is removed to afford 0.90 g (80% yield) of 1-isocyanato-2,4-dimethoxy-5-nitrobenzene as a dark gray-red solid sufficiently pure for further use. ¹H NMR (CDCl₃, 400 MHz) δ 7.72, 6.53, 4.05, 4.00.

1-Isocyanato-2,4-dimethoxy-5-nitrobenzene (0.155 g, 0.76 mmol), 5-(trifluoromethyl)-1,3,4-thiadiazol-2-amine (0.128 g, 0.78 mmol), a catalytic amount of DMAP (ca. 4 mg) and four 5 mm glass beads are placed in a 40 mL vial equipped with a PTFE-lined cap and dissolved in 5 mL THF. The mixture is heated at 50° C. for 60 h. The solvent is removed by vigorous nitrogen flow. The resultant solid is purified by chromatography (Biotage 40S, 100% EtOAc) and then recrystallized from CH₃CN to provide 98 mg (33% yield) of Example 135. HRMS (FAB) calculated for C₁₂H₁₀F₃N₅O₅S+H 394.0433, found 394.0440.

The following compounds are made from the corresponding aminoheterocycles according to the procedure of Example 135, making non-critical variations.

### Example 136

N-(2,4-dimethoxy-5-nitrophenyl)-N′-(5-methyl-1,3-thiazol-2-yl)urea. Yield 39%. HRMS (FAB) calculated for C₁₃H₁₄N₄O₅S+H 339.0763, found 339.0757.

### Example 137

N-(2,4-dimethoxy-5-nitrophenyl)-N′-(5-methylisoxazol-3-yl)urea. Yield 20%. HRMS (FAB) calculated for C₁₃H₁₄N₄O₆+H 323.0992, found 323.0997.

### Example 140

N-[2-methoxy-5-methyl-4-(2,2,2-trifluoroethoxy)phenyl]-N′-[5-(trifluoromethyl)-1,3,4-thiadiazol-2-yl]urea.

Sodium 2,2,2-trifluoroethoxide, prepared from sodium (0.24 g, 10.5 mmol) and 2,2,2-trifluoroethanol, is added dropwise to solution of 1-chloro-5-fluoro-4-methyl-2-nitrobenzene (2.0 g, 9.5 mmol) dissolved 2,2,2-trifluoroethanol. The mixture is heated at 83° C. After 2 days, the mixture is cooled to RT and 1M citric acid is added. The residue is extracted with EtOAc, dried (MgSO₄), filtered and concentrated. The residue is a 2:3 mixture of product:starting material by ¹H NMR analysis. The mixture is reacted with NaH (0.28 g, 7.1 mmol, 60% oil dispersion (disp.)) and 2,2,2-trifluoroethanol (0.51 mL, 7.1 mmol) in DMF at 150° C. After 1 day, the mixture is cooled, diluted with water and extracted with EtOAc. The combined organic layers are washed with water and brine, dried (MgSO₄), filtered and concentrated. The residue is purified by chromatography (Biotage 40M, 4:1 hexanes/EtOAc) to provide 1.37 g (53% yield) of 1-chloro-4-methyl-2-nitro-5-(2,2,2-trifluoroethoxy)benzene. MS (ESI) for C₉H₇ClF₃NO₃ (M+H)⁺ m/z 270.0.

1-Chloro-4-methyl-2-nitro-5-(2,2,2-trifluoroethoxy)benzene (0.87 g, 3.2 mmol) is reacted with NaH (0.14 g, 3.6 mmol, 60% oil disp.) and MeOH (0. 14 mL, 3.6 mmol) in DMF at 150° C. After 18 h, the mixture is cooled, diluted with water and extracted with EtOAc. The combined organic layers are washed with water and brine, dried (MgSO₄), filtered and concentrated. The residue is purified by chromatography (Biotage 40S, 5% EtOAc/hexanes) and recrystallized from hexanes/EtOAc to provide 0.35 g (40% yield) of 1-methoxy-4-methyl-2-nitro-5-(2,2,2-trifluoroethoxy)benzene. MS (ESI) for C₁₀H₁₀F₃NO₄ (M+H)⁺ m/z 266.0.

1-Methoxy-4-methyl-2-nitro-5-(2,2,2-trifluoroethoxy)benzene (0.27 g, 1.0 mmol) and a catalytic amount of 10% Pd/C are mixed in EtOH/EtOAc and shaken on a Parr hydrogenator apparatus in the presence of 40 psi H₂. After the reaction is complete, the mixture is filtered through a pad of Celite and the solvent is removed in vacuo to afford 0.24 g (99% yield) of 2-methoxy-5-methyl-4-(2,2,2-trifluoroethoxy)aniline. MS (ESI) for C₉H₉ClF₃NO (M+H)⁺ m/z 236.1.

2-Methoxy-5-methyl-4-(2,2,2-trifluoroethoxy)aniline (0.24 g, 1.0 mmol), dissolved in EtOAc, is added dropwise to excess phosgene (4.3 mL, 20% solution in toluene) dissolved in EtOAc. The solution is heated under reflux for 30 min, cooled and concentrated in vacuo to provide 0.26 g (98% yield) of 1-isocyanato-2-methoxy-5-methyl-4-(2,2,2-trifluoroethoxy)benzene. ¹H NMR (CDCl₃, 400 MHz) δ 6.82, 6.40, 4.46-4.40, 3.82, 2.12. Example 140 is obtained from 1-isocyanato-2-methoxy-5-methyl-4-(2,2,2-trifluoroethoxy)benzene and 5-(trifluoromethyl)-1,3,4-thiadiazol-2-amine according to Method A, making non-critical changes. Yield 7.5%. HRMS (FAB) calculated for C₁₄H₁₂F₆N₄O₃S+H 431.0612, found 431.0620.

### Example 141

N-(5-chloro-2,4-dimethoxyphenyl)-N′-(2-methyl-1,3-thiazol-5-yl)urea.

Ethyl dithioacetate (5.24 g, 0.044 mol) and aminoacetonitrile hydrogensulfate (7.4 g, 0.048 mol) are dissolved in EtOH. The pH is adjusted to pH 9 with NaOH. The reaction is stirred at RT for 24 h, then heated under reflux. After 8 h, the reaction is cooled, diluted with aqueous NaOH and extracted with EtOAc. The combined organic layers are dried (MgSO₄), filtered and concentrated. The residue is purified by chromatography (Biotage 40M, EtOAc) to provide 1.04 g of 5-amino-2-methylthiazole. MS (ESI) for C₄H₆N₂S (M+H)⁺ m/z 115. Example 141 is obtained from 5-chloro-2,4-dimethoxyphenyl isocyanate and 5-amino-2-methylthiazole according to Method A, making non-critical variations. Yield 17%. HRMS (FAB) calcd for C₁₃H₁₄ClN₃O₃S+H 328.0522, found 328.0528.

### Example 142

N-(2-methoxy-4-nitrophenyl)-N′-[5-(trifluoromethyl)-1,3,4-thiadiazol-2-yl]urea.

2-Methoxy-4-nitrophenyl isocyanate (0.19 g, 1 mmol), 2-amino-5-trifluoromethyl-1,3,4-thiadiazole (0.17 g, 1 mmol), and a catalytic amount of DMAP (2-5 mg) are dissolved in THF (5 mL) and heated at 50° C. for 48 h. The solvent is removed with a stream of nitrogen and the solid recrystallized (CH₃CN) to give the product as an off-white solid (0.28 g, 77% yield). HRMS calcd for C₁₁H₈F₃N₅O₄S+H 364.0327 found 364.0341.

### Example 143

N-[5-(trifluoromethyl)-1,3,4-thiadiazol-2-yl]-N′-(2,4,5-trimethoxyphenyl)urea.

Sodium (0.26 g, 11.5 mmol) is added in small portions to MeOH (25 mL). The solution of NaOMe is added to 4-chloro-2,5-dimethoxynitrobenzene (2.27 g, 10.4 mmol) in MeOH (50 mL) at 0° C. The reaction is refluxed for 48 h, cooled to RT and quenched with 1M citric acid (50 mL) and H₂O (50 mL). The MeOH is removed under reduced pressure and the aqueous is extracted with EtOAc (3×50 mL), dried (MgSO₄), and the solvent is removed. The product is purified by Biotage Flash Chromatography (40M) using 30% EtOAc:hexanes as the eluent to give starting material (1.41 g) and 2,4,5-trimethoxynitrobenzene (0.80 g, 95% based on recovered starting material) as a yellow solid.

2,4,5-Trimethoxynitrobenzene (0.44 g, 2.1 mmol) is dissolved in minimal EtOAc (5 mL) and diluted with EtOH (50 mL). 10% Pd/C catalyst is added as a slurry in EtOAc and the mixture put on the Parr apparatus in the presence of H₂ (45 psi to 33 psi) for 0.5 h. The reaction mixture is filtered over celite to remove the catalyst and the solvent is removed to give 2,4,5-trimethoxyaniline (0.34 g, 86% yield) as a light pink solid.

2,4,5-Trimethoxyaniline (0.33 g, 1.8 mmol) is added dropwise as a solution in EtOAc (25 mL) to phosgene (7.6 mL, 20% in toluene) in EtOAc (50 mL). After complete addition, the reaction is heated under reflux for 0.5 h. The reaction is cooled to RT and the solvent is removed under reduced pressure to give 1-isocyanato-2,4,5-trimethoxybenzene (0.37 g, 99% yield) as a light brown solid. Example 143 is obtained using the isocyanate according to Method A making non-critical variations. Yield 44%. HRMS calcd for C₁₃H₁₃F₃N₄O₄S+H 379.0688 found 379.0695.

### Example 144

N-[4-methoxy-2-(methylthio)phenyl]-N′-[5-(trifluoromethyl)-1,3,4-thiadiazol-2-yl]urea.

Sodium thiomethoxide (0.75 g, 10.7 mmol) is added dropwise as a solution in MeOH (25 mL) to 5-chloro-2-fluoronitrobenzene (1.9 g, 10.7 mmol) in MeOH (50 mL) at RT. The yellow-green solution is stirred for 2 h and then quenched with 1M citric acid (50 mL). The MeOH is removed and the residue is dissolved in EtOAc (50 mL) and washed with 1M citric acid (50 mL), 0.5M NaOH (50 mL), and brine (50 mL). The organics are separated, dried (MgSO₄), and the solvent is removed under reduced pressure to give 4-chloro-2-thiomethylnitrobenzene (2.2 g, 100% yield) as a yellow solid.

Sodium (0.25 g, 10.7 mmol) is added in small portions to MeOH (25 mL). The solution of NaOMe is then added to 4-chloro-2-thiomethylnitrobenzene (2.2 g, 10.7 mmol) in MeOH (50 mL) at RT. The reaction is heated under reflux for 24 h and cooled to RT. A second equivalent of NaOMe is added and the reaction is heated under reflux for an additional 6 h. The reaction is quenched with 1M citric acid (50 mL) and H₂O (50 mL). The MeOH is removed under reduced pressure and the aqueous is extracted with EtOAc (3×50 mL), dried (MgSO₄), and the solvent is removed. A 1:1 mixture of product:starting material remained. The material is dissolved in DMF (50 mL) and NaH (0.65 eq, 60% oil disp.) and MeOH (0.65 eq) are added and the reaction is heated to reflux for 24 h. The work up is repeated to give 4-methoxy-2-(methylthio)-1-nitrobenzene (91.8 g, 85% yield) as a dark yellow solid.

4-Methoxy-2-(methylthio)-1-nitrobenzene is dissolved in minimal EtOAc (5 mL) and diluted with EtOH (50 mL). 10% Pd/C catalyst is added as a slurry in EtOAc and the mixture put on the Parr apparatus in the presence of H₂ (44 psi to 32 psi) for 0.5 h. The reaction mixture is filtered over Celite to remove the catalyst and the solvent is removed to give 4-methoxy-2-(methylthio)aniline (0.26G, 61% yield) as an orange oil.

4-Methoxy-2-(methylthio)aniline (0.26 g, 1.5 mmol) is added dropwise as a solution in EtOAc (25 mL) to phosgene (13.0 mL, 20% solution in toluene) in EtOAc (50 mL). After complete addition, the reaction is heated under reflux for 0.5 h. The reaction is cooled to RT and the solvent is removed under reduced pressure to give 1-isocyanato-4-methoxy-2-(methylthio)benzene (0.29 g, 97% yield) as a brown oil. Example 144 is obtained using the isocyanate according to Method A, making non-critical variations. Yield 55%. HRMS calcd for C₁₂H₁₁F₃N₄O₂S₂+H 365.0354 found 365.0347.

### Example 145

N-(4-{[(1R)-1-methylpropyl]oxy}phenyl)-N′-[5-(trifluoromethyl)-1,3,4-thiadiazol-2-yl]urea.

(R)-(−)-2-Butanol (0.75 mL, 8.1 mmol) is added dropwise to NaH (0.33 g, 8.1 mmol, 60% oil disp.) in DMF (100 mL) at RT and stirred for 0.5 h. 4-Fluoronitrobenzene (0.96 g, 6.8 mmol) is added and the solution is heated under reflux for 6 h and cooled to RT. The reaction mixture is diluted with H₂O (100 mL) and extracted with EtOAc (3×50 mL). The organics are separated, washed with H₂O (50 mL), and brine (50 mL), dried (MgSO₄), and the solvent is removed to give (1R)-1-methylpropyl 4-nitrophenyl ether (0.95 g, 71% yield) as a yellow oil.

(1R)-1-Methylpropyl 4-nitrophenyl ether (0.51 g, 2.6 mmol) is dissolved in minimal EtOAc (5 mL) and diluted with EtOH (50 mL). 10% Pd/C catalyst is added as a slurry in EtOAc and the mixture put on the Parr apparatus in the presence of H₂ (45 psi to 32 psi) for 0.5 h. The reaction mixture is filtered over Celite to remove the catalyst and the solvent is removed to give 4-{[(1R)-1-methylpropyl]oxy}aniline (90.41 g, 96% yield) as a brown oil.

4-{[(1R)-1-methylpropyl]oxy}aniline (0.41 g, 2.5 mmol) is added dropwise as a solution in EtOAc (25 mL) to phosgene (10 mL, 20% solution in toluene) in EtOAc (50 mL). After complete addition, the reaction is heated to reflux for 0.5 h. The reaction is cooled to RT and the solvent is removed under reduced pressure to give 1-isocyanato-4-{[(1R)-1-methylpropyl]oxy}benzene (0.48 g, 99% yield) as a purple oil. Example 145 is obtained using the isocyanate according to Method A, making non-critical variations. Yield 30%. HRMS calcd for C₁₄H₁₅F₃N₄O₂S+H 361.0946 found 361.0941.

### Example 146

N-[4-(allyloxy)phenyl]-N′-[5-(trifluoromethyl)-1,3,4-thiadiazol-2-yl]urea.

Allyl bromide (1.2 mL, 14.4 mmol) is added dropwise to a solution of 4-nitrophenol (2.0 g, 14.4 mmol) and K₂CO₃ (2.0 g, 14.4 mmol) in CH₃CN at RT. The reaction is heated to 80° C. for 3 h and the solids are filtered and washed with EtOAc. The filtrate is concentrated and the residue dissolved in EtOAc and washed with 10% KOH (20 mL) and H₂O (2×20 mL), dried (MgSO₄), and the solvent is removed under reduced pressure to give 1-(allyloxy)-4-nitrobenzene (2.5 g, 98% yield) as a tan oil.

1-(Allyloxy)-4-nitrobenzene (0.55 g, 3.1 mmol), iron powder (2.5 g, 44.8 mmol), and AcOH (0.1 mL) are stirred in H₂O (20 mL) at 85° C. for 0.5 h. The mixture is neutralized with 2M Na₂CO₃. The mixture is filtered and then the filtrate is extracted with EtOAc (3×50 mL), dried (MgSO₄) and the solvent is removed under reduced pressure. The product is isolated from the remaining starting material by Biotage Flash Chromatography (40S) using 30% EtOAc/hexanes as the eluent to give 4-(allyloxy)aniline (0.28 g, 60% yield) as a brown oil.

4-(Allyloxy)aniline (0.28 g, 1.9 mmol) is added dropwise as a solution in EtOAc (25 mL) to phosgene (8 mL, 20% solution in toluene) in EtOAc (50 mL). After complete addition, the reaction is heated under reflux for 0.5 h. The reaction is cooled to RT and the solvent is removed under reduced pressure to give 1-isocyanato-4-(allyloxy)aniline (0.30, 91% yield) as a brown oil. Example 146 is obtained using the isocyanate according to Method A, making non-critical variations. Yield 24%. HRMS calcd for C₁₃H₁₁F₃N₄O₂S+H 345.0633 found 345.0637.

### Example 147

N-(4-propoxyphenyl)-N′-[5-(trifluoromethyl)-1,3,4-thiadiazol-2-yl]urea.

1-(Allyloxy)-4-nitrobenzene (0.51 g, 2.9 mmol) is dissolved in EtOH (50 mL), 10% Pd/C catalyst is added as a slurry in EtOAc and the mixture put on the Parr apparatus in the presence of H₂ (38 psi to 20 psi) for 0.5 h. The reaction mixture is filtered over Celite to remove the catalyst and the solvent is removed to give the 4-propyloxyaniline (423 mg, 98% yield) as a brown oil.

4-Propyloxyaniline (0.42 g, 2.8 mmol) is added dropwise as a solution in EtOAc (25 mL) to phosgene (11.8 mL, 20% solution in toluene) in EtOAc (50 mL). After complete addition, the reaction is heated under reflux for 0.5 h. The reaction is cooled to RT and the solvent is removed under reduced pressure to give 1-isocyanato-4-propyloxybenzene (0.49 g, 99% yield) as a brown oil. Example 147 is obtained using the isocyanate according to Method A, making non-critical variations. Yield 8%. HRMS calcd for C₁₃H₁₃F₃N₄O₂S+H 347.0789 found 347.0786.

### Example 148

N-(2-ethoxypyridin-3-yl)-N′-[5-(trifluoromethyl)-1,3,4-thiadiazol-2-yl]urea.

Sodium (0.17 g, 7.2 mmol) is added in small portions to EtOH (25 mL). The solution of NaOEt is added to 2-chloro-3-nitropyridine (1.0 g, 6.6 mmol) in EtOH (50 mL) at RT. The reaction is heated to 75° C. for 1 h, cooled to RT and quenched with 1M citric acid (50 mL) and H₂O (50 mL). The EtOH is removed under reduced pressure and the aqueous is extracted with EtOAc (3×30 mL), dried (MgSO₄), and the solvent is removed to give 2-ethoxy-3-nitropyridine (1.06 g, 96% yield) as an orange oil.

The 2-ethoxy-3-nitropyridine (1.05 g, 6.3 mmol) is dissolved in minimal EtOAc (5 mL) and diluted with EtOH (50 mL). 10% Pd/C catalyst is added as a slurry in EtOAc and the mixture put on the Parr apparatus in the presence of H₂ (40 psi to 11 psi) for 0.5 h. The reaction mixture is filtered over Celite to remove the catalyst and the solvent is removed to give 2-ethoxypyridin-3-amine (0.82 g, 95% yield) as a tan oil.

2-Ethoxypyridin-3-amine (0.21 g, 1.5 mmol) is added dropwise as a solution in EtOAc (25 mL) to phosgene (6 mL, 20% solution in toluene) in EtOAc (50 mL). After complete addition, the reaction is heated under reflux for 0.5 h. The reaction is cooled to RT and the solvent is removed under reduced pressure to give 2-ethoxy-3-isocyanatopyridine (0.24 g, 98% yield) as a tan oil. Example 148 is obtained using the isocyanate according to Method A, making non-critical variations. Yield 26%. HRMS calcd for C₁₁H₁₀F₃N₅O₂S+H 334.0585 found 334.0580.

### Example 149

N-(4-methoxy-2-methylphenyl)-N′-[4-(trifluoromethyl)-1,3-thiazol-2-yl]urea.

4-(Trifluoromethyl)-1,3-thiazol-2-amine (0.1117 g, 0.664 mmol) is prepared from thiourea and 3-bromo-1,1,1-trifluoroacetone by the procedure described in Biotechnology and Bioengineering (Combinatorial Chemistry), 2000, 71(1), 9. The free base is obtained by the procedure described in the preparation of N-(4-ethyl-1,3-thiazol-2-yl)-N′-(4-methoxy-2-methylphenyl)urea). The free base and DMAP (0.0041 g) are dissolved in THF (3 mL). 4-Methoxy-2-methylphenylisocyanate (0.097 mL, 0.108 g, 0.664 mmol) is added and the reaction mixture is stirred at 50° C. under N₂ for 6 days. The reaction mixture is cooled to RT and concentrated. The residue is taken up in CH₂Cl₂ and insoluble N,N′-bis(4-methoxy-2-methylphenyl)urea (0.0334 g) is collected by filtration. The filtrate is concentrated and the residue is chromatographed (SiO₂, 8:1 CHCl₃:EtOAc) to yield Example 149 (0.0791 g) in 36% yield. MS (ESI+) for C₁₃H₁₂F₃N₃O₂S m/z 332.1 (M+H)⁺.

### Example 150

N-(4-methoxy-2-methylphenyl)-N′-(3-phenyl-1,2,4-thiadiazol-5-yl)urea.

5-Amino-3-phenyl-1,2,4-thiadiazole (0.177 g, 1 mmol) is dissolved in THF (4 mL) in a teflon capped vial. DMAP (0.0060 g) and 4-methoxy-2-methylphenylisocyanate (0.163 g, 1 mmol) are added. The vial is placed in an orbital shaker at 50° C. for 16 h. After cooling to RT, the crude product is purified by reverse phase preparative HPLC (eluent: CH₃CN and 0.05% HCOOH/H₂O; column: Kromasil C18) to yield Example 150 (0.0140 g) in 4% yield. MS (ESI+) for C₁₇H₁₆N₄O₂S m/z 341.3 (M+H)⁺.

### Example 151

N-(5-ethyl-4-phenyl-1,3-thiazol-2-yl)-N′-(4-methoxy-2-methylphenyl)urea.

5-Ethyl-4-phenyl-1,3-thiazol-2-amine hydrochloride hydrate (0.0958 g, 0.398 mmol) is partitioned between EtOAc and 1N NaOH. The layers are separated and the organic layer is dried (MgSO₄), filtered and concentrated to yield crude 5-ethyl-4-phenyl-1,3-thiazol-2-amine (0.074 g, 0.362 mmol). The crude free base is dissolved in THF (3 mL) in a teflon capped vial. DMAP (0.0020 g) and 4-methoxy-2-methylphenylisocyanate (0.0530 mL, 0.0590 g, 0.362 mmol) are added. The vial is placed in an orbital shaker at 50° C. for 16 h. After cooling to RT, the solvent is removed. The crude is dissolved in a small amount of EtOAc and the crystalline product is collected by filtration, washed with a small amount of EtOAc and dried under vacuum to yield Example 151 (0.100 g) in 75% yield. MS (ESI+) for C₂₀H₂₁N₃O₂S m/z 368.3 (M+H)⁺.

### Example 152

N-(4-hydroxy-2-methylphenyl)-N′-[3-(trifluoromethyl)phenyl]urea.

N-(4-Hydroxy-2-methylphenyl)-N′-[3-(trifluoromethyl)phenyl]urea (from 4-amino-3-methylphenol and 1-isocyanato-3-(trifluoromethyl)benzene) is prepared following the Method A, making non-critical modifications. The resulting residue is diluted with MeOH (10 ml) and DOWEX 50WX2-400 ion-exchange resin (1.5 g) is added; the mixture is allowed to spin submerged in a water bath (35-40° C.) for 20 min, is filtered, and the resin washed with MeOH. The product is liberated from the resin by treatment with a solution of 20% NH₄OH/MeOH. The basic alcohol washes are concentrated in vacuo to give an off white solid, which is triturated with EtOAc/CH₂Cl₂ to give an off white solid 0.085 g (34% yield). HRMS (ESI) calcd for C₁₅H₁₃N₂O₂F₃+H 311.1007, found 311.1002.

### Example 153

N-(4-hydroxyphenyl)-N′-[3-(trifluoromethyl)phenyl]urea.

N-(4-Hydroxyphenyl)-N′-[3-(trifluoromethyl)phenyl]urea (from 4-aminophenol and 1-isocyanato-3-(trifluoromethyl)benzene) is prepared by following Method A, making non-critical modifications. The resulting residue is triturated with EtOAc/CH₂Cl₂ to afford an off white solid 0.102 g (56% yield). HRMS (ESI) calcd for C₁₄H, F₃N₂O₂+H 297.0851, found 297.0849.

### Example 154

N-[2-methyl-4-(methylthio)phenyl]-N′-[3-(trifluoromethyl)phenyl]urea.

To a solution of 3-fluoro-2-nitrotoluene (1.0 g, 6.45 mmol) in DMSO (45 ml) is added sodium thiomethoxide (0.904 g, 12.9 mmol). The reaction mixture is stirred at 80° C. for 4 h. The mixture is diluted with H₂O extracted with EtOAc, and the combined organic layers are dried (MgSO₄), filtered, and concentrated under vacuum. The residue is purified by silica gel chromatography (40% CH₂Cl₂/hexane) to give a yellow solid 0.88 g (74% yield). HRMS (EI) calcd for C₈H₉NO₂S 183.0358, found 183.0354.

A solution of 2-methyl-4-(methylthio)-1-nitrobenzene (0.20 g, 1.08 mmol) in EtOH (50 ml)/EtOAc (20 ml) is hydrogenated at RT and 40 psi, in the presence of 10% Pd-C (0.081 g, 0.076 mmol). The suspension is filtered through cellulose and washed with EtOH. The solution is concentrated under vacuum to give brown oil 0.16 g (96% yield). This compound is used without further purification in the next step. HRMS (ESI) calcd for C₈H₁₁NS+H 154.0690, found 154.0685.

N-[2-methyl-4-(methylthio)phenyl]-N′-[3-(trifluoromethyl)phenyl]urea (from 2-methyl-4-(methylthio)aniline and 1-isocyanato-3-(trifluoromethyl)benzene) is prepared by following Method A, making non-critical modifications. The resulting residue is triturated with CH₂Cl₂ to afford an off white solid 0.047 g (24% yield). HRMS (ESI) calcd for C₁₆H₁₅N₂OSF₃+H 341.0935, found 341.0948.

### Example 155

N-(2-ethyl-4-hydroxyphenyl)-N′-[3-(trifluoromethyl)phenyl]urea.

To the solution of 5-hydroxy-2-nitrobenzaldehyde (0.3 g, 1.8 mmol) in DMF (5.0 ml) are added Cs₂CO₃ (1.17 g, 3.6 mmol) and benzyl bromide (0.46 g, 2.7 mmol). The reaction mixture is stirred overnight at RT. The mixture is diluted with H₂O extracted with EtOAc, and the combined organic layers are dried (MgSO₄), filtered, and concentrated under vacuum. The residue is triturated with hexane to give a yellow solid 0.335 g (72% yield). MS (ESI+) for C₁₄H₁₁NO₄ m/z 258.1 (M+H)⁺.

To a suspension of methyltriphenylphosphonium bromide (0.51 g, 1.4 mmol) in THF (5.0 ml) is added potassium tert-butoxide (0.15 g, 1.3 mmol) and the mixture is stirred at RT for 30 min. The solution of 5-(benzyloxy)-2-nitrobenzaldehyde (0.335 g, 1.3 mmol) in THF (2.0 ml) is slowly added to the reaction mixture, which is stirred at RT for additional 2 hr. The solids are filtered and the filtrate is concentrated to give a brown oil, which is purified by silica gel chromatography (10% EtOAc/n-heptane) to afford a yellow oil 0.268 g (81% yield). ¹H NMR (400 MHz, CDCl₃) δ 8.09, 7.42, 7.35, 7.14, 6.98, 5.72, 5.67, 5.21.

Step 3

A solution of 4-(benzyloxy)-1-nitro-2-vinylbenzene (0.215 g, 0.84 mmol) in EtOH (50 ml)/EtOAc (50 ml) is hydrogenated at RT and 40 psi, in the presence of 10% Pd-C (0.063 g, 0.059 mmol). The suspension is filtered through cellulose and washed with EtOH. The resulting residue is triturated with CH₂Cl₂ to afford a brown solid 0.066 g (57% yield). MS (ESI+) for C₈H₁₁ NO m/z 138.1 (M+H)⁺.

N-(2-Ethyl-4-hydroxyphenyl)-N′-[3-(trifluoromethyl)phenyl]urea (from 4-amino-3-ethylphenol and 1-isocyanato-3-(trifluoromethyl)benzene) is prepared by following Method A, making non-critical modifications. The resulting residue is purified by silica gel chromatography (50% EtOAc/n-heptane) followed by recrystallization from EtOAc/n-heptane to give a white solid 0.07 g (46% yield). HRMS (ESI) calcd for C₁₆H₁₅N₂O₂F₃+H 325.1164, found 325.1170.

### Example 156

N-(4-amino-2-methylphenyl)-N′-[3-(trifluoromethyl)phenyl]urea.

N-(2-Methyl-4-nitrophenyl)-N′-[3-(trifluoromethyl)phenyl]urea (from 3-(trifluoromethyl) aniline and 1-isocyanato-2-methyl-4-nitrobenzene) is prepared by following the Method A, making non-critical modifications. The resulting solid is crystallized from CH₃CN to give a yellow solid 0.52 g (55% yield). MS (ESI−) for C₁₅H₁₂F₃N₃O₃ m/z 338.1 (M−H)⁻.

Example 156 (from N-(2-methyl-4-nitrophenyl)-N′-[3-(trifluoromethyl)phenyl]urea) is prepared by following Step 3 of Example 155, making non-critical modifications. The solution is concentrated under vacuum to give an off white solid 0.2 g (74% yield). HRMS (ESI) calcd for C₁₅H₁₄N₃OF₃+H 310.1167, found 310.1177.

### Example 157

N-(4-methoxyphenyl)-N′-[3-(trifluoromethyl)phenyl]urea.

Example 157 (from 4-methoxyaniline and 1-isocyanato-3-(trifluoromethyl)benzene) is prepared by following Method A, making non-critical modifications. The resulting residue is triturated with CH₂Cl₂ to afford a white solid 0.211 g (84% yield). HRMS (ESI) calcd for C₁₅H₁₃N₂O₂F₃+H 311.1007, found 311.1010

### Example 158

N-(5-hydroxy-2-methylphenyl)-N′-[3-(trifluoromethyl)phenyl]urea.

Example 158 (from 3-amino-4-methylphenol and 1-isocyanato-3-(trifluoromethyl)benzene) is prepared by following the Method A, making non-critical modifications. The residue is triturated with CH₂Cl₂ to afford a white solid 0.054 g (21% yield). HRMS (ESI) calcd for C₁₅H₁₃N₂O₂F₃+H 311.1007, found 311.1013.

### Example 159

N-(5-chloro-2,4-dimethoxyphenyl)-N′-[4-(trifluoromethyl)-1H-pyrazol-1-yl]urea.

3,3,3-Trifluoropropionic (5.64 g, 44.1 mmol) is combined with chlorornethylene dimethyl ammonium chloride (11.45 g, 96.6 mmol) in 42 mL 1,2-dichloroethane, is warmed to 75° C., and is stirred for 5 hrs under N₂. The reaction mixture is cooled, and volatiles removed in vacuo overnight to afford 10.82 g of the intermediate, which is then combined with hydrazine monohydrate (2.72 mL, 1.2 eq) in 145 mL CH₃CN, and allowed to stir for 1 hr. TFA (5.03 mL, 3 eq) is added to reaction mixture, which is then warmed to 70° C., and stirred for 1.5 hr under N₂. The reaction mixture is cooled, and concentrated in vacuo, and partitioned between 30 mL H₂O and 30 mL EtOAc. NaHCO₃ (3.6 g) is added to the vigorously stirring mixture. The layers are then separated and the aqueous layer is washed (×3) using EtOAc. Organics are combined and concentrated. Product is chromatographed (Biotage 40+S) using 20% EtOAc in hexanes. Appropriate fractions are concentrated in vacuo at R.T. (to discourage sublimation) to afford 1.75 g (29% yield) of 4-(trifluoromethyl)-1H-pyrazol-1-amine a light yellow solid. Anal. Calcd for C₄H₃F₃N₂: C, 35.31; H, 2.22; N, 20.59. Found: C, 35.34; H, 2.40; N, 20.55.

4-(Trifluoromethyl)-1H-pyrazol-1-amine (0.68 g, 5 mmol) is added to a stirring solution of hydroxyamine-o-sulfonic acid (0.679 g, 6 mmol) in 20 mL 12N NaOH, and stirred overnight. The reaction mixture is extracted and washed (3×) with Et₂O. The organic layers are combined, dried (MgSO₄), and concentrated to afford 0.38 g of 4-(trifluoromethyl)-1H-pyrazol-1-amine as a yellow oil.

4-(Trifluoromethyl)-1H-pyrazol-1-amine (154 mg, 1 mmol) is combined with 5-chloro-2,4-dimethoxyphenylisocyanate (213 mg, 1 mmol) in 8 mL THF, to which 5 mg DMAP is added. The reaction mixture is allowed to stir at RT for 2 days, and then concentrated to a brown solid and chromatographed (Biotage 25+S) using 50% EtOAc in hexanes to afford 66 mg (21% yield) of Example 159 as a white solid. MS (EI) m/z: 364 (M)⁺.

### Example 160

N-(4-bromo-1H-pyrazol-1-yl)-N′-(5-chloro-2,4-dimethoxyphenyl)urea. Yield 26%. HRMS (FAB) calcd for C₁₂H₁₂BrClN₄O₃ 373.9782, found 373.9783.

### Example 161

N-(4-methoxy-2-methylphenyl)-N′-[3-(trifluoromethyl)phenyl]thiourea.

The crude product is triturated with CH₂Cl₂/hexane to give an off white solid 0.160 g. Yield 65%. HRMS (ESI) calcd for C₁₆H₁₅F₃N₂OS+H 341.0935, found 341.0924.

### Example 162

N-(4-hydroxy-2-methylphenyl)-N′-[3-(trifluoromethyl)phenyl]thiourea.

N-(4-Hydroxy-2-methylphenyl)-N′-[3-(trifluoromethyl)phenyl]thiourea (from 4-amino-3-methylphenol and 1-isothiocyanato-3-(trifluoromethyl)benzene) is prepared by following Method A, making non-critical modifications. The resulting residue is triturated with CH₂Cl₂ to afford an off white solid 0.192 g (72% yield). HRMS (ESI) calcd for C₁₅H₁₃F₃N₂OS+H 327.0779, found 327.0793.

### METHOD B

### Example 200

N-(2,4-dimethoxy-5-methylphenyl)-N′-[3-(trifluoromethyl)isoxazol-5-yl]urea.

2,4-Dimethoxy-5-methylaniline (0.22 g, 1.3 mmol) is added dropwise as a solution in EtOAc (25 mL) to a phosgene solution (5.6 mL, 20% solution in toluene) in EtOAc (50 mL). After complete addition, the reaction is heated under reflux for 0.5 h. The reaction is cooled to RT and the solvent is removed under reduced pressure to give 1-isocyanato-2,4-dimethoxy-5-methylbenzene as a light brown solid (0.25 g, 98% yield).

Sodium hydride (44 mg, 1.1 mmol, 60% oil disp.) is added to 3-(trifluoromethyl)isoxazole-5-amine (0.17 g, 1.1 mmol) in THF (5 mL) at 0° C. After 0.5 h, a THF solution (5 mL) of 1-isocyanato-2,4-dimethoxy-5-methylbenzene (0.19 g, 1.0 mmol) is added dropwise. The reaction is warmed to RT. After 2 h, concentrated HCl is added until the pH<5 and the solvent is removed. The crude material is purified by chromatography (Biotage 40S, 1:1 EtOAc:hexanes). The solid is recrystallized (EtOAc/hexanes) to give Example 200 as an off-white solid (135 mg, 39% yield). HRMS calcd for C₁₄H₁₄F₃N₃O₄+H 346.1014 found 346.1016.

The following compounds are made from an aminoheterocycle, an aryl isocyanate or aryl isothiocyanate and a base according to Method B, making non-critical variations.

### Example 201

N-(4-ethoxyphenyl)-N′-[3-(trifluoromethyl)isoxazol-5-yl]urea. Yield 16%. HRMS calcd for C₁₃H₁₂F₃N₃O₃+H 316.0909 found 316.0921.

### Example 202

N-(2-ethoxyphenyl)-N′-[3-(trifluoromethyl)isoxazol-5-yl]urea. Yield 27%. HRMS calcd for C₁₃H₁₂F₃N₃O₃+H 316.0909 found 316.0913.

### Example 203

N-(2,4-dimethoxyphenyl)-N′-[3-(trifluoromethyl)isoxazol-5-yl]urea. Yield 34%. HRMS calcd for C₁₃H₁₂F₃N₃O₄+H 332.0858 found 332.0851.

### Example 204

N-(2,6-dimethoxypyridin-3-yl)-N′-(3-methylisoxazol-5-yl)urea. Yield 2%. HRMS calcd for C₁₂H₁₄N₄O₄+H 279.1093 found 279.1081.

### Example 205

N-(5-fluoro-2,4-dimethoxyphenyl)-N′-(3-methylisoxazol-5-yl)urea. Yield 23%. HRMS calcd for C₁₃H₁₄FN₃O₄+H 296.1046 found 296.1045.

### Example 206

N-(2,4-dimethoxyphenyl)-N′-(3-methylisoxazol-5-yl)urea. Yield 66%. HRMS calcd for C₁₃H₁₅N₃O₄+H 278.1140 found 278.1152.

### Example 207

N-(5-chloro-2,4-dimethoxyphenyl)-N′-[3-(trifluoromethyl)isoxazol-5-yl]thiourea. Yield 34%. HRMS calcd for C₁₃H₁₁ClF₃N₃O₃S+H 382.0240 found 382.0247.

### Example 208

N-(5-chloro-2,4-diethoxyphenyl)-N′-[3-(trifluoromethyl)isoxazol-5-yl]urea. Prepared according to Method B, except using KHMDS instead of NaH. Yield 29%. HRMS calcd for C₁₅H₁₅ClF₃N₃O₄+H 394.0781 found 394.0787.

### Example 209

N-(4-methoxy-2-nitrophenyl)-N′-[3-(trifluoromethyl)isoxazol-5-yl]urea. Prepared according to Method B, except using NaHMDS instead of NaH. Yield 25%. HRMS calcd for C₁₂H₉F₃N₄O₅+H 346.0525 found 346.0526.

### Example 210

N-(4-methoxy-2-methylphenyl)-N′-[4-(trifluoromethyl)-I,3-thiazol-2-yl]thiourea.

To a mixture of 4-(trifluoromethyl)-1,3-thiazol-2-amine (0.200 g, 1.19 mmol) and 1-isothiocyanato-4-methoxy-2-methylbenzene (0.213 g, 1.19 mmol) in THF (6.0 ml) is added NaH 60% oil disp. (0.047 g, 1.19 mmol). The reaction mixture is stirred at 50° C. for 2 hr. The mixture is neutralized with 0.1M HCl, extracted with CH₂Cl₂ (3×20 ml), and the combined organic layers are dried (MgSO₄), filtered, and concentrated under vacuum. The residue is triturated with CH₂Cl₂/hexane to afford an off white solid 0.198 g (48% yield). HRMS (ESI) calcd for C₁₃H₁₂F₃N_{3OS}₂+H 348.0452, found 348.0450.

### Example 211

N-(5-chloro-2,4-dimethoxyphenyl)-N′-[5-(trifluoromethyl)-1,3,4-thiadiazol-2-yl]thiourea.

To a mixture of 2-amino-5-methyl-1,3,4-thiadiazole (0.19 g, 1.1 mmol) in 12 mL of a 1:1 THF/CH₂Cl₂ solvent mixture is added NaH (44 mg, 1.1 mmol, 60% oil disp.). The mixture is stirred at rt for 20 min and then 5-chloro-2,4-dimethoxyphenyl isothiocyanate (0.25 g, 1.1 mmol) is added and the reaction stirred at 50° C. for 16 h. After cooling to rt, the mixture is treated with 5 drops (ca. 0.25 mL) of 0.1 N HCl. Water and CH₂Cl₂ (20 mL each) are added and the layers separated. The aqueous layer is extracted with CH₂Cl₂ (2×20 mL). The combined organic layers are dried (MgSO₄), filtered, and concentrated. The crude product is subjected to flash chromatography (EtOAc: hexane gradient) to give Example 211. ¹H NMR (400 MHz, d₆-DMSO) δ 8.69-8.73, 8.06-8.23, 7.30, 6.61, 3.99, 3.94.

### Example 212

N-(5-fluoro-2,4-dimethoxyphenyl)-N′-[5-(trifluoromethyl)-1,3,4-thiadiazol-2-yl]thiourea.

A solution of 2,4-dimethoxy-5-fluoroaniline (0.70 g, 4.1 mmol) in 50 mL of EtOAc is cooled to 0° C. and treated dropwise with thiophosgene (2.8 mL, 37 mmol). The solution is stirred at 0° C. for 2 h. The mixture is concentrated to give 5-fluoro-2,4-dimethoxyphenyl isothiocyanate, which is immediately used without further purification.

To a mixture of 2-amino-5-methyl-1,3,4-thiadiazole (0.69 g, 4.1 mmol) in 40 mL of THF is added NaH (160 mg, 4.1 mmol, 60% oil disp.). The mixture is stirred at rt for 20 min. 5-Fluoro-2,4-dimethoxyphenyl isothiocyanate (0.87 g, 4.1 mmol) is then added and the reaction mixture is stirred at 50° C. for 16 h. After cooling to rt, the mixture is treated with 5 drops (ca. 0.25 mL) of 0.1N HCl. Water and CH₂Cl₂ are added and the layers separated. The aqueous layer is extracted with CH₂Cl₂. The combined organic layers are dried (MgSO₄), filtered, and concentrated. The crude product is purified by flash column chromatography (EtOAc:hexane gradient) to give Example 212. ¹H NMR (400 MHz, d₆-DMSO) δ 9.90-9.97, 8.07, 7.63-7.75, 6.93, 3.90, 3.86.

### Example 213

N-[2-methoxy-4-(2-methoxyethoxy)phenyl]-N′-[5-(trifluoromethyl)-1,3,4-thiadiazol-2-yl]urea.

To a cooled solution (˜0° C.) of 2-methoxyethanol (0.8 mL) in CH₂Cl₂ (50 mL) and TEA (2 mL) is added dropwise methanesulfonyl chloride (1.3 mL). After 5 min, the bath is removed and the reaction mixture allowed to warm to RT and stir for 1 hour at which point it is washed with 1.0 N NaOH, brine, dried (MgSO₄), and concentrated to give an oil. Yield quantitative. MS (ESI+) for C₄H₁₀O₄S m/z 155.1 (M+H)⁺.

To a solution of 2-methoxyethyl methanesulfonate (1.54 g) in 2-butanone (150 mL) is added cesium carbonate (6.50 g) and 3-fluoro-4-nitrophenol (1.52 g). The resulting mixture is refluxed for 19 hours, cooled, filtered, concentrated, and purified using silica gel chromatography (EtOAc/Heptanes). Yield 98%. ¹H NMR (400 MHz, DMSO-d₆) δ 7.95, 6.82, 6.67, 4.25, 3.92, 3.68, 3.33, 3.31.

To a mixture of 2-methoxy-4-(2-methoxyethoxy)-1-nitrobenzene (2.0 g), 10% Pd/C (1.0 g) in MeOH (200 mL) is added conc. HCl (1.8 g). The resulting mixture is shaken at 40 psi H₂ for 15 minutes. The mixture is filtered and crystallized from EtOH/Et₂O to give 2-methoxy-4-(2-methoxyethoxy)aniline hydrochloride. Yield 76%. MS (ESI+) for C₁₀H₁₅NO₃ m/z 198.1 (M+H)⁺.

Example 156 (from 2-methoxy-4-(2-methoxyethoxy)aniline hydrochloride and 2-isocyanato-5-(trifluoromethyl)-1,3,4-thiadiazole) is prepared using Method B. Yield 50%. HRMS (ESI) calcd for C₁₄H₁₅N₄O₄SF₃+H 393.0844, found 393.0838.

### Example 214

N-(4-ethoxy-2-nitrophenyl)-N′-[3-(trifluoromethyl)isoxazol-5-yl]thiourea.

To a solution of 3-(trifluoromethyl)isoxazol-5-amine (0.77 g) in DMF (10 mL) is added NaH (60% oil disp.). The resulting mixture is stirred for 20 min and then 4-ethoxy-1-isothiocyanato-2-nitrobenzene (see Dyson, G. M.; George, H. J.; Hunter, R. F. J. Chem. Soc. 1927, 436-445) (0.22 g) is added. The resulting mixture is stirred for 30 min, concentrated to dryness, taken-up in EtOAc, washed with brine, and purified utilizing preparatory HPLC. Yield 9%. HRMS (EI) calcd for C₁₃H₁₁F₃N₄O₄S 376.0453, found 376.0450.

### Example 215

N-(4-hydroxy-2-methylphenyl)-N′-[4-(trifluoromethyl)-1,3-thiazol-2-yl]thiourea.

To a solution of 4-isothiocyanato-3-methylphenol (0.3 g, 1.82 mmol) and imidazole (0.132 g, 1.92 mmol) in DMF (3.5 ml) is added tert-butyl(chloro)dimethylsilane (0.289 g, 1.92 mmol). The reaction mixture is stirred at RT overnight. The mixture is diluted with H₂O, extracted with EtOAc, and the combined organic layers are dried (MgSO₄), filtered, and concentrated under vacuum. The residue is purified by silica gel chromatography (40% CH₂Cl₂/hexane) to give tert-butyl(4-isothiocyanato-3-methylphenoxy)dimethylsilane as a clear oil 0.45 g (89% yield). ¹H NMR (400 MHz, CDCl₃) δ 6.85, 6.48, 6.43, 2.13, 0.78, 0.01.

N-{2-Methyl-4-[1-methyl-1-(trimethylsilyl)ethoxy]phenyl}-N′-[4-(trifluoromethyl)-1,3-thiazol-2-yl]urea (from tert-butyl(4-isothiocyanato-3-methylphenoxy)dimethylsilane and 4-(trifluoromethyl)-1,3-thiazole-2-amine) is prepared by following Method B making non-critical modifications (0.64 g, 1.43 mmol). This is carried directly into the next step.

N-(4-{[tert-butyl(dimethyl)silyl]oxy}-2-methylphenyl)-N′-[4-(trifluoromethyl)-1,3-thiazol-2-yl]thiourea (0.64 g, 1.43 mmol) is dissolved in THF (7.0 ml) and 1.0M solution of tetra-butyl ammonium fluoride (4.3 ml, 4.29 mmol) is added. The reaction mixture is stirred at RT for 1 hr. The mixture is diluted with H₂O extracted with CH₂Cl₂, and the combined organic layers are dried (MgSO₄), filtered, and concentrated under vacuum. The residue is triturated to give Example 215 as an off white solid 0.116 g (24% yield). HRMS (ESI) calcd for C₁₂H₁₀N₃OS₂F₃+H 334.0295, found 334.0300.

### METHOD C

### Example 300

N-(3-chloro-4-methoxyphenyl)-N′-[5-(trifluoromethyl)-1,3,4-thiadiazol-2-yl]urea.

A solution of 5-(trifluoromethyl)-1,3,4-thiadiazol-2-amine (2.82 g, 16.67 mmol), dissolved in 70 mL EtOAc, is added dropwise over 1 h to excess phosgene (71.0 mL, 20% solution in toluene) dissolved in 25 mL EtOAc. The solution is heated under reflux for 30 min, cooled and concentrated in vacuo to provide 2.95 g (100% yield) of 2-isocyanato-5-trifluoromethylthiadiazole.

2-Isocyanato-5-trifluoromethylthiadiazole (0.175 g, 1.0 mmol), 3-chloro-4-methoxyaniline (0.157 g, 1.0 mmol), a catalytic amount of DMAP (ca. 4 mg) and four 5 mm glass beads are placed in a 40 mL vial equipped with a PTFE-lined cap and dissolved in 5 mL THF. The mixture is heated at 50° C. for 16 h. The solvent is removed in vacuo. The resultant solid is recrystallized from CH₃CN to provide 90 mg (26% yield) of Example 300. HRMS (FAB) calculated for C₁₁H₈ClF₃N₄O₂S+H 353.0087, found 353.0086.

The following compounds are made from the corresponding aniline according to the procedure of Method C, making non-critical variations.

### Example 301

N-(5-chloro-2-methoxyphenyl)-N′-[5-(trifluoromethyl)-1,3,4-thiadiazol-2-yl]urea. Yield 43%. HRMS (FAB) calculated for C₁₁H₈ClF₃N₄O₂S+H 353.0087, found 353.0087.

### Example 302

N-(4-methoxy-2-nitrophenyl)-N′-[5-(trifluoromethyl)-1,3,4-thiadiazol-2-yl]urea. Yield 41%. HRMS (FAB) calculated for C₁₁H₈F₃N₅O₄S+H 364.0327, found 364.0334.

### Example 303

N-(4-ethoxy-2-nitrophenyl)-N′-[5-(trifluoromethyl)-1,3,4-thiadiazol-2-yl]urea. Yield 27%. HRMS (FAB) calculated for C₁₂H₁₀F₃N₅O₄S+H 378.0484, found 378.0479.

### Example 304

N-(4-methoxy-2-methylphenyl)-N′-[5-(trifluoromethyl)-1,3,4-thiadiazol-2-yl]urea. Yield 43%. HRMS (FAB) calculated for C₁₂H₁₁F₃N₄O₂S+H 333.0633, found 333.0630.

### Example 305

N-(2,6-dimethoxypyridin-3-yl)-N′-[5-(trifluoromethyl)-1,3,4-thiadiazol-2-yl]urea. Yield 37%. HRMS (FAB) calculated for C₁₁H₁₀F₃N₅O₃S+H 350.0534, found 350.0517.

### Example 306

N-(4-hydroxyphenyl)-N′-[5-(trifluoromethyl)-1,3,4-thiadiazol-2-yl]urea. Yield 12%. HRMS (ESI) calculated for C₁₀H₇F₃N₄O₂S+H 305.0320, found 305.0329.

### Example 307

N-(5-ethoxypyridin-2-yl)-N′-[5-(trifluoromethyl)-1,3,4-thiadiazol-2-yl]urea.

2-Bromo-3-hydroxy pyridine(11.95 mmol), iodoethane (23.91 mmol), and K₂CO₃ (21.51 mmol) are diluted in DMF (20 mL) and heated to 80° C. for 2 h. The solvent is removed under reduced pressure and the residue diluted with H₂O (20 mL), extracted with EtOAc (3×25 mL). The combined organics are washed with H₂O (25 mL), brine (25 mL), dried (MgSO₄), and the solvent is removed to give 2-bromo-3-ethoxypyridin as an off-white solid (1.99 g, 83% yield).

2-Bromo-3-ethoxypyridine (0.01 mol) is added to fuming nitric acid (8 mL) and sulfuric acid (8 mL) at 0° C. The clear yellow solution is heated to 55° C. for 1 h, cooled back to RT and added dropwise to ice H₂O (400 mL). The solid is filtered to give 2-bromo-3-ethoxy-6-nitropyridine as a light yellow solid (1.40 g, 57% yield).

2-Bromo-3-ethoxy-6-nitropyridine is dissolved in a minimal amount of EtOAc (10 mL) and diluted with EtOH (55 mL). 10% Pd/C catalyst is added as a slurry in EtOAc and the mixture is then put on the Parr apparatus under hydrogen for 1 h (40 psi to 18 psi). The reaction mixture is filtered over Celite to remove the catalyst and the filtrate is concentrated. The residue is diluted with 6M HCl (50 mL), extracted with EtOAc (3×50 mL) and the solvent is removed. The residue is diluted with 1M NaOH (50 mL) and extracted with EtOAc (3×50 mL), dried (MgSO₄), and the solvent is removed to give 5-ethoxypyridin-2-amine as a brown oil (722 mg, 94% yield). Example 307 is obtained according to Method C, making non-critical variations. Yield 40%. HRMS (ESI) calculated for C₁₁H₁₀F₃N₅O₂S+H 335.0585 found 335.0584.

### Example 308

N-(4-ethoxy-2-morpholin-4-ylphenyl)-N′-[5-(trifluoromethyl)-1,3,4-thiadiazol-2-yl]urea.

To a cooled (0° C.) mixture of 3-fluoro-4-nitrophenol (8.28 g), potassium carbonate (13.2 g) in 2-butanone (50 mL) is added drop-wise iodoethane (7.7 mL). The resulting suspension is heated to 40° C. for 3 hours and then refluxed for 1 hour. The reaction mixture is concentrated, suspended in CH₂C₁₂, filtered, and concentrated to give a pale yellow solid. Yield 96%. ¹H NMR (400 MHz, CDCl₃) δ 8.11, 6.75, 4.13, 1.49.

Step 2

To a mixture of 4-ethoxy-2-fluoro-1-nitrobenzene (0.86 g) and potassium carbonate (1.3 g) in DMSO (6 mL) is added morpholine (0.45 g). The mixture is heated to 100° C. in a sealed tube for 1 h, cooled, filtered, extracted with water, and concentrated to give a solid. Yield 98%. MS (CI) m/z (rel intensity) 253 (M+H 21), 224 (14), 223 (99), 221 (4), 220 (9), 219 (67), 138 (6), 105 (5), 96 (4), 88 (18).

Step 3

A mixture of 4-(5-ethoxy-2-nitrophenyl)morpholine (0.75 g) and 10% Pd/C (0.16 g) in an approiate solent (either a mixture of EtOAc/MeOH or pure MeOH) is reacted under 45 psi H₂ for 2 h, and is then filtered, concentrated, and purified using silica gel chromatography. Yield 75%. HRMS (ESI) calcd for C₁₂H₁₈N₂O₂+H 223.1447, found 223.1449.

Example 308 (from 4-ethoxy-2-morpholin-4-ylaniline and 2-isocyanato-5-(trifluoromethyl)-1,3,4-thiadiazole) is prepared using Method C, making non-critical modifications to give a solid crystallized from MeCN. Yield 54%. HRMS (ESI) calcd for C₁₆H₁₈N₅O₃SF₃+H 418.1161, found 418.1171.

### Example 309

tert-butyl 4-{5-ethoxy-2-[({[5-(trifluoromethyl)-1,3,4-thiadiazol-2-yl]amino}carbonyl)amino]phenyl}piperazine-1-carboxylate.

tert-Butyl 4-(5-ethoxy-2-nitrophenyl)piperazine-1-carboxylate (from 4-ethoxy-2-fluoro-1-nitrobenzene and tert-butyl piperazine-1-carboxylate) is prepared using Step 2 of Example 308, making non-critical modifications to give a solid purified with silica gel chromatography (40% EtOAc/heptane). Yield quantitative. HRMS (ESI) calcd for C₁₇H₂₅N₃O₅+H 352.1872, found 352.1880.

tert-Butyl 4-(2-amino-5-ethoxyphenyl)piperazine-1-carboxylate is prepared using Step 3 of Example 308 with the following exception: no HCl is used, and the material is purified with silica gel chromatography (40% EtOAc/heptane). Yield 89%. MS (ESI+) for C₁₂H₁₈N₂OS m/z 322.3 (M+H)⁺.

Example 309 (from tert-butyl 4-(2-amino-5-ethoxyphenyl)piperazine-1-carboxylate and 2-isocyanato-5-(trifluoromethyl)-1,3,4-thiadiazole) is prepared using Method C, making non-critical modifications to give a solid crystallized from EtOAc/hexane. Yield 49%. HRMS (ESI) calcd for C₂₁H₂₇N₆O₄SF₃+H 517.1844, found 517.1827.

### Example 310

N-(2-chloro-6-methoxy-pyridin-3-yl)-N′-[5-(trifluoromethyl)-1,3,4-thiadiazol-2-yl]urea.

A solution of 2-chloro-6-methoxy-3-nitropyridine (1.0 g, 5.3 mmol) in EtOH (50 ml)/EtOAc (50 ml) is hydrogenated at RT and 40 psi, in the presence of 10% Pd-C (0.394 g, 0.37 mmol). The suspension is filtered through cellulose and washed with EtOH. The solution is concentrated under vacuum to give a brown oil 0.58 g (69% yield) that is used without further purification in the next step. MS (ESI+) for C₆H₇ClN₂O m/z 159.0 (M+H)⁺.

Example 310 (from 2-chloro-6-methoxypyridin-3-amine and 2-isocyanato-5-(trifluoromethyl)-1,3,4-thiadiazole) is prepared by following Method C, making non-critical modifications. The solid is triturated with CH₂Cl₂ to give a white solid 0.104 g (47% yield). HRMS (ESI) calcd for C₁₀H₇N₅O₂SClF₃+H 354.0039, found 354.0046.

### Example 311

N-[6-methoxy-2-(methylthio)pyridin-3-yl]-N′-[5-(trifluoromethyl)-1,3,4-thiadiazol-2-yl]urea.

To a solution of 2-chloro-6-methoxy-3-nitropyridine (2.0 g, 10.6 mmol) in DMSO (70 ml) is added sodium thiomethoxide (0.743 g, 10.6 mmol). The reaction mixture is stirred at 80° C. for 4 hr. The mixture is diluted with H₂O, extracted with EtOAc, and the combined organic layers are dried (MgSO₄), filtered, and concentrated under vacuum. The residue is purified by silica gel chromatography (20% EtOAc/n-heptane) followed by trituration with CH₂Cl₂/MeOH to afford a yellow solid 1.26 g (59% yield). HRMS (EI) calcd for C₇H₇N₂O₃S 200.0262, found 200.0262.

6-Methoxy-2-(methylthio)pyridin-3-amine (from 6-methoxy-2-(methylthio)-3-nitropyridine) is prepared by following Step 3 of Example 155, making non-critical modifications to afford a brown solid 0.6 g (86% yield). This compound is used without further purification in the next step. MS (ESI+) for C₇H₁₀N₂OS m/z 171.0 (M+H)⁺.

Example 311 (from 6-methoxy-2-(methylthio)pyridin-3-amine and 2-isocyanato-5-(trifluoromethyl)-1,3,4-thiadiazole) is prepared by following Method C, making non-critical modifications. The resulting residue is purified by silica gel chromatography (50% EtOAc/n-heptane) followed by trituration with CH₂Cl₂ to give a white solid 0.265 g (62% yield). HRMS (EI) calcd for C₁₁H₁₀F₃N₅O₂S₂ 365.0228, found 365.0230.

### Example 312

N-[6-methoxy-2-(methylsulfonyl)pyridin-3-yl]-N′-[5-(trifluoromethyl)-1,3,4-thiadiazol-2-yl]urea.

To a suspension of 6-methoxy-2-(methylthio)-3-nitropyridine (0.23 g, 1.14 mmol) in CH₂Cl₂ (20.0 ml), a solution of m-chloroperbenzoic acid 57-86% (0.841 g, 3.42 mmol) in CH₂Cl₂ (8.0 ml) is added dropwise via addition funnel. After stirring the reaction mixture at RT for 1 hr, sodium metabisulfite (0.2 g, 1.06 mmol) is added and the mixture is stirred for additional 10 min. The mixture is diluted with H₂O, extracted with CH₂Cl₂, and the combined organic layers are washed with 1N HCl and 1N NaOH, dried (MgSO₄), filtered, and concentrated under vacuum. The residue is purified by silica gel chromatography (30%EtOAc/n-heptane) to give a white solid 0.225 g (85% yield). HRMS (ESI) calcd for C₇H₈N₂O₅S+H 233.0232, found 233.0227.

A solution of 6-methoxy-2-(methylsulfonyl)-3-nitropyridine (0.22 g, 0.947 mmol) in EtOH (50 ml)/EtOAc (50 ml) is hydrogenated at RT and 40 psi, in the presence of 10% Pd—C (0.070 g, 0.066 mmol). The suspension is filtered through cellulose and washed with EtOH. The solution is concentrated under vacuum to give brown oil 0.18 g (94% yield). This compound is used without further purification in the next step. MS (ESI+) for C₇H₁₀N₂O₃S m/z 203.0 (M+H)⁺.

Example 312 (from 6-methoxy-2-(methylsulfonyl)pyridin-3-amine and 2-isocyanato-5-(trifluoromethyl)-1,3,4-thiadiazole) is prepared by following Method C making non-critical modifications. The residue is purified by silica gel chromatography (50% EtOAc/n-heptane) followed by trituration with CH₂Cl₂ to afford a white solid 0.04 g (26% yield). HRMS (ESI) calcd for C₁₁H₁₀N₅O₄S₂F₃+H 398.0204, found 398.0218.

### Example 313

N-[2-methoxy-4-(2-methoxyethoxy)phenyl]-N′-(5-methylisoxazol-3-yl)urea.

To a cooled solution (˜0° C.) of 2-methoxyethanol (0.8 mL) in CH₂Cl₂ (50 mL) and TEA (2 mL) is added dropwise methanesulfonyl chloride (1.3 mL). After 5 minutes, the bath is removed and the reaction mixture allowed to warm to RT and stir for 1 hour, it is washed with 1.0 N NaOH, brine, dried (MgSO₄), and concentrated to give an oil. Yield quantitative. MS (ESI+) for C₄H₁₀O₄S m/z 155.1 (M+H)⁺.

To a solution of 2-methoxyethyl methanesulfonate (1.54 g) in 2-butanone (150 mL) is added cesium carbonate (6.50 g) and 3-fluoro-4-nitrophenol (1.52 g). The resulting mixture is refluxed for 19 hours, cooled, filtered, concentrated, and purified using silica gel chromatography (EtOAc/Heptanes). Yield 98%. ¹H NMR (400 MHz, DMSO-d₆) δ 7.95, 6.82, 6.67, 4.25, 3.92, 3.68, 3.33, 3.31.

To a mixture of 2-methoxy-4-(2-methoxyethoxy)-1-nitrobenzene (2.0 g), 10% Pd/C (1.0 g) in MeOH (200 mL) is added conc. HCl (1.8 g). The resulting mixture is shaken at 40 psi H₂ for 15 minutes. The mixture is filtered and crystallized from EtOH/Et₂O. Yield 76%. MS (ESI+) for C₁₀H₁₅NO₃ m/z 198.1 (M+H)⁺.

Example 313 (from from 2-methoxy-4-(2-methoxyethoxy)aniline hydrochloride and phenyl 5-methylisoxazol-3-ylcarbamate) is prepared using Method C, making non-critical changes. Yield 91%. HRMS (ESI) calcd for C₁₅H₁₉N₃O₅+H 322.1403, found 322.1394

### METHOD D

### Example 400

N-(4-hydroxy-2-methylphenyl)-N′-[4-(trifluoromethyl)-1,3-thiazol-2-yl]urea.

To a solution of 4-(trifluoromethyl)-1,3-thiazol-2-amine (1 g, 5.95 mmol) in CH₂Cl₂ (150 mL) at 0° C. is added drop-wise phenyl chloroformate (2.2 mL, 17.9 mmol), followed by DMAP (36 mg, 0.3 mmol) and pyridine (0.47 mL, 5.95 mmol). After 20 min., additional pyridine (0.1 mL) is added. Upon completion of the reaction, the mixture is washed with 0.1N HCl followed by 5% NaHCO₃, brine and concentrated. The resulting solid is recrystallized from hexane to afford phenyl-4-(trifluoromethyl)-1,3-thiazol-2-yl-carbamate as a white crystalline solid (1.1 g, 65% yield). HRMS (ESI) calcd for C₁₁H₇F₃N₂O₂S +H 289.0258, found 289.0265.

To a solution of phenyl-4-(trifluoromethyl)-1,3-thiazol-2-yl-carbamate (0.23 g, 0.8 mmol) and 4-amino-m-cresol (0.1 g, 0.8 mmol) in THF (5 mL) is added TEA (0.11 mL, 0.8 mmol). The reaction is heated 50° C. overnight and the solvent is removed. The residue is purified by flash chromatography (SiO₂ gel, 30% EtOAc/hexanes) to afford Example 400 as a white solid (90 mg, 35% yield). HRMS (ESI) calcd for C₁₂H₁₀F₃N₃O₂S+H 318.0524, found 318.0517.

The following compounds are made from a phenyl carbamate and an aniline according to Method D, making non-critical variations.

### Example 401

N-(4-hydroxyphenyl)-N′-[4-(trifluoromethyl)-1,3-thiazol-2-yl]urea (from 4-aminophenol and phenyl 4-(trifluoromethyl)-1,3-thiazol-2-ylcarbamate). The crude product is triturated with CH₂Cl₂ to afford an off white solid 0.142 g (76% yield). HRMS (ESI) calcd for C₁₁H₈N₃O₂SF₃+H 304.0367, found 304.0378.

### Example 402

N-(5-hydroxy-2-methylphenyl)-N′-[4-(trifluoromethyl)-1,3-thiazol-2-yl]urea (from 3-amino-4-methylphenol and phenyl 4-(trifluoromethyl)-1,3-thiazol-2-ylcarbamate). The crude is triturated with CH₂Cl₂ to give a white solid 0.147 g (57% yield). HRMS (ESI) calcd for C₁₂H₁₀N₃O₂SF₃+H 318.0524, found 318.0539.

### Example 403

N-(3-hydroxy-2-methylphenyl)-N′-[4-(trifluoromethyl)-1,3-thiazol-2-yl]urea (from 3-amino-2-methylphenol and 4-(trifluoromethyl)-1,3-thiazol-2-ylcarbamate). The crude is diluted with MeOH and DOWEX 50WX2-400 ion exchange resin is added; the mixture is allowed to spin submerged in a water bath (35-40° C.) for 20 minutes, is filtered, and the resin washed with MeOH. The product is liberated from the resin by treatment with a solution of 20% NH₄OH/MeOH. The basic alcohol washes are concentrated in vacuo to give a brown solid, which is crystallized from CH₂Cl₂ to give a light brown solid 0.173 g (67% yield). HRMS (ESI) calcd for C₁₂H₁₀N₃O₂SF₃+H 318.0524, found 318.0517.

### Example 404

N-(6-cyanopyridin-3-yl)-N′-(5-fluoro-2,4-dimethoxyphenyl)urea.

To a solution of 5-amino-2-cyanopyridine (1.5 g, 12 mmol) and phenylchloroformate (1.9 mL, 15 mmol) in 100 mL of CH₂Cl₂ at 0° C. is added pyridine (1.2 mL, 15 mmol) and the solution is stirred for 2 h. The reaction is diluted with 0.1 M HCl and the organic layer is separated, washed with 5% sodium bicarbonate, brine, dried (MgSO₄), and the solvent is removed in vacuo to provide phenyl 6-cyanopyridin-3-ylcarbamate (0.75 g, 25% yield). ¹H NMR (400 MHz, CDCl₃) δ 8.63, 8.23, 7.69, 7.46-7.40, 7.36-7.26, 7.22-7.17. Example 404 is prepared according to Method D, making non-critical variations. Yield 61%. HRMS (ESI) calcd for C₁₅H₁₃FN₄O₃+H 317.1050 found 317.1062.

### Example 405

N-(4-hydroxy-2-methylphenyl)-N′-[3-(trifluoromethoxy)phenyl]urea.

Step 1

To a solution of 3-trifluoromethoxy aniline (0.5 g, 2.8 mmol) in CH₂Cl₂ (70 ml) is added dropwise, phenyl chloroformate (1.06 ml, 8.5 mmol) and pyridine (0.226 ml, 2.8 mmol) at 0° C. The reaction mixture is stirred at 0° C. for 30 min. The solution is washed with 0.1 N HCl, 5% NaHCO₃, brine, and concentrated under vacuum. The resulting solid is crystallized from n-heptane to give white needles 0.6 g (71% yield). HRMS (EI) calcd for C₁₄H₁₀F₃NO₃ 297.0613, found 297.0613.

Example 405 (from 4-amino-3-methylphenol and phenyl 3-(trifluoromethoxy)phenylcarbamate) is prepared by following Method D, making non-critical modifications. The resulting residue is triturated with CH₃CN to give an off white solid 0.07 g (33% yield). HRMS (ESI) calcd for C₁₅H₁₃N₂O₃F₃+H 327.0956, found 327.0954.

### Example 406

N-(4-hydroxy-2-methylphenyl)-N′-[4-(pentafluoroethyl)-1,3-thiazol-2-yl]urea.

To a solution of 4-amino-3-methylphenol (4.25 g, 33.5 mmol) and imidazole (2.53 g, 36.8 mmol) in THF (100 ml) is added tert-butyl(chloro)dimethylsilane (5.78 g, 37.1 mmol) at 0° C. The reaction mixture is warmed to RT and stirred for 3 hr. The mixture is concentrated under vacuum, diluted with Et₂O, and the combined organic layers are washed with satd. NaHCO₃, brine, and dried (MgSO₄), filtered, and concentrated under vacuum. The residue is purified by silica gel chromatography (50% EtOAc/n-heptane) to give a brown oil 7.2 g (91% yield). ¹H NMR (400 MHz, CDCl₃) δ 6.93, 6.61, 6.56, 2.18, 0.98, 0.18.

Phenyl 4-{[tert-butyl(dimethyl)silyl]oxy}-2-methylphenylcarbamate (from 4-{[tert-butyl(dimethyl)silyl]oxy}-2-methylaniline and phenyl chloroformate) is prepared by following Step 1 of Example 407, making non-critical modifications. The residue is purified by silica gel chromatography (CH₂C₁₂) followed by recrystallization from hexane to give a white solid 3.3 g (61% yield). HRMS (ESI) calcd for C₂₀H₂₇NO₃Si+H 358.1838, found 358.1848.

4-(Pentafluoroethyl)-1,3-thiazol-2-amine is prepared from thiourea and 1-bromo-3,3,4,4,4-pentafluoro-2-butanone by the general procedure described in Biotechnology and Bioengineering (Combinatorial Chemistry), 2000, 71(1), 9. The free base is obtained by the procedure described in the preparation of N-(4-ethyl-1,3-thiazol-2-yl)-N′-(4-methoxy-2-methylphenyl)urea. Pure 4-(pentafluoroethyl)-1,3-thiazol-2-amine may also be obtained by dissolving the crude reaction product in MeOH (150 mL) and adding Dowex O50WX2-400 acidic ion exchange resin (10 g) and stirring the mixture overnight at RT. The mixture is filtered and the resin is washed sequentially with MeOH (150 mL) and 20% aqueous NH₄OH in MeOH (200 mL). The 20% aqueous NH₄OH in MeOH wash is concentrated and the resulting light brown crystals are washed with cold hexane and dried to yield 4-(pentafluoroethyl)-1,3-thiazol-2-amine (1.8 g).

N-(4-{[tert-butyl(dimethyl)silyl]oxy}-2-methylphenyl)-N′-[4-(pentafluoroethyl)-1,3-thiazol-2-yl]urea (from 4-(pentafluoroethyl)-1,3-thiazol-2-amine and phenyl 4-{[tert-butyl(dimethyl)silyl]oxy}-2-methylphenylcarbamate) is prepared by following Method D, making non-critical modifications to give a brown solid (0.3 g), which is dissolved in THF (3.0 ml) and 1.0M solution of tetra-butyl ammonium fluoride (2.0 ml, 2.0 mmol) is added. The reaction mixture is stirred at RT for 15 min. The mixture is diluted with H₂O, extracted with CH₂Cl₂, and the combined organic layers are dried (MgSO₄), filtered, and concentrated under vacuum. The residue is purified by silica gel chromatography (30% EtOAc/n-heptane) followed by trituration with CH₂Cl₂ to give Example 406 as a white solid 0.110 g (43% yield). HRMS (ESI) calcd for C₁₃H₁₀N₃O₂SF₅+H 368.0492, found 368.0490.

### Example 407

N-(5-bromo-2,4-dimethoxyphenyl)-N′-(6-cyanopyridin-3-yl)urea. Yield 27%. MS (ESI) for C₁₅H₁₃BrN₄O₃ (M−H)³¹ mz 375.

### METHOD E cl Example 500

N-(5-chloro-2,4-dimethoxyphenyl)-N′-[3-(trifluoromethyl)isoxazol-5-yl]urea.

Pyridine (5.4 mL, 0.067 mol) is added dropwise to 5-chloro-2,4-dimethoxyaniline (10.45 g, 0.056 mol) and phenyl chloroformate (8.4 mL, 0.067 mol) in CH₂Cl₂ (500 mL) at 0° C. The reaction is stirred for 1.5 h and diluted with 0.1M HCl (100 mL). The organics are separated, washed w/5% NaHCO₃ (100 mL) and brine (150 mL), dried (MgSO₄), and the solvent is removed. The resulting solid is recrystallized (EtOAc/hexanes) to give phenyl 5-chloro-2,4-dimethoxyphenylcarbamate (16.72 g, 97% yield) as a purple solid.

Sodium hydride(1.82 g, 0.046 mol, 60% oil disp.) is added to 3-(trifluoromethyl)isoxazole-5-amine (6.93 g, 0.046 mol) in DMF (350 mL) at RT. After 0.5 h, a DMF solution (100 mL) of phenyl 5-chloro-2,4-dimethoxyphenylcarbamate (14.0 g, 0.046 mol) is added and the reaction warmed to RT. The reaction is heated at 50° C. for 1 h, cooled to RT and the solvent is removed. The residue is dissolved in EtOAc (150 mL), washed with 1M HCl (150 mL), H₂O (150 mL), and brine (150 mL). The organics are separated, dried (MgSO₄) and the solvent is removed under reduced pressure. The dark solid is dissolved in EtOH (500 mL) and stirred with Darco activated carbon (˜15) for 4 h. The mixture is filtered over Celite and the solvent is removed to give a solid, which is recrystallized (EtOAc/hexanes) to give Example 500 as a tan solid (10.47 g, 63% yield). HRMS (ESI) calcd for C₁₃H₁₁ClF₃N₃O₄+H 366.0468 found 366.0475.

The following compounds are made from an aminoheterocycle, a phenyl carbamate and a base according to Method E, making non-critical variations.

### Example 501

N-(5-fluoro-2,4-dimethoxyphenyl)-N′-[3-(trifluoromethyl)isoxazol-5-yl]urea. Yield 63%. HRMS (ESI) calcd for C₁₃H₁₁F₄N₃O₄+H 350.0764 found 350.0770.

### Example 502

### N-(2,6-dimethoxypyridin-3-yl)-N′-[3-(trifluoromethyl)isoxazol-5-yl]urea. Yield 47%. HRMS (ESI) calcd for C₁₂H₁₁F₃N₄O₄+H 333.0811 found 333.0808.

### Example 503

N-(4-ethoxy-2-nitrophenyl)-N′-[3-(trifluoromethyl)isoxazol-5-yl]urea. Yield 81%. HRMS (ESI) calcd for C₁₃H₁₁F₃N₄O₅+H 360.0681 found 360.0685.

### METHOD F

### Example 4

Alternative Procedure for Preparation.

A solution of 5-methylisoxazole-3-carbonyl chloride (4.9 g, 33.3 mmol) in 50 mL acetone is cooled to 0° C. and treated with sodium azide (2.5 g, 38.5 mmol) dissolved in 3 mL water. The solution is allowed to warm to RT. After 1 h, the solvent is removed, suspended in water, filtered and dried in vacuo to provide 4.65 g (92% yield) of 5-methylisoxazole-3-carbonyl azide. ¹H NMR (CDCl₃, 400 MHz) δ 2.51, 6.46 ppm.

A solution of 5-methylisoxazole-3-carbonyl azide (166 mg, 1.09 mmol) and 5-chloro-2,4-dimethoxyaniline (204 mg, 1.09 mmol) in 15 mL CH₃CN is heated under reflux. After 16 h, the reaction is cooled. The white precipitate is filtered, washed with ether and dried in vacuo to provide 264 mg (78% yield) of Example 4.

The following compounds are made from an acyl azide and an aniline according to Method F, making non-critical variations.

### Example 601

N-(5-chloro-2,4-dimethoxyphenyl)-N′-(5-chloroisoxazol-3-yl)urea.

To a solution of ethyl chlorooximidoacetate (11.0 g, 73 mmol) in 1,1-dichloroethylene (320 mL) is added Et₃N (25 mL, 181 mmol) in 1,1-dichloroethylene (90 mL) via addition funnel over 30 minutes. The reaction mixture is stirred at RT for 3 days, after which it is partitioned between water (150 mL) and CH₂Cl₂ (2×150 mL). The combined organic layers are washed with brine, dried (Na₂SO₄) and concentrated to give crude oil. Crude product is chromatographed (Biotage 40M, EtOAc/Hex:5/95) to yield 1.9 g (15% yield) of ethyl 5-chloroisoxazole-3-carboxylate as a yellow oil. 1H NMR (400 MHz, DMSO-d₆) δ 6.61, 4.46, 1.42.

An aqueous solution of LiOH (2.0 M, 11.4 mmol) is added to a solution of ethyl 5-chloroisoxazole-3-carboxylate (1.0 g, 5.7 mmol) in EtOH (25 mL) at RT. After 45 minutes, the volatiles are removed in vacuo. The residue is partitioned between 1 M HCl (40 mL) and EtOAc (2×40 mL). The combined organic layers are dried (Na₂SO₄), and concentrated in vacuo to give 0.72 g (86% yield) of 5-chloroisoxazole-3-carboxylic acid as a tan solid. ¹H NMR (400 MHz, DMSO-d₆) δ 7.13.

Oxalyl chloride (0.44 mL, 5.1 mmol) is added dropwise to a suspension of 5-chloroisoxazole-3-carboxylic acid (0.50 g, 3.4 mmol) and a catalytic amount of DMF in CH₂Cl₂ (20 mL). After 1 h, the volatiles are removed in vacuo and the remaining residue is dissolved in acetone. To this solution is added an aqueous solution of sodium azide (0.31 g, 4.8 mmol) at 0° C. with vigorous stirring. Volatiles are removed in vacuo and the residue washed with water and dried under nitrogen to yield 0.32 g (55% yield) of 5-chloroisoxazole-3-carbonyl azide as a white solid. ¹H NMR (400 MHz, CDCl₃) δ 6.65. Example 601 is obtained according to Method F, making non-critical modifications. Yield 10%. HRMS (ESI) calcd for C₁₂H₁₁C₁₂N₃O₄+H 332.0205, found 332.0201.

### Example 602

N-(5-chloro-4-methoxy-2-nitrophenyl)-N′-(5-methylisoxazol-3-yl)urea.

To a suspension of 3-chloro-p-anisidine (3.00 g, 19 mmol) in CH₂Cl₂ (10 mL) is added acetic anhydride (1.8 mL, 19 mmol) over 1 h at a temperature between 25-30° C. using a cold water bath. The mixture is then stirred for an additional 2 h at 25° C. Hexane (30 mL) is slowly added to the mixture over 1 h. The product is collected by filtration, concentrated in vacuo and dried in oven to give 3.38 g (89% yield) of N-(3-chloro-4-methoxyphenyl)acetamide as purple crystals. ¹H NMR (400 MHz, CDCl₃) δ 7.54, 7.37, 7.16, 6.87, 3.88, 2.16.

In a 100 mL, three-necked flask equipped with a thermometer and a dropping funnel chilled in an ice-salt bath, N-(3-chloro-4-methoxyphenyl)acetamide (1.50 g, 7.5 mmol) is dissolved in 98% sulfuric acid, care being taken that the temperature does not rise above 5° C. To this solution 1.2 mL of nitric acid is added, resulting in rise in temperature to 33° C. After the temperature drops below 5° C., the viscous orange mass is poured onto 25 g of cracked ice and the mixture is thoroughly stirred. The yellow crystals that separate are filtered off, thoroughly washed with water and recrystallized from ethanol to yield 0.45 g (24% yield) of N-(5-chloro-4-methoxy-2-nitrophenyl)acetamide as yellow crystals. ¹H NMR (400 MHz, DMSO-d₆) δ 10.15, 7.72, 7.66, 3.94, 2.04.

A suspension of N-(5-chloro-4-methoxy-2-nitrophenyl)acetamide (0.41 g, 1.7 mmol) in 6.5 mL of water, 6.5 mL of HCl and 3 mL of ethanol is refluxed for 30 minutes. On cooling, crystals separate and are recrystallized from ethanol to yield 0.21 g (61% yield) of 5-chloro-4-methoxy-2-nitroaniline as red-orange crystals. ¹H NMR (400 MHz, DMSO-d₆) δ 7.53, 7.33, 7.20. Example 602 is obtained according to Method F, making non-critical modifications. Yield 73%. HRMS(ESI) calcd for C₁₂H₁₁ClN₄O₅+H 327.0496 found 327.0509.

### Example 603

N-(5-fluoro-4-methoxy-2-nitrophenyl)-N′-(5-methylisoxazol-3-yl)urea.

To a stirred suspension of 3-fluoro-p-anisidine (3.00 g, 21 mmol) in CH₂Cl₂ (10 mL) is added acetic anhydride (2.0 mL, 21 mmol) over 1 h while maintaining the temperature between 25-30° C. using a cold water bath. The solution is then stirred for 2 h at 25° C. Additional 1 mL of acetic anhydride is added to the solution, which is then stirred for an additional 2 h. Hexane (30 mL) is slowly added to the mixture over 1 h. The product is collected by filtration, concentrated in vacuo and dried in oven to give 3.40 g (90% yield) of N-(3-fluoro-4-methoxyphenyl)acetamide as tan crystals. ¹H NMR (400 MHz, CDCl₃) δ 7.41, 7.19, 7.11, 6.90, 3.87, 2.16.

In a 100 mL, three-necked flask equipped with a thermometer and a dropping funnel chilled in an ice-salt bath, N-(3-fluoro-4-methoxyphenyl)acetamide (3.0 g, 16 mmol) is dissolved in 98% sulfuric acid, care being taken that the temperature does not rise above 5° C. To this solution 2.6 mL of nitric acid is added dropwise at such a rate that the temperature does not rise above 5° C. After all the nitric acid is added, the viscous orange mass is poured onto 50 g of cracked ice and the mixture is thoroughly stirred. The yellow crystals that separate are filtered off and thoroughly washed with water to yield 3.33 g (89% yield) of N-(5-fluoro-4-methoxy-2-nitrophenyl)acetamide as yellow crystals. ¹H NMR (400 MHz, DMSO-d₆) δ 10.60, 10.17, 7.75, 7.73, 7.62, 7.59, 4.03, 3.92, 2.06.

A suspension of N-(5-fluoro-4-methoxy-2-nitrophenyl)acetamide (3.0 g, 13 mmol) in 38 mL of water, 38 mL of HCl and 15 mL of ethanol is refluxed for 30 minutes. On cooling, crystals that separate are recrystallized from ethanol to yield 0.71 g (29% yield) of 5-fluoro-4-methoxy-2-nitroaniline as red crystals. ¹H NMR (400 MHz, CDCl₃) δ 7.68, 6.56, 6.04, 3.87. Example 603 is obtained according to Method F, making non-critical modifications. Yield 60%. HRMS (ESI) calcd for C₁₂H₁₁FN₄O₅+H 311.0792 found 311.0798.

### Example 604

N-[5-chloro-4-methoxy-2-(methylthio)phenyl]-N′-(5-methylisoxazol-3-yl)urea.

A solution of sodium thiomethoxide (1.64 g, 23 mmol) in methanol is added dropwise to a solution of 1,2-dichloro-4-fluoro-5-nitrobenzene (4.92 g, 23 mmol) in methanol. The resulting mixture is stirred for 2 h, quenched with 1 M citric acid, and volatiles are removed in vacuo. The crude solids are diluted with EtOAc, washed with 1 M citric acid, 1 M NaOH, brine, dried (MgSO₄) and concentrated in vacuo to yield 5.52 g (99% yield) of 1,2-dichloro-4-(methylthio)-5-nitrobenzene as a yellow solid. ¹H NMR (400 MHz, CDCl₃) δ 8.38, 7.41, 2.51.

Methanol (1 mL) is added to NaH (60% oil disp., 1.02 g, 26 mmol) in DMF at RT, followed by addition of 1,2-dichloro-4-(methylthio)-5-nitrobenzene. The mixture is refluxed for 2 days, cooled to RT, diluted with water and extracted with EtOAc (3×50 mL). The organics are washed with water, brine, dried (MgSO₄), and concentrated in vacuo to give the crude product. Crude solids are chromatographed (Biotage 40M, EtOAc/Hex:10/90) and recrystallized from acetonitrile to yield 0.43 g (9% yield) of 1-chloro-2-methoxy-4-(methylthio)-5-nitrobenzene as a yellow solid. ¹H NMR (400 MHz, DMSO-d₆) δ 12.01, 7.01, 2.46.

To a suspension of 1-chloro-2-methoxy-4-(methylthio)-5-nitrobenzene (0.41 g, 1.8 mmol) in water is added acetic acid (0.1 mL) and the mixture is heated to 85° C. Iron powder (2.5 g, 45 mmol) is added slowly to the mixture. After 3 h, the resulting slurry is neutralized with 10% Na₂CO₃ and filtered. The filtrate is extracted with EtOAc (3×50 mL), dried (MgSO₄) and concentrated in vacuo. The crude solids are treated with hot DMF and filtered. DMF is removed under reduced pressure to yield 0.26 g (71% yield) of 5-chloro-4-methoxy-2-(methylthio)aniline as an oil. ¹H NMR (400 MHz, DMSO-d₆) δ 6.95, 6.79, 4.96, 3.74, 2.36. Example 604 is obtained according to Method F, making non-critical modifications. Yield 31%. HRMS (ESI) calcd for C₁₃H₁₄ClN₃O₃S+H 328.0522 found 332.0522.

### Example 605

N-(5-chloro-2,4-dimethoxyphenyl)-N′-[5-(fluoromethyl)isoxazol-3-yl]urea.

Ethyl chlorooximidoacetate (10 g, 66 mmol) in CHCl₃ (80 mL) is slowly added to propargyl alcohol (4.7 mL, 81 mmol) and K₂CO₃ (27 g, 198 mmol) in CHCl₃ (80 mL). The addition is accompanied by an exothermic reaction which causes the chloroform to reflux. After being allowed to cool to RT, the mixture is stirred overnight. The reaction mixture is filtered and the residue is rinsed with chloroform and concentrated in vacuo. The crude product is purified by chromatohraphy (Biotage 40M, EtOAc/Hex:20/80) to yield 3.23 g (29% yield) of ethyl 5-(hydroxymethyl)isoxazole-3-carboxylate as an oil. ¹H NMR (400 MHz, CDCl₃) δ 6.69, 4.86, 4.45, 4.42.

A solution of diethylaminosulfur trifluoride (DAST) (2.8 mL, 21 mmol) in 30 mL CH₂Cl₂ is added dropwise to a solution of ethyl 5-(hydroxymethyl)isoxazole-3-carboxylate (3.0 g, 18 mmol) in 30 mL CH₂Cl₂ at −78° C. The mixture is stirred at −78° C. for 1 h and then warmed to RT over 3 h. Water (10 mL) and 30 mL of 2.5% aqueous sodium bicarbonate solution are added successively and the organic layer is separated, dried (MgSO₄) and evaporated in vacuo. The residue is purified by chromatography (Biotage 40M, EtOAc/Hex:20/80) to give 1.83 g (60% yield) of ethyl 5-(fluoromethyl)isoxazole-3-carboxylate as an oil. ¹H NMR (400 MHz, CDCl₃) δ 6.82, 5.54, 5.42, 4.47, 1.43.

10% Aqueous sodium hydroxide solution (5 mL) is added to a solution of ethyl 5-(fluoromethyl)isoxazole-3-carboxylate (1.82 g, 10 mmol) in ethanol (30 mL) at RT. The mixture is stirred for 2 h and the solvents are evaporated in vacuo. The residue is dissolved in water and acidified to pH 1 with 35% HCl. Ethanol is added, solvents are evaporated in vacuo and the residue is azeotroped with ethanol. Ethanol is added and the mixture filtered to remove inorganic solids. Evaporation in vacuo of the filtrate gives 0.95 g (63% yield) of 5-(fluoromethyl)isoxazole-3-carboxylic acid as a tan solid. ¹H NMR (400 MHz, DMSO-d₆) δ 7.05, 5.67, 5.56.

Oxalyl chloride (0.85 mL, 9.8 mmol) is added dropwise to a suspension of 5-(fluoromethyl)isoxazole-3-carboxylic acid (0.94 g, 6.5 mmol) and a catalytic amount of DMF in 20 mL CH₂Cl₂. After 1 h, the volatiles are removed in vacuo and the remaining residue is dissolved in acetone. To this solution is added an aqueous solution of sodium azide (0.59 g, 9.1 mmol) at 0° C. with vigorous stirring. Volatiles are removed in vacuo and the residue washed with water and dried under nitrogen to yield 0.57 g (51% yield) of 5-(fluoromethyl)isoxazole-3-carbonyl azide as a white solid. ¹H NMR (400 MHz, DMSO-d₆) δ 7.21, 5.71, 5.59. Example 605 is prepared according to Method F, making non-critical modifications. Yield 37%. HRMS (ESI) calcd for Cl₃H₁₃ClFN₃O₄+H 330.0657 found 330.0649

The following compounds are made from 5-(fluoromethyl)isoxazole-3-carbonyl azide, an aniline according to Method F, making non-critical variations.

### Example 606

N-(2,6-dimethoxypyridin-3-yl)-N′-[5-(fluoromethyl)isoxazol-3-yl]urea. Yield 42%. HRMS (ESI) calcd for C₁₂H₁₃FN₄O₄+H 297.0999 found 297.0991.

### Example 607

N-(5-fluoro-2,4-dimethoxyphenyl)-N′-[5-(fluoromethyl)isoxazol-3-yl]urea. Yield 9%. HRMS (ESI) calcd for C₁₃H₁₃F₂N₃O₄+H 314.0952 found 314.0941.

### Example 608

N-(5-bromo-2,4-dimethoxyphenyl)-N′-[5-(fluoromethyl)isoxazol-3-yl]urea. Yield 46%. HRMS (ESI) calcd for C₁₃H₁₃BrFN₃O₄+H 374.0152 found 374.0162.

### Example 609

N-(4-ethoxy-2-nitrophenyl)-N′-[5-(fluoromethyl)isoxazol-3-yl]urea. Yield 38%. HRMS (ESI) calcd for C₁₃H₁₃FN₄O₅+H 325.0948 found 325.0937.

### Example 610

N-(2,4-dimethoxy-5-methylphenyl)-N′-[5-(fluoromethyl)isoxazol-3-yl]urea. Yield 22%. HRMS (ESI) calcd for C14H16N3O4F +H 310.1203, found 310.1198.

### Example 611

N-(5-bromo-2,4-dimethoxyphenyl)-N′-[5-(hydroxymethyl)isoxazol-3-yl]urea.

A solution of ethyl chlorooximidoacetate (15 g, 0.1 mol) in CH₂Cl₂ is added dropwise over 4 h to propargyl alcohol (29 mL, 0.5 mol) and Et₃N (14 mL, 0.1 mmol) in 200 mL CH₂Cl₂. When the addition is complete, the reaction mixture is concentrated and triturated with Et₂O. The solid is filtered and the organics are concentrated again. The remaining oil is chromatographed over silica gel (EtOAc/Hex:20/80) to give ethyl 5-(hydroxymethyl)isoxazole-3-carboxylate an oil. The remaining propargyl alcohol is removed by azeotroping from n-heptane to yield 11.7 g (69% yield). ¹H NMR (400 MHz, CDCl₃) δ 6.69, 4.84, 4.44, 1.42.

To a solution of ethyl 5-(hydroxymethyl)isoxazole-3-carboxylate (4.0 g, 23 mmol) and TBSCl (3.7 g, 25 mmol) in DMF is added Et₃N (3.4 mL, 24 mmol) dropwise over 20 minutes. The reaction is allowed to stir for 30 minutes after which it is diluted with EtOAc (300 mL), washed with 1 M HCl (3×100 mL), 5% CuSO₄ (2×50 mL) and concentrated in vacuo to yield 6.91 g (>100% yield) of oily material with visible TBS impurities by NMR. ¹H NMR (400 MHz, CDCl₃) δ 6.49, 4.69, 4.31, 1.30, 0.80, 0.02.

A mixture of ethyl-5-({[tert-butyl(dimethylsilyl)]oxy}methyl)isoxazole-3-carboxylate (1.68 g, 5.9 mmol) and hydrazine hydrate (044 g, 8.8 mmol) in ethanol (30 mL) is heated to 60° C. for 4 h. The mixture is cooled to RT and the solvents are removed in vacuo to yield 1.40 g (87% yield) of orange crystals. ¹H NMR (400 MHz, DMSO-d₆) δ 9.95, 6.61, 4.74, 4.51, 0.79.

A mixture of 5-({[tert-butyl(dimethyl)silyl]oxy}methyl)isoxazole-3-carbohydrazide (1.32 g, 4.9 mmol) in concentrated hydrochloric acid (40 mL) is cooled to 0° C., followed by a dropwise addition of aqueous NaNO₂ (0.42 g, 6.1 mL), maintaining the temperature below 5° C. After 1 h, the yellow mixture is diluted with water (100 mL) and extracted with EtOAc (3×50 mL). Organics are dried (MgSO₄) and concentrated in vacuo to yield 0.93 g (>100% yield) of 5-(hydroxymethyl)isoxazole-3-carbonylazide as tan crystals. ¹H NMR (400 MHz, DMSO-d₆) δ 6.82, 4.64. Example 611 is obtained according to Method F. Yield 20%. MS (ESI) for C₁₃H₁₄BrN₃O₅ m/z 372 (M−H)−.

### Example 612

N-(5-fluoro-2,4-dimethoxyphenyl)-N′-(5-isopropylisoxazol-3-yl)urea.

Sodium (1.33 g, 57.8 mmol) is added to EtOH (30 mL) and at 0° C. and 3-methyl-2-butanone (6.2 mL, 57.7 mmol) and diethyl oxalate (7.9 mL, 57.8 mmol) are added. The resulting solid is allowed to stand for 1 h and then heated to 80° C. for 0.75 h. The reaction is cooled to RT and acidified to pH 2 with dilute sulfuric acid. Water (30 mL) is then added and the product extracted with EtOAc (2×50 mL), dried (MgSO₄), and the solvent is removed to give ethyl 5-methyl-2,4-dioxohexanoate as an orange oil (9.74 g, 90% yield). MS (ESI) for C₉H₁₄O₄ m/z 187 (M+H)⁺.

Hydroxylamine hydrochloride (10.9 g, 156.3 mmol) is added to ethyl 5-methyl-2,4-dioxohexanoate (9.7 g, 52.1 mmol) stirring in EtOH (200 mL) at RT. The reaction is heated to reflux for 1 h then cooled to RT and the solvent is removed. The residue is diluted with H₂O (50 mL) and extracted with EtOAc (3×50 mL) and dried (MgSO₄) and the solvent is removed to give ethyl 5-isopropylisoxazole-3-carboxylate as an orange oil (9.39 g, 98% yield). MS (ESI) for C₉H₁₃NO₃ m/z 184 (M+H)⁺.

Hydrazine hydrate (1.2 g, 24.7 mmol) is added to ethyl 5-isopropylisoxazole-3-carboxylate (3.0 g, 16.4 mmol) stirring in EtOH (30 mL) and the reaction is heated to 60° C. for 3 h. The reaction is cooled to RT and the solid filtered (157 mg). The filtrate is concentrated and the solid collected by filtering from petroleum ether to give 5-isopropylisoxazole-3-carbohydrazide as an off-white solid (2.46 g, 88% yield). MS (ESI) for C₇H₁₁N₃O₃ m/z 170 (M+H)⁺.

An aqueous solution of NaNO₂ (1.1 g in 10 mL H₂O) is added dropwise to 5-isopropylisoxazole-3-carbohydrazide (2.1 g, 12.5 mmol) in conc. HCl (60 mL). The reaction is stirred for 1.5 h and diluted with H₂O (25 mL). The precipitate is filtered, washed with H₂O (50 mL) and dried to give 5-isopropylisoxazole-3-carbonyl azide as a white solid (1.17 g, 52% yield). The aqueous is extracted with EtOAc (3×50 mL) to give a second crop of product (0.85 g, total yield=90% yield). Example 612 is obtained according to Method F, making non-critical modifications. Yield 71%. HRMS (ESI) calcd for C₁₅H₁₈FN₃O₄+H 324.1359 found 324.1354.

The following compounds are made from 5-isopropylisoxazole-3-carbonyl azide and an aniline according to Method F, making non-critical variations.

### Example 613

N-(5-chloro-2,4-dimethoxyphenyl)-N′-(5-isopropylisoxazol-3-yl)urea. Yield 71%. MS (ESI) for Cl₅H₁₈ClN₃O₄ m/z 338 (M−H)⁻.

### Example 614

N-(5-bromo-2,4-dimethoxyphenyl)-N′-(5-isopropylisoxazol-3-yl)urea. Yield 59%. HRMS (ESI) calcd for C₁₅H₁₈BrN₃O₄+H 384.0559 found 384.0558.

### Example 615

N-(2,6-dimethoxypyridin-3-yl)-N′-(5-isopropylisoxazol-3-yl)urea. Yield 69%. MS (ESI) for C₁₄H₁₈N₄O₄ m/z 305 (M−H)⁻.

### Example 616

N-(2,4-dimethoxy-5-methylphenyl)-N′-(5-isopropylisoxazol-3-yl)urea Yield 62%. MS (ESI) for C₁₆H₂₁N₃O₄ m/z 318 (M−H)⁻.

### Example 617

N-(5-fluoro-2,4-dimethoxyphenyl)-N′-[5-(methoxymethyl)isoxazol-3-yl]urea.

Ethyl chlorooximidoacetate (5.1 g, 33.7 mmol) is added dropwise as a solution in CH₂Cl₂ (60 mL) over 2 h to the methyl propargyl ether (14.2 mL, 168.4 mmol) and TEA (4.7 mL, 33.4 mmol) in CH₂Cl₂ (100 mL) at RT. The reaction is concentrated and the solids filtered and washed with ether. The filtrate is concentrated and purified by column chromatography (25% EtOAc/hexanes as eluent) to give ethyl 5-(methoxymethyl)isoxazole-3-carboxylateas a light yellow oil (2.67 g, 43% yield). MS (ESI) for C₈H₁₁NO₄ m/z 186 (M+H)⁺.

Hydrazine hydrate (0.6 mL, 12.1 mmol) is added to ethyl 5-(methoxymethyl)isoxazole-3-carboxylate (1.5 g, 8.1 mmol) precursor stirring in EtOH (20 mL) and the reaction is heated to 60° C. for 1.5 h. The solvent is removed and the solid collected by filtering from petroleum ether to give 5-(methoxymethyl)isoxazole-3-carbohydrazide as a tan solid (1.23 g, 90% yield). MS (ESI) for C₆H₉N₃O₃ m/z 172 (M+H)⁺.

An aqueous solution of NaNO₂ (0.60 g in 5 mL H₂O) is added dropwise to 5-(methoxymethyl)isoxazole-3-carbohydrazide (1.2 g, 7.0 mmol) in conc. HCl (35 mL) at 0° C. The reaction is stirred for 1.5 h and diluted with H₂O (25 mL). The aqueous is extracted with EtOAc (3×50 mL), dried (MgSO₄), and the solvent is removed to give 5-(methoxymethyl)isoxazole-3-carbonyl azide as a tan solid (1.21 g, 96% yield). Example 617 is obtained according to Method F, making non-critical modifications. Yield 15%. MS (ESI) for C₁₄H₁₆FN₃O₅ m/z 324 (M−H)⁻.

The following compounds are made from 5-(methoxymethyl)isoxazole-3-carbonyl azide and an aniline according to Method F, making non-critical variations.

### Example 618

N-(5-chloro-2,4-dimethoxyphenyl)-N′-[5-(methoxymethyl)isoxazol-3-yl]urea. Yield 15%. MS (ESI) for C₁₄H₁₆ClN₃O₅ (M−H)⁻ m/z 340.

### Example 619

N-(5-bromo-2,4-dimethoxyphenyl)-N′-[5-(methoxymethyl)isoxazol-3-yl]urea. Yield 10%. MS (ESI) for C₁₄H₁₆BrN₃O₅ (M−H)⁻ m/z 384.

### Example 620

N-(4-ethoxy-2-nitrophenyl)-N′-[5-(methoxymethyl)isoxazol-3-yl]urea. Yield 40%. MS (ESI) for C₁₄H₁₆N₄O₆ (M+H)⁺ m/z 337.

### Example 621

N-(5-chloro-2,4-dimethoxyphenyl)-N′-(5-cyclopropylisoxazol-3-yl)urea.

Sodium ethoxide (10 g, 147 mmol) is combined with absolute EtOH (60 mL) in an oven-dried flask, under nitrogen and heated to 70° C. to aid dissolution. The mixture is cooled to 0° C., treated drop-wise with a mixture of cyclopropyl methyl ketone (14.56 mL, 147 mmol) and diethyl oxylate (19.96 mL, 147 mmol) and warmed to RT. Stirring was difficult, so additional EtOH (60 mL) is added and the mixture is stirred for 1 h, then heated to 80° C. for 45 min. The mixture is cooled to RT and concentrated to dryness. The resulting solid is triturated with EtOAc, filtered, and rinsed with EtOAc and Et₂O to remove the reddish color. The solid is dissolved in water (300 mL), acidified to pH 2 with dilute H₂SO₄, extracted with Et₂O (400 mL total), dried (Na₂SO₄) and concentrated to afford 18.0 g (66% yield) of ethyl 4-cyclopropyl-2,4-dioxobutanoate as an amber oil. HRMS (ESI) calcd for C₉H₁₂O₄ +H: 185.0814, found 185.0821 (M+H)⁺.

Ethyl 4-cyclopropyl-2,4-dioxobutanoate (12.92 g, 70.1 mmol) is combined with hydroxylamine hydrochloride (14.62 g, 210.4 mmol) in EtOH (250 mL), heated to reflux for 1 h, cooled, and concentrated to dryness. The residue is partitioned between H₂O (250 mL) and EtOAc (2×250 mL) and the combined organics are dried (MgSO₄) and concentrated to an amber oil (13.89 g). The crude material is chromatographed over 500 g silica gel, eluting with 25% EtOAc/hexane. The appropriate fractions are combined and concentrated to afford 10.71 g (84% yield) of ethyl 5-cyclopropylisoxazole-3-carboxylate as a yellow oil. MS (CI) m/z: 182 (M+H)⁺.

Sodium hydroxide (1.76 g, 44.0 mmol) in H₂O (5 mL) is added to a solution of ethyl 5-cyclopropylisoxazole-3-carboxylate (1.97 g, 10.9 mmol) in MeOH (10 mL). The mixture is stirred at RT for 3 h, concentrated to remove the MeOH, and acidified to pH 2 with 5% HCl. The acid is extracted with CH₂Cl₂ (6×20 mL), dried (MgSO₄) and concentrated to afford 1.56 g (93% yield) of 5-cyclopropylisoxazole-3-carboxylic acid as a white solid. MS (Cl) m/z: 154 (M+H)⁺.

5-Cyclopropylisoxazole-3-carboxylic acid (1.53 g, 10 mmol) is dissolved in benzene (30 mL), treated with oxalyl chloride (3.46 mL, 40 mmol) and heated to reflux for 2 h. The mixture is cooled, concentrated to dryness and the residual benzene is azeotroped off with CH₂Cl₂. The resulting acid chloride is dissolved in Me₂CO (15 mL) and treated with a solution of NaN₃ (1.95 g, 30 mmol) in H₂O (7 mL). The mixture is vigorously stirred for 1 h, concentrated to remove the Me₂CO, triturated with H₂O, filtered, rinsed with water and dried under vacuum to afford 1.76 g (99% yield) of 5-cyclopropylisoxazole-3-carbonyl azide as an off-white solid. ¹H NMR (CDCl₃, 400 MHz): δ 1.02, 1.14, 2.10, 6.35 ppm.

5-Cyclopropylisoxazole-3-carbonyl azide (447 mg, 2.5 mmol) is combined with 5-chloro-2,4-dimethoxyaniline (471 mg, 2.5 mmol) in anhydrous MeCN (30 mL) and heated to reflux for 18 h. The mixture is cooled and the resulting solid is filtered, rinsed with Et₂O and dried in a vacuum oven to afford 619 mg (73% yield) of Example 621 as a very light purple solid. HRMS (ESI) calcd for C₁₅H₁₆N₃O₄Cl +H: 338.0907, found 338.0896 (M+H)⁺.

The following compounds are made from 5-cyclopropylisoxazole-3-carbonyl azide and an aniline according to Method F, making non-critical variations.

### Example 622

N-(5-cyclopropylisoxazol-3-yl)-N′-(5-fluoro-2,4-dimethoxyphenyl)urea. Yield 61%. MS (Cl) m/z: 322 (M+H)⁺.

### Example 623

N-(5-bromo-2,4-dimethoxyphenyl)-N′-(5-cyclopropylisoxazol-3-yl)urea. Yield 64%. MS (EI) m/z: 383 (M+H)⁺.

### Example 624

N-(5-cyclopropylisoxazol-3-yl)-N′-(2,4-dimethoxy-5-methylphenyl)urea. Yield 52%. MS (El) m/z: 317 (M)⁺.

### Example 625

N-(5-cyclopropylisoxazol-3-yl)-N′-(4-ethoxy-2-nitrophenyl)urea.

5-Cyclopropylisoxazole-3-carbonyl azide (178 mg, 1.0 mmol) is combined with 4-ethoxy-2-nitroaniline (182 mg, 1.0 mmol) in toluene (10 mL) in a 20 mL vial and heated to 70° C. on a shaker block for 18 h, then 100° C. for 20 h. The mixture is cooled, concentrated to dryness and chromatographed over 11 g silica gel, eluting with 5% EtOAc/CH₂Cl₂. The appropriate fractions are combined and concentrated to afford 90 mg (27% yield) of Example 625 as a bright yellow solid. MS (Cl) m/z: 333 (M+H)⁺.

### Example 626

N-(5-cyclopropylisoxazol-3-yl)-N′-(2,6-dimethoxypyridin-3-yl)urea.

5-Cyclopropylisoxazole-3-carbonyl azide (178 mg, 1.0 mmol) is combined with 3-amino-2,6-dimethoxypyridine (191 mg, 1.0 mmol) in anhydrous MeCN (10 mL) in a dry flask, under nitrogen and heated to reflux for 18 h. The mixture is cooled and the resulting solid is filtered and rinsed with Et₂O. The solid (207 mg) is adsorbed onto silica gel (500 mg) and chromatographed over 10 g slurry-packed silica gel, eluting with 12% EtOAc/CH₂Cl₂. The appropriate fractions are combined and concentrated to an off-white solid (164 mg) which is recrystallized in EtOAc to afford 118 mg (39% yield) of Example 626 as a white solid. MS for C₁₄Hl₆N₄O₄, (ESI): 305.0 (M+H)⁺.

### Example 627

N-(5-chloro-2,4-dimethoxyphenyl)-N′-(5-ethylisoxazol-3-yl)urea.

Sodium ethoxide (10 g, 147 mmol) is combined with absolute EtOH (65 mL) in a dry flask, under nitrogen and heated to 70° C. to aid dissolution. The mixture is cooled to 0° C., treated drop-wise with a mixture of 2-butanone (13.16 mL, 147 mmol) and diethyl oxylate (19.96 mL, 147 mmol) and warmed to RT. The mixture is stirred for 1 h, then heated to 80° C. for 45 min. The mixture is cooled to RT and concentrated to dryness. The resulting mixture is partitioned between water (200 ML) and EtOAc (3×70 mL). The aqueous layer is acidified to pH 2 with dilute H₂SO₄, extracted with Et₂O (3×50 mL), dried (Na₂SO₄) and concentrated to afford 16.7 g (66% yield) of ethyl 2,4-dioxohexanoate as an amber oil. MS (Cl) m/z: 173 (M+H)⁺.

Ethyl 2,4-dioxohexanoate (11.22 g, 65.15 mmol) is combined with hydroxylamine hydrochloride (13.58 g, 195.5 mmol) in EtOH (200 mL), heated to reflux for 1.5 h, cooled, and concentrated to dryness. The residue is partitioned between H₂O (150 mL) and EtOAc (2×150 mL) and the combined organics are dried (MgSO₄) and concentrated to an amber oil (10.57 g). The crude material is chromatographed over 400 g silica gel, eluting with 20% EtOAc/hexane. The appropriate fractions are combined and concentrated to afford 8.95 g (81% yield) of ethyl 5-ethylisoxazole-3-carboxylate as a pale oil. HRMS (ESI) calcd for C₈H₁₁NO₃ +H: 170.0817, found 170.0824 (M+H)⁺.

Sodium hydroxide (10.9 g, 273 mmol) in water (35 mL) is added to a solution of ethyl 5-ethylisoxazole-3-carboxylate (11.6 g, 68 mmol) in MeOH (70 mL). The mixture is stirred at RT for 3 h, concentrated to remove the MeOH, and acidified to pH 2 with concentrated HCl. The acid is extracted with CH₂Cl₂ (2×150 mL) then 10% MeOH/CH₂Cl₂ (4×150 mL), dried (MgSO₄) and concentrated to afford 5.65 g (58% yield) of 5-ethylisoxazole-3-carboxylic acid as a white solid. MS (Cl) m/z: 142 (M+H)⁺.

5-Ethylisoxazole-3-carboxylic acid (1.41 g, 10 mmol) is dissolved in benzene (30 mL), treated with oxalyl chloride (3.46 mL, 40 mmol) and heated to reflux for 2 h. The mixture is cooled, concentrated to dryness and the residual benzene is azeotroped off with CH₂Cl₂. The resulting acid chloride is dissolved in Me₂CO (15 mL) and treated with a solution of NaN₃ (1.95 g, 30 mmol) in water (7 mL). The mixture is vigorously stirred for 1 h, concentrated to remove the Me₂CO, triturated with water, filtered, rinsed with H₂O and dried under vacuum to afford 1.46 g (88% yield) of 5-ethylisoxazole-3-carbonyl azide as a white solid. ¹H NMR (CDCl₃, 400 MHz): δ 1.34, 2.85, 6.46 ppm.

5-Ethylisoxazole-3-carbonyl azide (294 mg, 1.8 mmol) is combined with 5-chloro-2,4-dimethoxyaniline (332 mg, 1.8 mmol) in anhydrous MeCN (20 mL) and heated to 70° C. for 20 h. The mixture is cooled and the resulting solid is filtered, rinsed with Et₂O and dried in a vacuum oven to afford 448 mg (78% yield) of Example 627 as a white solid. HRMS (ESI) calcd for C₁₄H₁₆N₃O₄Cl +H: 326.0907, found 326.0909 (M+H)⁺.

The following compounds are made from 5-ethylisoxazole-3-carbonyl azide and an aniline according to Method F, making non-critical variations.

### Example 628

N-(5-ethylisoxazol-3-yl)-N′-(5-fluoro-2,4-dimethoxyphenyl)urea. Yield 49%. HRMS (ESI) calcd for C₁₄H₁₆N₃O₄F +H: 310.1203, found 310.1211 (M+H)⁺.

### Example 629

N-(5-bromo-2,4-dimethoxyphenyl)-N′-(5-ethylisoxazol-3-yl)urea. Yield 59%. HRMS (ESI) calcd for C₁₄H₁₆N₃O₄Br +H: 370.0403, found 370.0399 (M+H)⁺.

### Example 630

N-(2,4-dimethoxy-5-methylphenyl)-N′-(5-ethylisoxazol-3-yl)urea. Yield 51%. HRMS (ESI) calcd for C₁₅H₁₉N₃O₄ +H: 306.1454, found 306.1468 (M+H)⁺.

### Example 631

N-(2,6-dimethoxypyridin-3-yl)-N′-(5-ethylisoxazol-3-yl)urea. Yield 57%. MS for C₁₄H₁₆N₄O₄, (ESI): 293.0 (M+H)⁺.

### Example 632

N-(4-Ethoxy-2-nitrophenyl)-N′-(5-ethylisoxazol-3-yl)urea. 5-Ethylisoxazole-3-carbonyl azide (166 mg, 1.0 mmol) is combined with 4-ethoxy-2-nitroaniline (182 mg, 1.0 mmol) in toluene (10 mL) in a 20 mL vial and heated to 70° C. on a shaker block for 18 h, then 100° C. for 20 h. The mixture is cooled, concentrated to dryness and chromatographed over 11 g silica gel, eluting with 7% EtOAc/CH₂Cl₂. The appropriate fractions are combined and concentrated to afford 91 mg (28% yield) of Example 632 as a bright yellow solid. HRMS (ESI) calcd for C₁₄H₁₆N₄O₅ +H: 321.1199, found 321.1208 (M+H)⁺.

### MISCELLANEOUS METHODS

### Example 700

N-(5-amino-2,4-dimethoxyphenyl)-N′-[5-(trifluoromethyl)-1,3,4-thiadiazol-2-yl]urea hydrochloride.

A DMF solution of the product from Example 135 (1.5 g, 3.86 mmol) is treated with tin dichloride dihydrate (4.35 g, 19.3 mmol) in DMF. After 72 h, the solvent is removed. The residue is taken up in THF and aqueous NaHCO₃, stirred for 12 h and filtered. The filtrate is concentrated, suspended in hot CH₃CN and re-filtered. The solvents are removed and the residue is converted into the HCl salt to provide 1.22 g (79% yield) of Example 700. HRMS (ESI) calcd for C₁₂Hl₂F₃N₅O₃S+H 364.0691, found 364.0692.

### Example 701

N-(5-azido-2,4-dimethoxyphenyl)-N′-[5-(trifluoromethyl)-1,3,4-thiadiazol-2-yl]urea.

A suspension of the product from Example 700 (0.3 g, 0.75 mmol) in 6N HCl at 0° C. is treated with sodium nitrite (69 mg, 1.0 mmol) dissolved in water. After 1 h, a solution of sodium azide (65 mg, 1.0 mmol) in water is added. The mixture is warmned to RT over 18 h. EtOAc is added and the mixture is extracted with EtOAc, dried (MgSO₄), filtered and concentrated. The residue is triturated with ether to provide 32 mg (11% yield) of Example 701. HRMS (ESI) calcd for C₁₂H₁₀F₃N₇O₃S+H 390.0596, found 390.0592.

### Example 702

N-(5-iodo-2,4-dimethoxyphenyl)-N′-[5-(trifluoromethyl)-1,3,4-thiadiazol-2-yl]urea.

A suspension of the product from Example 700 (0.46 g, 1.15 mmol) in H₂SO₄ at 0° C. is treated with sodium nitrite (123 mg, 1.8 mmol) dissolved in water. After 1 h, a solution of potassium iodide (338 mg, 2.0 mmol) in water is added. 3 mL of THF are added. The mixture is warmed to RT over 18 h. EtOAc is added and the mixture is extracted with EtOAc, dried (MgSO₄), filtered and concentrated. The residue is purified by chromatography (Biotage 40S, 40% EtOAc/hexanes) and recrystallized from EtOAc/hexanes to provide 28 mg (5% yield) of Example 702. ¹H NMR (400 MHz, DMSO) δ 11.95, 8.71, 8.35, 6.80, 3.94, 3.85.

### Example 703

N-(4-amino-2-methoxyphenyl)-N′-[5-(trifluoromethyl)-1,3,4-thiadiazol-2-yl]urea.

The product from Example 142 is heated in EtOAc/EtOH (10 mL) until completely dissolved. The solution is then diluted with EtOH (50 mL). 10% Pd/C catalyst is then added as a slurry in EtOAc and the reaction put on the Parr apparatus under hydrogen for 1 h (32 psi to 17 psi). The reaction mixture is filtered over Celite to remove the catalyst and the solvent is removed and the crude solid recrystallized (CH₃CN) to give the product as a tan solid (46 mg, 26% yield). HRMS (ESI) calcd for C₁₁H₁₀F₃N₅O₂S+H 397.0252 found 397.0253.

### Example 704

N-(4-methoxy-2-methylphenyl)-N′-[4-(pentafluoroethyl)-1,3-thiazol-2-yl]urea.

Thiourea (0.261 g, 3.43 mmol) is dissolved in DMF (2 mL) and the resulting solution is heated to 80° C. under N₂. 4-Methoxy-2-methylphenylisocyanate (0.50 mL, 0.561 g, 3.43 mmol) is added dropwise over a 7-minute period to the reaction mixture, which is stirred at 80° C. for 2 h. 1-Bromo-3,3,4,4,4-pentafluoro-2-butanone (0.457 mL, 0.826 g, 3.43 mmol) and TEA (0.478 mL, 0.347 g, 3.43 mmol) are added sequentially and the reaction mixture is heated at 80° C. for 19.5 h. The reaction mixture is cooled to RT and taken up in EtOAc. This solution is washed sequentially with 1N aqueous HCl, H₂O and brine. The organic layer is filtered to remove suspended solids and dried (MgSO₄), filtered and concentrated. The crude product is chromatographed (SiO₂, 9:1 CHCl₃:EtOAc) to yield Example 704 in 18% yield. MS (ESI+) for C₁₄H₁₂F₅N₃O₂S m/z 381.9 (M+H)⁺.

### Example 705

N-(5-chloro-2,4-dimethoxyphenyl)-N′-[5-(trifluoromethyl)isoxazol-3-yl]urea.

Ethyl chlorooximidoacetate (5.00 g, 33 mmol, 1 equiv.) and 3,3,3-trifluoro-2-bromopropene (35.9 mL, 57.7 g, 330 mmol, 10 eq) are dissolved in Et₂O (110 mL) and stirred at RT under N₂. A solution of TEA (13.8 mL, 10.0 g, 99 mmol, 3 equiv.) in Et₂O (86 mL) is added dropwise over 19 h to the reaction mixture using a syringe pump. After the addition is complete, the reaction mixture is stirred overnight. The reaction mixture is washed with H₂O and the layers are separated. The aqueous layer is extracted with Et₂O. The combined organic layers are dried (MgSO₄), filtered and partially concentrated. The crude product is chromatographed (SiO₂ 200 g, eluted with 8:1 hexane:EtOAc) to yield pure ethyl 5-(trifluoromethyl)isoxazole-3-carboxylate (4.4 g, 64% yield) as a solution in EtOAc. Due to the volatility of the isoxazole, no attempt is made to remove all of the solvent. The isoxazole solution is diluted with MeOH and the resulting solution is carefully, partially concentrated by rotary evaporation. This dilution with MeOH and partial concentration is repeated to remove all of the EtOAc prior to hydrolysis of the ethyl ester.

Ethyl 5-(trifluoromethyl)isoxazole-3-carboxylate (0.65 g) is dissolved in MeOH (7 mL) and 1N aqueous NaOH (7 mL) is added. The resulting mixture is stirred at rt for 1 h. The reaction mixture is partitioned between 1N aqueous HCl and CH₂Cl₂. The layers are separated and the aqueous layer is extracted with CH₂Cl₂. The combined organic layers are dried (MgSO₄), filtered and carefully concentrated to yield 5-(trifluoromethyl)isoxazole-3-carboxylic acid (0.60 g, 106% yield). ¹³C NMR (100 MHz, CD₃OD) δ 108.2, 119.5, 159.4, 161.2, 161.2; ¹H NMR (CD₃OD) δ 7.44.

To a solution of 5-(trifluoromethyl)isoxazole-3-carboxylic acid (0.399 g, 2.21 mmol) in Et₂O (6.6 mL) at 0° C. under N₂ is added N-methylmorpholine (0.32 mL, 0.290 g, 2.87 mmol) followed by ethyl chloroformate (0.25 g, 0.288 g, 2.65 mmol). After stirring for 20 min, the reaction mixture is filtered into a solution of NH₂OH in CH₃OH. [The solution of NH₂OH in CH₃OH is prepared by adding a solution of NH₂OH.HCl (0.453 g) in CH₃OH (4.4 mL) to a solution of KOH (0.366 g) in CH₃OH (1.8 mL) at 0° C. and filtering the resulting mixture to remove KCl.] The resulting mixture is stirred at RT for 1 h. The reaction mixture is partitioned between CH₂Cl₂ and 10% aqueous citric acid. The layers are separated and the aqueous layer is extracted with CH₂Cl₂. The combined organic layers are dried (MgSO₄), filtered and concentrated to yield N-hydroxy-5-(trifluoromethyl)isoxazole-3-carboxamide (0.140 g) in 32% yield. ¹H NMR (400 MHz, DMSO-d₆) δ 7.78, 9.63, 11.79.

N-Hydroxy-5-(trifluoromethyl)isoxazole-3-carboxamide (0.994 g, 0.507 mmol) is suspended in CH₂Cl₂ (2.5 mL) at RT under N₂. TEA (0.36 mL, 0.258 g, 2.55 mmol) and 2-chloro-1,3-dimethylimidazolinium chloride (0.104 g, 0.617 mmol) are added sequentially and the reaction mixture is stirred at RT for 30 min. 5-Chloro-2,4-dimethoxyaniline (0.114 g, 0.608 mmol) is added and the reaction mixture is stirred at RT for an additional 2.5 h. Acetonitrile (5 mL) is added and the reaction mixture is refluxed for 15.5 h. After cooling to RT, the reaction mixture is partitioned between EtOAc and 10% aqueous citric acid. The layers are separated and the aqueous layer is extracted with EtOAc. The combined organic layers are dried (MgSO₄), filtered and concentrated to give a crude product (0.620 g). The crude product is chromatographed (SiO₂ 62 g, eluted with 8:1 CHCl₃:EtOAc) to yield

Example 705 (0.0296 g) in 16% yield. MS (ESI+) for C₁₃H₁₁ClF₃N₃O₄ m/z 366.3 (M+H)⁺.

Any one or more of the following compounds are also within the scope of the invention, are for exemplification only and are not to limit the scope of the invention, and are prepared using methods discussed herein making non-critical changes:

N-(4-hydroxyphenyl)-N′-(5-isopropyl-1,3-thiazol-2-yl)urea;

N-(4-ethoxyphenyl)-N′-(5-methyl-1,3-thiazol-2-yl)urea;

N-[4-(4-chlorophenyl)-1,3-thiazol-2-yl]-N′-(4-iodophenyl)urea;

N-(2,6-dimethylphenyl)-N′-(6-fluoro-1,3-benzothiazol-2-yl)urea;

N-(4-chloro-1,3-benzothiazol-2-yl)-N′-(2-methoxyphenyl)urea;

N-(5-bromo-1,3-thiazol-2-yl)-N′-(2-ethoxyphenyl)urea;

N-(5-bromo-1,3-thiazol-2-yl)-N′-(2,4-dimethoxyphenyl)urea;

N-(5-chloro-1,3-thiazol-2-yl)-N′-(4-methoxy-2-methylphenyl)urea;

N-(5-bromo-1,3-thiazol-2-yl)-N′-(4-methoxy-2-methylphenyl)urea;

N-(5-chloro-2,4-dimethoxyphenyl)-N′-(1,3-thiazol-2-yl)urea;

N-(4-methoxy-2-methylphenyl)-N′-(4-methyl-1,3-thiazol-2-yl)urea;

N-(4-chloro-1,3-benzothiazol-2-yl)-N′-(4-methoxy-2-methylphenyl)urea;

ethyl 2-({[(4-methoxy-2-methylphenyl)amino]carbonyl}amino)-4-phenyl-1,3-thiazole-5-carboxylate;

ethyl 2-[({[4-(butoxycarbonyl)phenyl]amino}carbonyl)amino]-4-phenyl-1,3-thiazole-5-carboxylate;

N-(5-bromo-1,3-thiazol-2-yl)-N′-(4-ethoxyphenyl)urea;

N-[2-methoxy-4-(oxetan-3-yloxy)phenyl]-N′-[5-(trifluoromethyl)-1,3,4-thiadiazol-2-yl]urea;

N-[2-methoxy-4-(oxetan-3-yloxy)phenyl]-N′-[3-(trifluoromethyl)isoxazol-5-yl]urea;

N-[2-methoxy-4-(tetrahydrofuran-3-yloxy)phenyl]-N′-[5-(trifluoromethyl)-1,3,4-thiadiazol-2-yl]urea; and

N-[2-methoxy-4-(tetrahydrofuran-3-yloxy)phenyl]-N′-[3-(trifluoromethyl)isoxazol-5-yl]urea.

The compounds in the following tables are also within the scope of the present invention, are for exemplification only and are not intended to limit the scope of the invention, and are prepared using methods discussed herein making non-critical change.

TABLE 1

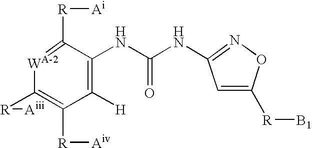

| | *R-Aⁱⁱⁱ | *W^{A-2} | *R-Aⁱ | *R-A^{iv} | R-B₁ |
|---|---|---|---|---|---|
| 1001 | H | (1) CH | (A) H | (a) H | (i) CH₃ |
| 1002 | OCH3 | (2) N | (B) CH₃ | (b) CH₃ | (ii) CF₃ |
| 1003 | OCH₂CH₃ | | (C) OCH₃ | (c) OCH₃ | (iii) CH₂F |
| 1004 | OCH₂—CH═CH₂ | | (D) OEt | (d) F | (iv) CHF₂ |
| 1005 | O-n-propyl | | (E) O-i-propyl | (e) Cl | (v) CH₂CH₃ |
| 1006 | O-i-propyl | | (F) O-n-propyl | (f) Br | (vi) CF₂CF₃ |
| 1007 | O-sec-butyl | | (G) SMe | | (vii) cyclopropyl |
| 1008 | 2-hydroxyethoxy | | (H) S(O)Me | | (viii) Cl |
| 1009 | 2-methoxyethoxy | | (I) SEt | | (ix) Br |
| 1010 | 2-ethoxyethoxy | | (J) S(O)Et | | (xi) CN |
| 1011 | 2-(ethylthio)- | | (K) NO₂ | | (xii) H |
| | ethoxy | | | | |
| 1012 | 2-(methylthio)- | | (L) C(═O)Me | | (xiii) CH₂OCH₃ |
| | ethoxy | | | | |
| 1013 | 2-(methyl | | (M) | | |
| | sulfinyl)ethoxy | | C(Me)(═NOMe) | | |
| 1014 | 2-(methyl | | | | |
| | sulfonyl)ethoxy | | | | |
| 1015 | 2-(methylamino)- | | | | |
| | ethoxy | | | | |
| 1016 | 2-(ethylamino)- | | | | |
| | ethoxy | | | | |
| 1017 | 2-(dimethyl | | | | |
| | amino)ethoxy | | | | |
| 1018 | 2-(diethyl | | | | |
| | amino)ethoxy | | | | |
| 1019 | OCH₂CH₂- | | | | |
| | N(Me)(Et) | | | | |
| 1020 | OCH₂CH₂- | | | | |
| | NHC(O)CH₃ | | | | |
| 1021 | OCH₂CH₂- | | | | |
| | NHS(O)₂CH₃ | | | | |
| 1022 | 2-thiomorpholin- | | | | |
| | 4-ylethoxy | | | | |
| 1023 | 2-(1,1-dioxido- | | | | |
| | thiomorpholin-4- | | | | |
| | yl)ethoxy | | | | |
| 1024 | 2-piperazin-1- | | | | |
| | ylethoxy | | | | |
| 1025 | 2-pyrrolidin-1- | | | | |
| | ylethoxy | | | | |
| 1026 | 2-piperidin-1- | | | | |
| | ylethoxy | | | | |
| 1027 | 2-morpholin-4- | | | | |
| | ylethoxy | | | | |
| 1028 | 2-1H-pyrazol-1- | | | | |
| | ylethoxy | | | | |
| 1029 | O-oxetan-3-yl | | | | |
| 1030 | O-tetrahydro- | | | | |
| | furan-3-yl | | | | |
| 1031 | O-1,1-dioxido- | | | | |
| | thietan-3-yl | | | | |
| 1032 | O-azetidin-3-yl | | | | |

*Provided that at least one of R-Aⁱ, R-Aⁱⁱⁱ, and R-A^{iv} is other than H.

Tables 1-5 are used in the same manner. One selection is made from each column to determine the compounds represented within each table. The compound number determines which moiety from column R-Aⁱⁱⁱ is selected; the number or letter within parentheses determines which moiety is selected from W^{A-2}, R-Aⁱ, R-A^{iv}, and R-B₁. As indicated by an*after each table, at least one of R-Aⁱ, R-Aⁱⁱⁱ, and R-A^{iv} is other than H. The compounds represented in these tables can be a free base or a pharmaceutically acceptable salt thereof. The following compounds are provided for exemplification, but not limitation, to show how compounds from Table 1 are identified:

1001(1)(D)(a)(i) is N-(2-ethoxyphenyl)-N′-(5-methylisoxazol-3-yl)urea;

1002(2)I(e)(xi) is N-(5-chloro-2,6-dimethoxypyridin-3-yl)-N′-(5-cyanoisoxazol-3-yl)urea;

1003(1)(D)(d)(iv) is N-(2,4-diethoxy-5-fluorophenyl)-N′-[5-(difluoromethyl)isoxazol-3-yl]urea;

1005(2)(G)(a)(vii) is N-(5-cyclopropylisoxazol-3-yl)-N′-[2-(methylthio)-6-propoxypyridin-3-yl]urea;

1009(1)(K)(a)(ii) is N-[4-(2-methoxyethoxy)-2-nitrophenyl]-N′-[5-(trifluoromethyl)isoxazol-3-yl]urea;

1014(2)I(a)(ix) is N-(5-bromoisoxazol-3-yl)-N′-{2-methoxy-6-[2-(methylsulfonyl)ethoxy]pyridin-3-yl}urea;

1018(2)(A)(d)(iii) is N-{6-[2-(diethylamino)ethoxy]-5-fluoropyridin-3-yl}-N′-[5-(fluoromethyl)isoxazol-3-yl]urea;

1020(1)(L)(f)(viii) is N-{2-[5-acetyl-2-bromo-4-({[(5-chloroisoxazol-3-yl)amino]carbonyl}amino)phenoxy]ethyl}acetamide;

1022(2)(H)(b)(v) is N-(5-ethylisoxazol-3-yl)-N′-[5-methyl-2-(methylsulfinyl)-6-(2-thiomorpholin-4-ylethoxy)pyridin-3-yl]urea; and

1027(1)(B)(c)(vi) is N-[5-methoxy-2-methyl-4-(2-morpholin-4-ylethoxy)phenyl]-N′-[5-(pentafluoroethyl)isoxazol-3-yl]urea.

TABLE 2

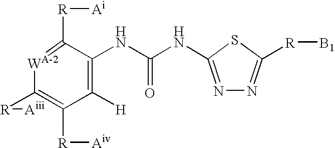

| | *R-Aⁱⁱⁱ | *W^{A-2} | *R-Aⁱ | *R-A^{iv} | R-B₁ |
|---|---|---|---|---|---|
| 1051 | H | (1) CH | (A) H | (a) H | (i) CH₃ |
| 1052 | OCH₃ | (2) N | (B) CH₃ | (b) CH₃ | (ii) CF₃ |
| 1053 | OCH₂CH₃ | | (C) OCH₃ | (c) OCH₃ | (iii) CH₂F |
| 1054 | OCH₂—CH═CH₂ | | (D) OEt | (d) F | (iv) CHF₂ |
| 1055 | O-n-propyl | | (E) O-i-propyl | (e) Cl | (v) Cl |
| 1056 | O-i-propyl | | (F) O-n-propyl | (f) Br | (vi) Br |
| 1057 | 0-sec-butyl | | (C) SMe | | (vii) CN |
| 1058 | 2-hydroxyethoxy | | (H) S(O)Me | | (viii) CF₂CF₃ |
| 1059 | 2-methoxyethoxy | | (I) SEt | | |
| 1060 | 2-ethoxyethoxy | | (J) S(O)Et | | |
| 1061 | 2-(ethylthio)- | | (K) NO₂ | | |
| | ethoxy | | | | |
| 1062 | 2-(methylthio)- | | (L) C(═O)Me | | |
| | ethoxy | | | | |
| 1063 | 2-(methyl | | (M) | | |
| | sulfinyl)ethoxy | | C(Me)(═NOMe) | | |
| 1064 | 2-(methyl | | | | |
| | sulfonyl)ethoxy | | | | |
| 1065 | 2-(methylamino)- | | | | |
| | ethoxy | | | | |
| 1066 | 2-(ethylamino)- | | | | |
| | ethoxy | | | | |
| 1067 | 2-(dimethyl | | | | |
| | amino)-ethoxy | | | | |
| 1068 | 2-(diethylamino)- | | | | |
| | ethoxy | | | | |
| 1069 | OCH₂CH₂- | | | | |
| | N(Me)(Et) | | | | |
| 1070 | OCH₂CH₂- | | | | |
| | NHC(O)CH₃ | | | | |
| 1071 | OCH₂CH₂- | | | | |
| | NHS(O)₂CH₃ | | | | |
| 1072 | 2-thiomorpholin- | | | | |
| | 4-ylethoxy | | | | |
| 1073 | 2-(1,1-dioxido- | | | | |
| | thiomorpholin-4- | | | | |
| | yl)ethoxy | | | | |
| 1074 | 2-piperazin-1- | | | | |
| | ylethoxy | | | | |
| 1075 | 2-pyrrolidin-1- | | | | |
| | ylethoxy | | | | |
| 1076 | 2-piperidin-1- | | | | |
| | ylethoxy | | | | |
| 1077 | 2-morpholin-4- | | | | |
| | ylethoxy | | | | |
| 1078 | 2-IH-pyrazol-1- | | | | |
| | ylethoxy | | | | |
| 1079 | O-oxetan-3-yl | | | | |
| 1080 | O-tetrahydro- | | | | |
| | furan-3-yl | | | | |
| 1081 | O-1,1-dioxido- | | | | |
| | thietan-3-yI | | | | |
| 1082 | O-azetidin-3-yl | | | | |

*provided that at least one of R-Aⁱ, R-Aⁱⁱⁱ, and R-A^{iv} is other than H.

TABLE 3

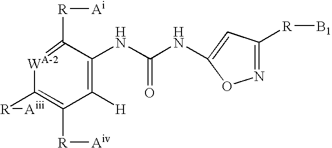

| | *R-Aⁱⁱⁱ | *W^{A-2} | *R-Aⁱ | *R-A^{iv} | R-B₁ |
|---|---|---|---|---|---|
| 1101 | H | (1) CH | (A) H | (a) H | (i) CH₃ |
| 1102 | OCH₃ | (2) N | (B) CH₃ | (b) CH₃ | (ii) CF₃ |
| 1103 | OCH₂CH₃ | | (C) OCH₃ | (c) OCH₃ | (iii) CH₂F |
| 1104 | OCH₂—CH═CH₂ | | (D) OEt | (d) F | (iv) CHF₂ |
| 1105 | O-n-propyl | | (B) O-i-propyl | (e) Cl | (v) Cl |
| 1106 | O-i-propyl | | (F) O-n-propyl | (f) Br | (vi) Br |
| 1107 | O-sec-butyl | | (G) SMe | | (vii) CN |
| 1108 | 2-hydroxyethoxy | | (H) S(O)Me | | (viii) CF₂CF₃ |
| 1109 | 2-methoxyethoxy | | (I) SEt | | |
| 1110 | 2-ethoxyethoxy | | (J) S(O)Et | | |
| 1111 | 2-(ethylthio)- | | (K) NO₂ | | |
| | ethoxy | | | | |
| 1112 | 2-(methylthio)- | | (L) C(═O)Me | | |
| | ethoxy | | | | |
| 1113 | 2-(methyl | | (M) | | |
| | sulfinyl)ethoxy | | C(Me)(═NOMe) | | |
| 1114 | 2-(methyl | | | | |
| | sulfonyl)ethoxy | | | | |
| 1115 | 2-(methylamino)- | | | | |
| | ethoxy | | | | |
| 1116 | 2-(ethylamino)- | | | | |
| | ethoxy | | | | |
| 1117 | 2-(dimethyl | | | | |
| | amino)ethoxy | | | | |
| 1118 | 2-(diethylamino)- | | | | |
| | ethoxy | | | | |
| 1119 | OCH₂CH₂- | | | | |
| | N(Me)(Et) | | | | |
| 1120 | OCH₂CH₂- | | | | |
| | NHC(O)CH₃ | | | | |
| 1121 | OCH₂CH₂- | | | | |
| | NHS(O)₂CH₃ | | | | |
| 1122 | 2-thiomorpholin- | | | | |
| | 4-ylethoxy | | | | |
| 1123 | 2-(1,1-dioxido | | | | |
| | thiomorpholin-4- | | | | |
| | yl)ethoxy | | | | |
| 1124 | 2-piperazin-1- | | | | |
| | ylethoxy | | | | |
| 1125 | 2-pyrrolidin-1- | | | | |
| | ylethoxy | | | | |
| 1126 | 2-piperidin-1- | | | | |
| | ylethoxy | | | | |
| 1127 | 2-morpholin-4- | | | | |
| | ylethoxy | | | | |
| 1128 | 2-1H-pyrazol-1- | | | | |
| | ylethoxy | | | | |
| 1129 | O-oxetan-3-yl | | | | |
| 1130 | O-tetrahydro- | | | | |
| | furan-3-yl | | | | |
| 1131 | O-1,1-dioxido- | | | | |
| | thietan-3-yl | | | | |
| 1132 | O-azetidin-3-yl | | | | |

*provided that at least one of R-Aⁱ, R-Aⁱⁱⁱ, and R-A^{iv} is other than H.

TABLE 4

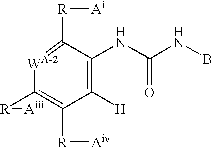

| | *R-Aⁱⁱⁱ | *W^{A-2} | *R-Aⁱ | *R-A^{iv} | B | R-B₁ |
|---|---|---|---|---|---|---|
| 1151 | H | (1) CH | (A) H | (a) H | 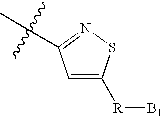 | (i) CH₃ |
| 1152 | OCH₃ | (2) N | (B) CH₃ | (b) CH₃ | 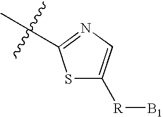 | (ii) CF₃ |
| 1153 | OCH₂CH₃ | | (C) OCH₃ | (c) OCH₃ | 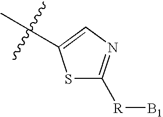 | (iii) Cl |
| 1154 | OCH₂—CH═CH₂ | | (D) OEt | (d) F | 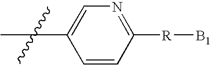 | (iv) Br |
| 1155 | 2-methoxy | | (E) SMe | (e) Cl | | (v) CN |
| | ethoxy | | | | | |
| 1156 | O-oxetan-3-yl | | (F) NO₂ | (f) Br | | |
| 1157 | O-tetrahydro- | | (G) | | | |
| | furan-3-yl | | C(═O)Me | | | |
| 1158 | O-1,1-dioxido- | | | | | |
| | thietan-3-yl | | | | | |
| 1159 | O-azetidin-3-yl | | | | | |

*Provided that at least one of R-Aⁱ, R-Aⁱⁱⁱ, and R-A^{iv} is other than H.

TABLE 5

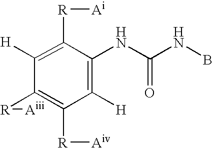

| | B | *R-Aⁱ | *R-Aⁱⁱⁱ | *R-A^{iv} |
|---|---|---|---|---|
| 1201 | 3-trifluorophenyl | (I) H | (A) H | (i) H |
| 1202 | 4-methyl-thiazol-2-yl | (II) CH₃ | (B) OH | (ii) OH |
| 1203 | 4-ethyl-thiazol-2-yl | | (C) OCH₃ | |
| 1204 | 4-trifluoromethyl-thiazol-2-yl | | | |
| 1205 | 4-(pentafluoroeth-1-yl)-thiazol-2-yl | | | |

*Provided that at least one of R-Aⁱ, R-Aⁱⁱⁱ, and R-A^{iv} is other than H.

Assay for positive allosteric modulators of α7 nAChR.

Both agonist and positive allosteric modulator activity of the α7 nAChR are assayed using a cell-based, calcium flux assay on FLIPR. SHEP-1 cells expressing a novel, mutated form of the α7 nAChR that permitted stable cell surface expression were used for these assays. The details of the mutated form of the α7 nAChR is described in WO 00/73431.

Cells were plated into each well of either a 96 or 384 well cell culture plates, they were transferred to a standard CO₂ incubator for at least 24 h to achieve confluence. The assay described below is for the 96 well assay. The 384-well assay is essentially the same, with the exception that the volumes of the reagents was reduced by a factor of 4. At confluence, the growth media was aspirated and replaced with 200 μl of new media containing a Calcium Green-1 AM to obtain a final dye concentration was 2 μM. Cells were incubated for 60 min at 37° C., then washed 4 times leaving 100 μl of assay buffer in each well. The details of the assay buffer were described in WO 00/73431. At this point, the cell culture plate containing the cells loaded with the calcium indicator dye was placed in FLIPR. FLIPR was configured to excite the Calcium Green at 488 nm and emission was read using a 520 nm filter set.

Compounds were prepared as a solutions in an assay buffer. The assay was initiated by collecting 10 baseline data points at 1.5 second intervals. After the baseline points were collected, 100 μl of compound was added to the well. The resulting 1:1 dilution achieved a final concentration 30 μM for each compound. An additional 3 min of data was collected. After 3 min measurements, acetylcholine was added at a final concentration of 100 μM. Acetylcholine produced a reproducible rapid and transient calcium flux. Positive allosteric modulator activity was defined as a compound that increased the acetylcholine response by greater than 4 standard deviations of the mean response.

The following compounds are active as positive allosteric modulators at 0.01-30 μM and are included within the present invention:

N-(4-hydroxyphenyl)-N′-(5-isopropyl-1,3-thiazol-2-yl)urea;

N-(4-ethoxyphenyl)-N′-(5-methyl-1,3-thiazol-2-yl)urea;

N-[4-(4-chlorophenyl)-1,3-thiazol-2-yl]-N′-(4-iodophenyl)urea;

N-(2,6-dimethylphenyl)-N′-(6-fluoro-1,3-benzothiazol-2-yl)urea;

N-(4-chloro-1,3-benzothiazol-2-yl)-N′-(2-methoxyphenyl)urea;

N-(5-bromo-1,3-thiazol-2-yl)-N′-(2-ethoxyphenyl)urea;

N-(5-bromo-1,3-thiazol-2-yl)-N′-(2,4-dimethoxyphenyl)urea;

N-(5-chloro-1,3-thiazol-2-yl)-N′-(4-methoxy-2-methylphenyl)urea;

N-(5-bromo-1,3-thiazol-2-yl)-N′-(4-methoxy-2-methylphenyl)urea;

N-(5-chloro-2,4-dimethoxyphenyl)-N′-(1,3-thiazol-2-yl)urea;

N-(4-methoxy-2-methylphenyl)-N′-(4-methyl-1,3-thiazol-2-yl)urea;

N-(5-chloro-2,4-dimethoxyphenyl)-N′-(5-methyl-1,3-thiazol-2-yl)urea;

N-(4-chloro-1,3-benzothiazol-2-yl)-N′-(4-methoxy-2-methylphenyl)urea;

ethyl 2-({[(4-methoxy-2-methylphenyl)amino]carbonyl}amino)-4-phenyl-1,3-thiazole-5-carboxylate;

ethyl 2-[({[4-(butoxycarbonyl)phenyl]amino}carbonyl)amino]-4-phenyl-1,3-thiazole-5-carboxylate;

N-(5-bromo-1,3-thiazol-2-yl)-N′-(4-ethoxyphenyl)urea;

N-(4-methoxy-2-methylphenyl)-N′-[3-(trifluoromethyl)phenyl]urea;

N-(5-chloro-2,4-dimethoxyphenyl)-N′-isoxazol-3-ylurea;

N-(5-chloro-2,4-dimethoxyphenyl)-N′-(5-methyl-1,3,4-thiadiazol-2-yl)urea;

N-(5-chloro-2,4-dimethoxyphenyl)-N′-(5-methylisoxazol-3-yl)urea;

N-(5-chloro-2,4-dimethoxyphenyl)-N′-(5-ethyl-1,3,4-thiadiazol-2-yl)urea;

N-(5-chloro-2,4-dimethoxyphenyl)-N′-[5-(trifluoromethyl)-1,3,4-thiadiazol-2-yl]urea;

N-(5-chloro-2,4-dimethoxyphenyl)-N′-(5-cyclopropyl-1,3,4-thiadiazol-2-yl)urea;

N-(5-chloro-2,4-dimethoxyphenyl)-N′-[5-(difluoromethyl)-1,3,4-thiadiazol-2-yl]urea;

N-(5-chloro-2,4-dimethoxyphenyl)-N′-(5-methoxy-1,3,4-thiadiazol-2-yl)urea;

N-(5-bromo-1,3,4-thiadiazol-2-yl)-N′-(5-chloro-2,4-dimethoxyphenyl)urea;

N-(5-chloro-1,3,4-thiadiazol-2-yl)-N′-(5-chloro-2,4-dimethoxyphenyl)urea;

N-(5-chloro-2,4-dimethoxyphenyl)-N′-(1,3,4-thiadiazol-2-yl)urea;

N-(4-ethoxyphenyl)-N′-(5-methylisoxazol-3-yl)urea;

N-(2,4-dimethoxyphenyl)-N′-(5-methylisoxazol-3-yl)urea;

N-(2,4-dimethoxyphenyl)-N′-[5-(trifluoromethyl)-1,3,4-thiadiazol-2-yl]urea;

N-(5-chloro-2-methoxyphenyl)-N′-(5-methylisoxazol-3-yl)urea;

N-(5-chloro-2-methoxyphenyl)-N′-(5-methyl-1,3,4-thiadiazol-2-yl)urea;

N-(4-isopropoxyphenyl)-N′-[5-(trifluoromethyl)-1,3,4-thiadiazol-2-yl]urea;

N-(2-ethoxyphenyl)-N′-[5-(trifluoromethyl)-1,3,4-thiadiazol-2-yl]urea;

N-(4-ethoxyphenyl)-N′-[5-(trifluoromethyl)-1,3,4-thiadiazol-2-yl]urea;

N-(4-methoxyphenyl)-N′-[5-(trifluoromethyl)-1,3,4-thiadiazol-2-yl]urea;

N-(4-butoxyphenyl)-N′-[5-(trifluoromethyl)-1,3,4-thiadiazol-2-yl]urea;

N-(2,3-dihydro-1-benzofuran-5-yl)-N′-[5-(trifluoromethyl)-1,3,4-thiadiazol-2-yl]urea;

N-(4-ethyl-1,3-thiazol-2-yl)-N′-(4-methoxy-2-methylphenyl)urea;

N-(5-chloro-2,4-dimethoxyphenyl)-N′-(1H-imidazol-2-yl)urea;

N-(5-bromo-1,3-thiazol-2-yl)-N′-(5-chloro-2,4-dimethoxyphenyl)urea;

N-(5-chloro-2,4-dimethoxyphenyl)-N′-(5-methyl-1,3-thiazol-2-yl)urea;

N-(5-chloro-2,4-dimethoxyphenyl)-N′-(3-methylisoxazol-5-yl)urea;

N-(5-chloro-2,4-dimethoxyphenyl)-N′-(6-cyanopyridin-3-yl)urea;

N-(5-chloro-2,4-dimethoxyphenyl)-N′-(5-chloro-1,3-thiazol-2-yl)urea;

N-(5-chloro-1,3-thiazol-2-yl)-N′-(2,4-dimethoxy-5-methylphenyl)urea;

N-(5-bromo-2,4-dimethoxyphenyl)-N′-(5-chloro-1,3-thiazol-2-yl)urea;

N-(5-bromo-1,3-thiazol-2-yl)-N′-(5-fluoro-2,4-dimethoxyphenyl)urea;

N-(5-chloro-1,3-thiazol-2-yl)-N′-(5-fluoro-2,4-dimethoxyphenyl)urea;

N-(3-chloro-4-fluorophenyl)-N′-(5-methylisoxazol-3-yl)urea;

N-(3-chloro-4-fluorophenyl)-N′-[5-(trifluoromethyl)-1,3,4-thiadiazol-2-yl]urea;

N-(2-ethoxyphenyl)-N′-(5-methylisoxazol-3-yl)urea;

N-(2-methoxyphenyl)-N′-[5-(trifluoromethyl)-1,3,4-thiadiazol-2-yl]urea;

N-(2-fluoro-4-methoxyphenyl)-N′-[5-(trifluoromethyl)-1,3,4-thiadiazol-2-yl]urea;

N-(5-chloro-2,4-dimethoxyphenyl)-N′-(5-mercapto-1,3,4-thiadiazol-2-yl)urea;

N-(4,5-dimethoxy-2-methylphenyl)-N′-[5-(trifluoromethyl)-1,3,4-thiadiazol-2-yl]urea;

N-(4-hydroxy-2-nitrophenyl)-N′-[5-(trifluoromethyl)-1,3,4-thiadiazol-2-yl]urea;

N-(2,6-dimethoxypyridin-3-yl)-N′-(5-methyl-1,3-thiazol-2-yl)urea;

N-(4-ethoxy-2-nitrophenyl)-N′-(5-methylisoxazol-3-yl)urea;

N-(4-methoxy-2-methylphenyl)-N′-[2-(trifluoromethyl) pyridin-4-yl]urea;

N-(5-fluoro-2,4-dimethoxyphenyl)-N′-(5-methylisoxazol-3-yl)urea;

N-(5-fluoro-2,4-dimethoxyphenyl)-N′-(5-methyl-1,3-thiazol-2-yl)urea;

N-(5-fluoro-2,4-dimethoxyphenyl)-N′-(5-methyl-1,3,4-thiadiazol-2-yl)urea;

N-(5-fluoro-2,4-dimethoxyphenyl)-N′-[5-(trifluoromethyl)-1,3,4-thiadiazol-2-yl]urea;

N-(5-bromo-2,4-dimethoxyphenyl)-N′-(5-methylisoxazol-3-yl)urea;

N-(5-bromo-2,4-dimethoxyphenyl)-N′-(5-methyl-1,3-thiazol-2-yl)urea;

N-(5-bromo-2,4-dimethoxyphenyl)-N′-(5-methyl-1,3,4-thiadiazol-2-yl)urea;

N-(5-bromo-2,4-dimethoxyphenyl)-N′-[5-(trifluoromethyl)-1,3,4-thiadiazol-2-yl]urea;

N-(3,5-difluoro-2,4-dimethoxyphenyl)-N′-(5-methylisoxazol-3-yl)urea;

N-(3,5-difluoro-2,4-di methoxyphenyl)-N′-[5-(trifluoromethyl)-1,3,4-thiadiazol-2-yl]urea;

N-[2,4-dimethoxy-5-(trifluoromethyl)phenyl]-N′-[5-(trifluoromethyl)-1,3,4-thiadiazol-2-yl]urea;

N-(5-chloro-2,4-diethoxyphenyl)-N′-(5-methylisoxazol-3-yl)urea;

N-(5-chloro-2,4-diethoxyphenyl)-N′-(5-methyl-1,3-thiazol-2-yl)urea;

N-(5-chloro-2,4-diethoxyphenyl)-N-(5-methyl-1,3,4-thiadiazol-2-yl)urea;

N-(5-chloro-2,4-diethoxyphenyl)-N′-[5-(trifluoromethyl)-1,3,4-thiadiazol-2-yl]urea;

N-(5-chloro-2,4-dipropoxyphenyl)-N-[5-(trifluoromethyl)-1,3,4-thiadiazol-2-yl]urea;

N-(2-chloro-4-methoxy-5-methylphenyl)-N′-(5-methylisoxazol-3-yl)urea;

N-(2-chloro-4-methoxy-5-methylphenyl)-N′-[5-(trifluoromethyl)-1,3,4-thiadiazol-2-yl]urea;

N-(2,4-dimethoxy-5-methylphenyl)-N′-(5-methylisoxazol-3-yl)urea;

N-(2,4-dimethoxy-5-methylphenyl)-N′-(5-methyl-1,3-thiazol-2-yl)urea;

N-(2,4-dimethoxy-5-methylphenyl)-N′-(5-methyl-1,3,4-thiadiazol-2-yl)urea;

N-(2,4-dimethoxy-5-methylphenyl)-N′-[5-(trifluoromethyl)-1,3,4-thiadiazol-2-yl]urea;

N-(4-ethoxy-2-methoxy-5-methylphenyl)-N′-[5-(trifluoromethyl)-1,3,4-thiadiazol-2-yl]urea;

N-(5-acetyl-2,4-dimethoxyphenyl)-N′-[5-(trifluoromethyl)-1,3,4-thiadiazol-2-yl]urea;

N-(2,4-dimethoxy-5-nitrophenyl)-N′-[5-(trifluoromethyl)-1,3,4-thiadiazol-2-yl]urea;

N-[2-methoxy-5-methyl-4-(2,2,2-trifluoroethoxy)phenyl]-N′-[5-(trifluoromethyl)-1,3,4-thiadiazol-2-yl]urea;

N-(5-chloro-2,4-dimethoxyphenyl)-N′-(2-methyl-1,3-thiazol-5-yl)urea;

N-(2-methoxy-4-nitrophenyl)-N′-[5-(trifluoromethyl)-1,3,4-thiadiazol-2-yl]urea;

N-[5-(trifluoromethyl)-1,3,4-thiadiazol-2-yl]-N′-(2,4,5-trimethoxyphenyl)urea;

N-[4-methoxy-2-(methylthio)phenyl]-N′-[5-(trifluoromethyl)-1,3,4-thiadiazol-2-yl]urea;

N-(4-{[(1R)-1-methylpropyl]oxy}phenyl)-N′-[5-(trifluoromethyl)-1,3,4-thiadiazol-2-yl]urea;

N-[4-(allyloxy)phenyl]-N′-[5-(trifluoromethyl)-1,3,4-thiadiazol-2-yl]urea;

N-(4-propoxyphenyl)-N′-[5-(trifluoromethyl)-1,3,4-thiadiazol-2-yl]urea;

N-(2-ethoxypyridin-3-yl)-N′-[5-(trifluoromethyl)-1,3,4-thiadiazol-2-yl]urea;

N-(4-methoxy-2-methylphenyl)-N′-[4-(trifluoromethyl)-1,3-thiazol-2-yl]urea;

N-(4-methoxy-2-methylphenyl)-N′-(3-phenyl-1,2,4-thiadiazol-5-yl)urea;

N-(5-ethyl-4-phenyl-1,3-thiazol-2-yl)-N′-(4-methoxy-2-methylphenyl)urea;

N-(4-hydroxy-2-methylphenyl)-N′-[3-(trifluoromethyl)phenyl]urea;

N-(4-hydroxyphenyl)-N′-[3-(trifluoromethyl)phenyl]urea;

N-[2-methyl-4-(methylthio)phenyl]-N′-[3-(trifluoromethyl)phenyl]urea;

N-(2-ethyl-4-hydroxyphenyl)-N′-[3-(trifluoromethyl)phenyl]urea;

N-(4-amino-2-methylphenyl)-N′-[3-(trifluoromethyl)phenyl]urea;

N-(4-methoxyphenyl)-N′-[3-(trifluoromethyl)phenyl]urea;

N-(5-hydroxy-2-methylphenyl)-N′-[3-(trifluoromethyl)phenyl]urea;

N-(5-chloro-2,4-dimethoxyphenyl)-N′-[4-(trifluoromethyl)-1H-pyrazol-1-yl]urea;

N-(4-bromo-1H-pyrazol-1-yl)-N′-(5-chloro-2,4-dimethoxyphenyl)urea;

N-(4-methoxy-2-methylphenyl)-N′-[3-(trifluoromethyl)phenyl]thiourea;

N-(4-hydroxy-2-methylphenyl)-N′-[3-(trifluoromethyl)phenyl]thiourea;

N-(2,4-dimethoxy-5-methylphenyl)-N′-[3-(trifluoromethyl)isoxazol-5-yl]urea;

N-(4-ethoxyphenyl)-N′-[3-(trifluoromethyl)isoxazol-5-yl]urea;

N-(2-ethoxyphenyl)-N′-[3-(trifluoromethyl)isoxazol-5-yl]urea;

N-(2,4-dimethoxyphenyl)-N′-[3-(trifluoromethyl)isoxazol-5-yl]urea;

N-(2,6-dimethoxypyridin-3-yl)-N′-(3-methylisoxazol-5-yl)urea;

N-(5-fluoro-2,4-dimethoxyphenyl)-N′-(3-methylisoxazol-5-yl)urea;

N-(2,4-dimethoxyphenyl)-N′-(3-methylisoxazol-5-yl)urea;

N-(5-chloro-2,4-dimethoxyphenyl)-N′-[3-(trifluoromethyl)isoxazol-5-yl]thiourea;

N-(5-chloro-2,4-diethoxyphenyl)-N′-[3-(trifluoromethyl)isoxazol-5-yl]urea;

N-(4-methoxy-2-nitrophenyl)-N′-[3-(trifluoromethyl)isoxazol-5-yl]urea;

N-(4-methoxy-2-methylphenyl)-N′-[4-(trifluoromethyl)-1,3-thiazol-2-yl]thiourea;

N-(5-chloro-2,4-dimethoxyphenyl)-N′-[5-(trifluoromethyl)-1,3,4-thiadiazol-2-yl]thiourea;

N-(5-fluoro-2,4-dimethoxyphenyl)-N′-[5-(trifluoromethyl)-1,3,4-thiadiazol-2-yl]thiourea;

N-[2-methoxy-4-(2-methoxyethoxy)phenyl]-N′-[5-(trifluoromethyl)-1,3,4-thiadiazol-2-yl]urea;

N-(4-ethoxy-2-nitrophenyl)-N′-[3-(trifluoromethyl)isoxazol-5-yl]thiourea;

N-(4-hydroxy-2-methylphenyl)-N′-[4-(trifluoromethyl)-1,3-thiazol-2-yl]thiourea;

N-(3-chloro-4-methoxyphenyl)-N′-[5-(trifluoromethyl)-1,3,4-thiadiazol-2-yl]urea;

N-(5-chloro-2-methoxyphenyl)-N′-[5-(trifluoromethyl)-1,3,4-thiadiazol-2-yl]urea;

N-(4-methoxy-2-nitrophenyl)-N′-[5-(trifluoromethyl)-1,3,4-thiadiazol-2-yl]urea;

N-(4-ethoxy-2-nitrophenyl)-N′-[5-(trifluoromethyl)-1,3,4-thiadiazol-2-yl]urea;

N-(4-methoxy-2-methylphenyl)-N′-[5-(trifluoromethyl)-1,3,4-thiadiazol-2-yl]urea;

N-(2,6-dimethoxypyridin-3-yl)-N′-[5-(trifluoromethyl)-1,3,4-thiadiazol-2-yl]urea;

N-(4-hydroxyphenyl)-N′-[5-(trifluoromethyl)-1,3,4-thiadiazol-2-yl]urea;

N-(5-ethoxypyridin-2-yl)-N′-[5-(trifluoromethyl)-1,3,4-thiadiazol-2-yl]urea;

N-(4-ethoxy-2-morpholin-4-ylphenyl)-N′-[5-(trifluoromethyl)-1,3,4-thiadiazol-2-yl]urea;

tert-butyl 4-{5-ethoxy-2-[({[5-(trifluoromethyl)-1,3,4-thiadiazol-2-yl]amino}carbonyl) amino]phenyl}piperazine-1-carboxylate;

N-(2-chloro-6-methoxy-pyridin-3-yl)-N′-[5-(trifluoromethyl)-1,3,4-thiadiazol-2-yl]urea;

N-[6-methoxy-2-(methylthio)pyridin-3-yl]-N′-[5-(trifluoromethyl)-1,3,4-thiadiazol-2-yl]urea;

N-[6-methoxy-2-(methylsulfonyl)pyridin-3-yl]-N′-[5-(trifluoromethyl)-1,3,4-thiadiazol-2-yl]urea;

N-[2-methoxy-4-(2-methoxyethoxy)phenyl]-N′-(5-methylisoxazol-3-yl)urea;

N-(4-hydroxy-2-methylphenyl)-N′-[4-(trifluoromethyl)-1,3-thiazol-2-yl]urea;

N-(4-hydroxyphenyl)-N′-[4-(trifluoromethyl)-1,3-thiazol-2-yl]urea;

N-(5-hydroxy-2-methylphenyl)-N′-[4-(trifluoromethyl)-1,3-thiazol-2-yl]urea;

N-(3-hydroxy-2-methylphenyl)-N′-[4-(trifluoromethyl)-1,3-thiazol-2-yl]urea;

N-(6-cyanopyridin-3-yl)-N′-(5-fluoro-2,4-dimethoxyphenyl)urea;

N-(4-hydroxy-2-methylphenyl)-N′-[3-(trifluoromethoxy)phenyl]urea;

N-(4-hydroxy-2-methylphenyl)-N′-[4-(pentafluoroethyl)-1,3-thiazol-2-yl]urea;

N-(5-bromo-2,4-dimethoxyphenyl)-N′-(6-cyanopyridin-3-yl)urea;

N-(5-chloro-2,4-dimethoxyphenyl)-N′-[3-(trifluoromethyl)isoxazol-5-yl]urea;

N-(5-fluoro-2,4-dimethoxyphenyl)-N′-[3-(trifluoromethyl)isoxazol-5-yl]urea;

N-(2,6-dimethoxypyridin-3-yl)-N′-[3-(trifluoromethyl)isoxazol-5-yl]urea;

N-(4-ethoxy-2-nitrophenyl)-N′-[3-(trifluoromethyl)isoxazol-5-yl]urea;

N-(5-chloro-2,4-dimethoxyphenyl)-N′-(5-chloroisoxazol-3-yl)urea;

N-(5-chloro-4-methoxy-2-nitrophenyl)-N′-(5-methylisoxazol-3-yl)urea;

N-(5-fluoro-4-methoxy-2-nitrophenyl)-N′-(5-methylisoxazol-3-yl)urea;

N-[5-chloro-4-methoxy-2-(methylthio)phenyl]-N′-(5-methylisoxazol-3-yl)urea;

N-(5-chloro-2,4-dimethoxyphenyl)-N′-[5-(fluoromethyl)isoxazol-3-yl]urea;

5 N-(2,6-dimethoxypyridin-3-yl)-N′-[5-(fluoromethyl)isoxazol-3-yl]urea;

N-(5-fluoro-2,4-dimethoxyphenyl)-N′-[5-(fluoromethyl)isoxazol-3-yl]urea;

N-(5-bromo-2,4-dimethoxyphenyl)-N′-[5-(fluoromethyl)isoxazol-3-yl]urea;

N-(4-ethoxy-2-nitrophenyl)-N′-[5-(fluoromethyl)isoxazol-3-yl]urea;

N-(2,4-dimethoxy-5-methylphenyl)-N′-[5-(fluoromethyl)isoxazol-3-yl]urea;

N-(5-bromo-2,4-dimethoxyphenyl)-N′-[5-(hydroxymethyl)isoxazol-3-yl]urea;

(5-fluoro-2,4-dimethoxyphenyl)-N′-(5-isopropylisoxazol-3-yl)urea;

N-(5-chloro-2,4-dimethoxyphenyl)-N′-(5-isopropylisoxazol-3-yl)urea;

N-(5-bromo-2,4-dimethoxyphenyl)-N′-(5-isopropylisoxazol-3-yl)urea;

N-(2,6-dimethoxypyridin-3-yl)-N′-(5-isopropylisoxazol-3-yl)urea;

N-(2,4-dimethoxy-5-methylphenyl)-N′-(5-isopropylisoxazol-3-yl)urea;

N-(5-fluoro-2,4-dimethoxyphenyl)-N′-[5-(methoxymethyl)isoxazol-3-yl]urea;

N-(5-chloro-2,4-dimethoxyphenyl)-N′-[5-(methoxymethyl)isoxazol-3-yl]urea;

N-(5-bromo-2,4-dimethoxyphenyl)-N′-[5-(methoxymethyl)isoxazol-3-yl]urea;

N-(4-ethoxy-2-nitrophenyl)-N′-[5-(methoxymethyl)isoxazol-3-yl]urea;

N-(5-chloro-2,4-dimethoxyphenyl)-N′-(5-cyclopropylisoxazol-3-yl)urea;

N-(5-cyclopropylisoxazol-3-yl)-N′-(5-fluoro-2,4-dimethoxyphenyl)urea;

N-(5-bromo-2,4-dimethoxyphenyl)-N′-(5-cyclopropylisoxazol-3-yl)urea;

N-(5-cyclopropylisoxazol-3-yl)-N′-(2,4-dimethoxy-5-methylphenyl)urea;

N-(5-cyclopropylisoxazol-3-yl)-N′-(4-ethoxy-2-nitrophenyl)urea;

N-(5-cyclopropylisoxazol-3-yl)-N′-(2,6-dimethoxypyridin-3-yl)urea;

N-(5-chloro-2,4-dimethoxyphenyl)-N′-(5-ethylisoxazol-3-yl)urea;

N-(5-ethylisoxazol-3-yl)-N′-(5-fluoro-2,4-dimethoxyphenyl)urea;

N-(5-bromo-2,4-dimethoxyphenyl)-N′-(5-ethylisoxazol-3-yl)urea;

N-(2,4-dimethoxy-5-methylphenyl)-N′-(5-ethylisoxazol-3-yl)urea;

N-(2,6-dimethoxypyridin-3-yl)-N′-(5-ethylisoxazol-3-yl)urea;

N-(4-ethoxy-2-nitrophenyl)-N′-(5-ethylisoxazol-3-yl)urea;

N-(5-amino-2,4-dimethoxyphenyl)-N′-[5-(trifluoromethyl)-1,3,4-thiadiazol-2-yl]urea hydrochloride;

N-(5-azido-2,4-dimethoxyphenyl)-N′-[5-(trifluoromethyl)-1,3,4-thiadiazol-2-yl]urea;

N-(5-iodo-2,4-dimethoxyphenyl)-N′-[5-(trifluoromethyl)-1,3,4-thiadiazol-2-yl]urea;

N-(4-amino-2-methoxyphenyl)-N′-[5-(trifluoromethyl)-1,3,4-thiadiazol-2-yl]urea;

N-(4-methoxy-2-methylphenyl)-N′-[4-(pentafluoroethyl)-1,3-thiazol -2-yl]urea;

N-(5-chloro-2,4-dimethoxyphenyl)-N′-[5-(trifluoromethyl)isoxazol-3-yl]urea;

N-[2-methoxy-4-(tetrahydrofuran-3-yloxy)phenyl]-N′-[5-(trifluoromethyl)-1,3,4-thiadiazol-2-yl]urea;

N-[2-methoxy-4-(oxetan-3-yloxy)phenyl]-N′-[5-(trifluoromethyl)-1,3,4-thiadiazol-2-yl]urea;

N-[2-methoxy-4-(oxetan-3-yloxy)phenyl]-N′-[3-(trifluoromethyl)isoxazol-5-yl]urea;

and N-[2-methoxy-4-(tetrahydrofuran-3-yloxy)phenyl]-N′-[3-(trifluoromethyl)isoxazol-5-yl]urea.

What is claimed:

1. A compound of Formula I: 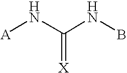
wherein X is O or S;
A is 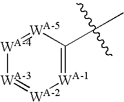
wherein each W^{1-A}, W^{A-2}, W^{A-3}, W^{A-4}, and W^{A-5} are independently N or CR_{A}, provided that no more than four of W^{A-1}, W^{A-2}, W^{A-3}, W^{A-4}, or W^{A-5} are simultaneously N;
Each R_{A} is independently H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₈, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂, provided that at least one R_{A} is other than H;
or when two R_{A} are on adjacent carbon atoms, the two R_{A} may combine to form a 5-8-membered ring fused to the 6-membered ring, wherein the 5-8-membered ring is saturated or unsaturated having up to two heteroatoms selected from —O—, —S—, —N(R_{A-2})—, or —N═ and further having substitution where valency allows on the 5-8-membered ring with up to 2 substitutents independently selected from R_{A-1};
Each R_{A-1} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;
R_{A-2} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
B is a five or six-membered aromatic ring having up to 4 heteroatoms selected from —O—, —N(R_{B-3})—, ═N—, or —S—, wherein B is 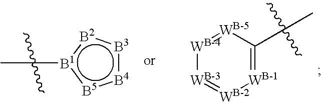
wherein B¹ is N, or C;
B², B³, B⁴, and B⁵ are independently N, O, S, C, provided that when valency allows, the N can have a third bond to R_{B-3}, and further provided that when valency allows, the C can have a fourth bond to R_{B-1};
Each R_{B-1} is independently H, halogen, alkyl, haloalkyl, substituted alkyl, cycloalkyl, halocycloalkyl, substituted cycloalkyl, alkenyl, haloalkenyl, substituted alkenyl, alkynyl, haloalkynyl, substituted alkynyl, heterocycloalkyl, haloheterocycloalkyl, substituted heterocycloalkyl, aryl, —CN, —N₃, —NO₂, —COR₁₀, —CO₂R₇, —CON(R₉)₂, —C(R₁₀)═NOR₇, —SCN, —OR₇, —N(R₉)₂, —SR₈, —SOR₈, —SO₂R₈, —SN(R₉)₂, —SON(R₉)₂, —SO₂N(R₉)₂;
when two R_{B-1} are on adjacent carbon atoms, the two R_{B-1} may combine to form a 5-7-membered ring fused to the 5 or 6 membered ring giving a fused-bicyclic-ring system; wherein the 5-7-membered ring is saturated or unsaturated having up to two heteroatoms selected from —O—, —S—, —N(R_{B-3})—, or —N=and further having substitution where valency allows on the 5-7-membered ring with up to 2 substitutents independently selected from R_{B-2};
Each R_{B-2} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;
R_{B-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
Each W^{B-1}, W^{B-2}, W^{B-3},W^{B-4}, and W^{B-5} are independently N or CR_{B-1}, provided that no more than 4 of W^{B-1}, W^{B-2}, W^{B-3}, W^{B-4}, or W^{B-5} are simultaneously N;
R₇ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
R₈ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
Each R₉ is independently H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
R₁₀ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
or pharmaceutical composition, pharmaceutically acceptable salt, racemic mixture, or pure enantiomer thereof;
provided that where B is thiadiazolyl and A is phenyl then at least one R_{A} is selected from other than H, methyl, isopropyl, —NO₂, —CF₃, methoxy, —OH, —CN, or halogen;
and further provided when B is isoxazol-3-yl optionally substituted at the four position with trifluoromethyl, O—C-₁₋₄alkyl, or alkyl substituted with hydroxy and A is phenyl, the phenyl cannot be substituted with alkyl, trifluoromethyl or halogen at either ortho position.

2. The compound of claim 1, wherein the compound is
N-(4-methoxy-2-methylphenyl)-N′-[3-(trifluoromethyl)phenyl]urea;
N-(5-chloro-2,4-dimethoxyphenyl)-N′-isoxazol-3-ylurea;
N-(5-chloro-2,4-dimethoxyphenyl)-N′-(5-methyl-1,3,4-thiadiazol-2-yl)urea;
N-(5-chloro-2,4-dimethoxyphenyl)-N′-(5-ethyl-1,3,4-thiadiazol-2-yl)urea;
N-(5-chloro-2,4-dimethoxyphenyl)-N′-[5-(trifluoromethyl)-1,3,4-thiadiazol-2-yl]urea;
N-(5-chloro-2,4-dimethoxyphenyl)-N′-(5-cyclopropyl-1,3,4-thiadiazol-2-yl)urea;
N-(5-chloro-2,4-dimethoxyphenyl)-N′-[5-(difluoromethyl)-1,3,4-thiadiazol-2-yl]urea;
N-(5-chloro-2,4-dimethoxyphenyl)-N′-(5-methoxy-1,3,4-thiadiazol-2-yl)urea;
N-(5-bromo-1,3,4-thiadiazol-2-yl)-N′-(5-chloro-2,4-dimethoxyphenyl)urea;
N-(5-chloro-1,3,4-thiadiazol-2-yl)-N′-(5-chloro-2,4-dimethoxyphenyl)urea;
N-(5-chloro-2,4-dimethoxyphenyl)-N′-[5-(2-phenylethyl)-1,3,4-thiadiazol-2-yl]urea;
N-(5-chloro-2,4-dimethoxyphenyl)-N′-(5-ethoxy-1,3,4-thiadiazol-2-yl)urea;
N-(5-chloro-2,4-dimethoxyphenyl)-N′-(1,3,4-thiadiazol-2-yl)urea;
N-(4-ethoxyphenyl)-N′-(5-methylisoxazol-3-yl)urea;
N-(2,4-dimethoxyphenyl)-N′-(5-methylisoxazol-3-yl)urea;
N-(2,4-dimethoxyphenyl)-N′-[5-(trifluoromethyl)-1,3,4-thiadiazol-2-yl]urea;
N-(5-chloro-2-methoxyphenyl)-N′-(5-methylisoxazol-3-yl)urea;
N-(5-chloro-2-methoxyphenyl)-N′-(5-methyl-1,3,4-thiadiazol-2-yl)urea;
N-(4-isopropoxyphenyl)-N′-[5-(trifluoromethyl)-1,3,4-thiadiazol-2-yl]urea;
N-(2-ethoxyphenyl)-N′-[5-(trifluoromethyl)-1,3,4-thiadiazol-2-yl]urea;
N-(4-ethoxyphenyl)-N′-[5-(trifluoromethyl)-1,3,4-thiadiazol-2-yl]urea;
N-(2,3-dihydro-1,4-benzodioxin-6-yl)-N′-[5-(trifluoromethyl)-1,3,4-thiadiazol-2-yl]urea;
N-(2,3-dihydro-1-benzofuran-5-yl)-N′-[5-(trifluoromethyl)-1,3,4-thiadiazol-2-yl]urea;
N-(4-ethyl-1,3-thiazol-2-yl)-N′-(4-methoxy-2-methylphenyl)urea;
N-(5-chloro-2,4-dimethoxyphenyl)-N′-(1H-imidazol-2-yl)urea;
N-(5-chloro-2,4-dimethoxyphenyl)-N′-(3-methylisoxazol-5-yl)urea;
N-(5-chloro-2,4-dimethoxyphenyl)-N′-(6-cyanopyridin-3-yl)urea;
N-(5-chloro-1,3-thiazol-2-yl)-N′-(2,4-dimethoxy-5-methylphenyl)urea;
N-(5-bromo-2,4-dimethoxyphenyl)-N′-(5-chloro-1,3-thiazol-2-yl)urea;
N-(5-bromo-1,3-thiazol-2-yl)-N′-(5-fluoro-2,4-dimethoxyphenyl)urea;
N-(5-chloro-1,3-thiazol-2-yl)-N′-(5-fluoro-2,4-dimethoxyphenyl)urea;
N-(3-chloro-4-fluorophenyl)-N′-(5-methylisoxazol-3-yl)urea;
N-(3-chloro-4-fluorophenyl)-N′-[5-(trifluoromethyl)-1,3,4-thiadiazol-2-yl]urea;
N-(2-ethoxyphenyl)-N′-(5-methylisoxazol-3-yl)urea;
N-(2-methoxyphenyl)-N′-[5-(trifluoromethyl)-1,3,4-thiadiazol-2-yl]urea;
N-(2-fluoro-4-methoxyphenyl)-N′-[5-(trifluoromethyl)-1,3,4-thiadiazol-2-yl]urea;
N-(5-chloro-2,4-dimethoxyphenyl)-N′-(5-mercapto-1,3,4-thiadiazol-2-yl)urea;
N-(4,5-dimethoxy-2-methylphenyl)-N′-[5-(trifluoromethyl)-1,3,4-thiadiazol-2-yl]urea;
N-(4-hydroxy-2-nitrophenyl)-N′-[5-(trifluoromethyl)-1,3,4-thiadiazol-2-yl]urea;
N-(2,6-dimethoxypyridin-3-yl)-N′-(5-methyl-1,3-thiazol-2-yl)urea;
N-(4-ethoxy-2-nitrophenyl)-N′-(5-methylisoxazol-3-yl)urea;
N-(4-methoxy-2-methylphenyl)-N′-[2-(trifluoromethyl) pyridin-4-yl]urea;
N-(5-fluoro-2,4-dimethoxyphenyl)-N′-(5-methylisoxazol-3-yl)urea;
N-(5-fluoro-2,4-dimethoxyphenyl)-N′-(5-methyl-1,3-thiazol-2-yl)urea;
N-(5-fluoro-2,4-dimethoxyphenyl)-N′-(5-methyl-1,3,4-thiadiazol-2-yl)urea;
N-(5-fluoro-2,4-dimethoxyphenyl)-N′-[5-(trifluoromethyl)-1,3,4-thiadiazol-2-yl]urea;
N-(5-bromo-2,4-dimethoxyphenyl)-N′-(5-methylisoxazol-3-yl)urea;
N-(5-bromo-2,4-dimethoxyphenyl)-N′-(5-methyl-1,3-thiazol-2-yl)urea;
N-(5-bromo-2,4-dimethoxyphenyl)-N′-(5-methyl-1,3,4-thiadiazol-2-yl)urea;
N-(5-bromo-2,4-dimethoxyphenyl)-N′-[5-(trifluoromethyl)-1,3,4-thiadiazol-2-yl]urea;
N-(3,5-difluoro-2,4-dimethoxyphenyl)-N′-(5-methylisoxazol-3-yl)urea;
N-(3,5-difluoro-2,4-dimethoxyphenyl)-N′-[5-(trifluoromethyl)-1,3,4-thiadiazol-2-yl]urea;
N-(3,5-difluoro-2,4-dimethoxyphenyl)-N′-(5-methyl-1,3-thiazol-2-yl)urea;
N-(3,5-difluoro-2,4-dimethoxyphenyl)-N′-(5-methyl-1,3,4-thiadiazol-2-yl)urea;
N-[2,4-dimethoxy-5-(trifluoromethyl)phenyl]-N′-[5-(trifluoromethyl)-1,3,4-thiadiazol-2-yl]urea;
N-[2,4-dimethoxy-5-(trifluoromethyl)phenyl]-N′-(5-methylisoxazol-3-yl)urea;
N-[2,4-dimethoxy-5-(trifluoromethyl)phenyl]-N′-(5-methyl-1,3-thiazol-2-yl)urea;
N-[2,4-dimethoxy-5-(trifluoromethyl)phenyl]-N′-(5-methyl-1,3,4-thiadiazol-2-yl)urea;
N-(5-chloro-2,4-diethoxyphenyl)-N′-(5-methylisoxazol-3-yl)urea;
N-(5-chloro-2,4-diethoxyphenyl)-N′-(5-methyl-1,3-thiazol-2-yl)urea;
N-(5-chloro-2,4-diethoxyphenyl)-N′-(5-methyl-1,3,4-thiadiazol-2-yl)urea;
N-(5-chloro-2,4-diethoxyphenyl)-N′-[5-(trifluoromethyl)-1,3,4-thiadiazol-2-yl]urea;
N-(5-chloro-2,4-dipropoxyphenyl)-N′-[5-(trifluoromethyl)-1,3,4-thiadiazol-2-yl]urea;
N-(5-chloro-2,4-dipropoxyphenyl)-N′-(5-methylisoxazol-3-yl)urea;
N-(5-chloro-2,4-diisopropoxyphenyl)-N′-(5-methyl-1,3,4-thiadiazol-2-yl)urea;
N-(5-chloro-2,4-diisoprooxyphenyl)-N′-[5-(trifluoromethyl)-1,3,4-thiadiazol-2-yl]urea;
N-(5-chloro-2,4-diisoprooxyphenyl)-N′-(5-methylisoxazol-3-yl)urea;
N-(2-chloro-4-methoxy-5-methylphenyl)-N′-(5-methylisoxazol-3-yl)urea;
N-(2-chloro-4-methoxy-5-methylphenyl)-N′-(5-methyl-1,3-thiazol-2-yl)urea;
N-(2-chloro-4-methoxy-5-methylphenyl)-N′-(5-methyl-1,3,4-thiadiazol-2-yl)urea;
N-(2-chloro-4-methoxy-5-methylphenyl)-N′-[5-(trifluoromethyl)-1,3,4-thiadiazol-2-yl]urea;
N-(2,4-dimethoxy-5-methylphenyl)-N′-(5-methylisoxazol-3-yl)urea;
N-(2,4-dimethoxy-5-methylphenyl)-N′-(5-methyl-1,3-thiazol-2-yl)urea;
N-(2,4-dimethoxy-5-methylphenyl)-N′-(5-methyl-1,3,4-thiadiazol-2-yl)urea;
N-(2,4-dimethoxy-5-methylphenyl)-N′-[5-(trifluoromethyl)-1,3,4-thiadiazol-2-yl]urea;
N-(4-ethoxy-2-methoxy-5-methylphenyl)-N′-[5-(trifluoromethyl)-1,3,4-thiadiazol-2-yl]urea;
N-(5-acetyl-2,4-dimethoxyphenyl)-N′-[5-(trifluoromethyl)-1,3,4-thiadiazol-2-yl]urea;
N-(5-acetyl-2,4-dimethoxyphenyl)-N′-(5-methyl-1,3,4-thiadiazol-2-yl)urea;
N-(5-acetyl-2,4-dimethoxyphenyl)-N′-(5-methylisoxazol-3-yl)urea;
N-(2,4-dimethoxy-5-nitrophenyl)-N′-[5-(trifluoromethyl)-1,3,4-thiadiazol-2-yl]urea;
N-(2,4-dimethoxy-5-nitrophenyl)-N′-(5-methyl-1,3-thiazol-2-yl)urea;
N-(2,4-dimethoxy-5-nitrophenyl)-N′-(5-methylisoxazol-3-yl)urea;
N-[2-methoxy-5-methyl-4-(2,2,2-trifluoroethoxy)phenyl]-N′-[5-(trifluoromethyl)-1,3,4-thiadiazol-2-yl]urea;
N-(5-chloro-2,4-dimethoxyphenyl)-N′-(2-methyl-1,3-thiazol-5-yl)urea;
N-(2-methoxy-4-nitrophenyl)-N′-[5-(trifluoromethyl)-1,3,4-thiadiazol-2-yl]urea;
N-[5-(trifluoromethyl)-1,3,4-thiadiazol-2-yl]-N′-(2,4,5-trimethoxyphenyl)urea;
N-[4-methoxy-2-(methylthio)phenyl]-N′-[5-(trifluoromethyl)-1,3,4-thiadiazol-2-yl]urea;
N-(4-{[(1R)-1-methylpropyl]oxy}phenyl)-N′-[5-(trifluoromethyl)-1,3,4-thiadiazol-2-yl]urea;
N-[4-(allyloxy)phenyl]-N′-[5-(trifluoromethyl)-1,3,4-thiadiazol-2-yl]urea;
N-(4-propoxyphenyl)-N′-[5-(trifluoromethyl)-1,3,4-thiadiazol-2-yl]urea;
N-(2-ethoxypyridin-3-yl)-N′-[5-(trifluoromethyl)-1,3,4-thiadiazol-2-yl]urea;
N-(4-methoxy-2-methylphenyl)-N′-[4-(trifluoromethyl)-1,3-thiazol-2-yl]urea;
N-(4-methoxy-2-methylphenyl)-N′-(3-phenyl-1,2,4-thiadiazol-5-yl)urea;
N-(5-ethyl-4-phenyl-1,3-thiazol-2-yl)-N′-(4-methoxy-2-methylphenyl)urea;
N-(4-hydroxy-2-methylphenyl)-N′-[3-(trifluoromethyl)phenyl]urea;
N-(4-hydroxyphenyl)-N′-[3-(trifluoromethyl)phenyl]urea;
N-[2-methyl-4-(methylthio)phenyl]-N′-[3-(trifluoromethyl)phenyl]urea;
N-(2-ethyl-4-hydroxyphenyl)-N′-[3-(trifluoromethyl)phenyl]urea;
N-(4-amino-2-methylphenyl)-N′-[3-(trifluoromethyl)phenyl]urea;
N-(4-methoxyphenyl)-N′-[3-(trifluoromethyl)phenyl]urea;
N-(5-hydroxy-2-methylphenyl)-N′-[3-(trifluoromethyl)phenyl]urea;
N-(5-chloro-2,4-dimethoxyphenyl)-N′-[4-(trifluoromethyl)-1H-pyrazol-1-yl]urea;
N-(4-bromo-1H-pyrazol-1-yl)-N′-(5-chloro-2,4-dimethoxyphenyl)urea;
N-(4-methoxy-2-methylphenyl)-N′-[3-(trifluoromethyl)phenyl]thiourea;
N-(4-hydroxy-2-methylphenyl)-N′-[3-(trifluoromethyl)phenyl]thiourea;
N-(2,4-dimethoxy-5-methylphenyl)-N′-[3-(trifluoromethyl)isoxazol-5-yl]urea;
N-(4-ethoxyphenyl)-N′-[3-(trifluoromethyl)isoxazol-5-yl]urea;
N-(2-ethoxyphenyl)-N′-[3-(trifluoromethyl)isoxazol-5-yl]urea;
N-(2,4-dimethoxyphenyl)-N′-[3-(trifluoromethyl)isoxazol-5-yl]urea;
N-(2,6-dimethoxypyridin-3-yl)-N′-(3-methylisoxazol-5-yl)urea;
N-(5-fluoro-2,4-dimethoxyphenyl)-N′-(3-methylisoxazol-5-yl)urea;
N-(2,4-dimethoxyphenyl)-N′-(3-methylisoxazol-5-yl)urea;
N-(5-chloro-2,4-dimethoxyphenyl)-N′-[3-(trifluoromethyl)isoxazol-5-yl]thiourea;
N-(5-chloro-2,4-diethoxyphenyl)-N′-[3-(trifluoromethyl)isoxazol-5-yl]urea;
N-(4-methoxy-2-nitrophenyl)-N′-[3-(trifluoromethyl)isoxazol-5-yl]urea;
N-(4-methoxy-2-methylphenyl)-N′-[4-(trifluoromethyl)-1,3-thiazol-2-yl]thiourea;
N-(5-chloro-2,4-dimethoxyphenyl)-N′-[5-(trifluoromethyl)-1,3,4-thiadiazol-2-yl]thiourea;
N-(5-fluoro-2,4-dimethoxyphenyl)-N′-[5-(trifluoromethyl)-1,3,4-thiadiazol-2-yl]thiourea;
N-[2-methoxy-4-(2-methoxyethoxy)phenyl]-N′-[5-(trifluoromethyl)-1,3,4-thiadiazol-2-yl]urea;
N-(4-ethoxy-2-nitrophenyl)-N′-[3-(trifluoromethyl)isoxazol-5-yl]thiourea;
N-(4-hydroxy-2-methylphenyl)-N′-[4-(trifluoromethyl)-1,3-thiazol-2-yl]thiourea;
N-(3-chloro-4-methoxyphenyl)-N′-[5-(trifluoromethyl)-1,3,4-thiadiazol-2-yl]urea;
N-(5-chloro-2-methoxyphenyl)-N′-[5-(trifluoromethyl)-1,3,4-thiadiazol-2-yl]urea;
N-(4-methoxy-2-nitrophenyl)-N′-[5-(trifluoromethyl)-1,3,4-thiadiazol-2-yl]urea;
N-(4-ethoxy-2-nitrophenyl)-N′-[5-(trifluoromethyl)-1,3,4-thiadiazol-2-yl]urea;
N-(4-methoxy-2-methylphenyl)-N′-[5-(trifluoromethyl)-1,3,4-thiadiazol-2-yl]urea;
N-(2,6-dimethoxypyridin-3-yl)-N′-[5-(trifluoromethyl)-1,3,4-thiadiazol-2-yl]urea;
N-(4-hydroxyphenyl)-N′-[5-(trifluoromethyl)-1,3,4-thiadiazol-2-yl]urea;
N-(5-ethoxypyridin-2-yl)-N′-[5-(trifluoromethyl)-1,3,4-thiadiazol-2-yl]urea;
N-(4-ethoxy-2-morpholin-4-ylphenyl)-N′-[5-(trifluoromethyl)-1,3,4-thiadiazol-2-yl]urea;
tert-butyl 4-{5-ethoxy-2-[({[5-(trifluoromethyl)-1,3,4-thiadiazol-2-yl]amino}carbonyl) amino]phenyl}piperazine-1-carboxylate;
N-(2-chloro-6-methoxy-pyridin-3-yl)-N′-[5-(trifluoromethyl)-1,3,4-thiadiazol-2-yl]urea;
N-[6-methoxy-2-(methylthio)pyridin-3-yl]-N′-[5-(trifluoromethyl)-1,3,4-thiadiazol-2-yl]urea;
N-[6-methoxy-2-(methylsulfonyl)pyridin-3-yl]-N′-[5-(trifluoromethyl)-1,3,4-thiadiazol-2-yl]urea;
N-[2-methoxy-4-(2-methoxyethoxy)phenyl]-N′-(5-methylisoxazol-3-yl)urea;
N-(4-hydroxy-2-methylphenyl)-N′-[4-(trifluoromethyl)-1,3-thiazol-2-yl]urea;
N-(4-hydroxyphenyl)-N′-[4-(trifluoromethyl)-1,3-thiazol-2-yl]urea;
N-(5-hydroxy-2-methylphenyl)-N′-[4-(trifluoromethyl)-1,3-thiazol-2-yl]urea;
N-(3-hydroxy-2-methylphenyl)-N′-[4-(trifluoromethyl)-1,3-thiazol-2-yl]urea;
N-(6-cyanopyridin-3-yl)-N′-(5-fluoro-2,4-dimethoxyphenyl)urea;
N-(4-hydroxy-2-methylphenyl)-N′-[3-(trifluoromethoxy)phenyl]urea;
N-(4-hydroxy-2-methylphenyl)-N′-[4-(pentafluoroethyl)-1,3-thiazol-2-yl]urea;
N-(5-bromo-2,4-dimethoxyphenyl)-N′-(6-cyanopyridin-3-yl)urea;
N-(5-chloro-2,4-dimethoxyphenyl)-N′-[3-(trifluoromethyl)isoxazol-5-yl]urea;
N-(5-fluoro-2,4-dimethoxyphenyl)-N′-[3-(trifluoromethyl)isoxazol-5-yl]urea;
N-(2,6-dimethoxypyridin-3-yl)-N′-[3-(trifluoromethyl)isoxazol-5-yl]urea;
N-(4-ethoxy-2-nitrophenyl)-N′-[3-(trifluoromethyl)isoxazol-5-yl]urea;
N-(5-chloro-2,4-dimethoxyphenyl)-N′-(5-chloroisoxazol-3-yl)urea;
N-(5-chloro-4-methoxy-2-nitrophenyl)-N′-(5-methylisoxazol-3-yl)urea;
N-(5-fluoro-4-methoxy-2-nitrophenyl)-N′-(5-methylisoxazol -3-yl)urea;
N-[5-chloro-4-methoxy-2-(methylthio)phenyl]-N′-(5-methylisoxazol-3-yl)urea;
N-(5-chloro-2,4-dimethoxyphenyl)-N′-[5-(fluoromethyl)isoxazol-3-yl]urea;
N-(2,6-dimethoxypyridin-3-yl)-N′-[5-(fluoromethyl)isoxazol-3-yl]urea;
N-(5-fluoro-2,4-dimethoxyphenyl)-N′-[5-(fluoromethyl)isoxazol-3-yl]urea;
N-(5-bromo-2,4-dimethoxyphenyl)-N′-[5-(fluoromethyl)isoxazol-3-yl]urea;
N-(4-ethoxy-2-nitrophenyl)-N′-[5-(fluoromethyl)isoxazol-3-yl]urea;
N-(2,4-dimethoxy-5-methylphenyl)-N′-[5-(fluoromethyl)isoxazol-3-yl]urea;
N-(5-bromo-2,4-dimethoxyphenyl)-N′-[5-(hydroxymethyl)isoxazol-3-yl]urea;
N-(5-fluoro-2,4-dimethoxyphenyl)-N′-(5-isopropylisoxazol-3-yl)urea;
N-(5-chloro-2,4-dimethoxyphenyl)-N′-(5-isopropylisoxazol-3-yl)urea;
N-(5-bromo-2,4-dimethoxyphenyl)-N′-(5-isopropylisoxazol-3-yl)urea;
N-(2,6-dimethoxypyridin-3-yl)-N′-(5-isopropylisoxazol-3-yl)urea;
N-(2,4-dimethoxy-5-methylphenyl)-N′-(5-isopropylisoxazol-3-yl)urea;
N-(5-fluoro-2,4-dimethoxyphenyl)-N′-[5-(methoxymethyl)isoxazol-3-yl]urea;
N-(5-chloro-2,4-dimethoxyphenyl)-N′-[5-(methoxymethyl)isoxazol-3-yl]urea;
N-(5-bromo-2,4-dimethoxyphenyl)-N′-[5-(methoxymethyl)isoxazol-3-yl]urea;
N-(4-ethoxy-2-nitrophenyl)-N′-[5-(methoxymethyl)isoxazol-3-yl]urea;
N-(5-chloro-2,4-dimethoxyphenyl)-N′-(5-cyclopropylisoxazol-3-yl)urea;
N-(5-cyclopropylisoxazol-3-yl)-N′-(5-fluoro-2,4-dimethoxyphenyl)urea;
N-(5-bromo-2,4-dimethoxyphenyl)-N′-(5-cyclopropylisoxazol-3-yl)urea;
N-(5-cyclopropylisoxazol-3-yl)-N′-(2,4-dimethoxy-5-methylphenyl)urea;
N-(5-cyclopropylisoxazol-3-yl)-N′-(4-ethoxy-2-nitrophenyl)urea;
N-(5-cyclopropylisoxazol-3-yl)-N′-(2,6-dimethoxypyridin-3-yl)urea;
N-(5-chloro-2,4-dimethoxyphenyl)-N′-(5-ethylisoxazol-3-yl)urea;
N-(5-ethylisoxazol-3-yl)-N′-(5-fluoro-2,4-dimethoxyphenyl)urea;
N-(5-bromo-2,4-dimethoxyphenyl)-N′-(5-ethylisoxazol-3-yl)urea;
N-(2,4-dimethoxy-5-methylphenyl)-N′-(5-ethylisoxazol-3-yl)urea;
N-(2,6-dimethoxypyridin-3-yl)-N′-(5-ethylisoxazol-3-yl)urea;
N-(4-ethoxy-2-nitrophenyl)-N′-(5-ethylisoxazol-3-yl)urea;
N-(5-amino-2,4-dimethoxyphenyl)-N′-[5-(trifluoromethyl)-1,3,4-thiadiazol-2-yl]urea;
N-(5-azido-2,4-dimethoxyphenyl)-N′-[5-(trifluoromethyl)-1,3,4-thiadiazol-2-yl]urea;
N-(5-iodo-2,4-dimethoxyphenyl)-N′-[5-(trifluoromethyl)-1,3,4-thiadiazol-2-yl]urea;
N-(4-amino-2-methoxyphenyl)-N′-[5-(trifluoromethyl)-1,3,4-thiadiazol-2-yl]urea;
N-(4-methoxy-2-methylphenyl)-N′-[4-(pentafluoroethyl)-1,3-thiazol-2-yl]urea;
N-(5-chloro-2,4-dimethoxyphenyl)-N′-[5-(trifluoromethyl)isoxazol-3-yl]urea;
N-[2-methoxy-4-(oxetan-3-yloxy)phenyl]-N′-[5-(trifluoromethyl)-1,3,4-thiadiazol-2-yl]urea;
N-[2-methoxy-4-(oxetan-3-yloxy)phenyl]-N′-[3-(trifluoromethyl)isoxazol-5-yl]urea;
N-[2-methoxy-4-(tetrahydrofuran-3-yloxy)phenyl]-N′-[5-(trifluoromethyl)-1,3,4-thiadiazol-2-yl]urea;
N-[2-methoxy-4-(tetrahydrofuran-3-yloxy)phenyl]-N′-[3-(trifluoromethyl)isoxazol-5-yl]urea; or a pharmaceutically acceptable salt thereof.

3. The compound of claim 1, wherein the compound is
N-(5-chloro-2,4-dimethoxyphenyl)-N′-(5-methylisoxazol-3-yl)urea;
N-(5-chloro-2,4-dimethoxyphenyl)-N′-(5-chloro-1,3-thiazol-2-yl)urea;
N-(5-bromo-1,3-thiazol-2-yl)-N′-(5-chloro-2,4-dimethoxyphenyl)urea;
N-(5-chloro-2,4-dimethoxyphenyl)-N′-(5-methyl-1,3-thiazol-2-yl)urea;
N-(4-ethoxyphenyl)-N′-(5-methyl-1,3-thiazol-2-yl)urea;
N-[4-(4-chlorophenyl)-1,3-thiazol-2-yl]-N′-(4-iodophenyl)urea;
N-(2,6-dimethylphenyl)-N′-(6-fluoro-1,3-benzothiazol-2-yl)urea;
N-(4-chloro-1,3-benzothiazol-2-yl)-N′-(2-methoxyphenyl)urea;
N-(5-bromo-1,3-thiazol-2-yl)-N′-(2-ethoxyphenyl)urea;
N-(5-bromo-1,3-thiazol-2-yl)-N′-(2,4-dimethoxyphenyl)urea;
N-(5-chloro-1,3-thiazol-2-yl)-N′-(4-methoxy-2-methylphenyl)urea;
N-(5-bromo-1,3-thiazol-2-yl)-N′-(4-methoxy-2-methylphenyl)urea;
N-(5-chloro-2,4-dimethoxyphenyl)-N′-(1,3-thiazol-2-yl)urea;
N-(4-methoxy-2-methylphenyl)-N′-(4-methyl-1,3-thiazol-2-yl)urea;
N-(4-chloro-1,3-benzothiazol-2-yl)-N′-(4-methoxy-2-methylphenyl)urea;
ethyl 2-({[(4-methoxy-2-methylphenyl)amino]carbonyl}amino)-4-phenyl-1,3-thiazole-5-carboxylate;
ethyl 2-[({[4-(butoxycarbonyl)phenyl]amino}carbonyl)amino]-4-phenyl-1,3-thiazole-5-carboxylate;
N-(5-bromo-1,3-thiazol-2-yl)-N′-(4-ethoxyphenyl)urea; or a pharmaceutically acceptable salt thereof.

4. The compound of claim 1, wherein the compound is
N-(5-chloro-2,4-dimethoxyphenyl)-N′-(5-chloro-1,3-thiazol-2-yl)urea;
N-(5-bromo-1,3-thiazol-2-yl)-N′-(5-chloro-2,4-dimethoxyphenyl)urea;
N-(5-chloro-2,4-dimethoxyphenyl)-N′-(5-methyl-1,3-thiazol-2-yl)urea;
N-(4-ethoxyphenyl)-N′-(5-methyl-1,3-thiazol-2-yl)urea;
N-[4-(4-chlorophenyl)-1,3-thiazol-2-yl]-N′-(4-iodophenyl)urea;
N-(2,6-dimethylphenyl)-N′-(6-fluoro-1,3-benzothiazol-2-yl)urea;
N-(4-chloro-1,3-benzothiazol-2-yl)-N′-(2-methoxyphenyl)urea;
N-(5-bromo-1,3-thiazol-2-yl)-N′-(2-ethoxyphenyl)urea;
N-(5-bromo-1,3-thiazol-2-yl)-N′-(2,4-dimethoxyphenyl)urea;
N-(5-chloro-1,3-thiazol-2-yl)-N′-(4-methoxy-2-methylphenyl)urea;
N-(5-bromo-1,3-thiazol-2-yl)-N′-(4-methoxy-2-methylphenyl)urea;
N-(5-chloro-2,4-dimethoxyphenyl)-N′-(1,3-thiazol-2-yl)urea;
N-(4-methoxy-2-methylphenyl)-N′-(4-methyl-1,3-thiazol-2-yl)urea;
N-(4-chloro-1,3-benzothiazol-2-yl)-N′-(4-methoxy-2-methylphenyl)urea;
ethyl 2-({[(4-methoxy-2-methylphenyl)amino]carbonyl}amino)-4-phenyl-1,3-thiazole-5-carboxylate;
ethyl 2-[({[4-(butoxycarbonyl)phenyl]amino}carbonyl)amino]-4-phenyl-1,3-thiazole-5-carboxylate;
N-(5-bromo-1,3-thiazol-2-yl)-N′-(4-ethoxyphenyl)urea; or a pharmaceutically acceptable salt thereof.

5. The compound of claim 1, wherein only up to one of W^{A-1}, W^{A-2}, W^{A-3}, W^{A-4}, and W^{A-5} is N and the other are C—R_{A}.

6. The compound of claim 5, wherein W^{A-5} is CH.

7. The compound of claim 6, wherein W^{A-1} is C—R_{A}, wherein R_{A} is H, lower alkyl, O-(lower alkyl), S-(lower alkyl), S(O)-(lower alkyl), NO₂, C(O)-(lower alkyl), and C(lower alkyl)(═N—O-(lower alkyl)).

8. The compound of claim 6, wherein W_{A-3} is C—R_{A}, wherein R_{A} is H, O-(lower alkyl), O-sec-butyl, O-(lower alkenyl), —O-(heterocycloalkyl), —O-(substituted heterocycloalkyl), or ethoxy substituted on C-2 with OH, OMe, OEt, SMe, SEt, S(O)Me, S(O)₂Me, NH-(lower alkyl), N-(lower alkyl)₂, NHC(O)-(lower alkyl), NHS(O)₂-(lower alkyl)₂, morpholinyl, thiomorpholinyl, 1,1-dioxidothiomorpholinyl, piperazinyl, pyrrolidinyl, 1H-pyrazolyl, and piperidinyl.

9. The compound of claim 6, wherein W^{A-4} is C—R_{A}, wherein R_{A} is H, lower alkyl, O-(lower alkyl), or halogen.

10. The compound of claim 6, wherein W^{A-3} is C—R_{A}, wherein R_{A} is H, O-(lower alkyl), O-sec-butyl, O-allyl, —O-(heterocycloalkyl), —O-(substituted heterocycloalkyl), and ethoxy substituted on C-2 with OH, OMe, OEt, SMe, SEt, S(O)Me, S(O)₂Me, NHMe, N(Me)₂, NHEt, N(Et)₂, N(Me)(Et), NHC(O)Me, NHS(O)₂(Me), morpholin-4-yl, thiomorpholin-4-yl, 1,1-dioxidothiomorpholin-4-yl, piperazin-1-yl, pyrrolidin-1-yl, 1H-pyrazol-1-yl, and piperidin-1-yl.

11. The compound of claim 10, wherein W^{A-1} is C—R_{A}, wherein R_{A} is H, Me, OMe, OEt, O-i-propyl, O-n-propyl, SMe, SOMe, SEt, S(O)Et, NO₂, C(O)Me, and C(CH₃)(═N—OMe).

12. The compound of claim 11, wherein W^{A-4} is C—R_{A}, wherein R_{A} is H, Me, OMe, F, Cl, and Br.

13. The compound of claim 12, wherein B is isoxazol-3-yl, optionally substituted at C-5 with lower alkyl, lower haloalkyl, CH₂OCH₃, lower cycloalkyl, halogen, and CN.

14. The compound of claim 13, wherein the optional substituent on isoxazol-3-yl is CH₃, CF₃, CH₂F, CHF₂, CH₂CH₃, CF₂CF₃, cyclopropyl, Cl, Br, CN, or CH₂OCH₃.

15. The compound of claim 14, wherein the compound is selected from Table 1, provided that at least one of R—Aⁱ, R—Aⁱⁱⁱ, and R—A^{iv} is other than H:
TABLE 1

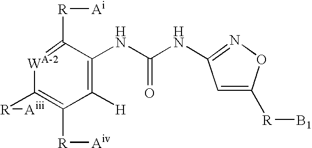

| | *R-Aⁱⁱⁱ | *W^{A-2} | *R-Aⁱ | *R-A^{iv} | R-B₁ |
|---|---|---|---|---|---|
| 1001 | H | (1) CH | (A) H | (a) H | (i) CH₃ |
| 1002 | OCH₃ | (2) N | (B) CH₃ | (b) CH₃ | (ii) CF₃ |
| 1003 | OCH₂CH₃ | | (C) OCH₃ | (c) OCH₃ | (iii) CH₂F |
| 1004 | OCH₂—CH═CH₂ | | (D) OEt | (d) F | (iv) CHF₂ |
| 1005 | O-n-propyl | | (E) O-i-propyl | (e) Cl | (v) CH₂CH₃ |
| 1006 | O-i-propyl | | (F) O-n-propyl | (f) Br | (vi) CF₂CF₃ |
| 1007 | O-sec-butyl | | (G) SMe | | (vii) cyclopropyl |
| 1008 | 2-hydroxyethoxy | | (H) S(O)Me | | (viii) Cl |
| 1009 | 2-methoxyethoxy | | (I) SEt | | (ix) Br |
| 1010 | 2-ethoxyethoxy | | (J) S(O)Et | | (xi) CN |
| 1011 | 2-(ethylthio)- | | (K) NO₂ | | (xii) H |
| | ethoxy | | | | |
| 1012 | 2-(methylthio)- | | (L) C(═O)Me | | (xiii) CH₂OCH₃ |
| | ethoxy | | | | |
| 1013 | 2-(methyl | | (M) | | |
| | sulfinyl)ethoxy | | C(Me)(═NOMe) | | |
| 1014 | 2-(methyl | | | | |
| | sulfonyl)ethoxy | | | | |
| 1015 | 2-(methylamino)- | | | | |
| | ethoxy | | | | |
| 1016 | 2-(ethylamino)- | | | | |
| | ethoxy | | | | |
| 1017 | 2-(dimethyl | | | | |
| | amino)ethoxy | | | | |
| 1018 | 2-(diethyl | | | | |
| | amino)ethoxy | | | | |
| 1019 | OCH₂CH₂- | | | | |
| | N(Me)(Et) | | | | |
| 1020 | OCH₂CH₂- | | | | |
| | NHC(O)CH₃ | | | | |
| 1021 | OCH₂CH₂— | | | | |
| | NHS(O)₂CH₃ | | | | |
| 1022 | 2-thiomorpholin- | | | | |
| | 4-ylethoxy | | | | |
| 1023 | 2-(1,1-dioxido- | | | | |
| | thiomorpholin-4- | | | | |
| | yl)ethoxy | | | | |
| 1024 | 2-piperazin-1- | | | | |
| | ylethoxy | | | | |
| 1025 | 2-pyrrolidin-1- | | | | |
| | ylethoxy | | | | |
| 1026 | 2-piperidin-1- | | | | |
| | ylethoxy | | | | |
| 1027 | 2-morpholin-4- | | | | |
| | ylethoxy | | | | |
| 1028 | 2-1H-pyrazol-1- | | | | |
| | ylethoxy | | | | |
| 1029 | O-oxetan-3-yl | | | | |
| 1030 | O-tetrahydro- | | | | |
| | furan-3-yl | | | | |
| 1031 | O-1,1-dioxido- | | | | |
| | thietan-3-yl | | | | |
| 1032 | O-azetidin-3-yl | | | | |

16. The compound of claim 15, wherein W^{A-2} is N.

17. The compound of claim 16, wherein the compound is selected from
N-(2,6-dimethoxypyridin-3-yl)-N′-[5-(fluoromethyl)isoxazol-3-yl]urea;
N-(5)-cyclopropylisoxazol-3-yl)-N′-(2,6-dimethoxypyridin-3-yl)urea;
N-(2,6-dimethoxypyridin-3-yl)-N′-(5-ethylisoxazol-3-yl)urea; or a pharmaceutically acceptable salt thereof.

18. The compound of claim 15, wherein W^{A-2} is CH.

19. The compound of claim 18, wherein the compound is selected from
N-5-chloro-2,4-dimethoxyphenyl)-N′-isoxazol-3-ylurea;
N-(5-chloro-2,4-dimethoxyphenyl)-N′-(5-methylisoxazol-3-yl)urea;
N-(4-ethoxyphenyl)-N′-(5-methylisoxazol-3-yl)urea;
N-(2,4-dimethoxyphenyl)-N′-(5-methylisoxazol-3-yl)urea;
N-(5-chloro-2-methoxyphenyl)-N′-(5-methylisoxazol-3-yl)urea;
N-(3-chloro-4-fluorophenyl)-N′-(5-methylisoxazol-3-yl)urea;
N-(2-ethoxyphenyl)-N′-(5-methylisoxazol-3-yl)urea;
N-(4-ethoxy-2-nitrophenyl)-N′-(5-methylisoxazol-3-yl)urea;
N-(5-fluoro-2,4-dimethoxyphenyl)-N′-(5-methylisoxazol-3-yl)urea;
N-(5-bromo-2,4-dimethoxyphenyl)-N′-(5-methylisoxazol-3-yl)urea.
N-(5-chloro-2,4-diethoxyphenyl)-N′-(5-methylisoxazol-3-yl)urea;
N-(2-chloro-4-methoxy-5-methylphenyl)-N′-(5-methylisoxazol-3-yl)urea;
N-(2,4-dimethoxy-5-methylphenyl)-N′-(5-methylisoxazol-3-yl)urea;
N-[2-methoxy-4-(2-methoxyethoxy)phenyl]-N′-(5-methylisoxazol-3-yl)urea;
N-(5-chloro-2,4-dimethoxyphenyl)-N′-(5-chloroisoxazol-3-yl)urea;
N-(5-chloro-4-methoxy-2-nitrophenyl)-N′-(5-methylisoxazol-3-yl)urea;
N-(5-fluoro-4-methoxy-2-nitrophenyl)-N′-(5-methylisoxazol-3-yl)urea;
N-[5-chloro-4-methoxy-2-(methylthio)phenyl]-N′-(5-methylisoxazol-3-yl)urea;
N-(5-chloro-2,4-dimethoxyphenyl)-N′-[5-(fluoromethyl)isoxazol-3-yl]urea;
N-(5-fluoro-2,4-dimethoxyphenyl)-N′-[5-(fluoromethyl)isoxazol-3-yl]urea;
N-(5-bromo-2,4-dimethoxyphenyl)-N′-[5-(fluoromethyl)isoxazol-3-yl]urea;
N-(4-ethoxy-2-nitrophenyl)-N′-[5-(fluoromethyl)isoxazol-3-yl]urea;
N-(5-chloro-2,4-dimethoxyphenyl)-N′-(5-cyclopropylisoxazol-3-yl)urea;
N-(5-cyclopropylisoxazol-3-yl)-N′-(5-fluoro-2,4-dimethoxyphenyl)urea;
N-(5-bromo-2,4-dimethoxyphenyl)-N′-(5-cyclopropylisoxazol-3-yl)urea;
N-(5-cyclopropylisoxazol-3-yl)-N′-(2,4-dimethoxy-5-methylphenyl)urea;
N-(5-cyclopropylisoxazol-3-yl)-N′-(4-ethoxy-2-nitrophenyl)urea;
N-(5-chloro-2,4-dimethoxyphenyl)-N′-(5-ethylisoxazol-3-yl)urea;
N-(5-ethylisoxazol-3-yl)-N′-(5-fluoro-2,4-dimethoxyphenyl)urea;
N-(5-bromo-2,4-dimethoxyphenyl)-N′-(5-ethylisoxazol-3-yl)urea;
N-(2,4-dimethoxy-5-methylphenyl)-N′-(5-ethylisoxazol-3-yl)urea;
N-(4-ethoxy-2-nitrophenyl)-N′-(5-ethylisoxazol-3-yl)urea;
N-(5-chloro-2,4-dimethoxyphenyl)-N′-[5-(trifluoromethyl)isoxazol-3-yl]urea;
N-(2,4-dimethoxy-5-methylphenyl)-N′-[5-(fluoromethyl)isoxazol-3-yl]urea; or a pharmaceutically acceptable salt thereof.

20. The compound of claim 18, wherein the compound is selected from
N-(5-chloro-2,4-dimethoxyphenyl)-N′-isoxazol-3-ylurea;
N-(4-ethoxyphenyl)-N′-(5-methylisoxazol-3-yl)urea;
N-(2,4-dimethoxyphenyl)-N′-(5-methylisoxazol-3-yl)urea;
N-(5-chloro-2-methoxyphenyl)-N′-(5-methylisoxazol-3-yl)urea;
N-(3-chloro-4-fluorophenyl)-N′-(5-methylisoxazol-3-yl)urea;
N-(2-ethoxyphenyl)-N′-(5-methylisoxazol-3-yl)urea;
N-(4-ethoxy-2-nitrophenyl)-N′-(5-methylisoxazol-3-yl)urea;
N-(5-fluoro-2,4-dimethoxyphenyl)-N′-(5-methylisoxazol-3-yl)urea;
N-(5-bromo-2,4-dimethoxyphenyl)-N′-(5-methylisoxazol-3-yl)urea.
N-(5-chloro-2,4-diethoxyphenyl)-N′-(5-methylisoxazol-3-yl)urea;
N-(2-chloro-4-methoxy-5-methylphenyl)-N′-(5-methylisoxazol-3-yl)urea;
N-(2,4-dimethoxy-5-methylphenyl)-N′-(5-methylisoxazol-3-yl)urea;
N-[2-methoxy-4-(2-methoxyethoxy)phenyl]-N′-(5-methylisoxazol-3-yl)urea;
N-(5-chloro-2,4-dimethoxyphenyl)-N′-(5-chloroisoxazol-3-yl)urea;
N-(5-chloro-4-methoxy-2-nitrophenyl)-N′-(5-methylisoxazol-3-yl)urea;
N-(5-fluoro-4-methoxy-2-nitrophenyl)-N′-(5-methylisoxazol-3-yl)urea;
N-[5-chloro-4-methoxy-2-(methylthio)phenyl]-N′-(5-methylisoxazol-3-yl)urea;
N-(5-chloro-2,4-dimethoxyphenyl)-N′-[5-(fluoromethyl)isoxazol-3-yl]urea;
N-(5-fluoro-2,4-dimethoxyphenyl)-N′-[5-(fluoromethyl)isoxazol-3-yl]urea;
N-(5-bromo-2,4-dimethoxyphenyl)-N′-[5-(fluoromethyl)isoxazol-3-yl]urea;
N-(4-ethoxy-2-nitrophenyl)-N′-[5-(fluoromethyl)isoxazol-3-yl]urea;
N-(5-chloro-2,4-dimethoxyphenyl)-N′-(5-cyclopropylisoxazol-3-yl)urea;
N-(5-cyclopropylisoxazol-3-yl)-N′-(5-fluoro-2,4-dimethoxyphenyl)urea;
N-(5-bromo-2,4-dimethoxyphenyl)-N′-(5-cyclopropylisoxazol-3-yl)urea;
N-(5-cyclopropylisoxazol-3-yl)-N′-(2,4-dimethoxy-5-methylphenyl)urea;
N-(5-cyclopropylisoxazol-3-yl)-N′-(4-ethoxy-2-nitrophenyl)urea;
N-(5-chloro-2,4-dimethoxyphenyl)-N′-(5-ethylisoxazol-3-yl)urea;
N-(5-ethylisoxazol-3-yl)-N′-(5-fluoro-2,4-dimethoxyphenyl)urea;
N-(5-bromo-2,4-dimethoxyphenyl)-N′-(5-ethylisoxazol-3-yl)urea;
N-2,4-dimethoxy-5-methylphenyl)-N′-(5-ethylisoxazol-3-yl)urea;
N-(4-ethoxy-2-nitrophenyl)-N′-(5-ethylisoxazol-3-yl)urea;
N-(5-chloro-2,4-dimethoxyphenyl)-N′-[5-(trifluoromethyl)isoxazol-3-yl]urea;
N(2,4-dimethoxy-5-methylphenyl)-N′-[5-(fluoromethyl)isoxazol-3-yl]urea; or a pharmaceutically acceptable salt thereof.

21. The compound of claim 12, wherein B is isoxazol-5-yl, substituted at C-3 with lower alkyl, lower haloalkyl, halogen, and CN.

22. The compound of claim 21, wherein the substituent is CH₃, CF₃, CH₂F, CHF₂, CF₂CF₃, Cl, Br, and CN.

23. The compound of claim 22, wherein the compound is selected from Table 3, provided that at least one of R—Aⁱ, R—Aⁱⁱⁱ, and R—A^{iv} is other than H:
TABLE 3

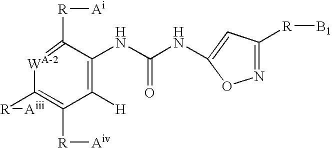

| | R-Aⁱⁱⁱ | W^{A-2} | R-Aⁱ | R-A^{iv} | R-B₁ |
|---|---|---|---|---|---|
| 1101 | H | (1) CH | (A) H | (a) H | (i) CH₃ |
| 1102 | OCH₃ | (2) N | (B) CH₃ | (b) CH₃ | (ii) CF₃ |
| 1103 | OCH₂CH₃ | | (C) OCH₃ | (c) OCH₃ | (iii) CH₂F |
| 1104 | OCH₂—CH═CH₂ | | (D) OEt | (d) F | (iv) CHF₂ |
| 1105 | O-n-propyl | | (E) O-i-propyl | (e) Cl | (v) Cl |
| 1106 | O-i-propyl | | (F) O-n-propyl | (f) Br | (vi) Br |
| 1107 | O-sec-butyl | | (G) SMe | | (vii) CN |
| 1108 | 2-hydroxyethoxy | | (H) S(O)Me | | (viii) CF₂CF₃ |
| 1109 | 2-methoxyethoxy | | (I) SEt | | |
| 1110 | 2-ethoxyethoxy | | (J) S(O)Et | | |
| 1111 | 2-(ethylthio)- | | (K) NO₂ | | |
| | ethoxy | | | | |
| 1112 | 2-(methylthio)- | | (L) C(═O)Me | | |
| | ethoxy | | | | |
| 1113 | 2-(methlyl | | (M) | | |
| | sulfinyl)ethoxy | | C(Me)(═NOMe) | | |
| 1114 | 2-(methyl | | | | |
| | sulfonyl)ethoxy | | | | |
| 1115 | 2-(methylamino)- | | | | |
| | ethoxy | | | | |
| 1116 | 2-(ethylamino)- | | | | |
| | ethoxy | | | | |
| 1117 | 2-(dimethyl | | | | |
| | amino)ethoxy | | | | |
| 1118 | 2-(diethylamino)- | | | | |
| | ethoxy | | | | |
| 1119 | OCH₂CH₂— | | | | |
| | N(Me)(Et) | | | | |
| 1120 | OCH₂CH₂— | | | | |
| | NHC(O)CH₃ | | | | |
| 1121 | OCH₂CH₂— | | | | |
| | NHS(O)₂CH₃ | | | | |
| 1122 | 2-thiomorpholin- | | | | |
| | 4-ylethoxy | | | | |
| 1123 | 2-(1,1-dioxido | | | | |
| | thiomorpholin-4- | | | | |
| | yl)ethoxy | | | | |
| 1124 | 2-piperazin-1- | | | | |
| | ylethoxy | | | | |
| 1125 | 2-pyrrolidin-1- | | | | |
| | ylethoxy | | | | |
| 1126 | 2-piperidin-1- | | | | |
| | ylethoxy | | | | |
| 1127 | 2-morpholin-4- | | | | |
| | ylethoxy | | | | |
| 1128 | 2-IH-pyrazol-1- | | | | |
| | ylethoxy | | | | |
| 1129 | O-oxetan-3-yl | | | | |
| 1130 | O-tetrahydro- | | | | |
| | furan-3-yl | | | | |
| 1131 | O-1,1-dioxido- | | | | |
| | thietan-3-yl | | | | |
| 1132 | O-azetidin-3-yl | | | | |

24. The compound of claim 23, wherein W^{A-2} is N.

25. The compound of claim 24, wherein the compound is selected from
N-(2,6-dimethoxypyridin-3-yl)-N′-(3-methylisoxazol-5-yl)urea;
N-(2,6-dimethoxypyridin-3-yl)-N′-[3-(trifluoromethyl)isoxazol-5-yl]urea; or a pharmaceutically acceptable salt thereof.

26. The compound of claim 23, wherein W^{A-2} is CH.

27. The compound of claim 26, wherein the compound is selected from
N-(5-chloro-2,4-dimethoxyphenyl)-N′-(3-methylisoxazol-5-yl)urea;
N-(2,4-dimethoxy-5-methylphenyl)-N′-[3-(trifluoromethyl)isoxazol-5-yl]urea;
N-(4-ethoxyphenyl)-N′-[3-(trifluoromethyl)isoxazol-5-yl]urea;
N-(2-ethoxyphenyl)-N′-[3-(trifluoromethyl)isoxazol-5-yl]urea;
N-(2,4-dimethoxyphenyl)-N′-[3-(trifluoromethyl)isoxazol-5-yl]urea;
N-(5-fluoro-2,4-dimethoxyphenyl)-N′-(3-methylisoxazol-5-yl)urea;
N-(2,4-dimethoxyphenyl)-N′-(3-methylisoxazol-5-yl)urea;
N-(5-chloro-2,4-diethoxyphenyl)-N′-[3-(trifluoromethyl)isoxazol-5-yl]urea;
N-(4-methoxy-2-nitrophenyl)-N′-[3-(trifluoromethyl)isoxazol-5-yl]urea;
N-(5-chloro-2,4-dimethoxyphenyl)-N′-[3-(trifluoromethyl)isoxazol-5-yl]urea;
N-(5-fluoro-2,4-dimethoxyphenyl)-N′-[3-(trifluoromethyl)isoxazol-5-yl]urea;
N-(4-ethoxy-2-nitrophenyl)-N′-[3-(trifluoromethyl)isoxazol-5-yl]urea;
N-[2-methoxy-4-(oxetan-3-yloxy)phenyl]-N′-[3-(trifluoromethyl)isoxazol-5-yl]urea;
N-[2-methoxy-4-(tetrahydrofuran-3-yloxy)phenyl]-N′-[3-(trifluoromethyl)isoxazol-5-yl]urea; or a pharmaceutically acceptable salt thereof.

28. The compound of claim 12, wherein B is 1,3,4-thiadiazol-2-yl substituted at C-5 with lower alkyl, lower haloalkyl, halogen, and CN.

29. The compound of claim 28, wherein the substituent is CH₃, CF₃, CH₂F, CHF₂, CF₂ CF₃, Cl, Br, and CN.

30. The compound of claim 29, wherein the compound is selected from Table 2, provided that at least one of R—Aⁱ, R—Aⁱⁱⁱ, and R—A^{iv} is other than H:
TABLE 2

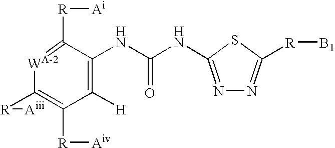

| | R-Aⁱⁱⁱ | W^{A-2} | R-Aⁱ | R-A^{iv} | R-B₁ |
|---|---|---|---|---|---|
| 1051 | H | (1) CH | (A) H | (a) H | (i) CH₃ |
| 1052 | OCH₃ | (2) N | (B) CH₃ | (b) CH₃ | (ii) CF₃ |
| 1053 | OCH₂CH₃ | | (C) OCH₃ | (c) OCH₃ | (iii) CH₂F |
| 1054 | OCH₂—CH═CH₉ | | (D) OEt | (d) F | (iv) CHF₂ |
| 1055 | O-n-propyl | | (E) O-i-propyl | (e) Cl | (v) Cl |
| 1056 | O-i-propyl | | (F) O-n-propyl | (f) Br | (vi) Br |
| 1057 | O-sec-butyl | | (G) SMe | | (vii) CN |
| 1058 | 2-hydroxyethoxy | | (H) S(O)Me | | (viii) CF₂CF₃ |
| 1059 | 2-methoxyethoxy | | (I) SEt | | |
| 1060 | 2-ethoxyethoxy | | (J) S(O)Et | | |
| 1061 | 2-(ethylthio)- | | (K) NO₂ | | |
| | ethoxy | | | | |
| 1062 | 2-(methylthio)- | | (L) C(═O)Me | | |
| | ethoxy | | | | |
| 1063 | 2-(methyl | | (M) | | |
| | sulfinyl)ethoxy | | C(Me)(═NOMe) | | |
| 1064 | 2-(methyl | | | | |
| | sulfonyl)ethoxy | | | | |
| 1065 | 2-(methylamino)- | | | | |
| | ethoxy | | | | |
| 1066 | 2-(ethylamino)- | | | | |
| | ethoxy | | | | |
| 1067 | 2-(dimethyl | | | | |
| | amino)-ethoxy | | | | |
| 1068 | 2-(diethylamino)- | | | | |
| | ethoxy | | | | |
| 1069 | OCH₂CH₂— | | | | |
| | N(Me)(Et) | | | | |
| 1070 | OCH₂CH₂— | | | | |
| | NHC(O)CH₃ | | | | |
| 1071 | OCH₂CH₂— | | | | |
| | NHS(O)₂CH₃ | | | | |
| 1072 | 2-thiomorpholin- | | | | |
| | 4-ylethoxy | | | | |
| 1073 | 2-(1,1-dioxido- | | | | |
| | thiomorpholin-4- | | | | |
| | yl)ethoxy | | | | |
| 1074 | 2-piperazin-1- | | | | |
| | ylethoxy | | | | |
| 1075 | 2-pyrrolidin-1- | | | | |
| | ylethoxy | | | | |
| 1076 | 2-piperidin-1- | | | | |
| | ylethoxy | | | | |
| 1077 | 2-morpholin-4- | | | | |
| | ylethoxy | | | | |
| 1078 | 2-1H-pyrazol-1- | | | | |
| | ylethoxy | | | | |
| 1079 | O-oxetan-3-yl | | | | |
| 1080 | O-tetrahydro- | | | | |
| | furan-3-yl | | | | |
| 1081 | O-1,1-dioxido- | | | | |
| | thietan-3-yl | | | | |
| 1082 | O-azetidin-3-yl | | | | |

31. The compound of claim 30, wherein W^{A-2} is N.

32. The compound of claim 31, wherein the compound is selected from
N-(2-ethoxypyridin-3-yl)-N′-[5-(trifluoromethyl)-1,3,4-thiadiazol-2-yl]urea;
N-(2,6-dimethoxypyridin-3-yl)-N′-[5-(trifluoromethyl)-1,3,4-thiadiazol-2-yl]urea; or pharmaceutically acceptable salt thereof.

33. The compound of claim 30, wherein W^{A-2} is CH.

34. The compound of claim 33, wherein the compound is selected from
N-(5-chloro-2,4-dimethoxyphenyl)-N′-(5-methyl-1,3,4-thiadiazol-2-yl)urea;
N-(5-chloro-2,4-dimethoxyphenyl)-N′-[5-(trifluoromethyl)-1,3,4-thiadiazol-2-yl]urea;
N-(5-chloro-2,4-dimethoxyphenyl)-N′-(5-cyclopropyl-1,3,4-thiadiazol-2-yl)urea;
N-(5-chloro-2,4-dimethoxyphenyl)-N′-[5-(difluoromethyl)-1,3,4-thiadiazol-2-yl]urea;
N-(5-bromo-1,3,4-thiadiazol-2-yl)-N′-(5-chloro-2,4-dimethoxyphenyl)urea;
N-(5-chloro-1,3,4-thiadiazol-2-yl)-N′-(5-chloro-2,4-dimethoxyphenyl)urea;
N-(2,4-dimethoxyphenyl)-N′-[5-(trifluoromethyl)-1,3,4-thiadiazol-2-yl]urea;
N-(2-methoxyphenyl)-N′-[5-(trifluoromethyl)-1,3,4-thiadiazol-2-yl]urea;
N-(4,5-dimethoxy-2-methylphenyl)-N′-[5-(trifluoromethyl)-1,3,4-thiadiazol-2-yl]urea;
N-(5-fluoro-2,4-dimethoxyphenyl)-N′-(5-methyl-1,3,4-thiadiazol-2-yl)urea;
N-(5-fluoro-2,4-dimethoxyphenyl)-N′-[5-(trifluoromethyl)-1,3,4-thiadiazol-2-yl]urea;
N-(5-bromo-2,4-dimethoxyphenyl)-N′-(5-methyl-1,3,4-thiadiazol-2-yl)urea;
N-(5-bromo-2,4-dimethoxyphenyl)-N′-[5-(trifluoromethyl)-1,3,4-thiadiazol-2-yl]urea;
N-[2,4-dimethoxy-5-(trifluoromethyl)phenyl]-N′-[5-(trifluoromethyl)-1,3,4-thiadiazol-2-yl]urea;
N-(5-chloro-2,4-diethoxyphenyl)-N′-(5-methyl-1,3,4-thiadiazol-2-yl)urea;
N-(5-chloro-2,4-diethoxyphenyl)-N′-[5-(trifluoromethyl)-1,3,4-thiadiazol-2-yl]urea;
N-(5-chloro-2,4-dipropoxyphenyl)-N′-[5-(trifluoromethyl)-1,3,4-thiadiazol-2-yl]urea;
N-(2,4-dimethoxy-5-methylphenyl)-N′-(5-methyl-1,3,4-thiadiazol-2-yl)urea;
N-(2,4-dimethoxy-5-methylphenyl)-N′-[5-(trifluoromethyl)-1,3,4-thiadiazol-2-yl]urea;
N-(4-ethoxy-2-methoxy-5-methylphenyl)-N′-[5-(trifluoromethyl)-1,3,4-thiadiazol-2-yl]urea;
N-[5-(trifluoromethyl)-1,3,4-thiadiazol-2-yl]-N′-(2,4,5-trimethoxyphenyl)urea;
N-[4-methoxy-2-(methylthio)phenyl]-N′-[5-(trifluoromethyl)-1,3,4-thiadiazol-2-yl]urea;
N-(4-{[(1R)-1-methylpropyl]oxy}phenyl)-N′-[5-(trifluoromethyl)-1,3,4-thiadiazol-2yl]urea;
N-[4-(allyloxy)phenyl]-N′-[5-(trifluoromethyl)-1,3,4-thiadiazol-2-yl]urea;
N-(4-propoxyphenyl)-N′-[5-(trifluoromethyl)-1,3,4-thiadiazol-2-yl]urea;
N-[2-methoxy-4-(2-methoxyethoxy)phenyl]-N′-[5-(trifluoromethyl)-1,3,4-thiadiazol-2-yl]urea;
N-(3-chloro-4-methoxyphenyl)-N′-[5-(trifluoromethyl)-1,3,4-thiadiazol-2-yl]urea;
N-(5-chloro-2-methoxyphenyl)-N′-[5-(trifluoromethyl)-1,3,4-thiadiazol-2-yl]urea;
N-(4-methoxy-2-nitrophenyl)-N′-[5-(trifluoromethyl)-1,3,4-thiadiazol-2-yl]urea;
N-(4-ethoxy-2-nitrophenyl)-N′-[5-(trifluoromethyl)-1,3,4-thiadiazol-2-yl]urea;
N-(4-methoxy-2-methylphenyl)-N′-[5-(trifluoromethyl)-1,3,4-thiadiazol-2-yl]urea;
N-[2-methoxy-4-(oxetan-3-yloxy)phenyl]-N′-[5-(trifluoromethyl)-1,3,4-thiadiazol-2-yl]urea;
N-[2-methoxy-4-(tetrahydrofuran-3-yloxy)phenyl]-N′-[5-(trifluoromethyl)-1,3,4-thiadiazol-2-yl]urea; or pharmaceutically acceptable salt thereof.

35. The compound of claim 12, wherein the compound is selected from Table 4, provided that at least one of R—Aⁱ, R—Aⁱⁱⁱ, and R—A^{iv} is other than H:
TABLE 4

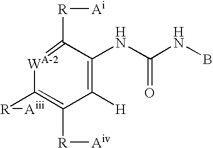

| | R-Aⁱⁱⁱ | W^{A-2} | R-Aⁱ | R-A^{iv} | B | R-B₁ |
|---|---|---|---|---|---|---|
| 1151 | H | (1) CH | (A) H | (a) H | 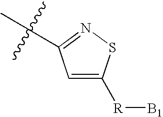 | (i) CH₃ |
| 1152 | OCH₃ | (2) N | (B) CH₃ | (b) CH₃ | 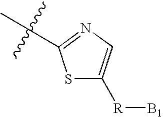 | (ii) CF₃ |
| 1153 | OCH₂CH₃ | | (C) OCH₃ | (c) OCH₃ | 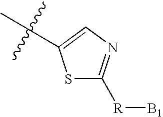 | (iii) Cl |
| 1154 | OCH₂—CH═CH₂ | | (D) OEt | (d) F | 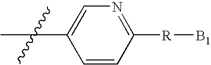 | (iv) Br |
| 1155 | 2-methoxy | | (E) SMe | (e) Cl | | (v) CN |
| | ethoxy | | | | | |
| 1156 | O-oxetan-3-yl | | (F) NO₂ | (f) Br | | |
| 1157 | O-tetrahydro- | | (G) | | | |
| | furan-3-yl | | C(═O)Me | | | |
| 1158 | O-1,1-dioxido- | | | | | |
| | thietan-3-yl | | | | | |
| 1159 | O-azetidin-3-yl | | | | | |

36. The compound of claim 35, wherein the compound is selected from
N-(5-bromo-1,3-thiazol-2-yl)-N′-(5-chloro-2,4-dimethoxyphenyl)urea;
N-(5-chloro-2,4-dimethoxyphenyl)-N′-(5-methyl-1,3-thiazol-2-yl)urea;
N-(5-chloro-2,4-dimethoxyphenyl)-N′-(6-cyanopyridin-3-yl)urea;
N-(5-chloro-2,4-dimethoxyphenyl)-N′-(5-chloro-1,3-thiazol-2-yl)urea;
N-(5-chloro-1,3-thiazol-2-yl)-N′-(2,4-dimethoxy-5-methylphenyl)urea;
N-(5-bromo-2,4-dimethoxyphenyl)-N′-(5-chloro-1,3-thiazol-2-yl)urea;
N-(5-bromo-1,3-thiazol-2-yl)-N′-(5-fluoro-2,4-dimethoxyphenyl)urea;
N-(5-chloro-1,3-thiazol-2-yl)-N′-(5-fluoro-2,4-dimethoxyphenyl)urea;
N-(2,6-dimethoxypyridin-3-yl)-N′-(5-methyl-1,3-thiazol-2-yl)urea;
N-(5-fluoro-2,4-dimethoxyphenyl)-N′-(5-methyl-1,3-thiazol-2-yl)urea;
N-(5-bromo-2,4-dimethoxyphenyl)-N′-(5-methyl-1,3-thiazol-2-yl)urea;
N-(5-chloro-2,4-diethoxyphenyl)-N′-(5-methyl-1,3-thiazol-2-yl)urea;
N-(2,4-dimethoxy-5-methylphenyl)-N′-(5-methyl-1,3-thiazol-2-yl)urea;
N-(5-chloro-2,4-dimethoxyphenyl)-N′-(2-methyl-1,3-thiazol-5-yl)urea;
N-(6-cyanopyridin-3-yl)-N′-(5-fluoro-2,4-dimethoxyphenyl)urea;
N-(5-bromo-2,4-dimethoxyphenyl)-N′-(6-cyanopyridin-3-yl)urea;
N-(4-ehoxyphenyl)-N′-(5-methyl-1,3-thiazol-2-yl)urea;
N-(5-bromo-1,3-thiazol-2-yl)-N′-(2-ethoxyphenyl)urea;
N-(5-bromo-1,3-thiazol-2-yl)-N′-(2,4-dimethoxyphenyl)urea;
N-(5-chloro-1,3-thiazol-2-yl)-N′-(4-methoxy-2-methylphenyl)urea;
N-(5-bromo-1,3-thiazol-2-yl)-N′-(4-methoxy-2-methylphenyl)urea;
N-(5-bromo-1,3-thiazol-2-yl)-N′-(4-ethoxyphenyl)urea; or pharmaceutically acceptable salts thereof.

37. The compound of claim 35, wherein the compound is selected from
N-(5-chloro-2,4-dimethoxyphenyl)-N′-(6-cyanopyridin-3-yl)urea;
N-(5-chloro-1,3-thiazol-2-yl)-N′-(2,4-dimethoxy-5-methylphenyl)urea;
N-(5-bromo-2,4-dimethoxyphenyl)-N′-(5-chloro-1,3-thiazol-2-yl)urea;
N-(5-bromo-1,3-thiazol-2-yl)-N′-(5-fluoro-2,4-dimethoxyphenyl)urea;
N-(5-chloro-1,3-thiazol-2-yl)-N′-(5-fluoro-2,4-dimethoxyphenyl)urea;
N-(2,6-dimethoxypyridin-3-yl)-N′-(5-methyl-1,3-thiazol-2-yl)urea;
N-(5-fluoro-2,4-dimethoxyphenyl)-N′-(5-methyl-1,3-thiazol-2-yl)urea;
N-(5-bromo-2,4-dimethoxyphenyl)-N′-(5-methyl-1,3-thiazol-2-yl)urea;
N-(5-chloro-2,4-diethoxyphenyl)-N′-(5-methyl-1,3-thiazol-2-yl)urea;
N-(2,4-dimethoxy-5-methylphenyl)-N′-(5-methyl-1,3-thiazol-2-yl)urea;
N-(5-chloro-2,4-dimethoxyphenyl)-N′-(2-methyl-1,3-thiazol-5-yl)urea;
N-(6-cyanopyridin-3-yl)-N′-(5-fluoro-2,4-dimethoxyphenyl)urea;
N-(5-bromo-2,4-dimethoxyphenyl)-N′-(6-cyanopyridin-3-yl)urea; or pharmaceutically acceptable salts thereof.

38. The compound of claim 12, wherein B is thiazolyl substituted with lower alkyl and lower haloalkyl, and 3-trifluorophenyl.

39. The compound of claim 38, wherein the compound is selected from Table 5, provided that at least one of R—Aⁱ, R—Aⁱⁱⁱ and R—A^{iv} is other than H:
TABLE 5

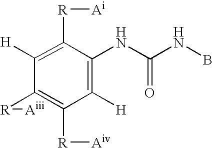

| | B | *R-Aⁱ | *R-Aⁱⁱⁱ | *R-A^{iv} |
|---|---|---|---|---|
| 1201 | 3-trifluorophenyl | (I) H | (A) H | (i) H |
| 1202 | 4-methyl-thiazol-2-yl | (II) CH₃ | (B) OH | (ii) OH |
| 1203 | 4-ethyl-thiazol-2-yl | | (C) OCH₃ | |
| 1204 | 4-trifluoromethyl-thiazol-2-yl | | | |
| 1205 | 4-(pentafluoroeth-1-yl)-thiazol-2-yl | | | |

40. The compound of claim 39, wherein the compound is selected from
N-(4-methoxy-2-methylphenyl)-N′-[3-(trifluoromethyl)phenyl]urea;
N-(4-ethyl-1,3-thiazol-2-yl)-N′-(4-methoxy-2-methylphenyl)urea;
N-(4-methoxy-2-methylphenyl)-N′-[4-(trifluoromethyl)-1,3-thiazol-2-yl]urea;
N-(4-hydroxy-2-methylphenyl)-N′-[3-(trifluoromethyl)phenyl]urea;
N-(4-hydroxyphenyl)-N′-[3-(trifluoromethyl)phenyl]urea;
N-(2-ethyl-4-hydroxyphenyl)-N′-[3-(trifluoromethyl)phenyl]urea;
N-(4-methoxyphenyl)-N′-[3-(trifluoromethyl)phenyl]urea;
N-(5-hydroxy-2-methylphenyl)-N′-[3-(trifluoromethyl)phenyl]urea;
N-(4-methoxy-2-methylphenyl)-N′-[4-(trifluoromethyl)-1,3-thiazol-2-yl]thiourea;
N-(4-hydroxy-2-methylphenyl)-N′-[4-(trifluoromethyl)-1,3-thiazol-2-yl]thiourea;
N-(4-hydroxy-2-methylphenyl)-N′-[4-(trifluoromethyl)-1,3-thiazol-2-yl]urea;
N-(4-hydroxyphenyl)-N′-[4-(trifluoromethyl)-1,3-thiazol-2-yl]urea;
N-(5-hydroxy-2-methylphenyl)-N′-[4-(trifluoromethyl)-1,3-thiazol-2-yl]urea;
N-(4-hydroxy-2-methylphenyl)-N′-[4-(pentafluoroethyl)-1,3-thiazol-2-yl]urea;
N-(4-methoxy-2-methylphenyl)-N′-[4-(pentafluoroethyl)-1,3-thiazol-2-yl]urea;
N-(4-methoxy-2-methylphenyl)-N′-(4-methyl-1,3-thiazol-2-yl)urea; or pharmaceutically acceptable salts thereof.

41. The compound of claim 39, wherein the compound is selected from
N-(4-methoxy-2-methylphenyl)-N′-[3-(trifluoromethyl)phenyl]urea;
N-(4-ethyl-1,3-thiazol-2-yl)-N′-(4-methoxy-2-methylphenyl)urea;
N-(4-methoxy-2-methylphenyl)-N′-[4-(trifluoromethyl)-1,3-thiazol-2-yl]urea;
N-(4-hydroxy-2-methylphenyl)-N′-[3-(trifluoromethyl)phenyl]urea;
N-(4-hydroxyphenyl)-N′-[3-(trifluoromethyl)phenyl]urea;
N-(2-ethyl-4-hydroxyphenyl)-N′-[3-(trifluoromethyl)phenyl]urea;
N-(4-methoxyphenyl)-N′-[3-(trifluoromethyl)phenyl]urea;
N-(5-hydroxy-2-methylphenyl)-N′-[3-(trifluoromethyl)phenyl]urea;
N-(4-methoxy-2-methylphenyl)-N′-[4-(trifluoromethyl)-1,3-thiazol-2-yl]thiourea;
N-(4-hydroxy-2-methylphenyl)-N′-[4-(trifluoromethyl)-1,3-thiazol-2-yl]thiourea;
N-(4-hydroxy-2-methylphenyl)-N′-[4-(trifluoromethyl)-1,3-thiazol-2-yl]urea;
N-(4-hydroxyphenyl)-N′-[4-(trifluoromethyl)-1,3-thiazol-2-yl]urea;
N-(5-hydroxy-2-methylphenyl)-N′-[4-(trifluoromethyl)-1,3-thiazol-2-yl]urea;
N-(4-hydroxy-2-methylphenyl)-N′-[4-(pentafluoroethyl)-1,3-thiazol-2-yl]urea;
N-(4-methoxy-2-methylphenyl)-N′-[4-(pentafluoroethyl)-1,3-thiazol-2-yl]urea;
or pharmaceutically acceptable salts thereof.

42. A compound of claim 1, wherein the compound has an isotopic label.

43. A compound of claim 1, wherein the compound contains a photoaffinity label wherein the compound becomes irreversibly incorporated into the nAChR upon exposure to ultraviolet light.

44. A pharmaceutical composition comprising a compound of claim 1 and optionally comprising an alpha 7 nAChR agonist.

45. The pharmaceutical composition of claim 44 further comprising an anti-psychotic agent.

46. The pharmaceutical composition of claim 44 further comprising an agent that increases the level of ACh in the brain.

47. The pharmaceutical composition of claim 46, wherein the agent increasing ACh levels inhibits the activity of acetylcholinesterase or activates the production of ACh.

48. The pharmaceutical composition of claim 44, further comprising at least one of a monoamine reuptake inhibitor or psychostimulant.

49. The pharmaceutical composition of claim 48, wherein the psychostimulant is methylphenidate (Ritalin), dextroamphetamine (Dexedrine), amphetamine (Adderall), and pemoline (Cylert) and the monoamine reuptake inhibitor is desipramine (Norpramin), nortriptyline, atomoxetine (Strattera), reboxetine, fluoxetine (Prozac), tomoxetine, bupropion (Wellbutrin), and modaphonil (Provigil).

50. A method for treating a disease or condition in a mammal in need thereof, wherein the mammal receives symptomatic relief from activation of an alpha 7 nAChR comprising the administration of a therapeutically effective amount of a compound of claim 1 and optionally co-administration of an alpha 7 nAChR agonist for the therapeutically effective interval.

51. The method of claim 50, wherein the compound is selected from
N-(4-hydroxyphenyl)-N′-(5-isopropyl-1,3-thiazol-2-yl)urea;
N-(4-ethoxyphenyl)-N′-(5-methyl-1,3-thiazol-2-yl)urea;
N-[4-(4-chlorophenyl)-1,3-thiazol-2-yl]-N′-(4-iodophenyl)urea;
N-(2,6-dimethylphenyl)-N′-(6-fluoro-1,3-benzothiazol-2-yl)urea;
N-(4-chloro-1,3-benzothiazol-2-yl)-N′-(2-methoxyphenyl)urea;
N-(5-bromo-1,3-thiazol-2-yl)-N′-(2-ethoxyphenyl)urea;
N-(5-bromo-1,3-thiazol-2-yl)-N′-(2,4-dimethoxyphenyl)urea;
N-(5-chloro-1,3-thiazol-2-yl)-N′-(4-methoxy-2-methylphenyl)urea;
N-(5-bromo-1,3-thiazol-2-yl)-N′-(4-methoxy-2-methylphenyl)urea;
N-(5-chloro-2,4-dimethoxyphenyl)-N′-(1,3-thiazol-2-yl)urea;
N-(4-methoxy-2-methylphenyl)-N′-(4-methyl-1,3-thiazol-2-yl)urea;
N-(5-chloro-2,4-dimethoxyphenyl)-N′-(5-methyl-1,3-thiazol-2-yl)urea;
N-(4-chloro-1,3-benzothiazol-2-yl)-N′-(4-methoxy-2-methylphenyl)urea;
ethyl 2-({[(4-methoxy-2-methylphenyl)amino]carbonyl}amino)-4-phenyl-1,3-thiazole-5-carboxylate;
ethyl 2-[({[4-(butoxycarbonyl)phenyl] amino}carbonyl)amino]-4-phenyl-1,3-thiazole-5-carboxylate;
N-(5-bromo-1,3-thiazol-2-yl)-N′-(4-ethoxyphenyl)urea;
N-(4-methoxy-2-methylphenyl)-N′-[3-(trifluoromethyl)phenyl]urea;
N-(5-chloro-2,4-dimethoxyphenyl)-N′-isoxazol-3-ylurea;
N-(5-chloro-2,4-dimethoxyphenyl)-N′-(5-methyl-1,3,4-thiadiazol-2-yl)urea;
N-(5-chloro-2,4-dimethoxyphenyl)-N′-(5-methylisoxazol-3-yl)urea;
N-(5-chloro-2,4-dimethoxyphenyl)-N′-(5-ethyl-1,3,4-thiadiazol-2-yl)urea;
N-(5-chloro-2,4-dimethoxyphenyl)-N′-[5-(trifluoromethyl)-1,3,4-thiadiazol-2-yl]urea;
N-(5-chloro-2,4-dimethoxyphenyl)-N′-(5-cyclopropyl-1,3,4-thiadiazol-2-yl)urea;
N-(5-chloro-2,4-dimethoxyphenyl)-NN′-[5-(difluoromethyl)-1,3,4-thiadiazol-2-yl]urea;
N-(5-chloro-2,4-dimethoxyphenyl)-N′-(5-methoxy-1,3,4-thiadiazol-2-yl)urea;
N-(5-bromo-1,3,4-thiadiazol-2-yl)-N′-(5-chloro-2,4-dimethoxyphenyl)urea;
N-(5-chloro-1,3,4-thiadiazol-2-yl)-N′-(5-chloro-2,4-dimethoxyphenyl)urea;
N-(5-chloro-2,4-dimethoxyphenyl)-N′-(1,3,4-thiadiazol-2-yl)urea;
N-(4-ethoxyphenyl)-N′-(5-methylisoxazol-3-yl)urea;
N-(2,4-dimethoxyphenyl)-N′-(5-methylisoxazol-3-yl)urea;
N-(2,4-dimethoxyphenyl)-N′-[5-(trifluoromethyl)-1,3,4-thiadiazol-2-yl]urea;
N-(5-chloro-2-methoxyphenyl)-N′-(5-methylisoxazol-3-yl)urea;
N-(5-chloro-2-methoxyphenyl)-N′-(5-methyl-1,3,4-thiadiazol-2-yl)urea;
N-(4-isopropoxyphenyl)-N′-[5-(trifluoromethyl)-1,3,4-thiadiazol-2-yl]urea;
N-(2-ethoxyphenyl)-N′-[5-(trifluoromethyl)-1,3,4-thiadiazol-2-yl]urea;
N-(4-ethoxyphenyl)-N′-[5-(trifluoromethyl)-1,3,4-thiadiazol-2-yl]urea;
N-(4-methoxyphenyl)-N′-[5-(trifluoromethyl)-1,3,4-thiadiazol-2-yl]urea;
N-(4-butoxyphenyl)-N′-[5-(trifluoromethyl)-1,3,4-thiadiazol-2-yl]urea;
N-(2,3-dihydro-1-benzofuran-5-yl)-N′-[5-(trifluoromethyl)-1,3,4-thiadiazol-2-yl]urea;
N-(4-ethyl-1,3-thiazol-2-yl)-N′-(4-methoxy-2-methylphenyl)urea;
N-(5-chloro-2,4-dimethoxyphenyl)-N′-(1H-imidazol-2-yl)urea;
N-(5-bromo-1,3-thiazol-2-yl)-N′-(5-chloro-2,4-dimethoxyphenyl)urea;
N-(5-chloro-2,4-dimethoxyphenyl)-N′-(5-methyl-1,3-thiazol-2-yl)urea;
N-(5-chloro-2,4-dimethoxyphenyl)-N′-(3-methylisoxazol-5-yl)urea;
N-(5-chloro-2,4-dimethoxyphenyl)-N′-(6-cyanopyridin-3-yl)urea;
N-(5-chloro-2,4-dimethoxyphenyl)-N′-(5-chloro-1,3-thiazol-2-yl)urea;
N-(5-chloro-1,3-thiazol-2-yl)-N′-(2,4-dimethoxy-5-methylphenyl)urea;
N-(5-bromo-2,4-dimethoxyphenyl)-N′-(5-chloro-1,3-thiazol-2-yl)urea;
N-(5-bromo-1,3-thiazol-2-yl)-N′-(5-fluoro-2,4-dimethoxyphenyl)urea;
N-(5-chloro-1,3-thiazol-2-yl)-N′-(5-fluoro-2,4-dimethoxyphenyl)urea;
N-(3-chloro-4-fluorophenyl)-N′-(5-methylisoxazol-3-yl)urea;
N-(3-chloro-4-fluorophenyl)-N′-[5-(trifluoromethyl)-1,3,4-thiadiazol-2-yl]urea;
N-(2-ethoxyphenyl)-N′-(5-methylisoxazol-3-yl)urea;
N-(2-methoxyphenyl)-N′-[5-(trifluoromethyl)-1,3,4-thiadiazol-2-yl]urea;
N-(2-fluoro-4-methoxyphenyl)-N′-[5-(trifluoromethyl)-1,3,4-thiadiazol-2-yl]urea;
N-(5-chloro-2,4-dimethoxyphenyl)-N′-(5-mercapto-1,3,4-thiadiazol-2-yl)urea;
N-(4,5-dimethoxy-2-methylphenyl)-N′-[5-(trifluoromethyl)-1,3,4-thiadiazol-2-yl]urea;
N-(4-hydroxy-2-nitrophenyl)-N′-[5-(trifluoromethyl)-1,3,4-thiadiazol-2-yl]urea;
N-(2,6-dimethoxypyridin-3-yl)-N′-(5-methyl-1,3-thiazol-2-yl)urea;
N-(4-ethoxy-2-nitrophenyl)-N′-(5-methylisoxazol-3-yl)urea;
N-(4-methoxy-2-methylphenyl)-N′-[2-(trifluoromethyl) pyridin-4-yl]urea;
N-(5-fluoro-2,4-dimethoxyphenyl)-N′-(5-methylisoxazol-3-yl)urea;
N-(5-fluoro-2,4-dimethoxyphenyl)-N′-(5-methyl-1,3-thiazol-2-yl)urea;
N-(5-fluoro-2,4-dimethoxyphenyl)-N′-(5-methyl-1,3,4-thiadiazol-2-yl)urea;
N-(5-fluoro-2,4-dimethoxyphenyl)-N′-[5-(trifluoromethyl)-1,3,4-thiadiazol-2-yl]urea;
N-(5-bromo-2,4-dimethoxyphenyl)-N′-(5-methylisoxazol-3-yl)urea;
N-(5-bromo-2,4-dimethoxyphenyl)-N′-(5-methyl-1,3-thiazol-2-yl)urea;
N-(5-bromo-2,4-dimethoxyphenyl)-N′-(5-methyl-1,3,4-thiadiazol-2-yl)urea;
N-(5-bromo-2,4-dimethoxyphenyl)-N′-[5-(trifluoromethyl)-1,3,4-thiadiazol-2-yl]urea;
N-(3 ,5-difluoro-2,4-dimethoxyphenyl)-N′-(5-methylisoxazol-3-yl)urea;
N-(3,5-difluoro-2,4-dimethoxyphenyl)-N′-[5-(trifluoromethyl)-1,3,4-thiadiazol-2-yl]urea;
N-[2,4-dimethoxy-5-(trifluoromethyl)phenyl]-N′-[5-(trifluoromethyl)-1,3,4-thiadiazol-2-yl]urea;
N-(5-chloro-2,4-diethoxyphenyl)-N′-(5-methylisoxazol-3-yl)urea;
N-(5-chloro-2,4-diethoxyphenyl)-N′-(5-methyl-1,3-thiazol-2-yl)urea;
N-(5-chloro-2,4-diethoxyphenyl)-N′-(5-methyl-1,3,4-thiadiazol-2-yl)urea;
N-(5-chloro-2,4-diethoxyphenyl)-N′-[5-(trifluoromethyl)-1,3,4-thiadiazol-2-yl]urea;
N-(5-chloro-2,4-dipropoxyphenyl)-N′-[5-(trifluoromethyl)-1,3,4-thiadiazol-2-yl]urea;
N-(2-chloro-4-methoxy-5-methylphenyl)-N′-(5-methylisoxazol-3-yl)urea;
N-(2-chloro-4-methoxy-5-methylphenyl)-N′-[5-(trifluoromethyl)-1,3,4-thiadiazol-2-yl]urea;
N-(2,4-dimethoxy-5-methylphenyl)-N′-(5-methylisoxazol-3-yl)urea;
N-(2,4-dimethoxy-5-methylphenyl)-N′-(5-methyl-1,3-thiazol-2-yl)urea;
N-(2,4-dimethoxy-5-methylphenyl)-N′-(5-methyl-1,3,4-thiadiazol-2-yl)urea;
N-(2,4-dimethoxy-5-methylphenyl)-N′-[5-(trifluoromethyl)-1,3,4-thiadiazol-2-yl]urea;
N-(4-ethoxy-2-methoxy-5-methylphenyl)-N′-[5-(trifluoromethyl)-1,3,4-thiadiazol-2-yl]urea;
N-(5-acetyl-2,4-dimethoxyphenyl)-N′-[5-(trifluoromethyl)-1,3,4-thiadiazol-2-yl]urea;
N-(2,4-dimethoxy-5-nitrophenyl)-N′-[5-(trifluoromethyl)-1,3,4-thiadiazol-2-yl]urea;
N-[2-methoxy-5-methyl-4-(2,2,2-trifluoroethoxy)phenyl]-N′-[5-(trifluoromethyl)-1,3,4-thiadiazol-2-yl]urea;
N-(5-chloro-2,4-dimethoxyphenyl)-N′-(2-methyl-1,3-thiazol-5-yl)urea;
N-(2-methoxy-4-nitrophenyl)-N′-[5-(trifluoromethyl)-1,3,4-thiadiazol-2-yl]urea;
N-[5-(trifluoromethyl)-1,3,4-thiadiazol-2-yl]-N′-(2,4,5-trimethoxyphenyl)urea;
N-[4-methoxy-2-(methylthio)phenyl]-N′-[5-(trifluoromethyl)-1,3,4-thiadiazol-2-yl]urea;
N-(4-{[(1R)-1-methylpropyl]oxy}phenyl)-N′-[5-(trifluoromethyl)-1,3,4-thiadiazol-2-yl]urea;
N-[4-(allyloxy)phenyl]-N′-[5-(trifluoromethyl)-1,3,4-thiadiazol-2-yl]urea;
N-(4-propoxyphenyl)-N′-[5-(trifluoromethyl)-1,3,4-thiadiazol-2-yl]urea;
N-(2-ethoxypyridin-3-yl)-N′-[5-(trifluoromethyl)-1,3,4-thiadiazol-2-yl]urea;
N-(4-methoxy-2-methylphenyl)-N′-[4-(trifluoromethyl)-1,3-thiazol-2-yl]urea;
N-(4-methoxy-2-methylphenyl)-N′-(3-phenyl-1,2,4-thiadiazol-5-yl)urea;
N-(5-ethyl-4-phenyl-1,3-thiazol-2-yl)-N′-(4-methoxy-2-methylphenyl)urea;
N-(4-hydroxy-2-methylphenyl)-N′-[3-(trifluoromethyl)phenyl]urea;
N-(4-hydroxyphenyl)-N′-[3-(trifluoromethyl)phenyl]urea;
N-[2-methyl-4-(methylthio)phenyl]-N′-[3-(trifluoromethyl)phenyl]urea;
N-(2-ethyl-4-hydroxyphenyl)-N′-[3-(trifluoromethyl)phenyl]urea;
N-(4-amino-2-methylphenyl)-N′-[3-(trifluoromethyl)phenyl]urea;
N-(4-methoxyphenyl)-N′-[3-(trifluoromethyl)phenyl]urea;
N-(5-hydroxy-2-methylphenyl)-N′-[3-(trifluoromethyl)phenyl]urea;
N-(5-chloro-2,4-dimethoxyphenyl)-N′-[4-(trifluoromethyl)-1H-pyrazol-1-yl]urea;
N-(4-bromo-1H-pyrazol-1-yl)-N′-(5-chloro-2,4-dimethoxyphenyl)urea;
N-(4-methoxy-2-methylphenyl)-N′-[3-(trifluoromethyl)phenyl]thiourea;
N-(4-hydroxy-2-methylphenyl)-N′-[3-(trifluoromethyl)phenyl]thiourea;
N-(2,4-dimethoxy-5-methylphenyl)-N′-[3-(trifluoromethyl)isoxazol-5-yl]urea;
N-(4-ethoxyphenyl)-N′-[3-(trifluoromethyl)isoxazol-5-yl]urea;
N-(2-ethoxyphenyl)-N′-[3-(trifluoromethyl)isoxazol-5-yl]urea;
N-(2,4-dimethoxyphenyl)-N′-[3-(trifluoromethyl)isoxazol-5-yl]urea;
N-(2,6-dimethoxypyridin-3-yl)-N′-(3-methylisoxazol-5-yl)urea;
N-(5-fluoro-2,4-dimethoxyphenyl)-N′-(3-methylisoxazol-5-yl)urea;
N-(2,4-dimethoxyphenyl)-N′-(3-methylisoxazol-5-yl)urea;
N-(5-chloro-2,4-dimethoxyphenyl)-N′-[3-(trifluoromethyl)isoxazol-5-yl]thiourea;
N-(5-chloro-2,4-diethoxyphenyl)-N′-[3-(trifluoromethyl)isoxazol-5-yl]urea;
N-(4-methoxy-2-nitrophenyl)-N′-[3-(trifluoromethyl)isoxazol-5-yl]urea;
N-(4-methoxy-2-methylphenyl)-N′-[4-(trifluoromethyl)-1,3-thiazol-2-yl]thiourea;
N-(5-chloro-2,4-dimethoxyphenyl)-N′-[5-(trifluoromethyl)-1,3,4-thiadiazol-2-yl]thiourea;
N-(5-fluoro-2,4-dimethoxyphenyl)-N′-[5-(trifluoromethyl)-1,3,4-thiadiazol-2-yl]thiourea;
N-[2-methoxy-4-(2-methoxyethoxy)phenyl]-N′-[5-(trifluoromethyl)-1,3,4-thiadiazol-2-yl]urea;
N-(4-ethoxy-2-nitrophenyl)-N′-[3-(trifluoromethyl)isoxazol-5-yl]thiourea;
N-(4-hydroxy-2-methylphenyl)-N′-[4-(trifluoromethyl)-1,3-thiazol-2-yl]thiourea;
N-(3-chloro-4-methoxyphenyl)-NN′-[5-(trifluoromethyl)-1,3,4-thiadiazol-2-yl]urea;
N-(5-chloro-2-methoxyphenyl)-N′-[5-(trifluoromethyl)-1,3,4-thiadiazol-2-yl]urea;
N-(4-methoxy-2-nitrophenyl)-N′-[5-(trifluoromethyl)-1,3,4-thiadiazol-2-yl]urea;
N-(4-ethoxy-2-nitrophenyl)-N′-[5-(trifluoromethyl)-1,3,4-thiadiazol-2-yl]urea;
N-(4-methoxy-2-methylphenyl)-N′-[5-(trifluoromethyl)-1,3,4-thiadiazol-2-yl]urea;
N-(2,6-dimethoxypyridin-3-yl)-N′-[5-(trifluoromethyl)-1,3,4-thiadiazol-2-yl]urea;
N-(4-hydroxyphenyl)-N′-[5-(trifluoromethyl)-1,3,4-thiadiazol-2-yl]urea;
N-(5-ethoxypyridin-2-yl)-N′-[5-(trifluoromethyl)-1,3,4-thiadiazol-2-yl]urea;
N-(4-ethoxy-2-morpholin-4-ylphenyl)-N′-[5-(trifluoromethyl)-1,3,4-thiadiazol-2-yl]urea;
tert-butyl 4-{5-ethoxy-2-[({[5-(trifluoromethyl)-1,3,4-thiadiazol-2-yl]amino}carbonyl) amino]phenyl}piperazine-1-carboxylate;
N-(2-chloro-6-methoxy-pyridin-3-yl)-N′-[5-(trifluoromethyl)-1,3,4-thiadiazol-2-yl]urea;
N-[6-methoxy-2-(methylthio)pyridin-3-yl]-N′-[5-(trifluoromethyl)-1,3,4-thiadiazol-2-yl]urea;
N-[6-methoxy-2-(methylsulfonyl)pyridin-3-yl]-N′-[5-(trifluoromethyl)-1,3,4-thiadiazol-2-yl]urea;
N-[2-methoxy-4-(2-methoxyethoxy)phenyl]-N′-(5-methylisoxazol-3-yl)urea;
N-(4-hydroxy-2-methylphenyl)-N′-[4-(trifluoromethyl)-1,3-thiazol-2-yl]urea;
N-(4-hydroxyphenyl)-N′-[4-(trifluoromethyl)-1,3-thiazol-2-yl]urea;
N-(5-hydroxy-2-methylphenyl)-N′-[4-(trifluoromethyl)-1,3-thiazol-2-yl]urea;
N-(3-hydroxy-2-methylphenyl)-N′-[4-(trifluoromethyl)-1,3-thiazol-2-yl]urea;
N-(6-cyanopyridin-3-yl)-N′-(5-fluoro-2,4-dimethoxyphenyl)urea;
N-(4-hydroxy-2-methylphenyl)-N′-[3-(trifluoromethoxy)phenyl]urea;
N-(4-hydroxy-2-methylphenyl)-N′-[4-(pentafluoroethyl)-1,3-thiazol-2-yl]urea;
N-(5-bromo-2,4-dimethoxyphenyl)-N′-(6-cyanopyridin-3-yl)urea;
N-(5-chloro-2,4-dimethoxyphenyl)-N′-[3-(trifluoromethyl)isoxazol-5-yl]urea;
N-(5-fluoro-2,4-dimethoxyphenyl)-N′-[3-(trifluoromethyl)isoxazol-5-yl]urea;
N-(2,6-dimethoxypyridin-3-yl)-N′-[3-(trifluoromethyl)isoxazol-5-yl]urea;
N-(4-ethoxy-2-nitrophenyl)-N′-[3-(trifluoromethyl)isoxazol-5-yl]urea;
N-(5-chloro-2,4-dimethoxyphenyl)-N′-(5-chloroisoxazol-3-yl)urea;
N-(5-chloro-4-methoxy-2-nitrophenyl)-N′-(5-methylisoxazol-3-yl)urea;
N-(5-fluoro-4-methoxy-2-nitrophenyl)-N′-(5-methylisoxazol-3-yl)urea;
N-[5-chloro-4-methoxy-2-(methylthio)phenyl]-N′-(5-methylisoxazol-3-yl)urea;
N-(5-chloro-2,4-dimethoxyphenyl)-N′-[5-(fluoromethyl)isoxazol-3-yl]urea;
N-(2,6-dimethoxypyridin-3-yl)-N′-[5-(fluoromethyl)isoxazol-3-yl]urea;
N-(5-fluoro-2,4-dimethoxyphenyl)-N′-[5-(fluoromethyl)isoxazol-3-yl]urea;
N-(5-bromo-2,4-dimethoxyphenyl)-N′-[5-(fluoromethyl)isoxazol-3-yl]urea;
N-(4-ethoxy-2-nitrophenyl)-N′-[5-(fluoromethyl)isoxazol-3-yl]urea;
N-(2,4-dimethoxy-5-methylphenyl)-N′-[5-(fluoromethyl)isoxazol-3-yl]urea;
N-(5-bromo-2,4-dimethoxyphenyl)-N′-[5-(hydroxymethyl)isoxazol-3-yl]urea;
N-(5-fluoro-2,4-dimethoxyphenyl)-N′-(5-isopropylisoxazol-3-yl)urea;
N-(5-chloro-2,4-dimethoxyphenyl)-N′-(5-isopropylisoxazol-3-yl)urea;
N-(5-bromo-2,4-dimethoxyphenyl)-N′-(5-isopropylisoxazol-3-yl)urea;
N-(2,6-dimethoxypyridin-3-yl)-N′-(5-isopropylisoxazol-3-yl)urea;
N-(2,4-dimethoxy-5-methylphenyl)-N′-(5-isopropylisoxazol-3-yl)urea;
N-(5-fluoro-2,4-dimethoxyphenyl)-N′-[5-(methoxymethyl)isoxazol-3-yl]urea;
N-(5-chloro-2,4-dimethoxyphenyl)-N′-[5-(methoxymethyl)isoxazol-3-yl]urea;
N-(5-bromo-2,4-dimethoxyphenyl)-N′-[5-(methoxymethyl)isoxazol-3-yl]urea;
N-(4-ethoxy-2-nitrophenyl)-N′-[5-(methoxymethyl)isoxazol-3-yl]urea;
N-(5-chloro-2,4-dimethoxyphenyl)-N′-(5-cyclopropylisoxazol-3-yl)urea;
N-(5-cyclopropylisoxazol-3-yl)-N′-(5-fluoro-2,4-dimethoxyphenyl)urea;
N-(5-bromo-2,4-dimethoxyphenyl)-N′-(5-cyclopropylisoxazol-3-yl)urea;
N-(5-cyclopropylisoxazol-3-yl)-N′-(2,4-dimethoxy-5-methylphenyl)urea;
N-(5-cyclopropylisoxazol-3-yl)-N′-(4-ethoxy-2-nitrophenyl)urea;
N-(5-cyclopropylisoxazol-3-yl)-N′-(2,6-dimethoxypyridin-3-yl)urea;
N-(5-chloro-2,4-dimethoxyphenyl)-N′-(5-ethylisoxazol-3-yl)urea;
N-(5-ethylisoxazol-3-yl)-N′-(5-fluoro-2,4-dimethoxyphenyl)urea;
N-(5-bromo-2,4-dimethoxyphenyl)-N′-(5-ethylisoxazol-3-yl)urea;
N-(2,4-dimethoxy-5-methylphenyl)-N′-(5-ethylisoxazol-3-yl)urea;
N-(2,6-dimethoxypyridin-3-yl)-N′-(5-ethylisoxazol-3-yl)urea;
N-(4-ethoxy-2-nitrophenyl)-N′-(5-ethylisoxazol-3-yl)urea;
N-(5-amino-2,4-dimethoxyphenyl)-N′-[5-(trifluoromethyl)-1,3,4-thiadiazol-2-yl]urea;
N-(5-azido-2,4-dimethoxyphenyl)-N′-[5-(trifluoromethyl)-1,3,4-thiadiazol-2-yl]urea;
N-(5-iodo-2,4-dimethoxyphenyl)-N′-[5-(trifluoromethyl)-1,3,4-thiadiazol-2-yl]urea;
N-(4-amino-2-methoxyphenyl)-N′-[5-(trifluoromethyl)-1,3,4-thiadiazol-2-yl]urea;
N-(4-methoxy-2-methylphenyl)-N′-[4-(pentafluoroethyl)-1,3-thiazol-2-yl]urea;
N-(5-chloro-2,4-dimethoxyphenyl)-N′-[5-(trifluoromethyl)isoxazol-3-yl]urea;
N-[2-methoxy-4-(tetrahydrofuran-3-yloxy)phenyl]-N′-[5-(trifluoromethyl)-1,3,4-thiadiazol-2-yl]urea;
N-[2-methoxy-4-(oxetan-3-yloxy)phenyl]-N′-[5-(trifluoromethyl)-1,3,4-thiadiazol-2-yl]urea;
N-[2-methoxy-4-(oxetan-3-yloxy)phenyl]-N′-[3-(trifluoromethyl)isoxazol-5-yl]urea;
N-[2-methoxy-4-(tetrahydrofuran-3-yloxy)phenyl]-N′-[3-(trifluoromethyl)isoxazol-5-yl]urea; or pharmaceutically acceptable salts thereof.

52. The method of claim 50, wherein the disease or condition is cognitive and attention deficit symptoms of Alzheimer's, neurodegeneration associated with diseases such as Alzheimer's disease, pre-senile dementia (mild cognitive impairment), or senile dementia.

53. The method of claim 50, wherein the disease or condition is schizophrenia or psychosis and related cognitive deficits associated therewith.

54. The method of claim 53, wherein the mammal receives symptomatic relief from co-administration of an anti-psychotic agent for a therapeutically effective interval.

55. The method of claim 50, wherein the disease or condition is attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, or symptoms associated with pain.

56. The method of claim 55, wherein the disease or condition is attention deficit hyperactivity disorder and wherein the mammal receives symptomatic relief from co-administration of at least one of a monoamine reuptake inhibitor, or psychostimulant for a therapeutically effective interval.

57. The method of claim 56, wherein the psychostimulant is methylphenidate (Ritalin) administered at about 0.01 to about 0.85 mg/kg/day; dextroamphetamine (Dexedrine) administered at about 0.07 to about 0.85 mg/kg/day; amphetamine (Adderall) administered at about 0.05 to about 0.6 mg/kg/day; and pemoline (Cylert) administered at about 0.1 to about 1.6 mg/kg/day; and wherein the monoamine reuptake inhibitor is desipramine (Norpramin) administered at about 0.5 to about 5.0 mg/kg/day; nortriptyline administered at about 0.1 to about 3.0 mg/kg/day; atomoxetine (Strattera) administered at about 0.1 to about 3.0 mg/kg/day; reboxetine administered at about 0.03 to about 3.0 mg/kg/day; fluoxetine (Prozac) at about 0.2 to about 20 mg/kg/day; tomoxetine administered at about at about 0.1 to about 1.1 mg/kg/day; bupropion (Wellbutrin) administered at about at about 1.0 to about 1.1 mg/kg/day; and modaphonil (Provigil) administered at about at about 1.0 to about 5.7 mg/kg/day.

58. The method of claim 50, wherein the mammal receives therapeutic relief from co-administration of an agent that inhibits the activity of acetylcholinesterase.

59. The method of claim 58, wherein the agent inhibiting acetylcholinesterase is Aricept and Reminyl.

60. The method of claim 50, wherein the mammal receives therapeutic relief from co-administration of an agent that increases levels of ACh in the brain.

61. The method of claim 60, wherein the agent is choline or is a nutritional supplement increasing ACh in the brain.

62. The method of claim 50, wherein the compound of the present invention and any other agent(s) are independently administered rectally, topically, orally, sublingually or parentarally.

63. The method of claim 62, wherein the compound of the present invention is administered in an amount of from about 0.001 to about 100 mg/kg of body weight of said mammal per day.

64. The method of claim 62, wherein the compound of the present invention is administered in an amount of from about 0.01 to about 50 mg/kg of body weight of said mammal per day.

65. A method for treating a disease or condition in a mammal in need thereof, wherein the mammal receives symptomatic relief from decreasing the level of TNF-αcomprising administering a compound of claim 1.

66. The method of claim 65, wherein the compound is selected from
N-(4-hydroxyphenyl)-N′-(5-isopropyl-1,3-thiazol-2-yl)urea;
N-(4-ethoxyphenyl)-N′-(5-methyl-1,3-thiazol-2-yl)urea;
N-[4-(4-chlorophenyl)-1,3-thiazol-2-yl]-N′-(4-iodophenyl)urea;
N-(2,6-dimethylphenyl)-N′-(6-fluoro-1,3-benzothiazol-2-yl)urea;
N-(4-chloro-1,3-benzothiazol-2-yl)-N′-(2-methoxyphenyl)urea;
N-(5-bromo-1,3-thiazol-2-yl)-N′-(2-ethoxyphenyl)urea;
N-(5-bromo-1,3-thiazol-2-yl)-N′-(2,4-dimethoxyphenyl)urea;
N-(5-chloro-1,3-thiazol-2-yl)-N′-(4-methoxy-2-methylphenyl)urea;
N-(5-bromo-1,3-thiazol-2-yl)-N′-(4-methoxy-2-methylphenyl)urea;
N-(5-chloro-2,4-dimethoxyphenyl)-N′-(1,3-thiazol-2-yl)urea;
N-(4-methoxy-2-methylphenyl)-N′-(4-methyl-1,3-thiazol-2-yl)urea;
N-(5-chloro-2,4-dimethoxyphenyl)-N′-(5-methyl-1,3-thiazol-2-yl)urea;
N-(4-chloro-1,3-benzothiazol-2-yl)-N′-(4-methoxy-2-methylphenyl)urea;
ethyl 2-({[(4-methoxy-2-methylphenyl)amino]carbonyl}amino)-4-phenyl-1,3-thiazole-5-carboxylate;
ethyl 2-[({[4-(butoxycarbonyl)phenyl]amino}carbonyl)amino]-4-phenyl-1,3-thiazole-5-carboxylate;
N-(5-bromo-1,3-thiazol-2-yl)-N′-(4-ethoxyphenyl)urea;
N-(4-methoxy-2-methylphenyl)-N′-[3-(trifluoromethyl)phenyl]urea;
N-(5-chloro-2,4-dimethoxyphenyl)-N′-isoxazol-3-ylurea;
N-(5-chloro-2,4-dimethoxyphenyl)-N′-(5-methyl-1,3,4-thiadiazol-2-yl)urea;
N-(5-chloro-2,4-dimethoxyphenyl)-N′-(5-methylisoxazol-3-yl)urea;
N-(5-chloro-2,4-dimethoxyphenyl)-N′-(5-ethyl-1,3,4-thiadiazol-2-yl)urea;
N-(5-chloro-2,4-dimethoxyphenyl)-N′-[5-(trifluoromethyl)-1,3,4-thiadiazol-2-yl]urea;
N-(5-chloro-2,4-dimethoxyphenyl)-N′-(5-cyclopropyl-1,3,4-thiadiazol-2-yl)urea;
N-(5-chloro-2,4-dimethoxyphenyl)-N′-[5-(difluoromethyl)-1,3,4-thiadiazol-2-yl]urea;
N-(5-chloro-2,4-dimethoxyphenyl)-N′-(5-methoxy-1,3,4-thiadiazol-2-yl)urea;
N-(5-bromo-1,3,4-thiadiazol-2-yl)-N′-(5-chloro-2,4-dimethoxyphenyl)urea;
N-(5-chloro-1,3,4-thiadiazol-2-yl)-N′-(5-chloro-2,4-dimethoxyphenyl)urea;
N-(5-chloro-2,4-dimethoxyphenyl)-N′-(1,3,4-thiadiazol-2-yl)urea;
N-(4-ethoxyphenyl)-N′-(5-methylisoxazol-3-yl)urea;
N-(2,4-dimethoxyphenyl)-N′-(5-methylisoxazol-3-yl)urea;
N-(2,4-dimethoxyphenyl)-N′-[5-(trifluoromethyl)-1,3,4-thiadiazol-2-yl]urea;
N-(5-chloro-2-methoxyphenyl)-N′-(5-methylisoxazol-3-yl)urea;
N-(5-chloro-2-methoxyphenyl)-N′-(5-methyl-1,3,4-thiadiazol-2-yl)urea;
N-(4-isopropoxyphenyl)-N′-[5-(trifluoromethyl)-1,3,4-thiadiazol-2-yl]urea;
N-(2-ethoxyphenyl)-N′-[5-(trifluoromethyl)-1,3,4-thiadiazol-2-yl]urea;
N-(4-ethoxyphenyl)-N′-[5-(trifluoromethyl)-1,3,4-thiadiazol-2-yl]urea;
N-(4-methoxyphenyl)-N′-[5-(trifluoromethyl)-1,3,4-thiadiazol-2-yl]urea;
N-(4-butoxyphenyl)-NN′-[5-(trifluoromethyl)-1,3,4-thiadiazol-2-yl]urea;
N-(2,3-dihydro-1-benzofuran-5-yl)-N′-[5-(trifluoromethyl)-1,3,4-thiadiazol-2-yl]urea;
N-(4-ethyl-1,3-thiazol-2-yl)-N-(4-methoxy-2-methylphenyl)urea;
N-(5-chloro-2,4-dimethoxyphenyl)-N′-(1H-imidazol-2-yl)urea;
N-(5-bromo-1,3-thiazol-2-yl)-N′-(5-chloro-2,4-dimethoxyphenyl)urea;
N-(5-chloro-2,4-dimethoxyphenyl)-N′-(5-methyl-1,3-thiazol-2-yl)urea;
N-(5-chloro-2,4-dimethoxyphenyl)-N′-(3-methylisoxazol-5-yl)urea;
N-(5-chloro-2,4-dimethoxyphenyl)-N′-(6-cyanopyridin-3-yl)urea;
N-(5-chloro-2,4-dimethoxyphenyl)-N′-(5-chloro-1,3-thiazol-2-yl)urea;
N-(5-chloro-1,3-thiazol-2-yl)-N′-(2,4-dimethoxy-5-methylphenyl)urea;
N-(5-bromo-2,4-dimethoxyphenyl)-N′-(5-chloro-1,3-thiazol-2-yl)urea;
N-(5-bromo-1,3-thiazol-2-yl)-N′-(5-fluoro-2,4-dimethoxyphenyl)urea;
N-(5-chloro-1,3-thiazol-2-yl)-N′-(5-fluoro-2,4-dimethoxyphenyl)urea;
N-(3-chloro-4-fluorophenyl)-N′-(5-methylisoxazol-3-yl)urea;
N-(3-chloro-4-fluorophenyl)-N′-[5-(trifluoromethyl)-1,3,4-thiadiazol-2-yl]urea;
N-(2-ethoxyphenyl)-N′-(5-methylisoxazol-3-yl)urea;
N-(2-methoxyphenyl)-N′-[5-(trifluoromethyl)-1,3,4-thiadiazol-2-yl]urea;
N-(2-fluoro-4-methoxyphenyl)-N′-[5-(trifluoromethyl)-1,3,4-thiadiazol-2-yl]urea;
N-(5-chloro-2,4-dimethoxyphenyl)-N′-(5-mercapto-1,3,4-thiadiazol-2-yl)urea;
N-(4,5-dimethoxy-2-methylphenyl)-N′-[5-(trifluoromethyl)-1,3,4-thiadiazol-2-yl]urea;
N-(4-hydroxy-2-nitrophenyl)-N′-[5-(trifluoromethyl)-1,3,4-thiadiazol-2-yl]urea;
N-(2,6-dimethoxypyridin-3-yl)-N′-(5-methyl-1,3-thiazol-2-yl)urea;
N-(4-ethoxy-2-nitrophenyl)-N′-(5-methylisoxazol-3-yl)urea;
N-(4-methoxy-2-methylphenyl)-N′-[2-(trifluoromethyl) pyridin-4-yl]urea;
N-(5-fluoro-2,4-dimethoxyphenyl)-N′-(5-methylisoxazol-3-yl)urea;
N-(5-fluoro-2,4-dimethoxyphenyl)-N′-(5-methyl-1,3-thiazol-2-yl)urea;
N-(5-fluoro-2,4-dimethoxyphenyl)-N′-(5-methyl-1,3,4-thiadiazol-2-yl)urea;
N-(5-fluoro-2,4-dimethoxyphenyl)-N′-[5-(trifluoromethyl)-1,3,4-thiadiazol-2-yl]urea;
N-(5-bromo-2,4-dimethoxyphenyl)-N′-(5-methylisoxazol-3-yl)urea;
N-(5-bromo-2,4-dimethoxyphenyl)-N′-(5-methyl-1,3-thiazol-2-yl)urea;
N-(5-bromo-2,4-dimethoxyphenyl)-N′-(5-methyl-1,3,4-thiadiazol-2-yl)urea;
N-(5-bromo-2,4-dimethoxyphenyl)-N′-[5-(trifluoromethyl)-1,3,4-thiadiazol-2-yl]urea;
N-(3,5-difluoro-2,4-dimethoxyphenyl)-N′-(5-methylisoxazol-3-yl)urea;
N-(3,5-difluoro-2,4-dimethoxyphenyl)-N′-[5-(trifluoromethyl)-1,3,4-thiadiazol-2-yl]urea;
N-[2,4-dimethoxy-5-(trifluoromethyl)phenyl]-N′-[5-(trifluoromethyl)-1,3,4-thiadiazol-2-yl]urea;
N-(5-chloro-2,4-diethoxyphenyl)-N′-(5-methylisoxazol-3-yl)urea;
N-(5-chloro-2,4-diethoxyphenyl)-N′-(5-methyl-1,3-thiazol-2-yl)urea;
N-(5-chloro-2,4-diethoxyphenyl)-N′-(5-methyl-1,3,4-thiadiazol-2-yl)urea;
N-(5-chloro-2,4-diethoxyphenyl)-N′-[5-(trifluoromethyl)-1,3,4-thiadiazol-2-yl]urea;
N-(5-chloro-2,4-dipropoxyphenyl)-N′-[5-(trifluoromethyl)-1,3,4-thiadiazol-2-yl]urea;
N-(2-chloro-4-methoxy-5-methylphenyl)-N′-(5-methylisoxazol-3-yl)urea;
N-(2-chloro-4-methoxy-5-methylphenyl)-N′-[5-(trifluoromethyl)-1,3,4-thiadiazol-2-yl]urea;
N-(2,4-dimethoxy-5-methylphenyl)-N′-(5-methylisoxazol-3-yl)urea;
N-(2,4-dimethoxy-5-methylphenyl)-N′-(5-methyl-1,3-thiazol-2-yl)urea;
N-(2,4-dimethoxy-5-methylphenyl)-N′-(5-methyl-1,3,4-thiadiazol-2-yl)urea;
N-(2,4-dimethoxy-5-methylphenyl)-N′-[5-(trifluoromethyl)-1,3,4-thiadiazol-2-yl]urea;
N-(4-ethoxy-2-methoxy-5-methylphenyl)-N′-[5-(trifluoromethyl)-1,3,4-thiadiazol-2-yl]urea;
N-(5-acetyl-2,4-dimethoxyphenyl)-N′-[5-(trifluoromethyl)-1,3,4-thiadiazol-2-yl]urea;
N-(2,4-dimethoxy-5-nitrophenyl)-N′-[5-(trifluoromethyl)-1,3,4-thiadiazol-2-yl]urea;
N-[2-methoxy-5-methyl-4-(2,2,2-trifluoroethoxy)phenyl]-N′-[5-(trifluoromethyl)-1,3,4-thiadiazol-2-yl]urea;
N-(5-chloro-2,4-dimethoxyphenyl)-N′-(2-methyl-1,3-thiazol-5-yl)urea;
N-(2-methoxy-4-nitrophenyl)-N′-[5-(trifluoromethyl)-1,3,4-thiadiazol-2-yl]urea;
N-[5-(trifluoromethyl)-1,3,4-thiadiazol-2-yl]-N′-(2,4,5-trimethoxyphenyl)urea;
N-[4-methoxy-2-(methylthio)phenyl]-N′-[5-(trifluoromethyl)-1,3,4-thiadiazol-2-yl]urea;
N-(4-{[(1R)-1-methylpropyl]oxy}phenyl)-N′-[5-(trifluoromethyl)-1,3,4-thiadiazol-2-yl]urea;
N-[4-(allyloxy)phenyl]-N′-[5-(trifluoromethyl)-1,3,4-thiadiazol-2-yl]urea;
N-(4-propoxyphenyl)-N′-[5-(trifluoromethyl)-1,3,4-thiadiazol-2-yl]urea;
N-(2-ethoxypyridin-3-yl)-N′-[5-(trifluoromethyl)-1,3,4-thiadiazol-2-yl]urea;
N-(4-methoxy-2-methylphenyl)-N′-[4-(trifluoromethyl)-1,3-thiazol-2-yl]urea;
N-(4-methoxy-2-methylphenyl)-N′-(3-phenyl-1,2,4-thiadiazol-5-yl)urea;
N-(5-ethyl-4-phenyl-1,3-thiazol-2-yl)-N′-(4-methoxy-2-methylphenyl)urea;
N-(4-hydroxy-2-methylphenyl)-N′-[3-(trifluoromethyl)phenyl]urea;
N-(4-hydroxyphenyl)-N′-[3-(trifluoromethyl)phenyl]urea;
N-[2-methyl-4-(methylthio)phenyl]-N′-[3-(trifluoromethyl)phenyl]urea;
N-(2-ethyl-4-hydroxyphenyl)-N′-[3-(trifluoromethyl)phenyl]urea;
N-(4-amino-2-methylphenyl)-N′-[3-(trifluoromethyl)phenyl]urea;
N-(4-methoxyphenyl)-N′-[3-(trifluoromethyl)phenyl]urea;
N-(5-hydroxy-2-methylphenyl)-N′-[3-(trifluoromethyl)phenyl]urea;
N-(5-chloro-2,4-dimethoxyphenyl)-N′-[4-(trifluoromethyl)-1H-pyrazol-1-yl]urea;
N-(4-bromo-1H-pyrazol-1-yl)-N′-(5-chloro-2,4-dimethoxyphenyl)urea;
N-(4-methoxy-2-methylphenyl)-N′-[3-(trifluoromethyl)phenyl]thiourea;
N-(4-hydroxy-2-methylphenyl)-N′-[3-(trifluoromethyl)phenyl]thiourea;
N-(2,4-dimethoxy-5-methylphenyl)-N′-[3-(trifluoromethyl)isoxazol-5-yl]urea;
N-(4-ethoxyphenyl)-N′-[3-(trifluoromethyl)isoxazol-5-yl]urea;
N-(2-ethoxyphenyl)-N′-[3-(trifluoromethyl)isoxazol-5-yl]urea;
N-(2,4-dimethoxyphenyl)-N′-[3-(trifluoromethyl)isoxazol-5-yl]urea;
N-(2,6-dimethoxypyridin-3-yl)-N′-(3-methylisoxazol-5-yl)urea;
N-(5-fluoro-2,4-dimethoxyphenyl)-N′-(3-methylisoxazol-5-yl)urea;
N-(2,4-dimethoxyphenyl)-N′-(3-methylisoxazol-5-yl)urea;
N-(5-chloro-2,4-dimethoxyphenyl)-N′-[3-(trifluoromethyl)isoxazol-5-yl]thiourea;
N-(5-chloro-2,4-diethoxyphenyl)-N′-[3-(trifluoromethyl)isoxazol-5-yl]urea;
N-(4-methoxy-2-nitrophenyl)-N′-[3-(trifluoromethyl)isoxazol-5-yl]urea;
N-(4-methoxy-2-methylphenyl)-N′-[4-(trifluoromethyl)-1,3-thiazol-2-yl]thiourea;
N-(5-chloro-2,4-dimethoxyphenyl)-N′-[5-(trifluoromethyl)-1,3,4-thiadiazol-2-yl]thiourea;
N-(5-fluoro-2,4-dimethoxyphenyl)-N′-[5-(trifluoromethyl)-1,3,4-thiadiazol-2-yl]thiourea;
N-[2-methoxy-4-(2-methoxyethoxy)phenyl]-N′-[5-(trifluoromethyl)-1,3,4-thiadiazol-2-yl]urea;
N-(4-ethoxy-2-nitrophenyl)-N′-[3-(trifluoromethyl)isoxazol-5-yl]thiourea;
N-(4-hydroxy-2-methylphenyl)-N′-[4-(trifluoromethyl)-1,3-thiazol-2-yl]thiourea;
N-(3-chloro-4-methoxyphenyl)-N′-[5-(trifluoromethyl)-1,3,4-thiadiazol-2-yl]urea;
N-(5-chloro-2-methoxyphenyl)-N′-[5-(trifluoromethyl)-1,3,4-thiadiazol-2-yl]urea;
N-(4-methoxy-2-nitrophenyl)-N′-[5-(trifluoromethyl)-1,3,4-thiadiazol-2-yl]urea;
N-(4-ethoxy-2-nitrophenyl)-N′-[5-(trifluoromethyl)-1,3,4-thiadiazol-2-yl]urea;
N-(4-methoxy-2-methylphenyl)-N′-[5-(trifluoromethyl)-1,3,4-thiadiazol-2-yl]urea;
N-(2,6-dimethoxypyridin-3-yl)-N′-[5-(trifluoromethyl)-1,3,4-thiadiazol-2-yl]urea;
N-(4-hydroxyphenyl)-N′-[5-(trifluoromethyl)-1,3,4-thiadiazol-2-yl]urea;
N-(5-ethoxypyridin-2-yl)-N′-[5-(trifluoromethyl)-1,3,4-thiadiazol-2-yl]urea;
N-(4-ethoxy-2-morpholin-4-ylphenyl)-N′-[5-(trifluoromethyl)-1,3,4-thiadiazol-2-yl]urea;
tert-butyl 4-{5-ethoxy-2-[({[5-(trifluoromethyl)-1,3,4-thiadiazol-2-yl]amino}carbonyl)amino]phenyl}piperazine-1-carboxylate;
N-(2-chloro-6-methoxy-pyridin-3-yl)-N′-[5-(trifluoromethyl)-1,3,4-thiadiazol-2-yl]urea;
N-[6-methoxy-2-(methylthio)pyridin-3-yl]-N′-[5-(trifluoromethyl)-1,3,4-thiadiazol-2-yl]urea;
N-[6-methoxy-2-(methylsulfonyl)pyridin-3-yl]-N′-[5-(trifluoromethyl)-1,3,4-thiadiazol-2-yl]urea;
N-[2-methoxy-4-(2-methoxyethoxy)phenyl]-N′-(5-methylisoxazol-3-yl)urea;
N-(4-hydroxy-2-methylphenyl)-N′-[4-(trifluoromethyl)-1,3-thiazol-2-yl]urea;
N-(4-hydroxyphenyl)-N′-[4-(trifluoromethyl)-1,3-thiazol-2-yl]urea;
N-(5-hydroxy-2-methylphenyl)-N′-[4-(trifluoromethyl)-1,3-thiazol-2-yl]urea;
N-(3-hydroxy-2-methylphenyl)-N′-[4-(trifluoromethyl)-1,3-thiazol-2-yl]urea;
N-(6-cyanopyridin-3-yl)-N′-(5-fluoro-2,4-dimethoxyphenyl)urea;
N-(4-hydroxy-2-methylphenyl)-N′-[3-(trifluoromethoxy)phenyl]urea;
N-(4-hydroxy-2-methylphenyl)-N′-[4-(pentafluoroethyl)-1,3-thiazol-2-yl]urea;
N-(5-bromo-2,4-dimethoxyphenyl)-N′-(6-cyanopyridin-3-yl)urea;
N-(5-chloro-2,4-dimethoxyphenyl)-N′-[3-(trifluoromethyl)isoxazol-5-yl]urea;
N-(5-fluoro-2,4-dimethoxyphenyl)-N′-[3-(trifluoromethyl)isoxazol-5-yl]urea;
N-(2,6-dimethoxypyridin-3-yl)-N′-[3-(trifluoromethyl)isoxazol-5-yl]urea;
N-(4-ethoxy-2-nitrophenyl)-N′-[3-(trifluoromethyl)isoxazol-5-yl]urea;
N-(5-chloro-2,4-dimethoxyphenyl)-N′-(5-chloroisoxazol-3-yl)urea;
N-(5-chloro-4-methoxy-2-nitrophenyl)-N′-(5-methylisoxazol-3-yl)urea;
N-(5-fluoro-4-methoxy-2-nitrophenyl)-N′-(5-methylisoxazol-3-yl)urea;
N-[5-chloro-4-methoxy-2-(methylthio)phenyl]-N′-(5-methylisoxazol-3-yl)urea;
N-(5-chloro-2,4-dimethoxyphenyl)-N′-[5-(fluoromethyl)isoxazol-3-yl]urea;
N-(2,6-dimethoxypyridin-3-yl)-N′-[5-(fluoromethyl)isoxazol-3-yl]urea;
N-(5-fluoro-2,4-dimethoxyphenyl)-N′-[5-(fluoromethyl)isoxazol-3-yl]urea;
N-(5-bromo-2,4-dimethoxyphenyl)-N′-[5-(fluoromethyl)isoxazol-3-yl]urea;
N-(4-ethoxy-2-nitrophenyl)-N′-[5-(fluoromethyl)isoxazol-3-yl]urea;
N-(2,4-dimethoxy-5-methylphenyl)-N′-[5-(fluoromethyl)isoxazol-3-yl]urea;
N-(5-bromo-2,4-dimethoxyphenyl)-N′-[5-(hydroxymethyl)isoxazol-3-yl]urea;
N-(5-fluoro-2,4-dimethoxyphenyl)-N′-(5-isopropylisoxazol-3-yl)urea;
N-(5-chloro-2,4-dimethoxyphenyl)-N′-(5-isopropylisoxazol-3-yl)urea;
N-(5-bromo-2,4-dimethoxyphenyl)-N′-(5-isopropylisoxazol-3-yl)urea;
N-(2,6-dimethoxypyridin-3-yl)-N′-(5-isopropylisoxazol-3-yl)urea;
N-(2,4-dimethoxy-5-methylphenyl)-N′-(5-isopropylisoxazol-3-yl)urea;
N-(5-fluoro-2,4-dimethoxyphenyl)-N′-[5-(methoxymethyl)isoxazol-3-yl]urea;
N-(5-chloro-2,4-dimethoxyphenyl)-N′-[5-(methoxymethyl)isoxazol-3-yl]urea;
N-(5-bromo-2,4-dimethoxyphenyl)-N′-[5-(methoxymethyl)isoxazol-3-yl]urea;
N-(4-ethoxy-2-nitrophenyl)-N′-[5-(methoxymethyl)isoxazol-3-yl]urea;
N-(5-chloro-2,4-dimethoxyphenyl)-N′-(5-cyclopropylisoxazol-3-yl)urea;
N-(5-cyclopropylisoxazol-3-yl)-N′-(5-fluoro-2,4-dimethoxyphenyl)urea;
N-(5-bromo-2,4-dimethoxyphenyl)-N′-(5-cyclopropylisoxazol-3-yl)urea;
N-(5-cyclopropylisoxazol-3-yl)-N′-(2,4-dimethoxy-5-methylphenyl)urea;
N-(5-cyclopropylisoxazol-3-yl)-N′-(4-ethoxy-2-nitrophenyl)urea;
N-(5-cyclopropylisoxazol-3-yl)-N′-(2,6-dimethoxypyridin-3-yl)urea;
N-(5-chloro-2,4-dimethoxyphenyl)-N′-(5-ethylisoxazol-3-yl)urea;
N-(5-ethylisoxazol-3-yl)-N′-(5-fluoro-2,4-dimethoxyphenyl)urea;
N-(5-bromo-2,4-dimethoxyphenyl)-N′-(5-ethylisoxazol-3-yl)urea;
N-(2,4-dimethoxy-5-methylphenyl)-N′-(5-ethylisoxazol-3-yl)urea;
N-(2,6-dimethoxypyridin-3-yl)-N′-(5-ethylisoxazol-3-yl)urea;
N-(4-ethoxy-2-nitrophenyl)-N′-(5-ethylisoxazol-3-yl)urea;
N-(5-amino-2,4-dimethoxyphenyl)-N′-[5-(trifluoromethyl)-1,3,4-thiadiazol-2-yl]urea;
N-(5-azido-2,4-dimethoxyphenyl)-N′-[5-(trifluoromethyl)-1,3,4-thiadiazol-2-yl]urea;
N-(5-iodo-2,4-dimethoxyphenyl)-N′-[5-(trifluoromethyl)-1,3,4-thiadiazol-2-yl]urea;
N-(4-amino-2-methoxyphenyl)-N′-[5-(trifluoromethyl)-1,3,4-thiadiazol-2-yl]urea;
N-(4-methoxy-2-methylphenyl)-N′-[4-(pentafluoroethyl)-1,3-thiazol-2-yl]urea;
N-(5-chloro-2,4-dimethoxyphenyl)-N′-[5-(trifluoromethyl)isoxazol-3-yl]urea;
N-[2-methoxy-4-(tetrahydrofuran-3-yloxy)phenyl]-N′-[5-(trifluoromethyl)-1,3,4-thiadiazol-2-yl]urea;
N-[2-methoxy-4-(oxetan-3-yloxy)phenyl]-N′-[5-(trifluoromethyl)-1,3,4-thiadiazol-2-yl]urea;
N-[2-methoxy-4-(oxetan-3-yloxy)phenyl]-N′-[3-(trifluoromethyl)isoxazol-5-yl]urea;
N-[2-methoxy-4-(tetrahydrofuran-3-yloxy)phenyl]-N′-[3-(trifluoromethyl)isoxazol-5-yl]urea; or pharmaceutically acceptable salts thereof.

67. The method of claim 65, wherein the symptomatic relief would be to treat the mammal for pain, inflammation, cancer, or diabetes.

68. The method of claim 67, wherein pain or inflammation is caused by rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome; Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, HIV-3; cytomegalovirus; influenza; adenovirus; a herpes virus; or herpes zoster.

69. The method of claim 68, wherein the mammal receives symptomatic relief from co-administration of an antiviral or antibacterial agent for a therapeutically effective interval.

70. The method of claim 67, wherein cancer is multiple myeloma; acute and chronic myelogenous leukemia; or cancer-associated cachexia.

71. The method of claim 70, wherein the mammal receives symptomatic relief from co-administration of at least one of an anticancer agent or antiemetic agent for a therapeutically effective interval.

72. The method of claim 67, wherein diabetes is type I and type II diabetes.

73. The method of claim 72, wherein the mammal receives symptomatic relief from co-administration of at least one agent for the treatment of diabetes for a therapeutically effective interval.

74. The method of claim 67, wherein diabetes is associated with pancreatic beta cell destruction.

75. The method of claim 74, wherein the mammal receives symptomatic relief from co-administration of at least one agent for the treatment of diabetes for a therapeutically effective interval.

76. The method of claim 65, wherein the compound of the present invention and any other agent(s) are independently administered rectally, topically, orally, sublingually or parentarally.

77. The method of claim 76, wherein the compound of the present invention is administered in an amount of from about 0.001 to about 100 mg/kg of body weight of said mammal per day.

78. The method of claim 76, wherein the compound of the present invention is administered in an amount of from about 0.01 to about 50 mg/kg of body weight of said mammal per day.

79. A method for treating a disease or condition in a mammal in need thereof, wherein the mammal receives symptomatic relief from increasing vascular angiogensis comprising administrating a compound of claim 1.

80. The method of claim 79, wherein the compound is selected from
N-(4-hydroxyphenyl)-N′-(5-isopropyl-1,3-thiazol-2-yl)urea;
N-(4-ethoxyphenyl)-N′-(5-methyl-1,3-thiazol-2-yl)urea;
N-[4-(4-chlorophenyl)-1,3-thiazol-2-yl]-N′(4-iodophenyl)urea;
N-(2,6-dimethylphenyl)-N′-(6-fluoro-1,3-benzothiazol-2-yl)urea;
N-(4-chloro-1,3-benzothiazol-2-yl)-N′-(2-methoxyphenyl)urea;
N-(5-bromo-1,3-thiazol-2-yl)-N′-(2-ethoxyphenyl)urea;
N-(5-bromo-1,3-thiazol-2-yl)-N′-(2,4-dimethoxyphenyl)urea;
N-(5-chloro-1,3-thiazol-2-yl)-N′-(4-methoxy-2-methylphenyl)urea;
N-(5-bromo-1,3-thiazol-2-yl)-N′-(4-methoxy-2-methylphenyl)urea;
N-(5-chloro-2,4-dimethoxyphenyl)-N′-(1,3-thiazol-2-yl)urea;
N-(4-methoxy-2-methylphenyl)-N′-(4-methyl-1,3-thiazol-2-yl)urea;
N-(5-chloro-2,4-dimethoxyphenyl)-N′-(5-methyl-1,3-thiazol-2-yl)urea;
N-(4-chloro-1,3-benzothiazol-2-yl)-N′-(4-methoxy-2-methylphenyl)urea;
ethyl 2-({[(4-methoxy-2-methylphenyl)amino]carbonyl}amino)-4-phenyl-1,3-thiazole-5-carboxylate;
ethyl 2-[({[4-(butoxycarbonyl)phenyl]amino}carbonyl)amino]-4-phenyl-1,3-thiazole-5-carboxylate;
N-(5-bromo-1,3-thiazol-2-yl)-N′-(4-ethoxyphenyl)urea;
N-(4-methoxy-2-methylphenyl)-N′-[3-(trifluoromethyl)phenyl]urea;
N-(5-chloro-2,4-dimethoxyphenyl)-N′-isoxazol-3-ylurea;
N-(5-chloro-2,4-dimethoxyphenyl)-N′-(5-methyl-1,3,4-thiadiazol-2-yl)urea;
N-(5-chloro-2,4-dimethoxyphenyl)-N′-(5-methylisoxazol-3-yl)urea;
N-(5-chloro-2,4-dimethoxyphenyl)-N′-(5-ethyl-1,3,4-thiadiazol-2-yl)urea;
N-(5-chloro-2,4-dimethoxyphenyl)-N′-[5-(trifluoromethyl)-1,3,4-thiadiazol-2-yl]urea;
N-(5-chloro-2,4-dimethoxyphenyl)-N′-(5-cyclopropyl-1,3,4-thiadiazol-2-yl)urea;
N-(5-chloro-2,4-dimethoxyphenyl)-N′-[5-(difluoromethyl)-1,3,4-thiadiazol-2-yl]urea;
N-(5-chloro-2,4-dimethoxyphenyl)-N′-(5-methoxy-1,3,4-thiadiazol-2-yl)urea;
N-(5-bromo-1,3,4-thiadiazol-2-yl)-N′-(5-chloro-2,4-dimethoxyphenyl)urea;
N-(5-chloro-1,3,4-thiadiazol-2-yl)-N′-(5-chloro-2,4-dimethoxyphenyl)urea;
N-(5-chloro-2,4-dimethoxyphenyl)-N′-(1,3,4-thiadiazol-2-yl)urea;
N-(4-ethoxyphenyl)-N′-(5-methylisoxazol-3-yl)urea;
N-(2,4-dimethoxyphenyl)-N′-(5-methylisoxazol-3-yl)urea;
N-(2,4-dimethoxyphenyl)-N′-[5-(trifluoromethyl)-1,3,4-thiadiazol-2-yl]urea;
N-(5-chloro-2-methoxyphenyl)-N′-(5-methylisoxazol-3-yl)urea;
N-(5-chloro-2-methoxyphenyl)-N′-(5-methyl-1,3,4-thiadiazol-2-yl)urea;
N-(4-isopropoxyphenyl)-N′-[5-(trifluoromethyl)-1,3,4-thiadiazol-2-yl]urea;
N-(2-ethoxyphenyl)-N′-[5-(trifluoromethyl)-1,3,4-thiadiazol-2-yl]urea;
N-(4-ethoxyphenyl)-N′-[5-(trifluoromethyl)-1,3,4-thiadiazol-2-yl]urea;
N-(4-methoxyphenyl)-N′-[5-(trifluoromethyl)-1,3,4-thiadiazol-2-yl]urea;
N-(4-butoxyphenyl)-N′-[5-(trifluoromethyl)-1,3,4-thiadiazol-2-yl]urea;
N-(2,3-dihydro-1-benzofuran-5-yl)-N′-[5-(trifluoromethyl)-1,3,4-thiadiazol-2-yl]urea;
N-(4-ethyl-1,3-thiazol-2-yl)-N′-(4-methoxy-2-methylphenyl)urea;
N-(5-chloro-2,4-dimethoxyphenyl)-N′-(1H-imidazol-2-yl)urea;
N-(5-bromo-1,3-thiazol-2-yl)-N′-(5-chloro-2,4-dimethoxyphenyl)urea;
N-(5-chloro-2,4-dimethoxyphenyl)-N′-(5-methyl-1,3-thiazol-2-yl)urea;
N-(5-chloro-2,4-dimethoxyphenyl)-N′-(3-methylisoxazol-5-yl)urea;
N-(5-chloro-2,4-dimethoxyphenyl)-N′-(6-cyanopyridin-3-yl)urea;
N-(5-chloro-2,4-dimethoxyphenyl)-N′-(5-chloro-1,3-thiazol-2-yl)urea;
N-(5-chloro-1,3-thiazol-2-yl)-N′-(2,4-dimethoxy-5-methylphenyl)urea;
N-(5-bromo-2,4-dimethoxyphenyl)-N′-(5-chloro-1,3-thiazol-2-yl)urea;
N-(5-bromo-1,3-thiazol-2-yl)-N′-(5-fluoro-2,4-dimethoxyphenyl)urea;
N-(5-chloro-1,3-thiazol-2-yl)-N′-(5-fluoro-2,4-dimethoxyphenyl)urea;
N-(3-chloro-4-fluorophenyl)-N′-(5-methylisoxazol-3-yl)urea;
N-(3-chloro-4-fluorophenyl)-N′-[5-(trifluoromethyl)-1,3,4-thiadiazol-2-yl]urea;
N-(2-ethoxyphenyl)-N′-(5-methylisoxazol-3-yl)urea;
N-(2-methoxyphenyl)-N′-[5-(trifluoromethyl)-1,3,4-thiadiazol-2-yl]urea;
N-(2-fluoro-4-methoxyphenyl)-N′-[5-(trifluoromethyl)-1,3,4-thiadiazol-2-yl]urea;
N-(5-chloro-2,4-dimethoxyphenyl)-N′-(5-mercapto-1,3,4-thiadiazol-2-yl)urea;
N-(4,5-dimethoxy-2-methylphenyl)-N′-[5-(trifluoromethyl)-1,3,4-thiadiazol-2-yl]urea;
N-(4-hydroxy-2-nitrophenyl)-N′-[5-(trifluoromethyl)-1,3,4-thiadiazol-2-yl]urea;
N-(2,6-dimethoxypyridin-3-yl)-N′-(5-methyl-1,3-thiazol-2-yl)urea;
N-(4-ethoxy-2-nitrophenyl)-N′-(5-methylisoxazol-3-yl)urea;
N-(4-methoxy-2-methylphenyl)-N′-[2-(trifluoromethyl) pyridin-4-yl]urea;
N-(5-fluoro-2,4-dimethoxyphenyl)-N′-(5-methylisoxazol-3-yl)urea;
N-(5-fluoro-2,4-dimethoxyphenyl)-N′-(5-methyl-1,3-thiazol-2-yl)urea;
N-(5-fluoro-2,4-dimethoxyphenyl)-N′-(5-methyl-1,3,4-thiadiazol-2-yl)urea;
N-(5-fluoro-2,4-dimethoxyphenyl)-N′-[5-(trifluoromethyl)-1,3,4-thiadiazol-2-yl]urea;
N-(5-bromo-2,4-dimethoxyphenyl)-N′-(5-methylisoxazol-3-yl)urea;
N-(5-bromo-2,4-dimethoxyphenyl)-N′-(5-methyl-1,3-thiazol-2-yl)urea;
N-(5-bromo-2,4-dimethoxyphenyl)-N′-(5-methyl-1,3,4-thiadiazol-2-yl)urea;
N-(5-bromo-2,4-dimethoxyphenyl)-N′-[5-(trifluoromethyl)-1,3,4-thiadiazol-2-yl]urea;
N-(3,5-difluoro-2,4-dimethoxyphenyl)-N′-(5-methylisoxazol-3-yl)urea;
N-(3,5-difluoro-2,4-dimethoxyphenyl)-N′-[5-(trifluoromethyl)-1,3,4-thiadiazol-2-yl]urea;
N-[2,4-dimethoxy-5-(trifluoromethyl)phenyl]-N′-[5-(trifluoromethyl)-1,3,4-thiadiazol-2-yl]urea;
N-(5-chloro-2,4-diethoxyphenyl)-N′-(5-methylisoxazol-3-yl)urea;
N-(5-chloro-2,4-diethoxyphenyl)-N′-(5-methyl-1,3-thiazol-2-yl)urea;
N-(5-chloro-2,4-diethoxyphenyl)-N′-(5-methyl-1,3,4-thiadiazol-2-yl)urea;
N-(5-chloro-2,4-diethoxyphenyl)-N′-[5-(trifluoromethyl)-1,3,4-thiadiazol-2-yl]urea;
N-(5-chloro-2,4-dipropoxyphenyl)-N′-[5-(trifluoromethyl)-1,3,4-thiadiazol-2-yl]urea;
N-(2-chloro-4-methoxy-5-methylphenyl)-N′-(5-methylisoxazol-3-yl)urea;
N-(2-chloro-4-methoxy-5-methylphenyl)-N′-[5-(trifluoromethyl)-1,3,4-thiadiazol-2-yl]urea;
N-(2,4-dimethoxy-5-methylphenyl)-N′-(5-methylisoxazol-3-yl)urea;
N-(2,4-dimethoxy-5-methylphenyl)-N′-(5-methyl-1,3-thiazol-2-yl)urea;
N-(2,4-dimethoxy-5-methylphenyl)-N′-(5-methyl-1,3,4-thiadiazol-2-yl)urea;
N-(2,4-dimethoxy-5-methylphenyl)-N′-[5-(trifluoromethyl)-1,3,4-thiadiazol-2-yl]urea;
N-(4-ethoxy-2-methoxy-5-methylphenyl)-N′-[5-(trifluoromethyl)-1,3,4-thiadiazol-2-yl]urea;
N-(5-acetyl-2,4-dimethoxyphenyl)-N′-[5-(trifluoromethyl)-1,3,4-thiadiazol-2-yl]urea;
N-(2,4-dimethoxy-5-nitrophenyl)-N′-[5-(trifluoromethyl)-1,3,4-thiadiazol-2-yl]urea;
N-[2-methoxy-5-methyl-4-(2,2,2-trifluoroethoxy)phenyl]-N′-[5-(trifluoromethyl)-1,3,4-thiadiazol-2-yl]urea;
N-(5-chloro-2,4-dimethoxyphenyl)-N′-(2-methyl-1,3-thiazol-5-yl)urea;
N-(2-methoxy-4-nitrophenyl)-N′-[5-(trifluoromethyl)-1,3,4-thiadiazol-2-yl]urea;
N-[5-(trifluoromethyl)-1,3,4-thiadiazol-2-yl]-N′-(2,4,5-trimethoxyphenyl)urea;
N-[4-methoxy-2-(methylthio)phenyl]-N′-[5-(trifluoromethyl)-1,3,4-thiadiazol-2-yl]urea;
N-(4-{[(1R)-1-methylpropyl]oxy}phenyl)-N′-[5-(trifluoromethyl)-1,3,4-thiadiazol-2-yl]urea;
N-[4-(allyloxy)phenyl]-N′-[5-(trifluoromethyl)-1,3,4-thiadiazol-2-yl]urea;
N-(4-propoxyphenyl)-N′-[5-(trifluoromethyl)-1,3,4-thiadiazol-2-yl]urea;
N-(2-ethoxypyridin-3-yl)-N′-[5-(trifluoromethyl)-1,3,4-thiadiazol-2-yl]urea;
N-(4-methoxy-2-methylphenyl)-N′-[4-(trifluoromethyl)-1,3-thiazol-2-yl]urea;
N-(4-methoxy-2-methylphenyl)-N′-(3-phenyl-1,2,4-thiadiazol-5-yl)urea;
N-(5-ethyl-4-phenyl-1,3-thiazol-2-yl)-N′-(4-methoxy-2-methylphenyl)urea;
N-(4-hydroxy-2-methylphenyl)-N′-[3-(trifluoromethyl)phenyl]urea;
N-(4-hydroxyphenyl)-N′-[3-(trifluoromethyl)phenyl]urea;
N-[2-methyl-4-(methylthio)phenyl]-N′-[3-(trifluoromethyl)phenyl]urea;
N-(2-ethyl-4-hydroxyphenyl)-N′-[3-(trifluoromethyl)phenyl]urea;
N-(4-amino-2-methylphenyl)-N′-[3-(trifluoromethyl)phenyl]urea;
N-(4-methoxyphenyl)-N′-[3-(trifluoromethyl)phenyl]urea;
N-(5-hydroxy-2-methylphenyl)-N′-[3-(trifluoromethyl)phenyl]urea;
N-(5-chloro-2,4-dimethoxyphenyl)-N′-[4-(trifluoromethyl)-1H-pyrazol-1-yl]urea;
N-(4-bromo-1H-pyrazol-1-yl)-N′-(5-chloro-2,4-dimethoxyphenyl)urea;
N-(4-methoxy-2-methylphenyl)-N′-[3-(trifluoromethyl)phenyl]thiourea;
N-(4-hydroxy-2-methylphenyl)-N′-[3-(trifluoromethyl)phenyl]thiourea;
N-(2,4-dimethoxy-5-methylphenyl)-N′-[3-(trifluoromethyl)isoxazol-5-yl]urea;
N-(4-ethoxyphenyl)-N′-[3-(trifluoromethyl)isoxazol-5-yl]urea;
N-(2-ethoxyphenyl)-N′-[3-(trifluoromethyl)isoxazol-5-yl]urea;
N-(2,4-dimethoxyphenyl)-N′-[3-(trifluoromethyl)isoxazol-5-yl]urea;
N-(2,6-dimethoxypyridin-3-yl)-N′-(3-methylisoxazol-5-yl)urea;
N-(5-fluoro-2,4-dimethoxyphenyl)-N′-(3-methylisoxazol-5-yl)urea;
N-(2,4-dimethoxyphenyl)-N′-(3-methylisoxazol-5-yl)urea;
N-(5-chloro-2,4-dimethoxyphenyl)-N′-[3-(trifluoromethyl)isoxazol-5-yl]thiourea;
N-(5-chloro-2,4-diethoxyphenyl)-N′-[3-(trifluoromethyl)isoxazol-5-yl]urea;
N-(4-methoxy-2-nitrophenyl)-N′-[3-(trifluoromethyl)isoxazol-5-yl]urea;
N-(4-methoxy-2-methylphenyl)-N′-[4-(trifluoromethyl)-1,3-thiazol-2-yl]thiourea;
N-(5-chloro-2,4-dimethoxyphenyl)-N′-[5-(trifluoromethyl)-1,3,4-thiadiazol-2-yl]thiourea;
N-(5-fluoro-2,4-dimethoxyphenyl)-N′-[5-(trifluoromethyl)-1,3,4-thiadiazol-2-yl]thiourea;
N-[2-methoxy-4-(2-methoxyethoxy)phenyl]-N′-[5-(trifluoromethyl)-1,3,4-thiadiazol-2-yl]urea;
N-(4-ethoxy-2-nitrophenyl)-N′-[3-(trifluoromethyl)isoxazol-5-yl]thiourea;
N-(4-hydroxy-2-methylphenyl)-N′-[4-(trifluoromethyl)-1,3-thiazol-2-yl]thiourea;
N-(3-chloro-4-methoxyphenyl)-N′-[5-(trifluoromethyl)-1,3,4-thiadiazol-2-yl]urea;
N-(5-chloro-2-methoxyphenyl)-N′-[5-(trifluoromethyl)-1,3,4-thiadiazol-2-yl]urea;
N-(4-methoxy-2-nitrophenyl)-N′-[5-(trifluoromethyl)-1,3,4-thiadiazol-2-yl]urea;
N-(4-ethoxy-2-nitrophenyl)-N′-[5-(trifluoromethyl)-1,3,4-thiadiazol-2-yl]urea;
N-(4-methoxy-2-methylphenyl)-N′-[5-(trifluoromethyl)-1,3,4-thiadiazol-2-yl]urea;
N-(2,6-dimethoxypyridin-3-yl)-N′-[5-(trifluoromethyl)-1,3,4-thiadiazol-2-yl]urea;
N-(4-hydroxyphenyl)-N′-[5-(trifluoromethyl)-1,3,4-thiadiazol-2-yl]urea;
N-(5-ethoxypyridin-2-yl)-N′-[5-(trifluoromethyl)-1,3,4-thiadiazol-2-yl]urea;
N-(4-ethoxy-2-morpholin-4-ylphenyl)-N′-[5-(trifluoromethyl)-1,3,4-thiadiazol-2-yl]urea;
tert-butyl 4-{5-ethoxy-2-[({[5-(trifluoromethyl)-1,3,4-thiadiazol-2-yl]amino}carbonyl) amino]phenyl}piperazine-1-carboxylate;
N-(2-chloro-6-methoxy-pyridin-3-yl)-N′-[5-(trifluoromethyl)-1,3,4-thiadiazol-2-yl]urea;
N-[6-methoxy-2-(methylthio)pyridin-3-yl]-N′-[5-(trifluoromethyl)-1,3,4-thiadiazol-2-yl]urea;
N-[6-methoxy-2-(methylsulfonyl)pyridin-3-yl]-N′-[5-(trifluoromethyl)-1,3,4-thiadiazol-2-yl]urea;
N-[2-methoxy-4-(2-methoxyethoxy)phenyl]-N′-(5-methylisoxazol-3-yl)urea;
N-(4-hydroxy-2-methylphenyl)-N′-[4-(trifluoromethyl)-1,3-thiazol-2-yl]urea;
N-(4-hydroxyphenyl)-N′-[4-(trifluoromethyl)-1,3-thiazol-2-yl]urea;
N-(5-hydroxy-2-methylphenyl)-N′-[4-(trifluoromethyl)-1,3-thiazol-2-yl]urea;
N-(3-hydroxy-2-methylphenyl)-N′-[4-(trifluoromethyl)-1,3-thiazol-2-yl]urea;
N-(6-cyanopyridin-3-yl)-N′-(5-fluoro-2,4-dimethoxyphenyl)urea;
N-(4-hydroxy-2-methylphenyl)-N′-[3-(trifluoromethoxy)phenyl]urea;
N-(4-hydroxy-2-methylphenyl)-N′-[4-(pentafluoroethyl)-1,3-thiazol-2-yl]urea;
N-(5-bromo-2,4-dimethoxyphenyl)-N′-(6-cyanopyridin-3-yl)urea;
N-(5-chloro-2,4-dimethoxyphenyl)-N′-[3-(trifluoromethyl)isoxazol-5-yl]urea;
N-(5-fluoro-2,4-dimethoxyphenyl)-N′-[3-(trifluoromethyl)isoxazol-5-yl]urea;
N-(2,6-dimethoxypyridin-3-yl)-N′-[3-(trifluoromethyl)isoxazol-5-yl]urea;
N-(4-ethoxy-2-nitrophenyl)-N′-[3-(trifluoromethyl)isoxazol-5-yl]urea;
N-(5-chloro-2,4-dimethoxyphenyl)-N′-(5-chloroisoxazol-3-yl)urea;
N-(5-chloro-4-methoxy-2-nitrophenyl)-N′-(5-methylisoxazol-3-yl)urea;
N-(5-fluoro-4-methoxy-2-nitrophenyl)-N′-(5-methylisoxazol-3-yl)urea;
N-[5-chloro-4-methoxy-2-(methylthio)phenyl]-N′-(5-methylisoxazol-3-yl)urea;
N-(5-chloro-2,4-dimethoxyphenyl)-N′-[5-(fluoromethyl)isoxazol-3-yl]urea;
N-(2,6-dimethoxypyridin-3-yl)-N′-[5-(fluoromethyl)isoxazol-3-yl]urea;
N-(5-fluoro-2,4-dimethoxyphenyl)-N′-[5-(fluoromethyl)isoxazol-3-yl]urea;
N-(5-bromo-2,4-dimethoxyphenyl)-N′-[5-(fluoromethyl)isoxazol-3-yl]urea;
N-(4-ethoxy-2-nitrophenyl)-N′-[5-(fluoromethyl)isoxazol-3-yl]urea;
N-(2,4-dimethoxy-5-methylphenyl)-N′-[5-(fluoromethyl)isoxazol-3-yl]urea;
N-(5-bromo-2,4-dimethoxyphenyl)-N′-[5-(hydroxymethyl)isoxazol-3-yl]urea;
N-(5-fluoro-2,4-dimethoxyphenyl)-N′-(5-isopropylisoxazol-3-yl)urea;
N-(5-chloro-2,4-dimethoxyphenyl)-N′-(5-isopropylisoxazol-3-yl)urea;
N-(5-bromo-2,4-dimethoxyphenyl)-N′-(5-isopropylisoxazol-3-yl)urea;
N-(2,6-dimethoxypyridin-3-yl)-N′-(5-isopropylisoxazol-3-yl)urea;
N-(2,4-dimethoxy-5-methylphenyl)-N′-(5-isopropylisoxazol-3-yl)urea;
N-(5-fluoro-2,4-dimethoxyphenyl)-N′-[5-(methoxymethyl)isoxazol-3-yl]urea;
N-(5-chloro-2,4-dimethoxyphenyl)-N′-[5-(methoxymethyl)isoxazol-3-yl]urea;
N-(5-bromo-2,4-dimethoxyphenyl)-N′-[5-(methoxymethyl)isoxazol-3-yl]urea;
N-(4-ethoxy-2-nitrophenyl)-N′-[5-(methoxymethyl)isoxazol-3-yl]urea;
N-(5-chloro-2,4-dimethoxyphenyl)-N′-(5-cyclopropylisoxazol-3-yl)urea;
N-(5-cyclopropylisoxazol-3-yl)-N′-(5-fluoro-2,4-dimethoxyphenyl)urea;
N-(5-bromo-2,4-dimethoxyphenyl)-N′-(5-cyclopropylisoxazol-3-yl)urea;
N-(5-cyclopropylisoxazol-3-yl)-N′-(2,4-dimethoxy-5-methylphenyl)urea;
N-(5-cyclopropylisoxazol-3-yl)-N′-(4-ethoxy-2-nitrophenyl)urea;
N-(5-cyclopropylisoxazol-3-yl)-N′-(2,6-dimethoxypyridin-3-yl)urea;
N-(5-chloro-2,4-dimethoxyphenyl)-N′-(5-ethylisoxazol-3-yl)urea;
N-(5-ethylisoxazol-3-yl)-N′-(5-fluoro-2,4-dimethoxyphenyl)urea;
N-(5-bromo-2,4-dimethoxyphenyl)-N′-(5-ethylisoxazol-3-yl)urea;
N-(2,4-dimethoxy-5-methylphenyl)-N′-(5-ethylisoxazol-3-yl)urea;
N-(2,6-dimethoxypyridin-3-yl)-N′-(5-ethylisoxazol-3-yl)urea;
N-(4-ethoxy-2-nitrophenyl)-N′-(5-ethylisoxazol-3-yl)urea;
N-(5-amino-2,4-dimethoxyphenyl)-N′-[5-(trifluoromethyl)-1,3,4-thiadiazol-2-yl]urea;
N-(5-azido-2,4-dimethoxyphenyl)-N′-[5-(trifluoromethyl)-1,3,4-thiadiazol-2-yl]urea;
N-(5-iodo-2,4-dimethoxyphenyl)-N′-[5-(trifluoromethyl)-1,3,4-thiadiazol-2-yl]urea;
N-(4-amino-2-methoxyphenyl)-N′-[5-(trifluoromethyl)-1,3,4-thiadiazol-2-yl]urea;
N-(4-methoxy-2-methylphenyl)-N′-[4-(pentafluoroethyl)-1,3-thiazol-2-yl]urea;
N-(5-chloro-2,4-dimethoxyphenyl)-N′-[5-(trifluoromethyl)isoxazol-3-yl]urea;
N-[2-methoxy-4-(tetrahydrofuran-3-yloxy)phenyl]-N′-[5-(trifluoromethyl)-1,3,4-thiadiazol-2-yl]urea;
N-[2-methoxy-4-(oxetan-3-yloxy)phenyl]-N′-[5-(trifluoromethyl)-1,3,4-thiadiazol-2-yl]urea;
N-[2-methoxy-4-(oxetan-3-yloxy)phenyl]-N′-[3-(trifluoromethyl)isoxazol-5-yl]urea;
N-[2-methoxy-4-(tetrahydrofuran-3-yloxy)phenyl]-N′-[3-(trifluoromethyl)isoxazol-5-yl]urea; or pharmaceutically acceptable salts thereof.

81. The method of claim 79, wherein the disease or condition is wound healing, healing bone fracture, ischemic heart disease, or stable angina pectoris.

82. The method of claim 81, wherein the wound is from surgery or burn.

83. A method for diagnosing disease in a mammal, comprising administering to the mammal a detectably labeled compound of claim 42 and detecting the binding of that compound to an alpha 7 nAChR.

84. The method of claim 83, wherein the compound is detected using position emission topography.

85. The method of claim 83, wherein the compound is detected using single-photon emission computed tomography.

86. The method of claim 83, wherein the disease is Alzheimer's disease, neurodegeneration associated with diseases such as Alzheimer's disease, pre-senile dementia (mild cognitive impairment), senile dementia, Parkinson's disease, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, diabetic retinopathy, or symptoms associated with pain.
